(19) United States
(12) Patent Application Publication
(10) Pub. No.: US 2010/0173871 A1
(43) Pub. Date: Jul. 8, 2010

(54) PHOSPHORYLATED DENDRIMERS AS ANTIINFLAMMATORY DRUGS

(75) Inventors: Mary Poupot, Aigrefeuille (FR); Remy Poupot, Aigrefeuille (FR); Jean-Jacques Fournie, Corronsac (FR); Damien Portevin, London (GB); Severine Fruchon, Toulouse (FR); Jean-Luc Davignon, Beauzelle (FR); Cedric-Olivier Davignon, Toulouse (FR); Anne-Marie Caminade, Toulouse (FR); Jean-Pierre Majoral, Ramonville-Saint-Agne (FR); Oliver Rolland, Toulouse (FR)

Correspondence Address: YOUNG & THOMPSON, 209 Madison Street, Suite 500, Alexandria, VA, 22314, US

(73) Assignee: CENTRE NATIONAL DE LA RECHERCHE SCIENTIFIQUE, Paris Cedex 16 (FR)

(21) Appl. No.: 12/525,416

(22) PCT Filed: Aug. 1, 2008
(86) PCT No.: PCT/IB08/02547

§ 371 Date: Nov. 17, 2009

(51) Int. Cl. A61K 31/66; A61K 31/695; A61K 31/675; A61K 31/6615

(52) U.S. Cl. 514/89; 514/63; 514/93; 514/102; 514/105; 514/107

(57) ABSTRACT

A method for treating inflammatory diseases through an anti-inflammatory type activation of monocytes, the method includes the administration to a patient in need thereof of an effective quantity of at least one dendrimer with mono- or biphosphonic terminations.

The present invention relates to the use of phosphorylated dendrimers for the treatment of uncontrolled inflammatory processes.

Dendrimers are macromolecules constituted by monomers which combine according to a tree-like process around a plurifunctional central core.

Dendrimers, also called “cascade molecules”, are highly branched functional polymers with a defined structure. These macromolecules are effectively polymers since they are based on the combination of repeating units. However, dendrimers differ fundamentally from standard polymers to the extent that they have specific properties due to their tree-like construction. The molecular weight and the form of the dendrimers can be precisely controlled (Fréchet, J. M. J. et al. (2001) Dendrimers and other dendritic polymers, John Wiley and Sons, New York, N.Y.; Newkome:, G. R. et al. (2001) Dendrimers and dendrons: concepts, syntheses, applications 2nd ed Ed., Wiley-VCH, Weinheim).

The dendrimers are constructed stepwise, by the repetition of a sequence of reactions allowing the multiplication of each repeating unit and terminal functions. Each sequence of reactions forms what is called a “new generation”. The tree-like construction is carried out by the repetition of a sequence of reactions which makes it possible to obtain a new generation and a growing number of identical branches at the end of each reaction cycle. After a few generations, the dendrimer generally assumes a highly branched and plurifunctionalized globular form due to the numerous terminal functions present at the periphery. These structural features, together with the high density of chemical reactive functions on the outer shell of these molecules enhanced their use in biology and medicine (Cloninger, M. J. (2002) Curr Opin Chem Biol 6(6), 742-748). Dendrimers can be used as drug carriers that are able to enhance aqueous solubility, improve drug transit across biological barriers and target the drug delivery to injured tissues (Lee, C. C. et al. (2005) Nat Biotechnol 23(12), 1517-1526; Cheng, Y. et al. (2008) Front Biosci 13, 1447-1471). Dendrimers also act as drugs themselves. Among others, dendrimers glucosamine conjugates were safely used to prevent scar tissue formation via immuno-modulatory and anti-angiogenic effects (Shaunak, S. et al. (2004) Nat Biotechnol 22(8), 977-984). Multiple antigenic peptide dendrimers have proved to be promising compounds for immune response modification or immuno-diagnosis (Crespo, L. et al. (2005) Chem Rev 105(5), 1663-1681; Singh, P. (2007) Biotechnol Appl Biochem 48(Pt 1), 1-9). Polyanionic (with sulfonate groups) dendrimers were shown to provide an efficient microbicide activity against different viruses (McCarthy, T. D. et al. (2005) Mol Pharm 2(4), 312-318). Cationic phosphorus-containing dendrimers reduce the replication of the abnormal scrapie isoform of the prion protein in mice (Solassol, J. et al. (2004) J Gen Virol 85(Pt 6), 1791-1799). Recently the inventors reported the effects of synthetic molecules belonging to the dendrimer family on the innate immune system (Poupot, M. et al. (2006) Faseb J 20(13), 2339-2351; Griffe, L. et al. (2007) Angew Chem Int Ed Engl 46(14), 2523-2526; Rolland, O. et al. (2008) Chem. Eur. J., 14(16), 4836-4850).

Mononuclear phagocytes, including monocytes and macrophages (MΦ, the tissue-counterpart of the former), are essential in innate immunity as a first line of defence against bacterial and parasitic infections. They also ensure the engaging of the delayed adaptive immune response. Monocytes/MΦ are a heterogeneous population encompassing a large spectrum of phenotypes from pro-inflammatory to anti-inflammatory responses, depending on the stimulus they receive. Indeed, MΦ activation can take several aspects. Besides the classical activation pathway, a so-called alternative activation mechanism emerged. The classical activation pathway of MΦ is mediated by interferon (IFN)-γ as primer and then triggering by tumor necrosis factor (TNF) or bacterial lipopolysaccharides (LPS). These MΦ produce mediators making them effector cells in type Th-1 cellular immune responses and are cytotoxic effectors against intracellular pathogens. The “alternative activation” of MΦ describes closely related responses induced either by interleukin (IL)-4 or IL-13 or by IL-10 or glucocorticoids. These MΦ appear to be involved in immuno-suppression and tissue repair. This dual classification with pro-inflammatory classically-activated MΦ and anti-inflammatory alternatively-activated MΦ has been recently broadened with the proposition of type 2-activated MΦ. Although they are activated, after IFN-γ priming or not, by ligation of Fc_{γ}R and Toll-like receptors (TLR) triggering, type 2 MΦ rather display anti-inflammatory responses. The closely interlinked pathways of MΦ activation and the intricate responses displayed by these cells explain the still confusing classification of polarized mononuclear phagocytes. During infection, MΦ are critical mediators of inflammatory processes aimed at the removal of pathogens. However, inflammation is also associated with deleterious effects for the tissues and must be repressed to allow complete healing. Due to their dual pattern of activation, MΦ play a pivotal role both in triggering and resolving inflammation.

The inventors surprisingly discovered that some phosphorylated dendrimers lead to an anti-inflammatory type activation of the monocytes.

Thus one of the purposes of the present invention is to propose a novel anti-inflammatory treatment using synthetic and essentially non-toxic compounds with low production costs.

Thus, the present invention relates to dendrimers with monophosphonic or bisphosphonic terminations for the treatment of inflammatory diseases, in particular auto-immune diseases.

Said treatment is mediated by an anti-inflammatory type activation of the monocytes.

Some dendrimers used according to the present invention and their preparation are disclosed in the international application WO 2006/024769.

According to a particular embodiment of the invention, the dendrimers with monophosphonic or bisphosphonic terminations are of generation n and comprise a central core § with a valency of m which can establish m−2 bonds, providing that m is greater than 2, or m−1 bonds, providing that m is greater than 1, or m bonds with linkage chains, preferably identical to each other, m representing an integer from 1 to 20, in particular of 1 to 10 and more particularly of 1 to 8 and n representing an integer from 0 to 12, which linkage chains are constituted by:
generation chains attached in a tree-like structure around the core on each of the bonds, when n is greater than or equal to 1, a generation chain of a given generation being linked to
   a generation chain of the generation immediately below the given generation, or to the core when the given generation is 1, and to
   at least 2 generation chains of the generation immediately above the given generation, or optionally to at least one intermediate chain when the given generation is n,
a terminal group being attached to the end of each generation chain of generation n, or if appropriate to the end of each intermediate chain, or
intermediate chains attached around the core on each of the bonds, when n is 0, a terminal group being attached to the end of each intermediate chain;

said terminal group being represented by the formula:

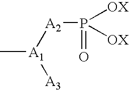

where
A₁ represents N; a P═Y group, where Y represents O, S, or no atom; an N—R group; or a C—R group; R representing H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a —NR′R″ group, an alkoxy group of 1 to 16 carbon atoms, an alkyl group of 1 to 16 carbon atoms, a halogen atom, a —NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol, R′ and R″ representing independently of each other H or an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
A₂ represents a single bond or a linear, branched or cyclic hydrocarbon chain with 1 to 6 members, each of said members optionally containing one or more heteroatoms, said heteroatoms being preferably chosen from a sulphur, oxygen, phosphorus or nitrogen atom, more preferably nitrogen, and being optionally substituted by at least one substituent chosen from H, an alkyl group of 1 to 16 carbon atoms, a halogen, a NO₂ group, a NRR′ group, a —CN group, a —CF₃ group, a hydroxyl group, an alkoxy group of 1 to 16 carbon atoms, an aryl or heteroaryl group of 1 to 24 carbon atoms, the heteroelement being preferably chosen from oxygen, nitrogen or sulphur, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R and R′ representing independently of each other H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
A₃ represents H, or a linear, branched or cyclic hydrocarbon chain with 1 to 6 members, each of said members being optionally chosen from a heteroatom, said heteroatom being preferably chosen from sulphur, nitrogen, phosphorus or silicon, more preferably nitrogen, each member being able to be optionally substituted by at least one group chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹ or

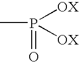

in particular A₃ can represent

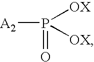

each A₂ being identical or different;
each OX, identical or different for each phosphonic group, represents OH, Oalkyl, where the alkyl group comprises from 1 to 16 carbon atoms, Oaryl, where the aryl group comprises from 6 to 24 carbon atoms, Oaralkyl, where the aralkyl group comprises from 7 to 24 carbon atoms, Oalkylaryl, where the alkylaryl group comprises from 7 to 24 carbon atoms, OSiR′₁R′₂R′₃, where R′₁, R′₂ and R′₃, identical or different, represent an alkyl group of 1 to 16 carbon atoms, or O⁻M⁺, where M⁺ is a cation of elements of group IA, IB, HA, IIB or IIIA, IIIB of the periodic table of the elements, preferably M⁺ is chosen from the cations of the sodium, potassium, copper, calcium, barium, zinc, magnesium, lithium and aluminium atoms, or hydrocarbon groups of 1 to 100 carbon atoms, or nitrogenous groups of 0 to 100 carbon atoms, such as NR₁R₂R₃R₄⁺, where, independently of each other R₁, R₂, R₃ and R₄ represent H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other II or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹,
m represents an integer from 1 to 20, in particular from 1 to 10 and more particularly from 1 to 8;
n represents an integer from 0 to 12,

said central core § representing a group comprising from 1 to 500 atoms, and optionally containing one or more heteroatoms, said heteroatoms being preferably chosen from oxygen, sulphur, nitrogen, phosphorus or silicon.

According to another particular embodiment of the invention, the core § establishes m bonds with m identical linkage chains constituted:
either by generation chains attached in a tree-like structure around the core on each of the bonds, the end of each chain generation furthest from the central core being attached either to a terminal group or to an intermediate chain, the end of each intermediate chain being attached to a terminal group,
or by intermediate chains attached around the core on each of the bonds, the end of each intermediate chain being attached to a terminal group.

According to this embodiment, for a given dendrimer, all of the generation chains of the generation furthest from the central core are attached to an identical substituent which can be either a terminal group or an intermediate chain.

According to an alternative embodiment of the invention, the core establishes m−2 or m−1 bonds, m representing an integer from 3 to 20, in particular from 3 to 10 and more particularly from 3 to 8, with respectively m−2 or m−1 identical linkage chains constituted:
either by generation chains attached in a tree-like structure around the core on each of the bonds, the end of each chain generation furthest from the central core being attached either to a terminal group or to an intermediate chain, and the end of each intermediate chain being attached to a terminal group,
or by intermediate chains attached around the core on each of the bonds, the end of each intermediate chain being attached to a terminal group;
   the 1 or 2 remaining bonds being attached to linkage groups, identical or different, optionally linked together, in particular by means of a covalent bond, constituted:
either by part of the linkage chains defined above,
or by a hydrogen atom,
or by hydrocarbon groups comprising from 1 to 500 carbon atoms, said hydrocarbon groups being in particular constituted by H or by a linear, branched or cyclic hydrocarbon chain with 1 to 200 members, optionally containing one or more double or triple bonds, each of said members being optionally chosen from a heteroatom, said heteroatom being preferably chosen from a nitrogen, oxygen, phosphorus, silicon or sulphur atom, an aryl group of 6 to 24 carbon atoms, a heteroaryl group of 1 to 24 carbon atoms, a carboxyl group, a >C═NR group, each member being able to be optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, the first member of said hydrocarbon chain preferably being oxygen or nitrogen.

In particular the hydrocarbon groups comprising from 1 to 500 carbon atoms defined above can be fluorophores, or any functional chemical group.

According to another particular embodiment of the invention, the generation chains are chosen from any linear, branched or cyclic hydrocarbon chain with 1 to 12 members, optionally containing one or more double or triple bonds, each of said members being optionally chosen from a heteroatom, said heteroatom being preferably chosen from nitrogen, oxygen, sulphur, phosphorus or silicon, an aryl group of 6 to 24 carbon atoms, a heteroaryl group of 1 to 24 carbon atoms, the heteroelement being preferably chosen from oxygen, nitrogen or sulphur, a carboxyl group, a >C═NR group, each member being able to be optionally substituted by at least one substituent chosen from an alkyl group of 1 to 16 carbon atoms, a halogen atom, an —NO₂ group, an —NRR′ group, a —CN group, a —CF₃ group, a hydroxyl group, an alkoxy group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹.

According to yet another particular embodiment of the invention, the intermediate chains are chosen from the groups corresponding to the formula:

-J-K-L-

where
J represents an oxygen or sulphur atom, or a NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹,
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol¹ to 3000 g.mol¹;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;

According to a more particular embodiment of the invention, the core is chosen from:
a nitrogen or silicon atom;
a group of formula

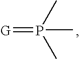

in which G represents an oxygen, nitrogen, sulphur, selenium, tellurium atom or an ═NR group, R representing H or an alkyl group of 1 to 16 carbon atoms, an aryl group of 6 to 24 carbon atoms, an aralkyl group of 7 to 16 carbon atoms, such as the thiophosphoryl group of formula

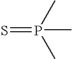

or an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹
a bis-phenyloxy group of formula

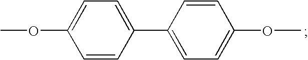

a 1,2-diamino-ethane group of formula

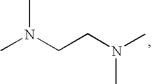

a 1,4-diamino-butane group of formula

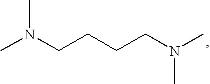

a cyclotriphosphazene group of formula

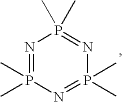

also denoted N₃P₃ or P₃N₃,
a cyclotetraphosphazene group of formula

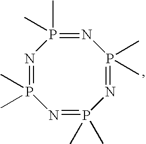

also denoted N₄P₄ or P₄N₄.

The present invention relates in particular to the use as defined above, of dendrimers of structure PAMAM, DAB or PMMH.

The dendrimers of structure PAMAM are in particular described by Tomalia, D. A. et al. (1985) Polym. J. (Tokyo) 17, 117; Tomalia, D. A. et al. (1986) Macromolecules 19, 2466.

The dendrimers of structure DAB are in particular described by de Brabander-van den Berg, E. M. M. et al. (1993) Angew. Chem. Int. Ed. Engl. 32, 1308.

The dendrimers of structure PMMH are in particular described in “A general synthetic strategy for neutral phosphorus containing dendrimers” by Launay, N. et al. (1994) Angew. Chem. 106, 1682/Angew. Chem. Int. Ed. Engl. 33, 1589 and in “Synthesis of bowl-shaped dendrimers from generation 1 to generation 8” Launay, N. et al. (1997) J. Organomet. Chem. 529, 51.

An example of a dendrimer of type PAMAM for which n=4 and m=4 is represented below:

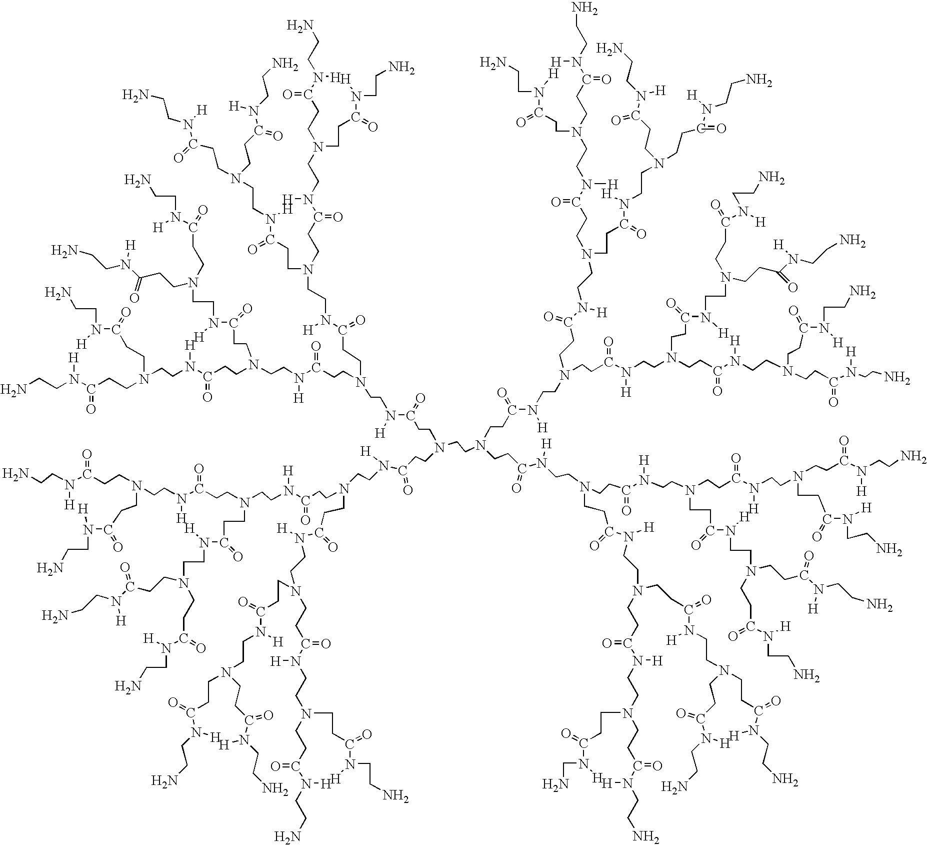

An example of a dendrimer of type DAB, for which n=5 and m=4 is represented below:

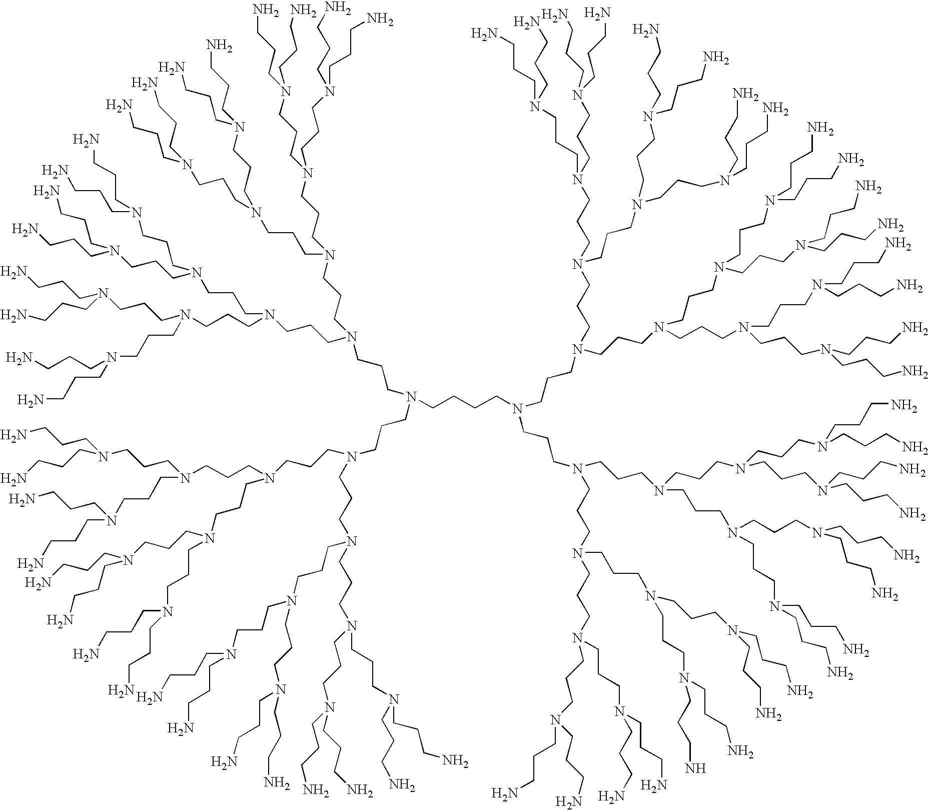

An example of a dendrimer of type PMMH with a thiophosphoryl core, for which n=4 and m=3 is represented below:

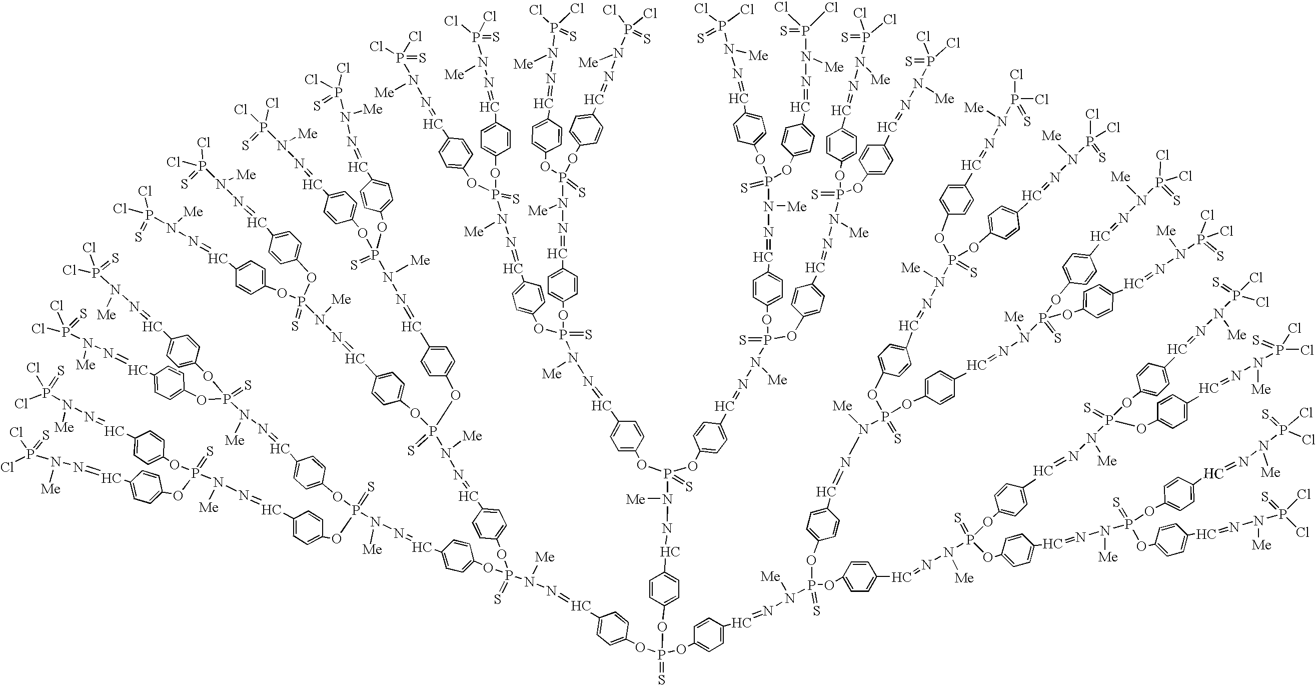

An example of a dendrimer of type PMMH with a cyclotriphosphazene core, for which n=2 and m=6, without an intermediate chain, is represented below:

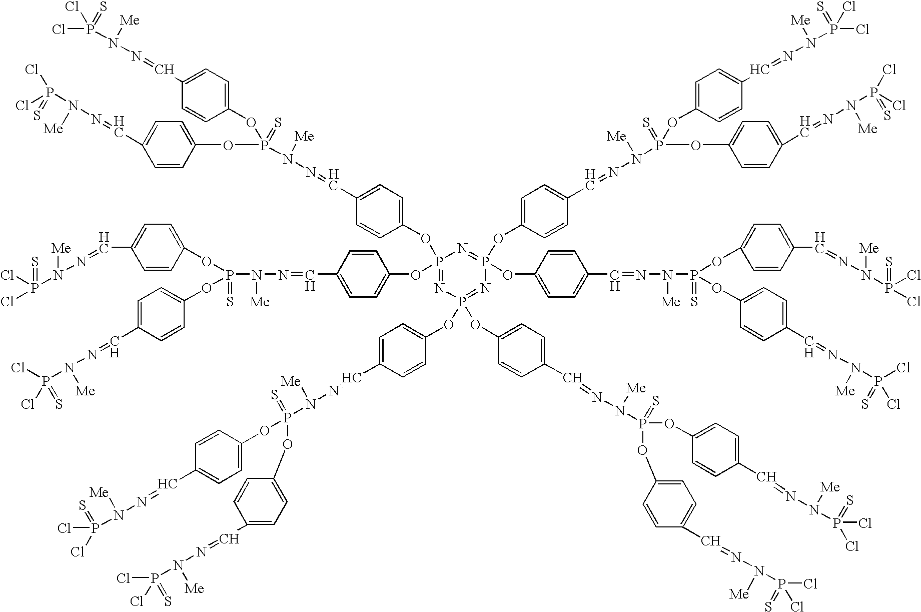

Another example of a dendrimer of type PMMH with a cyclotriphosphazene core, for which n=2 and m=6, with an intermediate chain, is represented below:

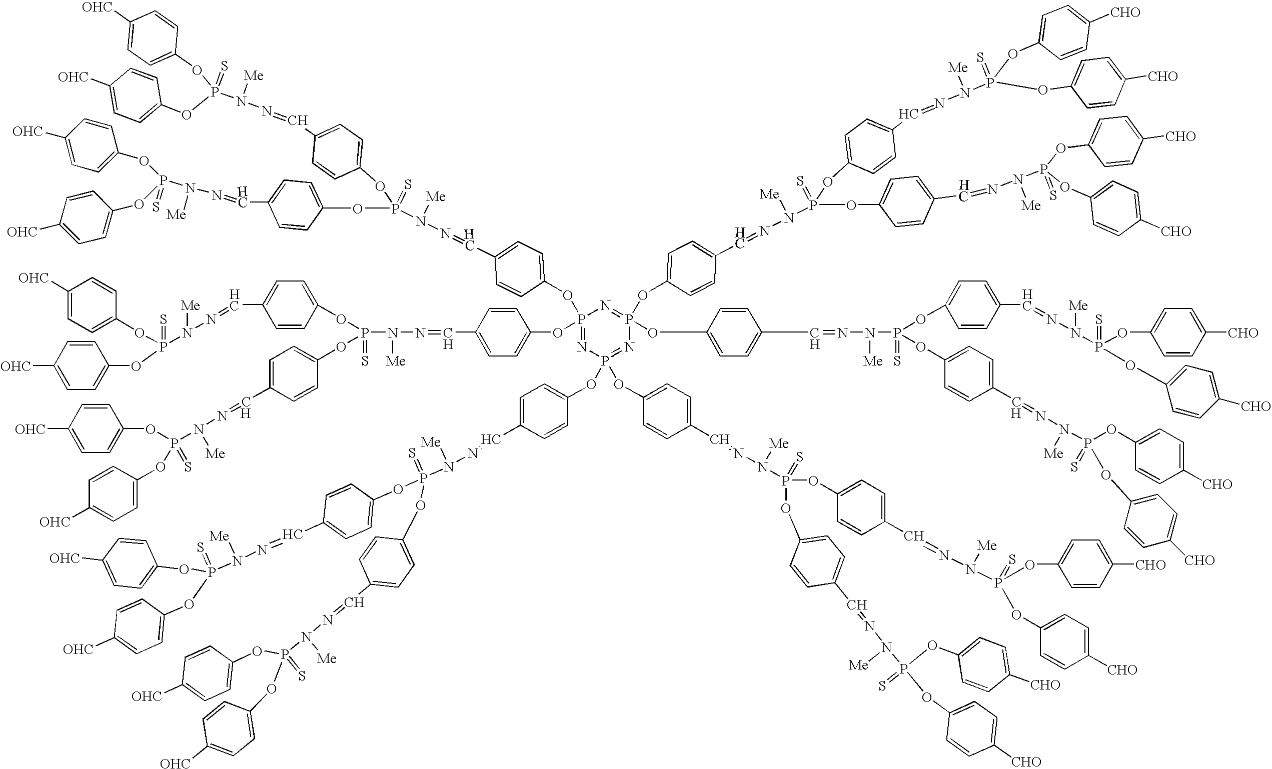

In a particular embodiment, the invention relates to the use of dendrimers with monophosphonic or bisphosphonic terminations corresponding to the following general formula (1a):

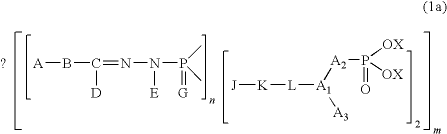

where n represents an integer from 0 to 3, namely:
when n=0, formula (1a) corresponds to the following formula (2a).

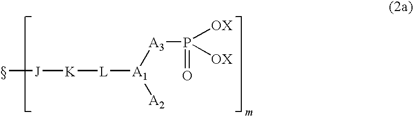

when n=1, formula (1a) corresponds to the following formula (3a),

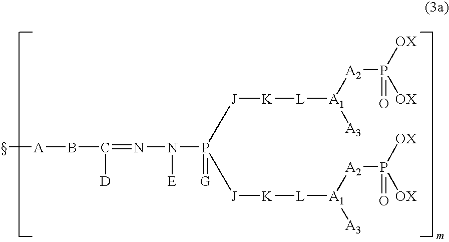

when n=2, formula (1a) corresponds to the following formula (4a),

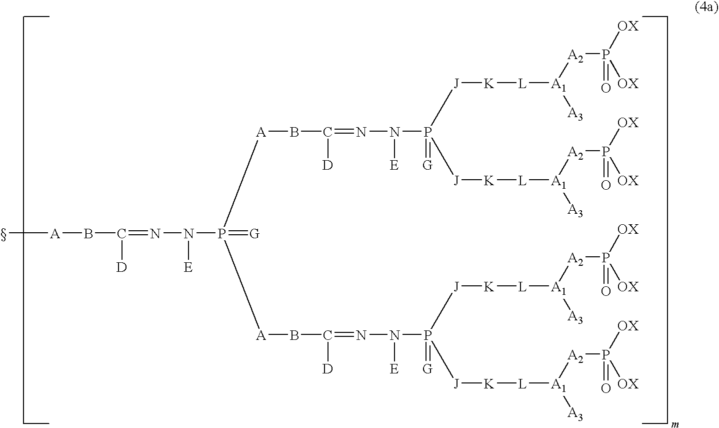

and when n=3, formula (1a) corresponds to the following formula (5a),

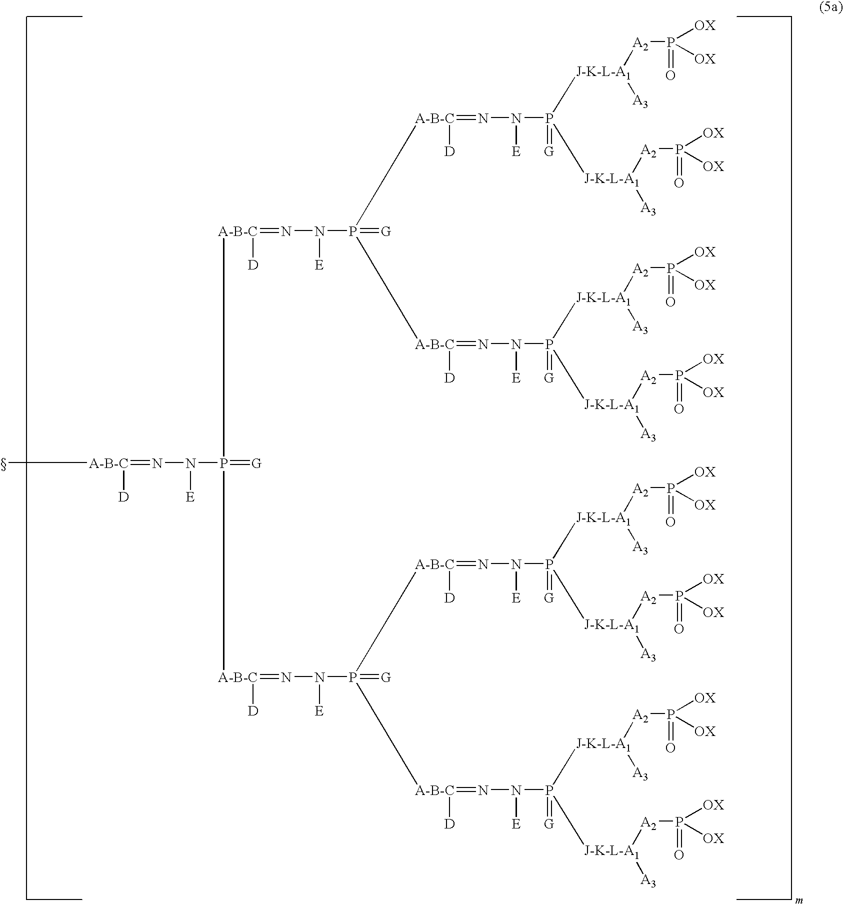

and in which formulae:
the central core § is chosen from the following groups:

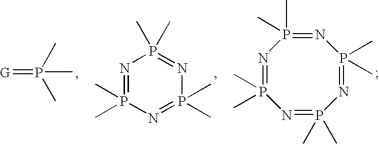

m represents 3, 6 or 8;
the generation chain corresponds to the formula:

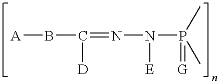

where
A represents an oxygen, sulphur, phosphorus atom or a NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹; according to the invention, when B represents two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group selected from the groups comprising C₆H₄—O—C₆H₄ and C₆H₄—(CH₂CH₂O)q-C₆H₄ with q=1 to 12.
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a —NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group; R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:

-J-K-L-

where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, optionally containing one or more double or triple bonds, each of said members being able to be optionally a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula:

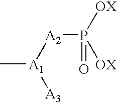

where A₁, A₂, A₃ and X have been defined previously, each X being identical or different.

In a more particular embodiment, the invention relates to the use of a dendrimer of general formula (1a) in which A₃ represents:

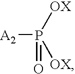

said general formula (1a) then corresponding to the following general formula (1):

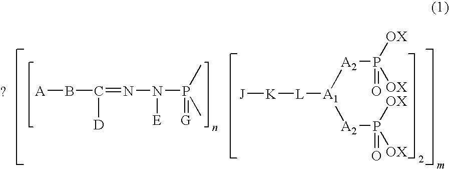

§, A, B, C, D, E, G, J, K, L, A₁, A₂, X, m and n being as defined above.

In a particular embodiment of the invention, the dendrimers with bisphosphonic terminations correspond to the following general formula (1) where n represents an integer from 0 to 3, namely:
when n=0, formula (1) corresponds to the following formula (2),

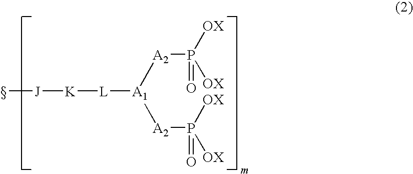

when n=1, formula (1) corresponds to the following formula (3),

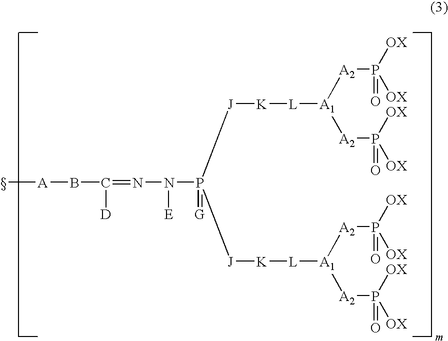

when n=2, formula (1) corresponds to the following formula (4),

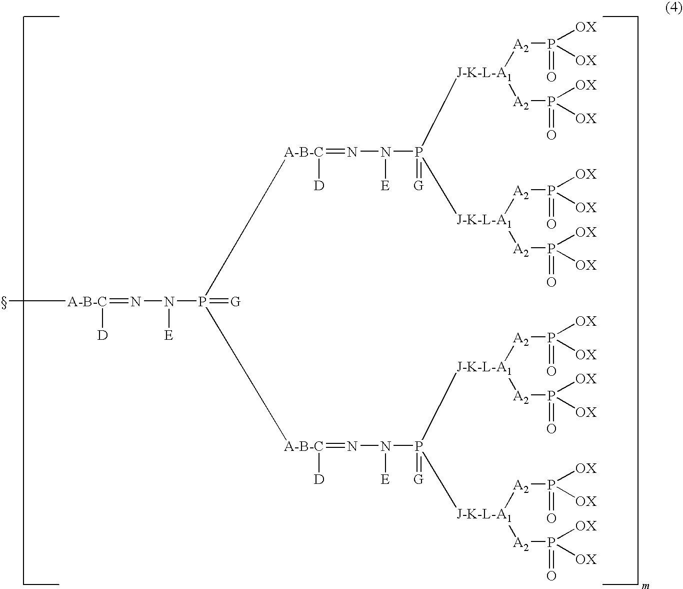

and when n=3, formula (1) corresponds to the following formula (5),

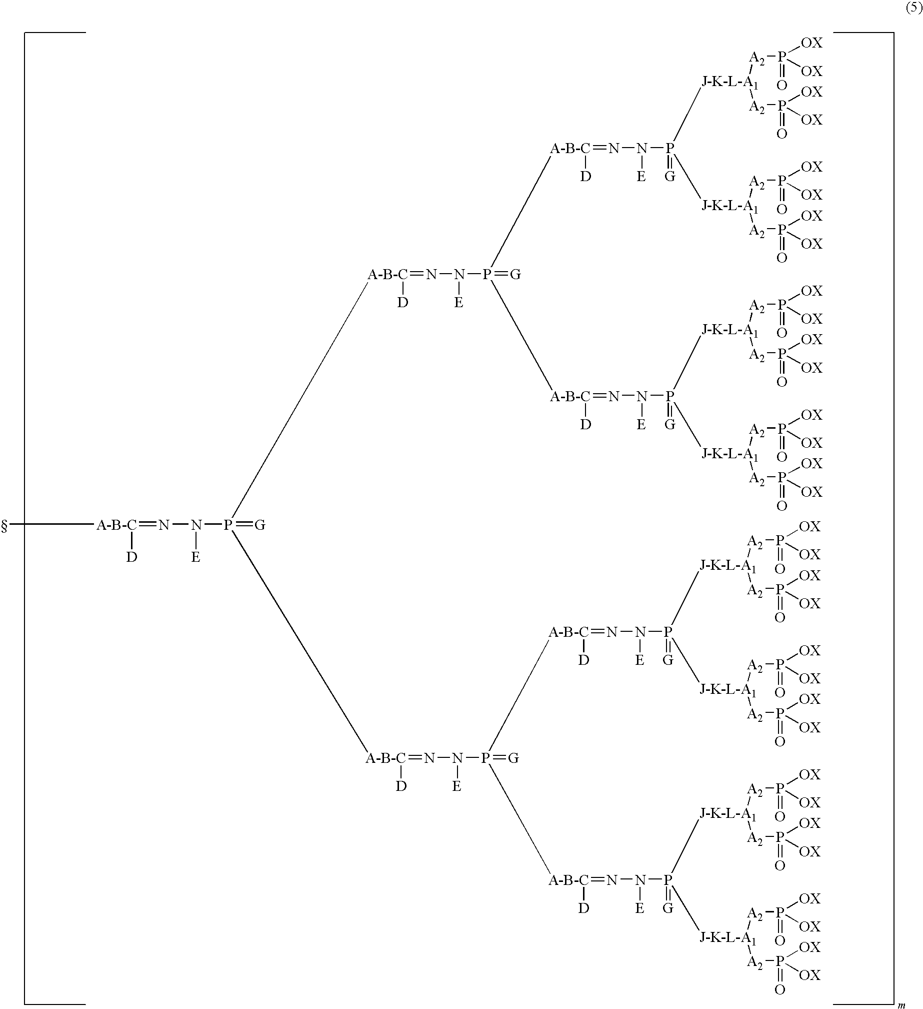

and in which formulae:
the central core § is chosen from the following groups:

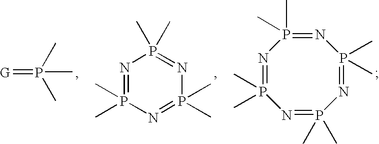

m represents 3, 6 or 8;
the generation chain corresponds to the formula:

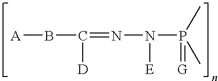

where
A represents an oxygen, sulphur, phosphorus atom or a NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹; According to the invention, when B represents two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group selected from the groups comprising C₆H₄—O—C₆H₄ and C₆H₄—(CH₂CH₂O)q-C₆H₄ with q=1 to 12.
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹; each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group; R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:

-J-K-L-

where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula:

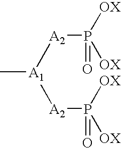

where A₁, A₂ and X have been defined previously, each X being identical or different.

According to a preferred embodiment, the invention relates to the use as defined above of a dendrimer of general formula (1) of structure PMMH, in which
§ represents

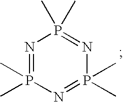

m represents 6;
n represents 0, 1, or 2;
A represents an oxygen atom;
B represents a benzene group;
D represents hydrogen;
E represents a methyl group;
G represents a sulphur atom;
J represents an oxygen atom;
K represents a benzene group;
L represents a non-substituted linear saturated hydrocarbon chain with two carbon atoms;
A₁ represents a nitrogen atom;
A₂ represents a CH₂ group;
X represents a methyl group, or a hydrogen or sodium atom;
   said dendrimer being designated GCn, n being defined above.

According to a particularly preferred embodiment, the invention relates to the use as defined above of compounds of the following formulae:

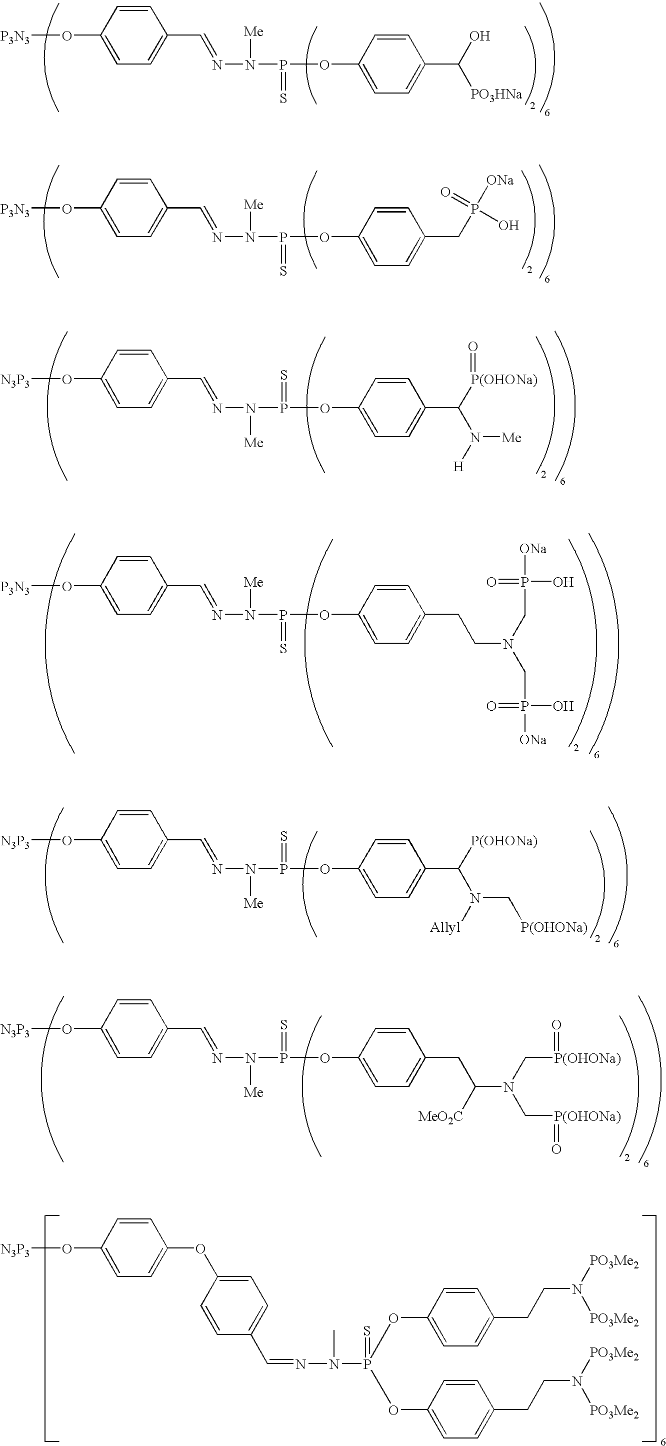

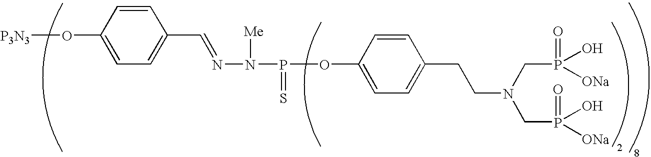

or of GC1 compounds of the following formulae:

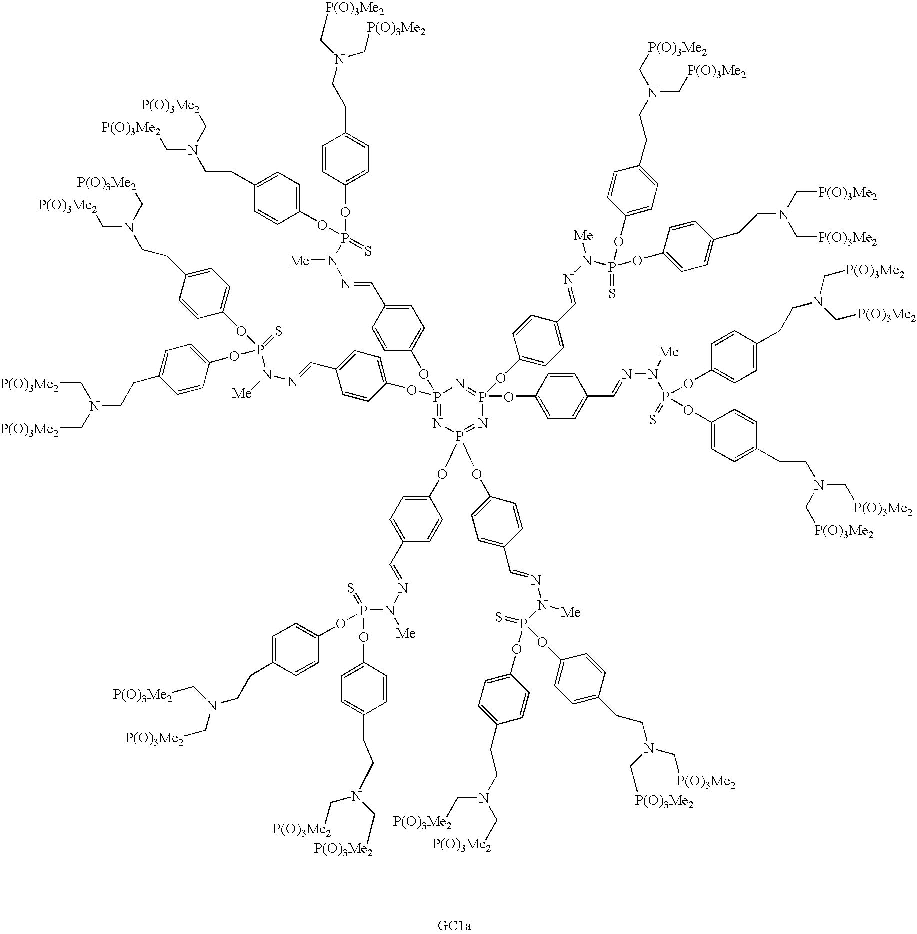

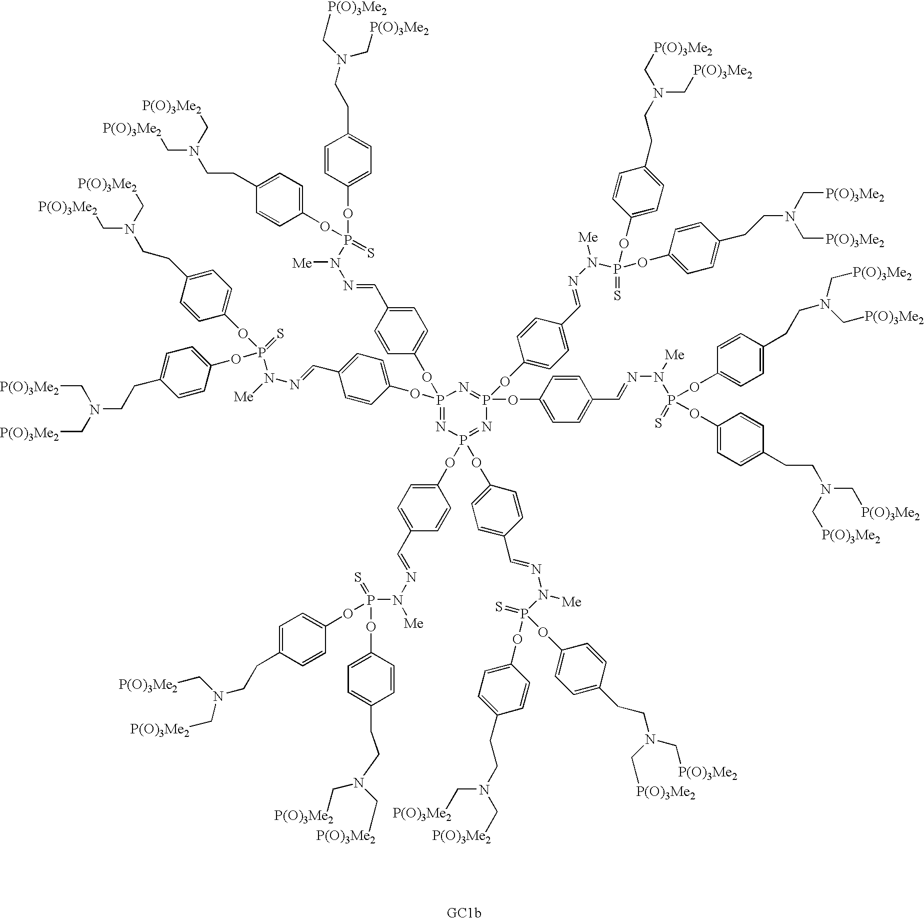

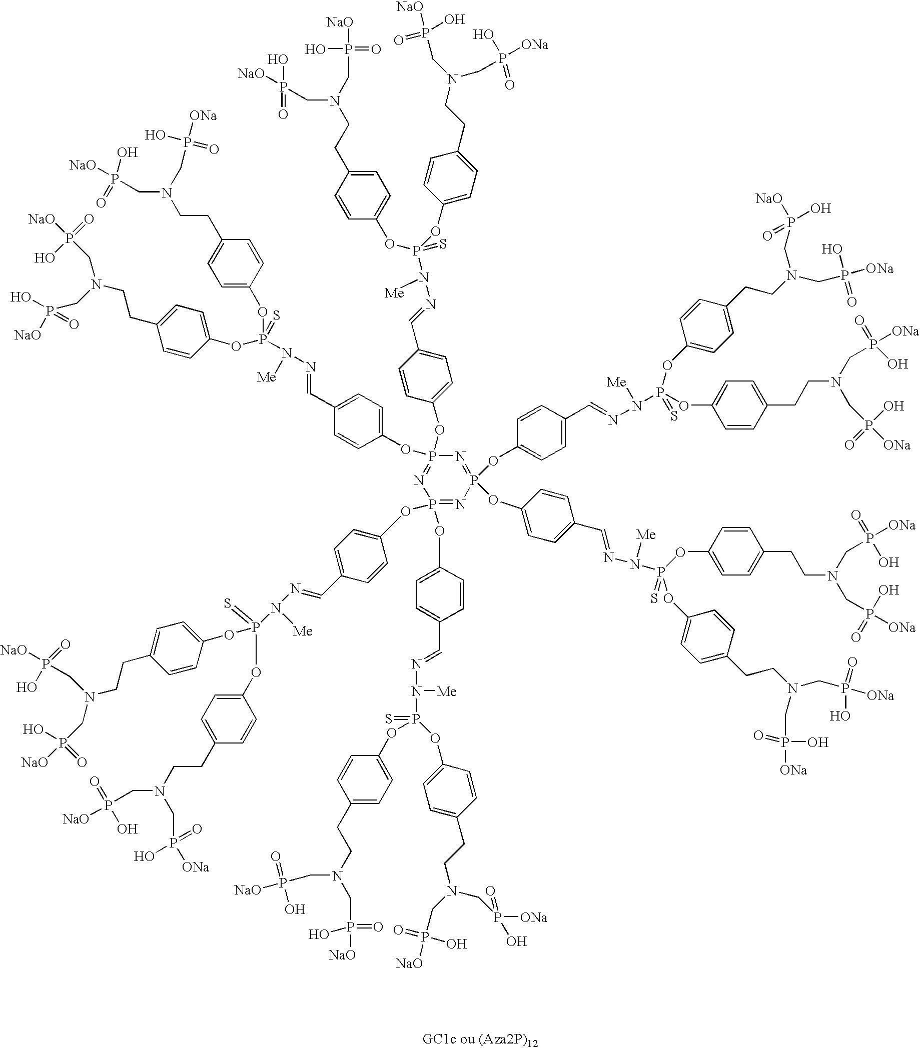

The present invention also relates to the use of dendrimers with bisphosphonic terminations corresponding to the following general formula (7):

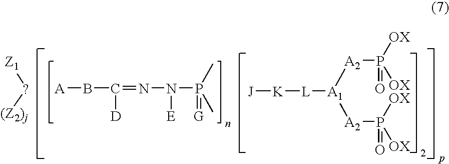

where n represents an integer from 0 to 3, m represents 3, 6 or 8, p represents m−1 or m−2, and j represents 0 when p represents m−1 and 1 when p represents m−2, namely:
when p=m−1, formula (7) corresponds to the following formula (8):

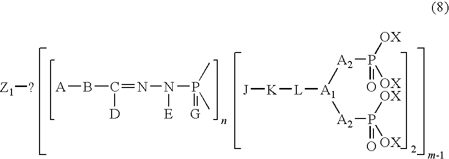

when p=m−2, formula (7) corresponds to the following formula (9):

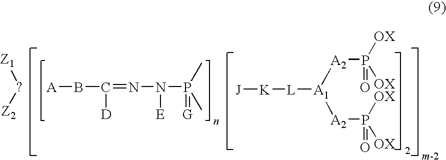

and in which formulae:
the central core § is chosen from the following groups:

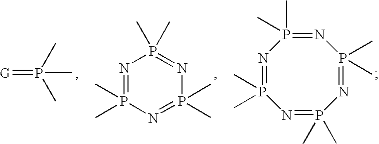

the generation chain corresponds to the formula:

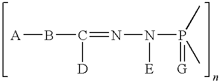

where
A represents an oxygen, sulphur, phosphorus atom or a NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹. According to the invention, when B represents two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group selected from the groups comprising C₆H₄—O—C₆H₄ and C₆H₄—(CH₂CH₂O)q-C₆H₄ with q=1 to 12.
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group; R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:

-J-K-L-

where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹; R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula:

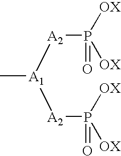

where A1, A2 and X have been defined previously, each X being identical or different;
Z₁ and Z₂ being identical or different, optionally linked together, in particular by means of a covalent bond, and representing H or a linear, branched or cyclic hydrocarbon chain with 1 to 16 members, optionally containing one or more double or triple bonds, each of said members being optionally chosen from a heteroatom, said heteroatom being preferably chosen from a nitrogen, oxygen, phosphorus, silicon or sulphur atom, an aryl group of 6 to 24 carbon atoms, a heteroaryl group of 1 to 24 carbon atoms, a carboxyl group, a >C═NR group, each member being able to be optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, the first member of said hydrocarbon chain preferably being oxygen or nitrogen.

The invention relates more particularly to the use as defined above, of a dendrimer of general formula (8) of structure PMMH, in which
§ represents

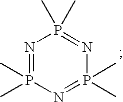

m represents 6;
p represents 5;
n represents 0, 1, or 2;
A represents an oxygen atom;
B represents a benzene group;
D represents hydrogen;
E represents a methyl group;
G represents a sulphur atom;
J represents an oxygen atom;
K represents a benzene group;
L represents a non-substituted linear saturated hydrocarbon chain with two carbon atoms;
A₁ represents a nitrogen atom;
A₂ represents a CH₂ group;
X represents a methyl group, or a hydrogen or sodium atom;
Z₁ represents a phenyloxy group;
   said dendrimer being designated GCn′, n being defined above.

Preferably, the present invention relates in particular to the use as defined above:
of the compounds of the following formulae:

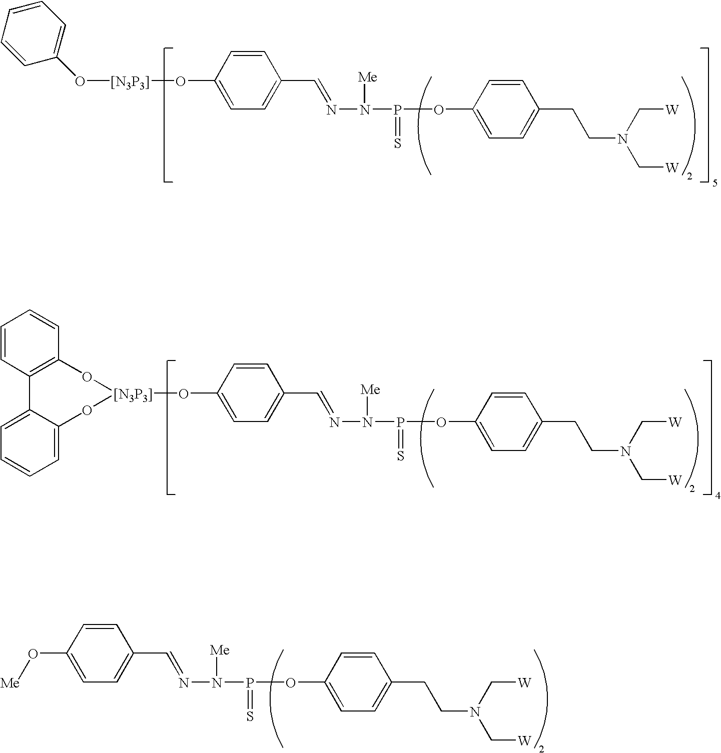

in which W represents PO₃Me₂, PO₃HNa, PO₃H₂, said compounds corresponding, in particular, to compound GC1′ of the following formula (10):

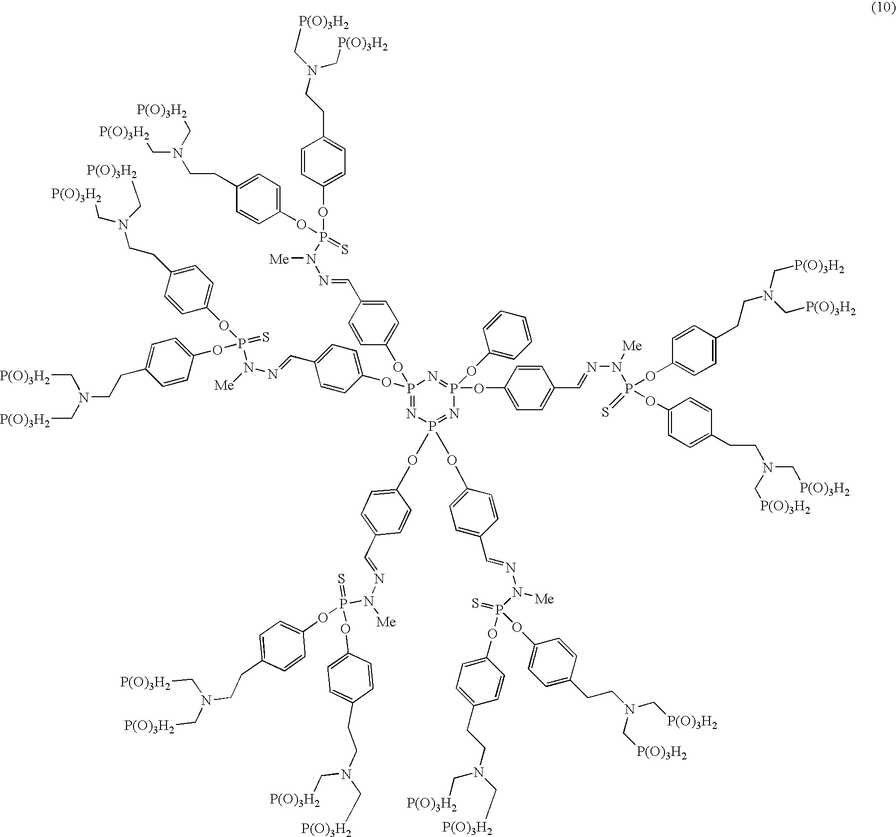

or of the formula (10a)

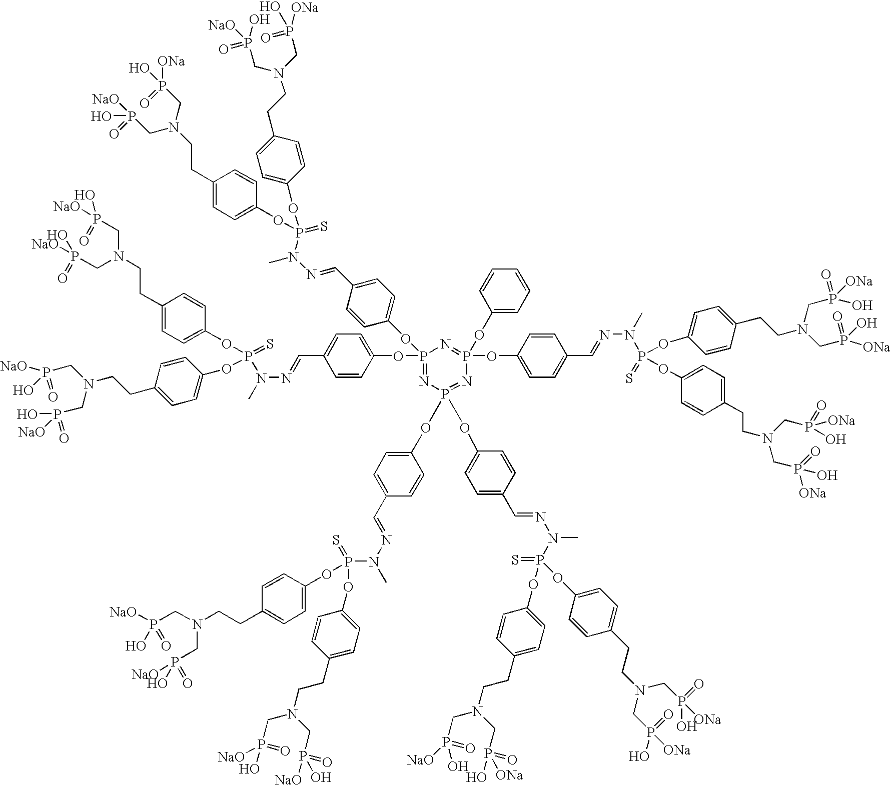

or compounds of the following formula:

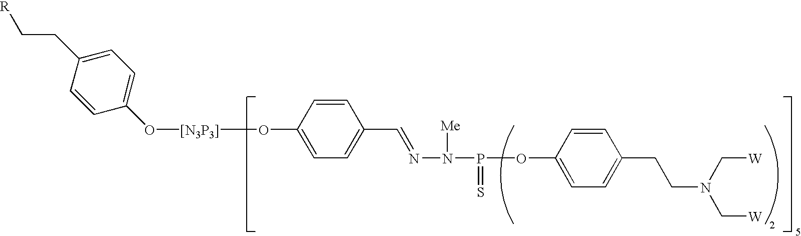

in which W represents PO₃Me₂, PO₃HNa, or PO₃H₂ and R is selected from the group comprising (a) fluorescent groups chosen from:

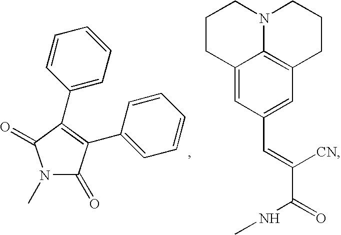

and (b) a biotinyl group of formula

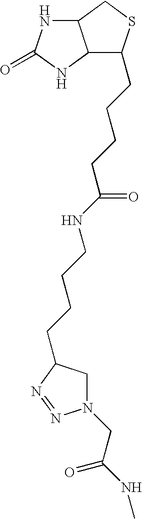

said compounds corresponding in particular to the compounds of the following formulae:

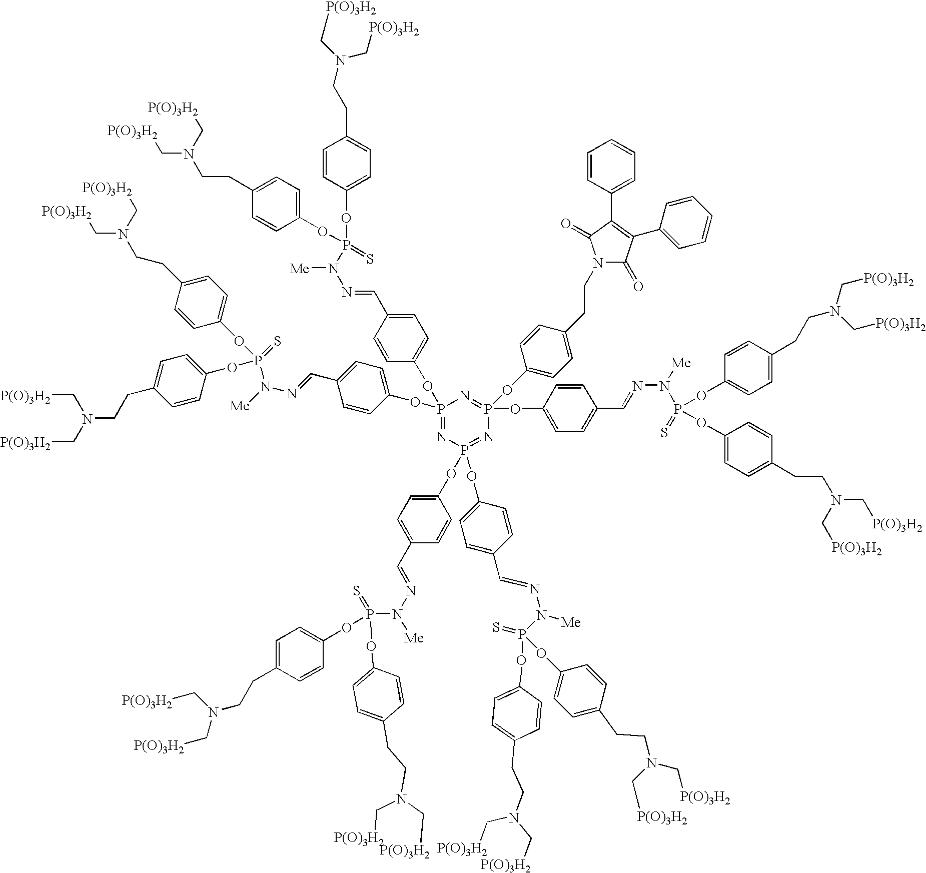

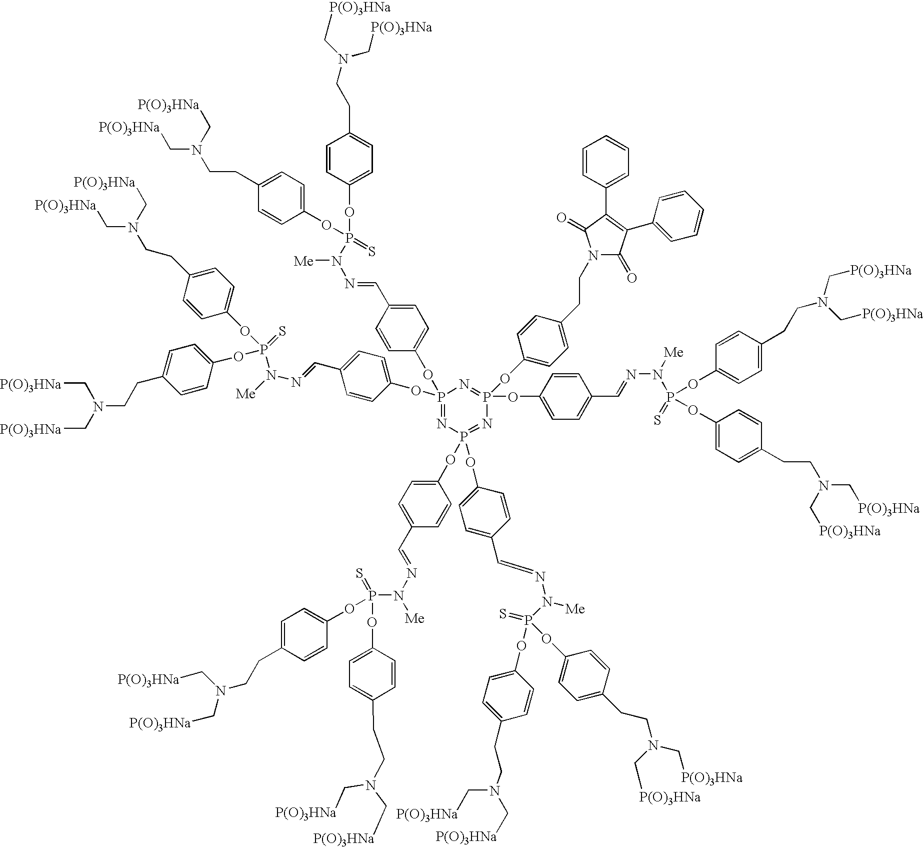

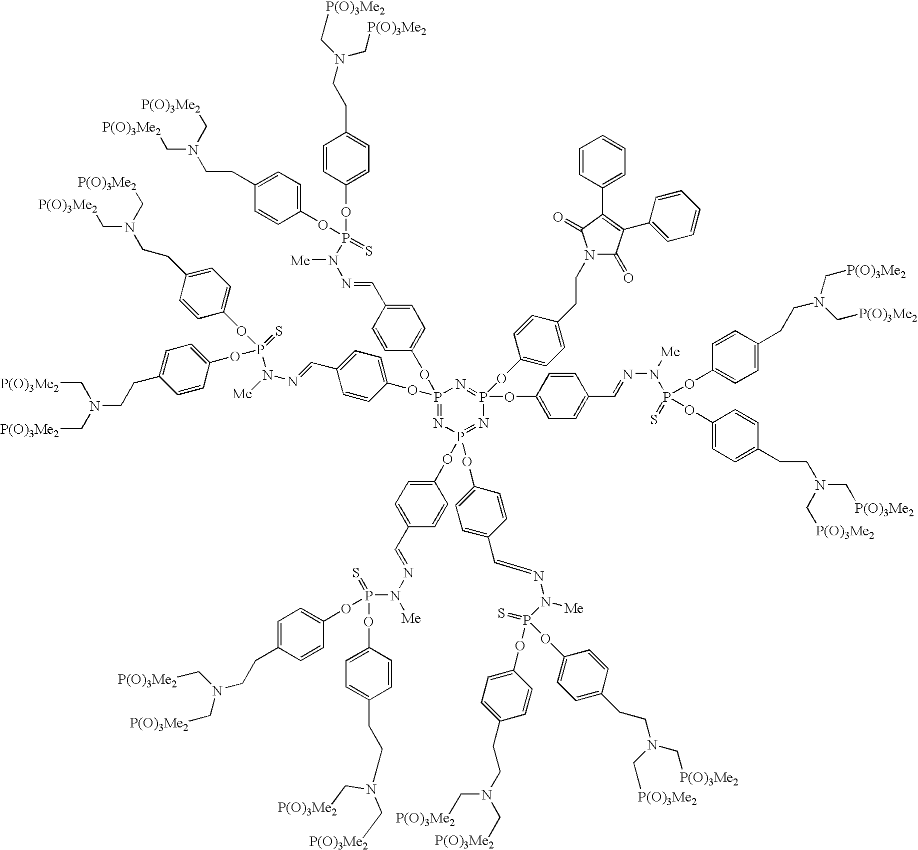

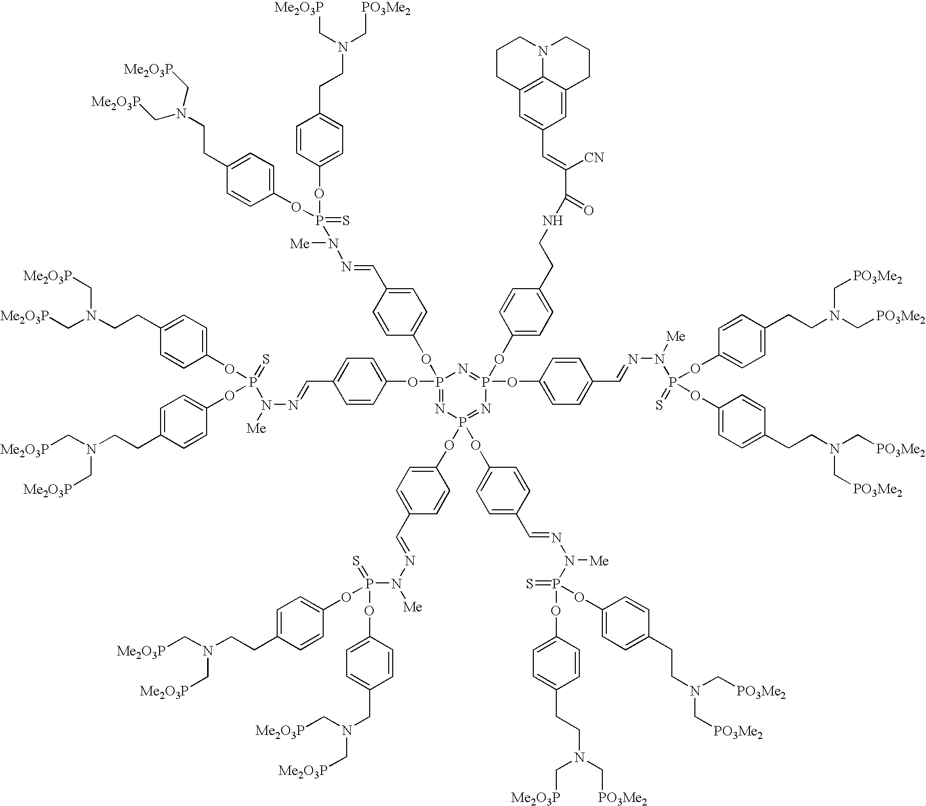

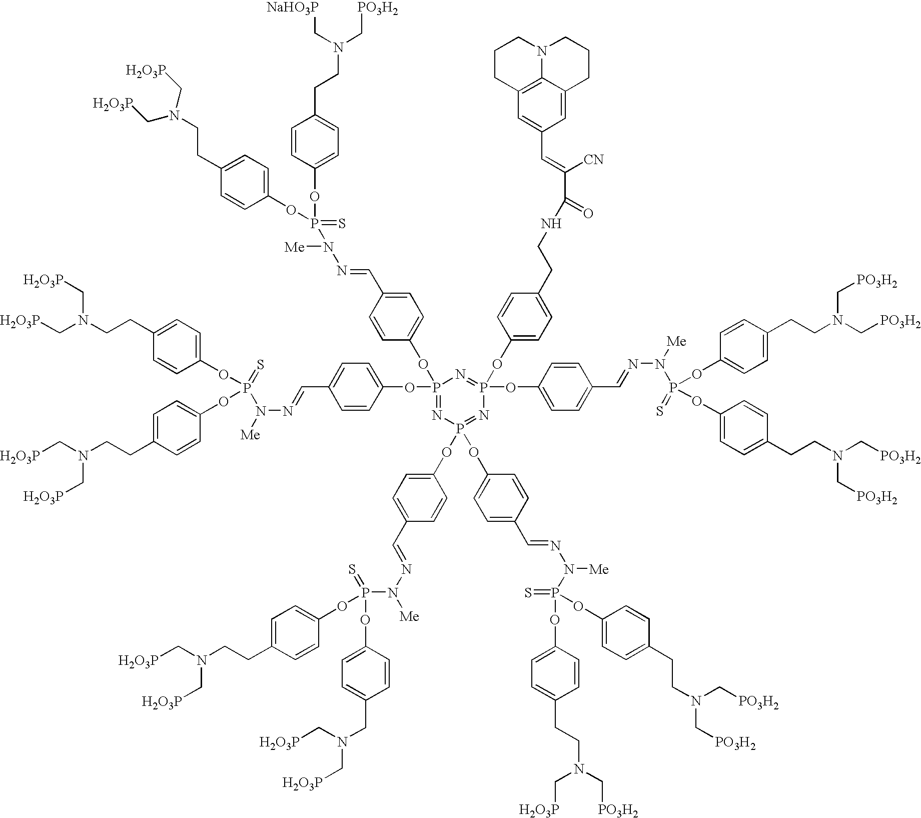

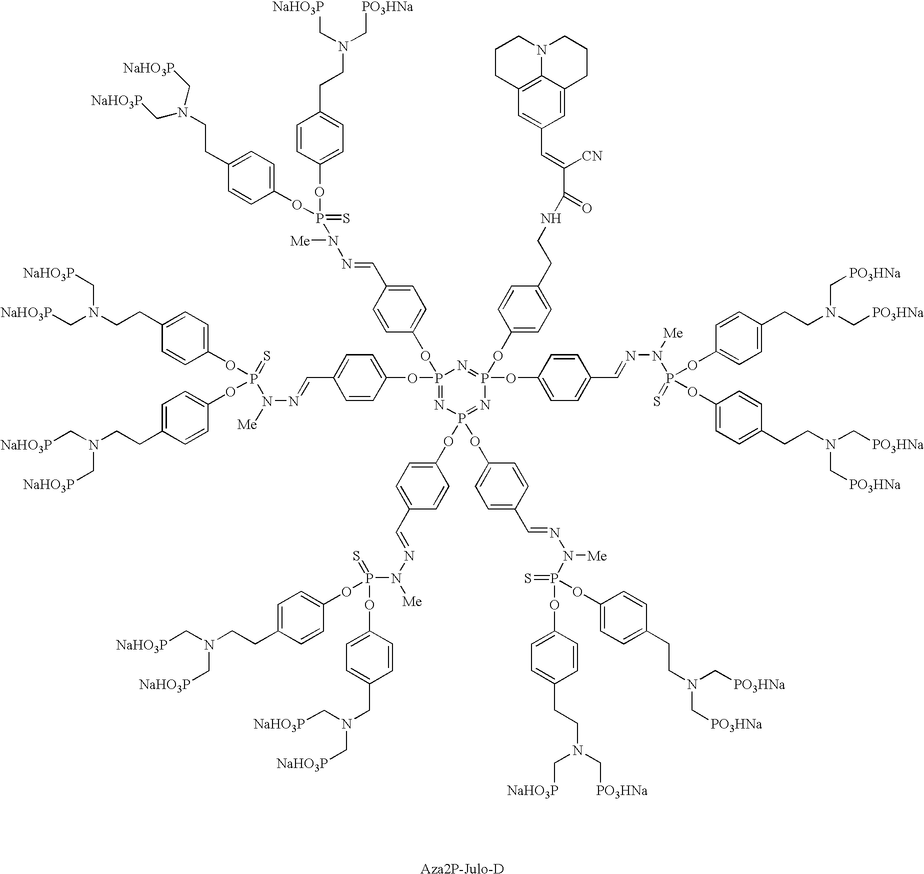

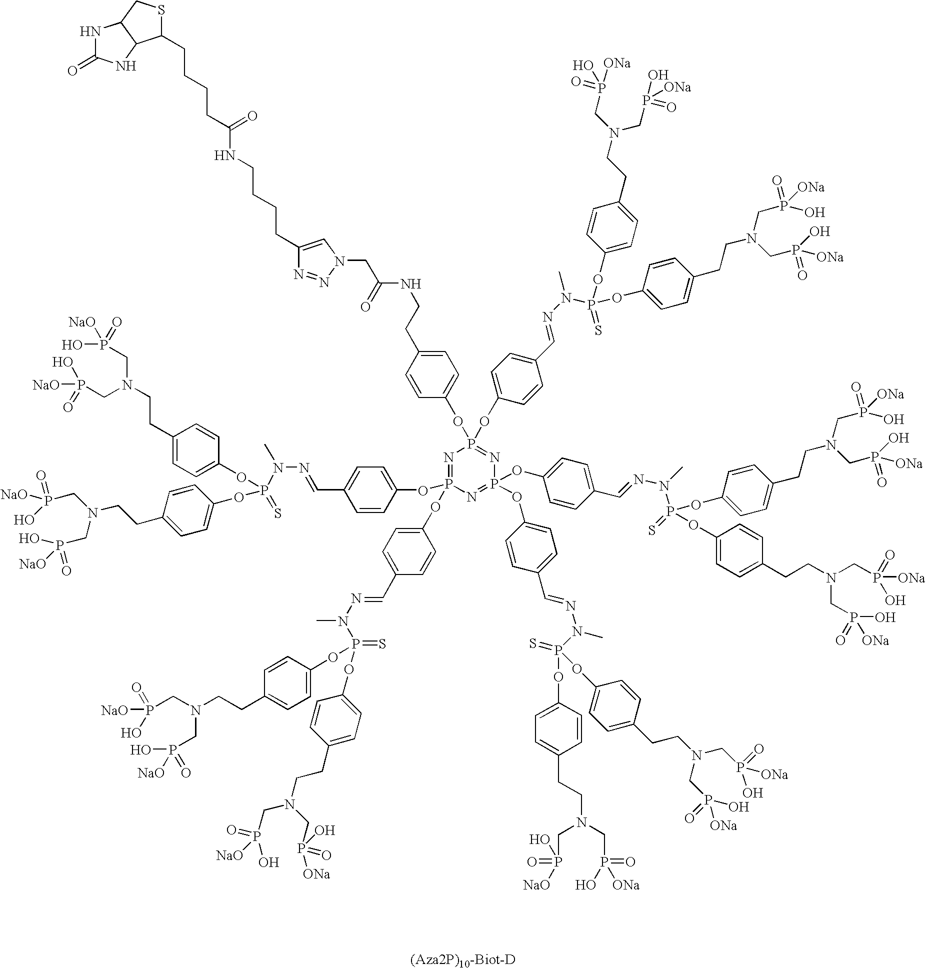

The present invention also relates to the use of dendrimers with bisphophonic terminations of the following formula:

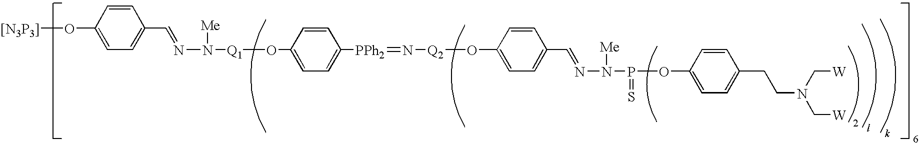

in which W represents PO₃Me₂, PO₃HNa, or PO₃H₂, Q₁ and Q₂, identical or different, represent P═S or cyclotriphosphazene (N₃P₃), 1 represents 2 when Q₂ represents P═S or 5 when Q₂ represents N₃P₃ and k represents 2 when Q₁ represents P═S or 5 when Q₁ represents N₃P₃, said dendrimers being in particular represented by the following formulae:

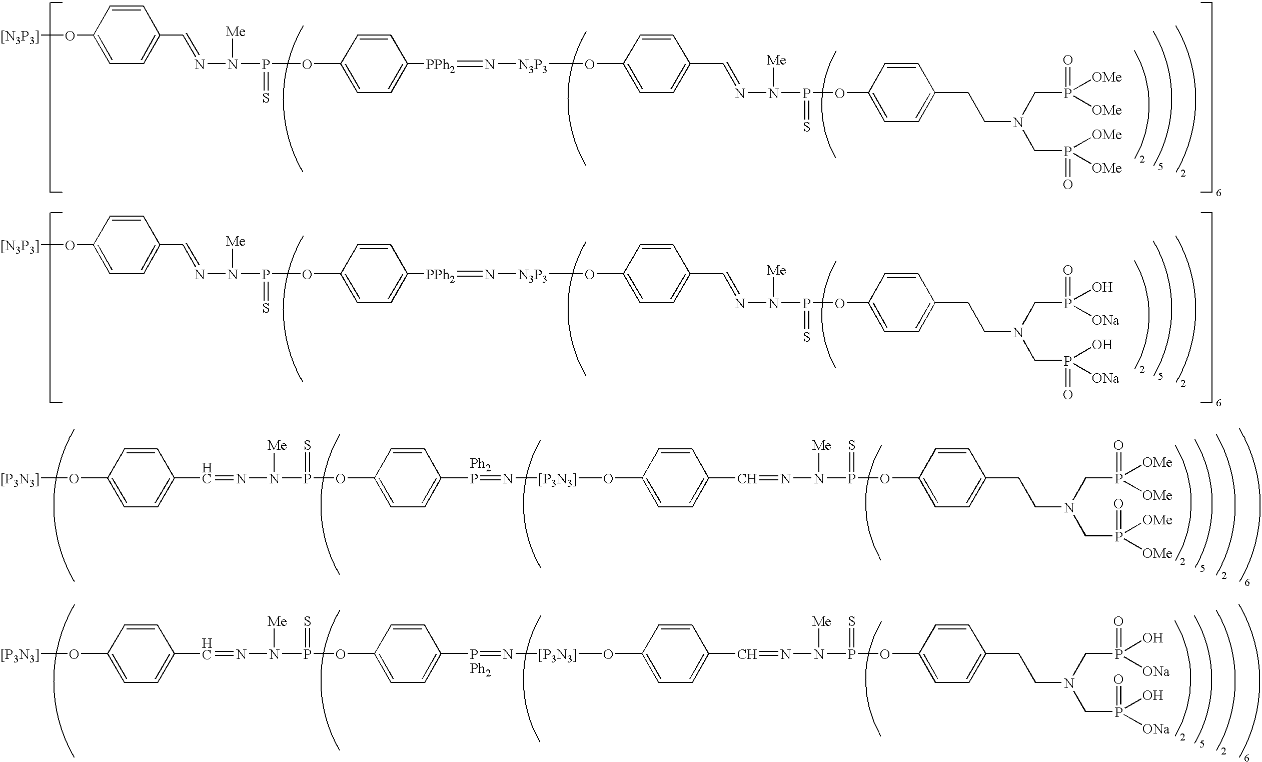

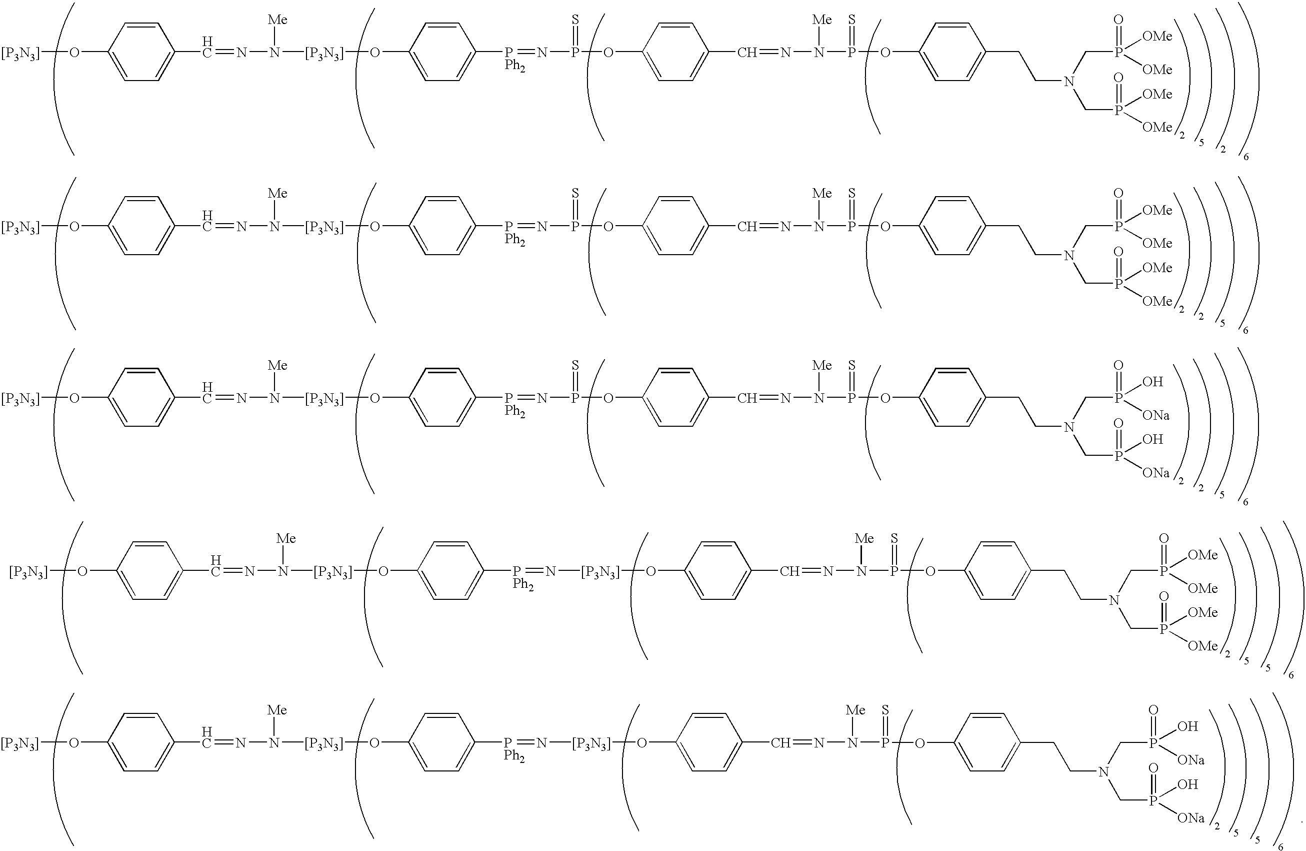

The present invention also relates to the use as defined above of dendrimers with monophosphonic or bisphosphonic terminations of the following formula:

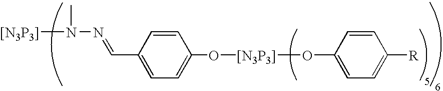

in which R represents a group chosen from

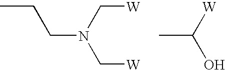

where W represents PO₃Me₂, PO₃HNa, or PO₃H₂.

The present invention also relates to the use as defined above of dendrimers with bisphosphonic terminations of the following formula:

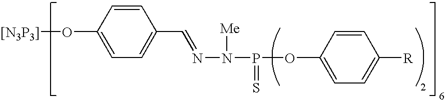

in which R represents a group chosen from:

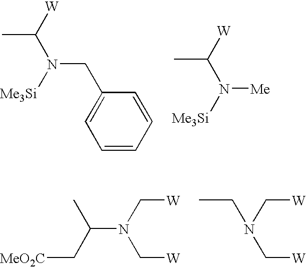

where W represents PO₃Si₂Me₆, PO₃Me₂, PO₃HNa, or PO₃H₂.

The present invention also relates to the use as defined above of dendrimers with monophosphonic terminations of the following formula:

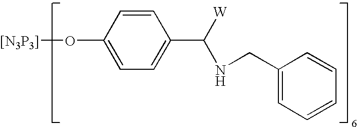

in which W represents PO₃Me₂, PO₃HNa, or PO₃H₂.

The present invention also relates to the use as defined above of dendrimers with bisphosphonic terminations of the following formula:

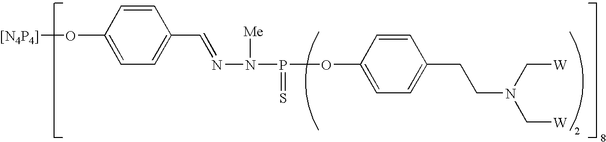

in which W represents PO₃Me₂, PO₃HNa, or PO₃H₂.

The present invention also relates to the use of dendrimers with bisphosphonic terminations of the following formula:

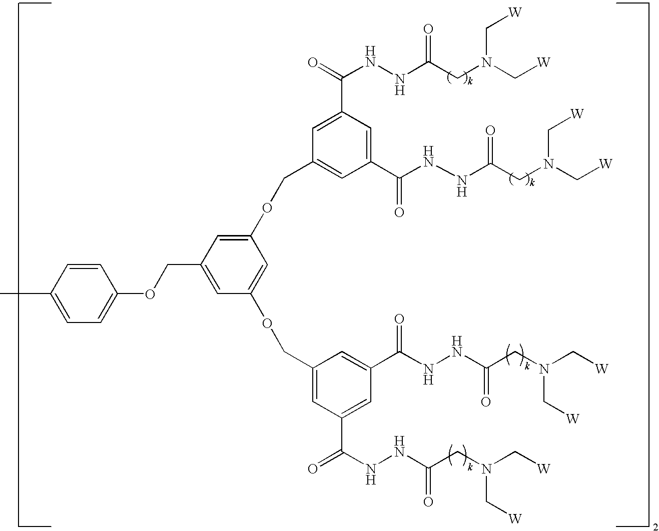

in which W represents PO₃Me₂, PO₃HNa, or PO₃H₂ and k represents 1, 2 or 3.

The present invention also relates to the use of dendrimers with bisphosphonic terminations of the following formula:

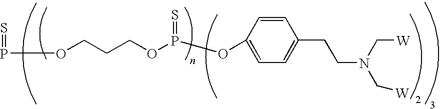

in which W represents PO₃Me₂, PO₃HNa, or PO₃H₂ and n represents 0, 1 or 2.

In a specific embodiment of the invention, the dendrimer with bisphosphonic terminations has the following formula:

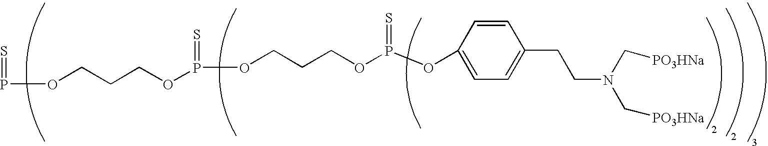

The present invention also relates to the use as defined above of dendrimers with bisphosphonic terminations of the following formula:

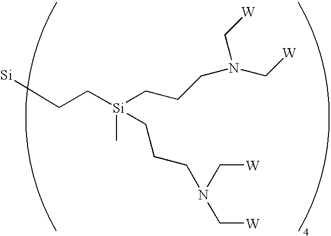

in which W represents PO₃Me₂, PO₃HNa, or PO₃H₂, in particular compound of the following formula:

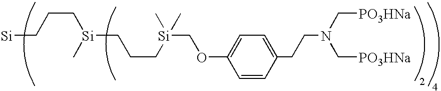

The present invention also relates to the use of dendrimers with bisphosphonic terminations of the following formula:

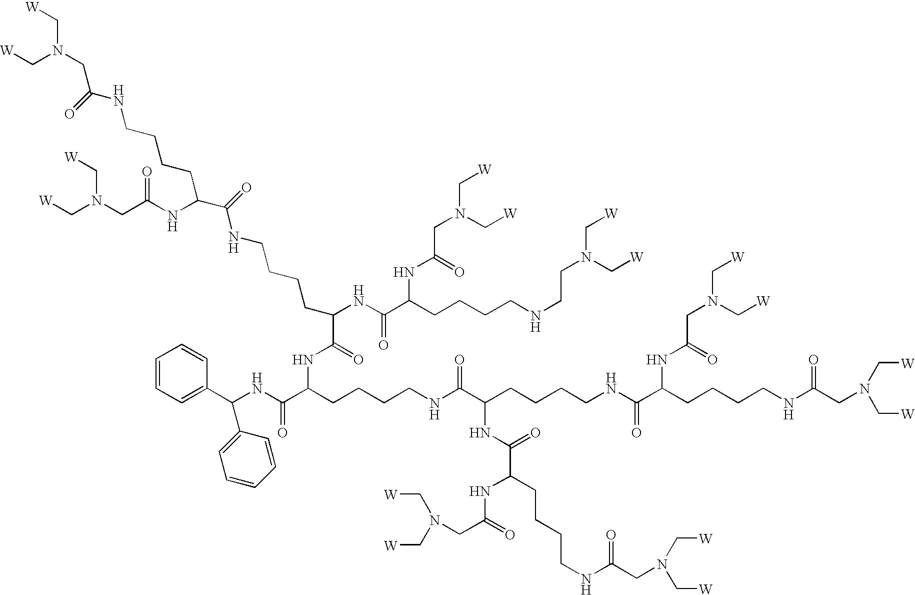

in which W represents PO₃Me₂, PO₃HNa, or PO₃H₂.

In a particular embodiment, the present invention relates to the use of the compounds of the following formulae:

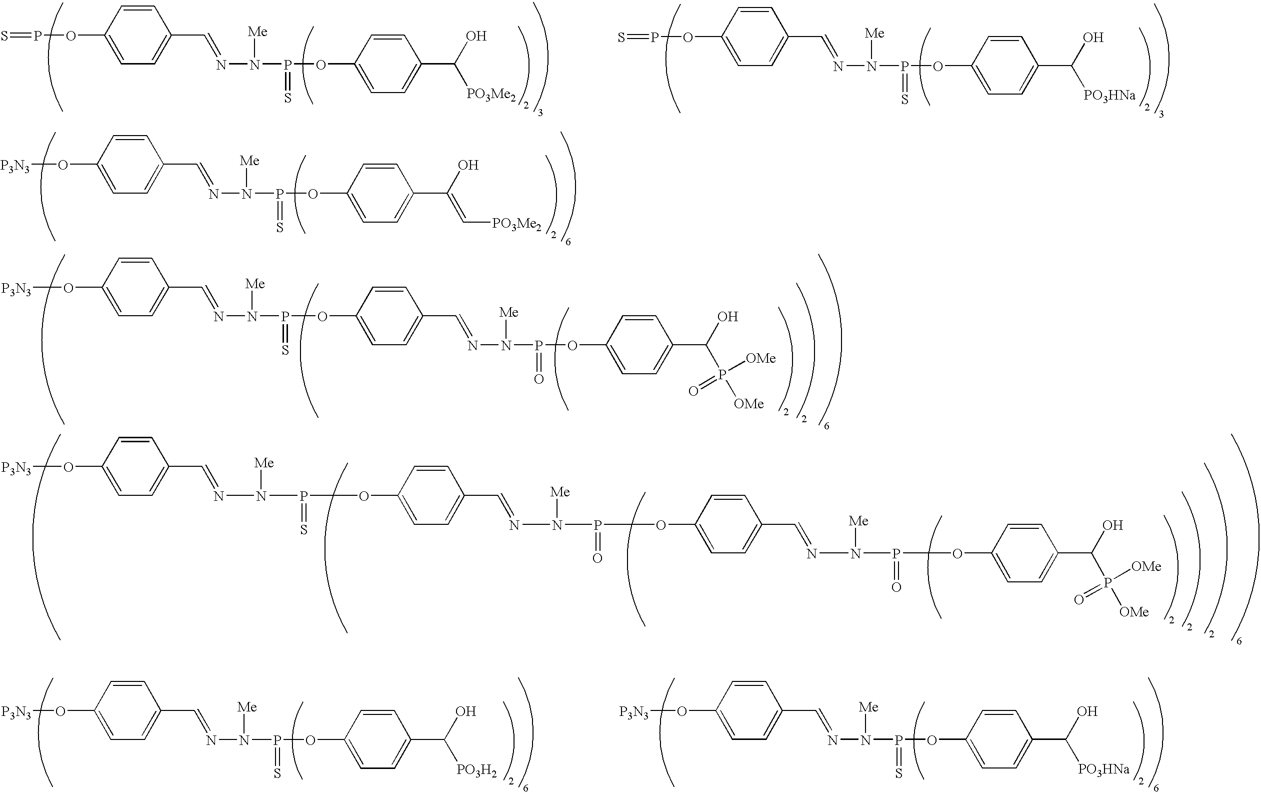

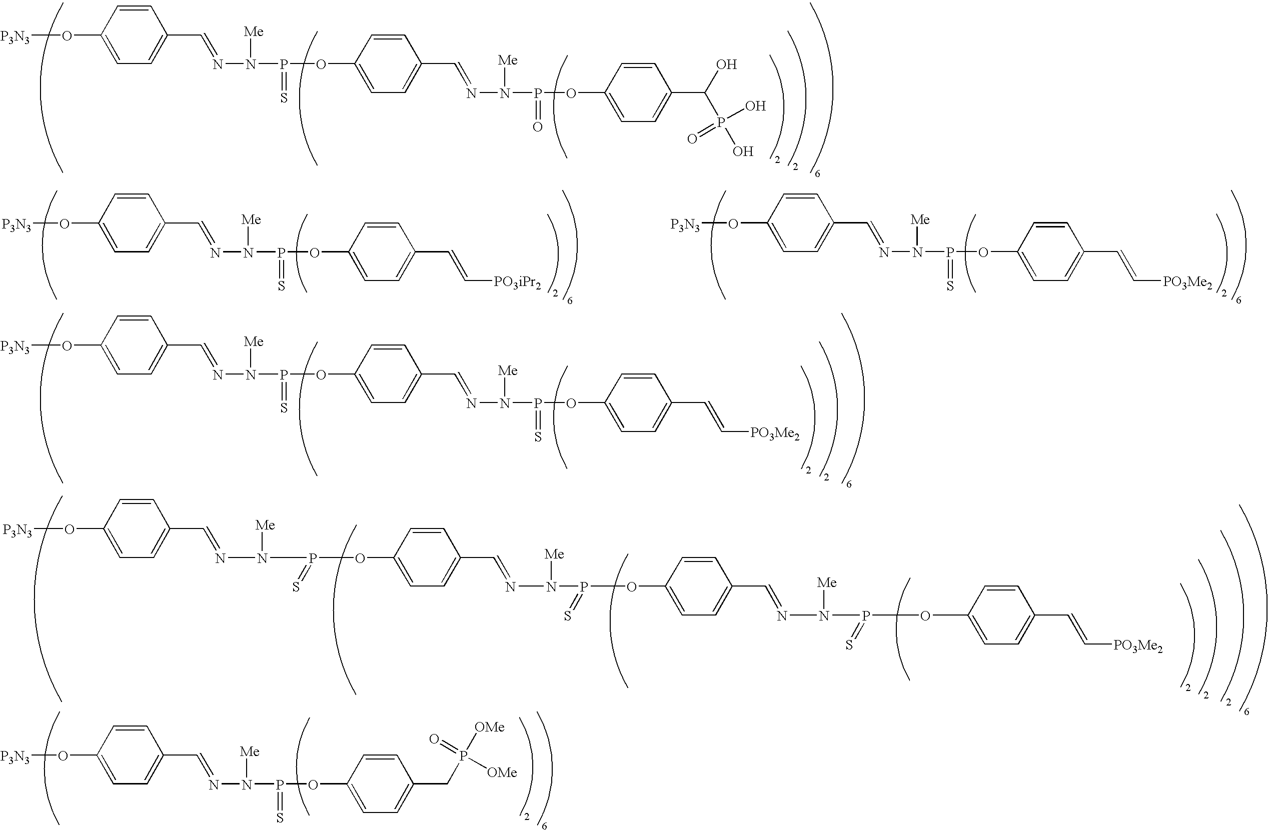

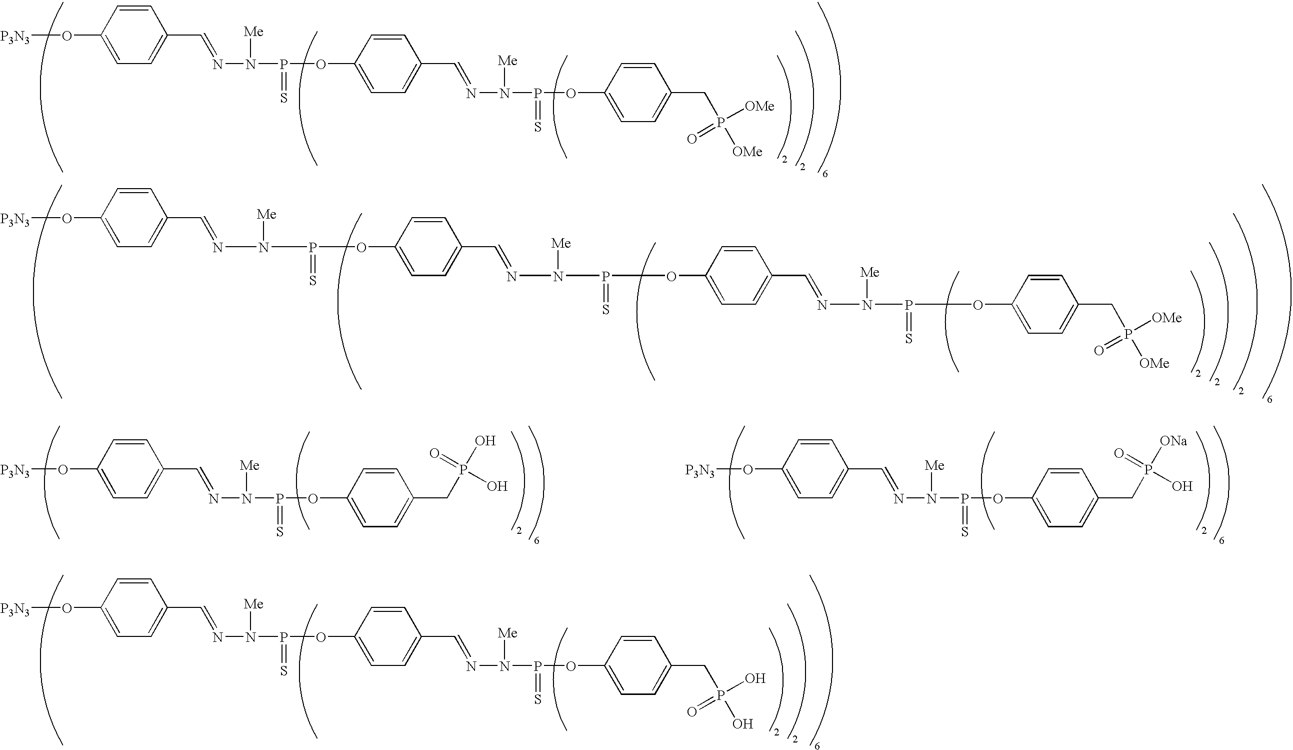

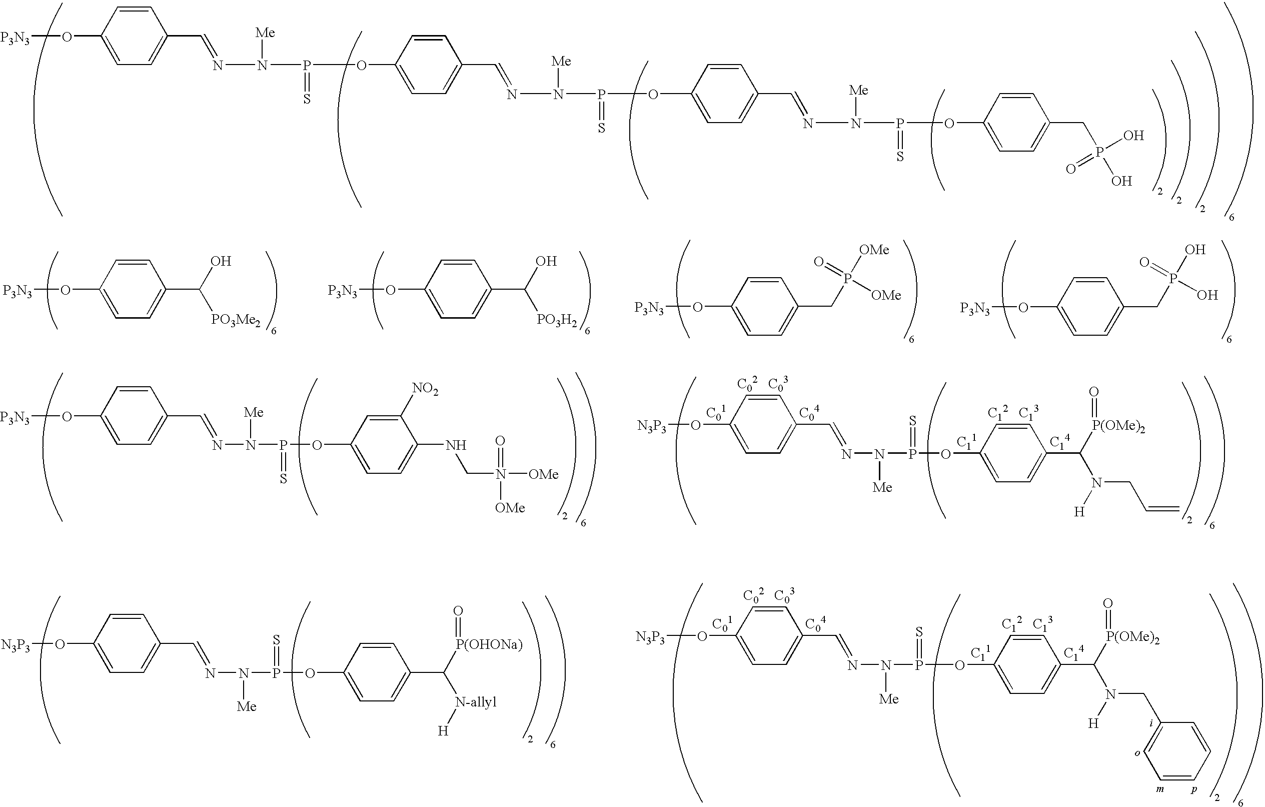

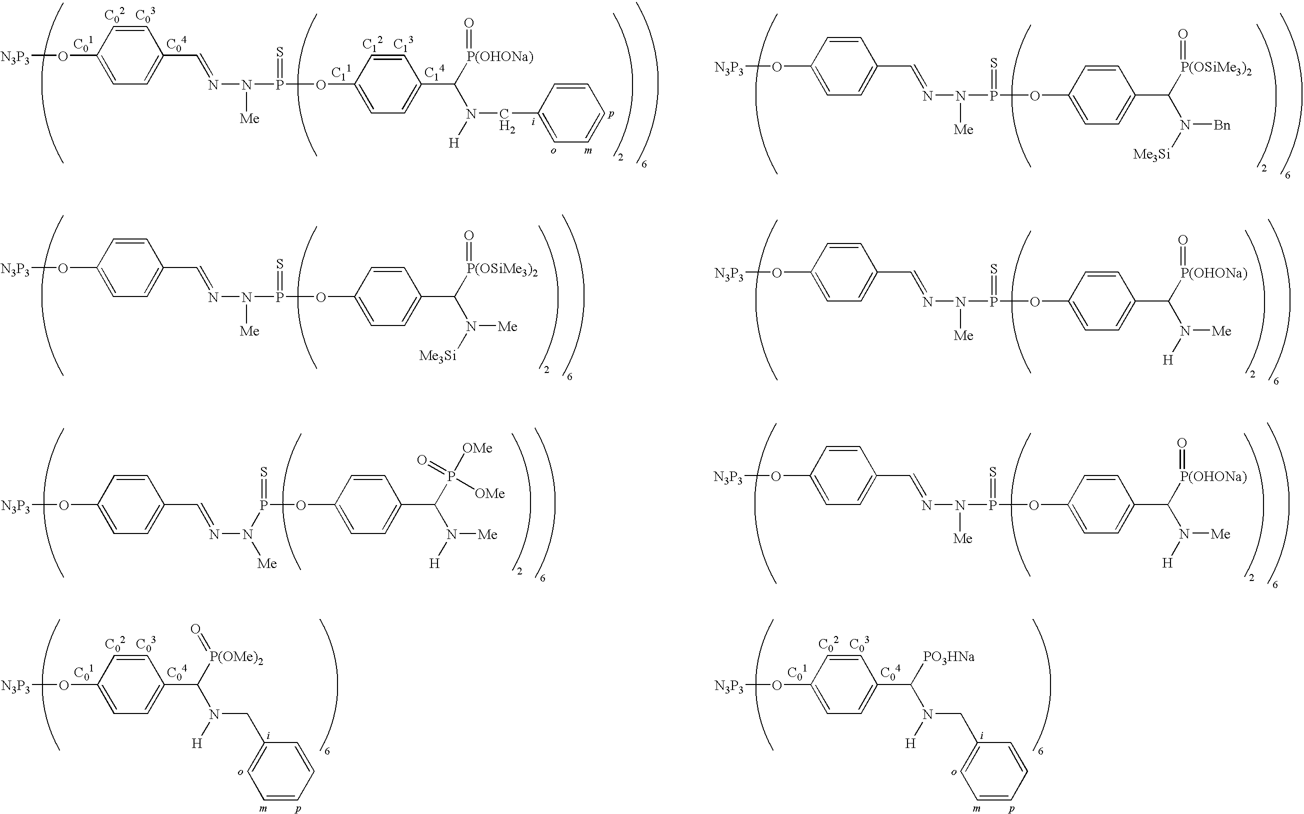

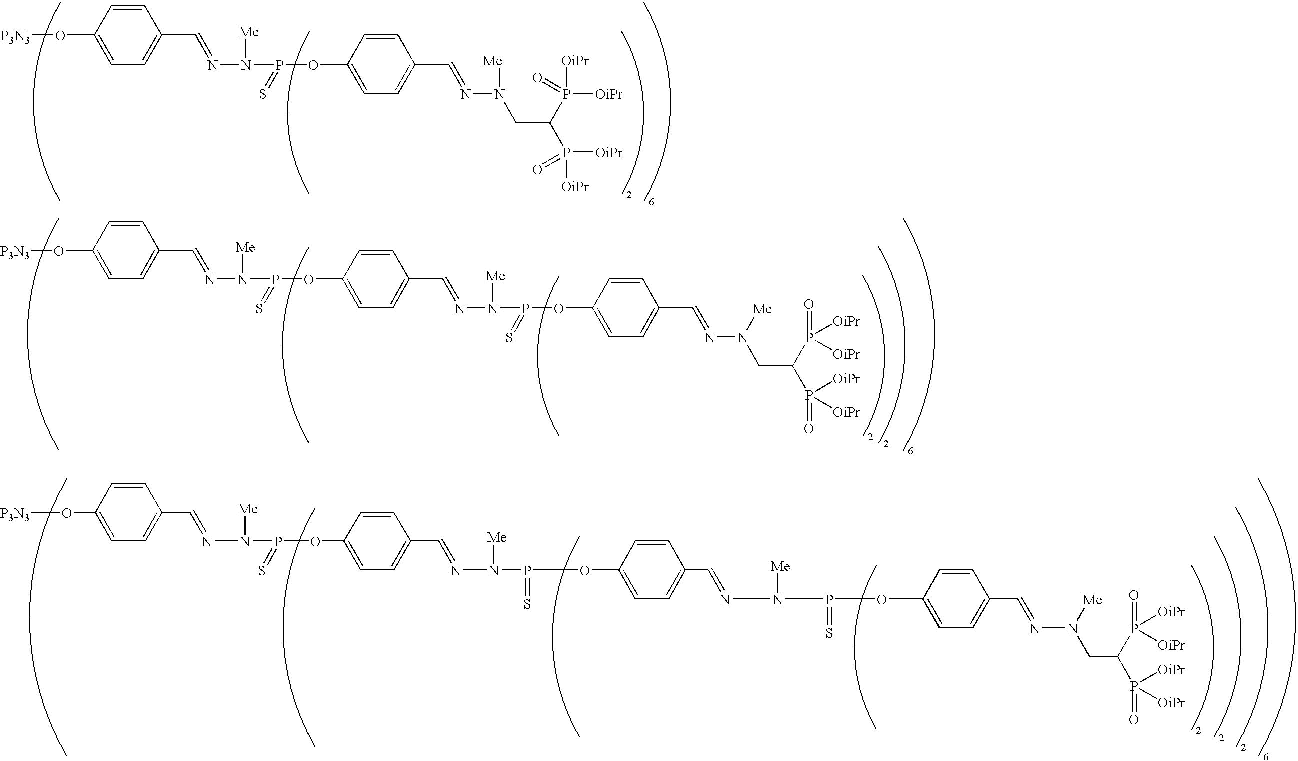

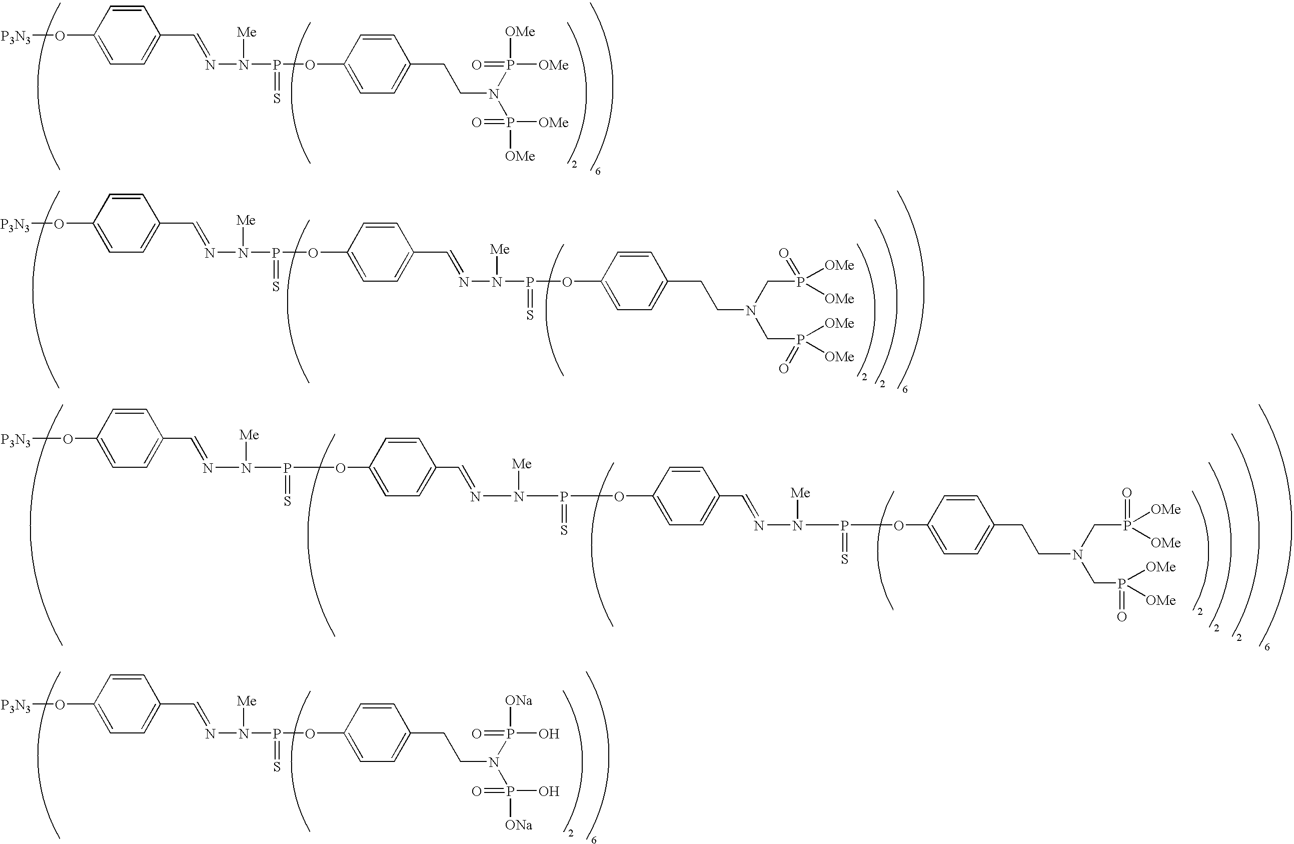

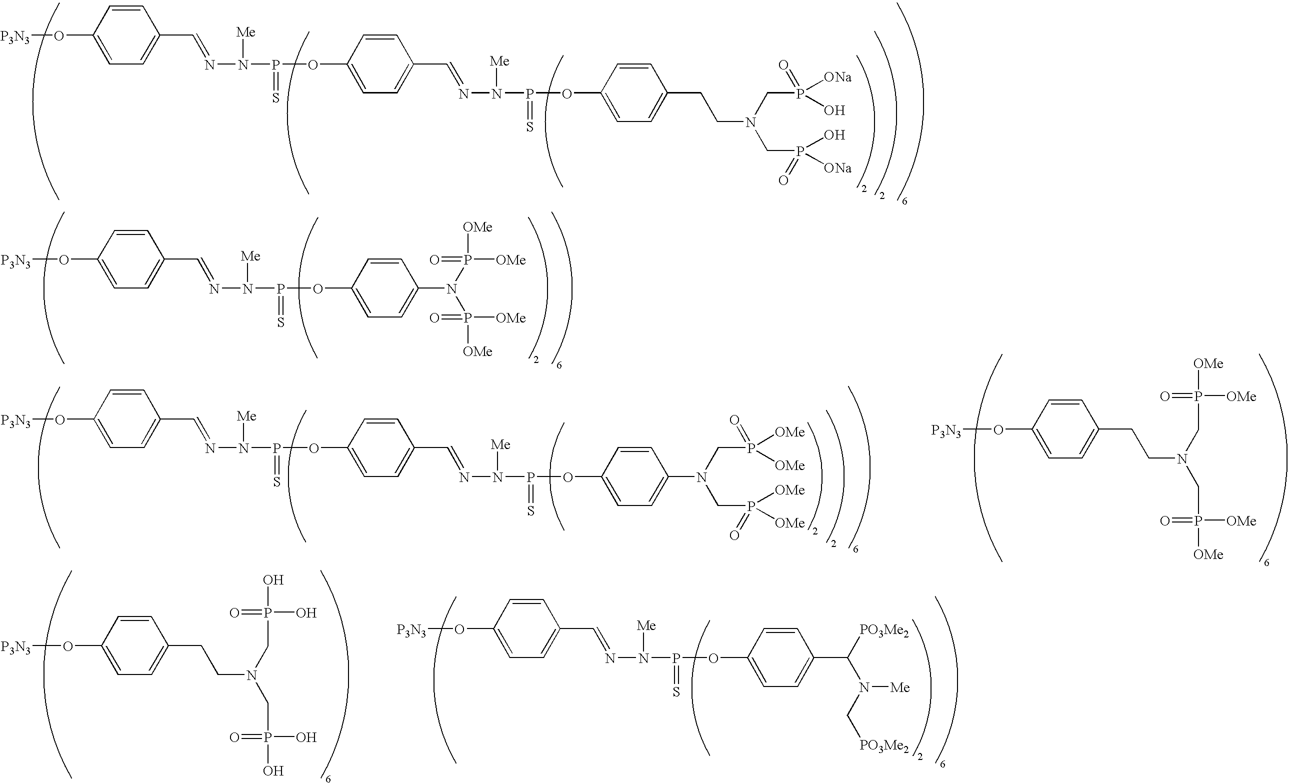

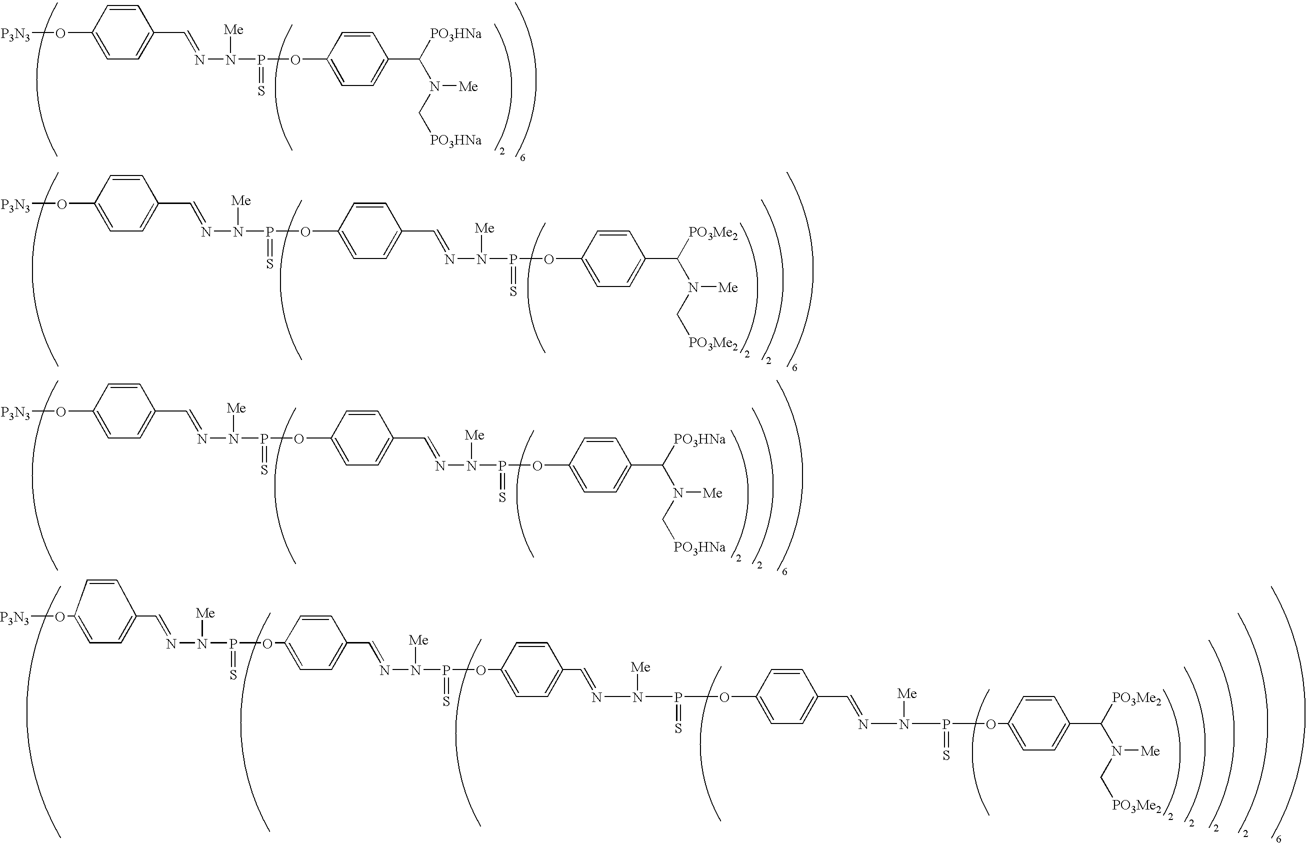

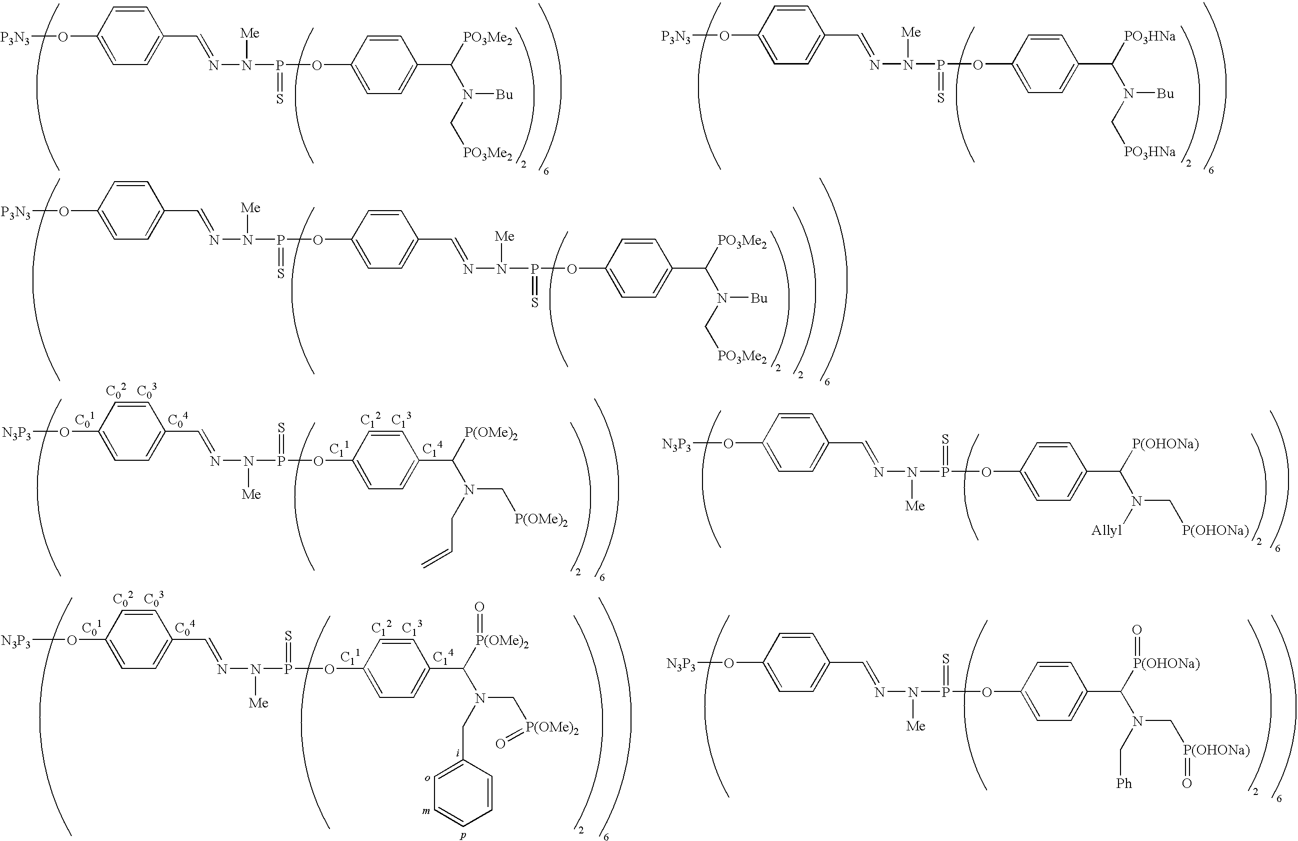

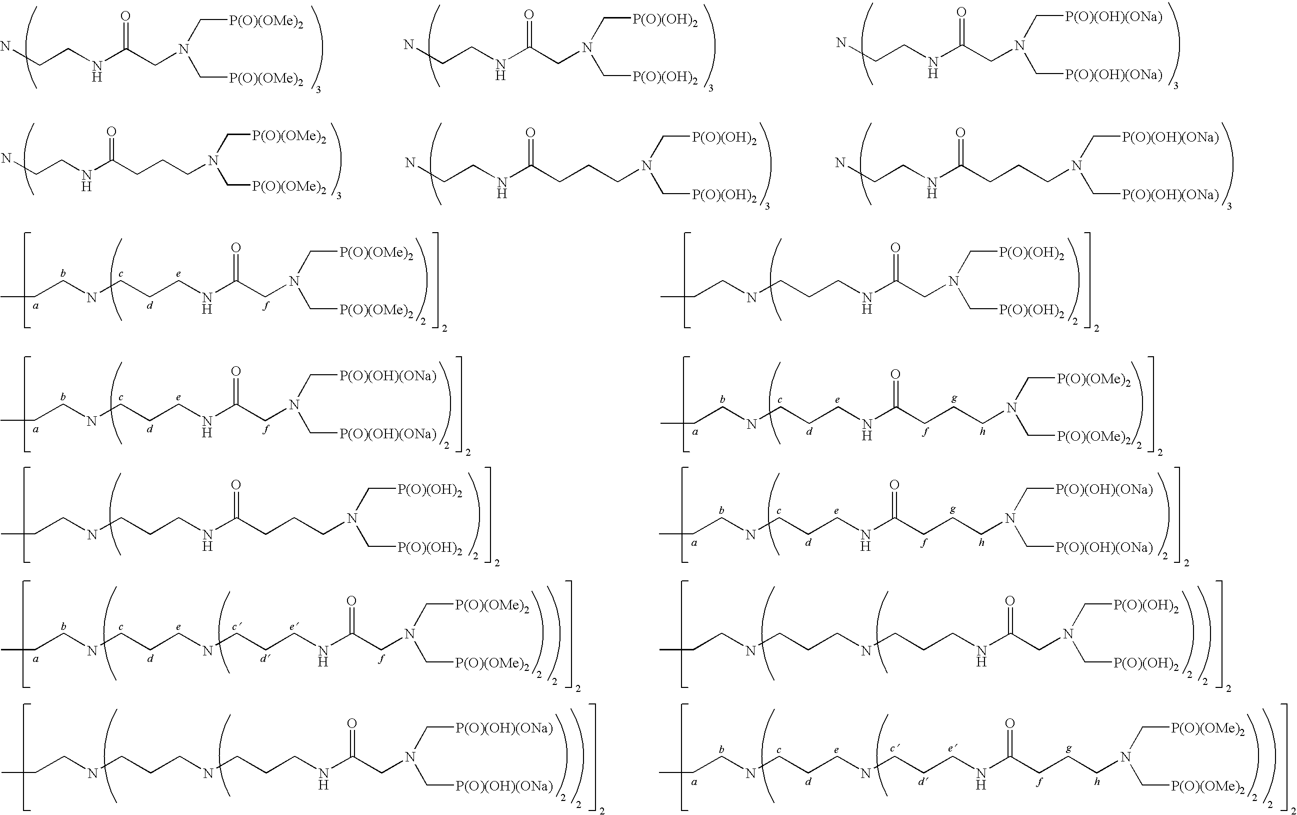

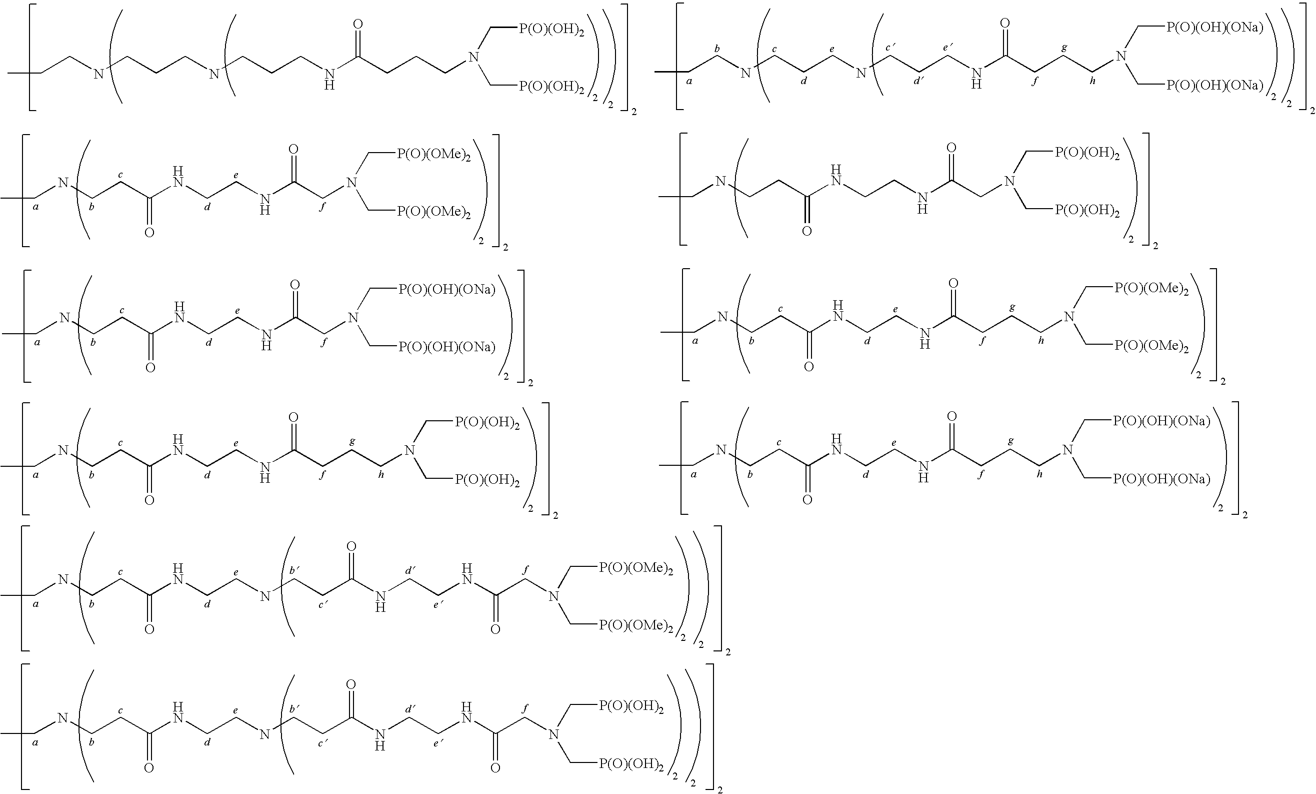

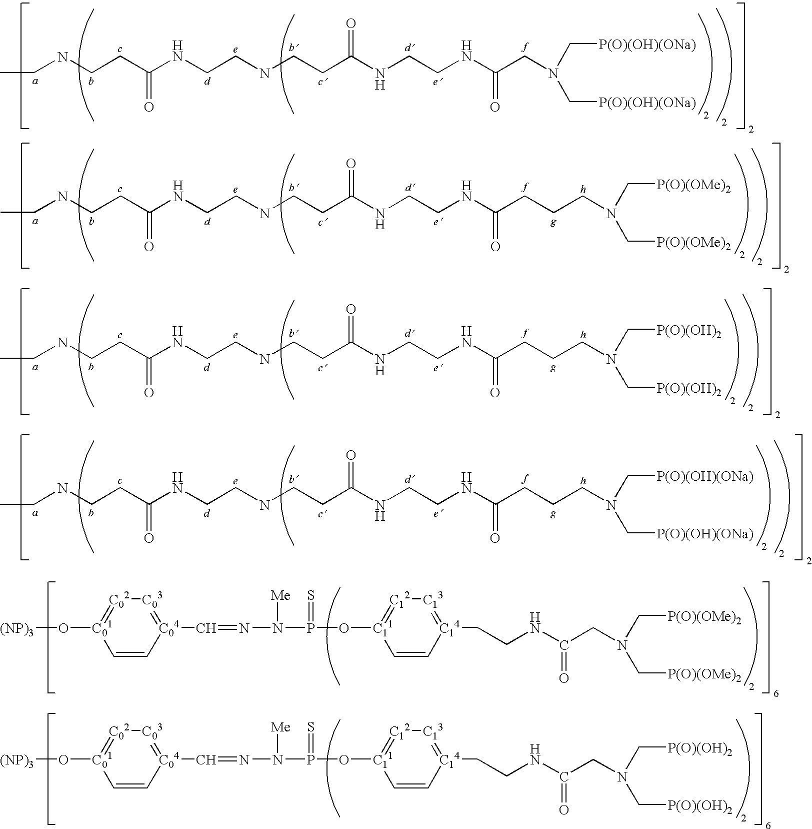

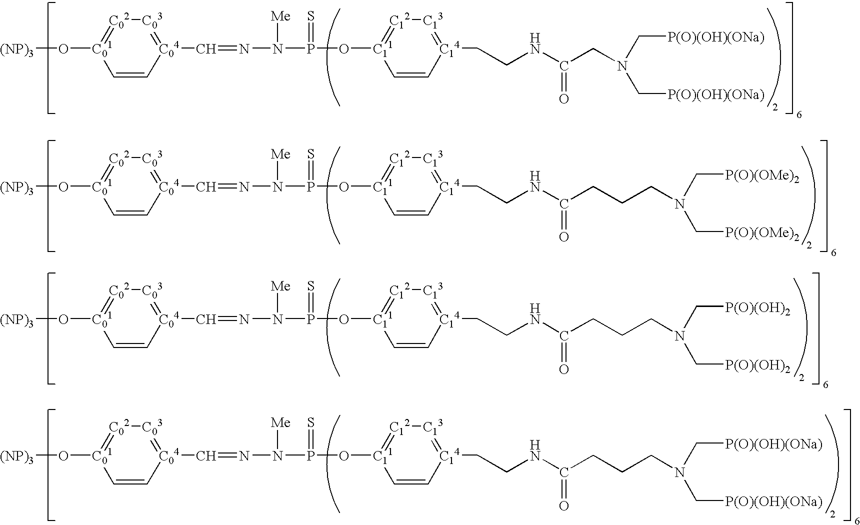

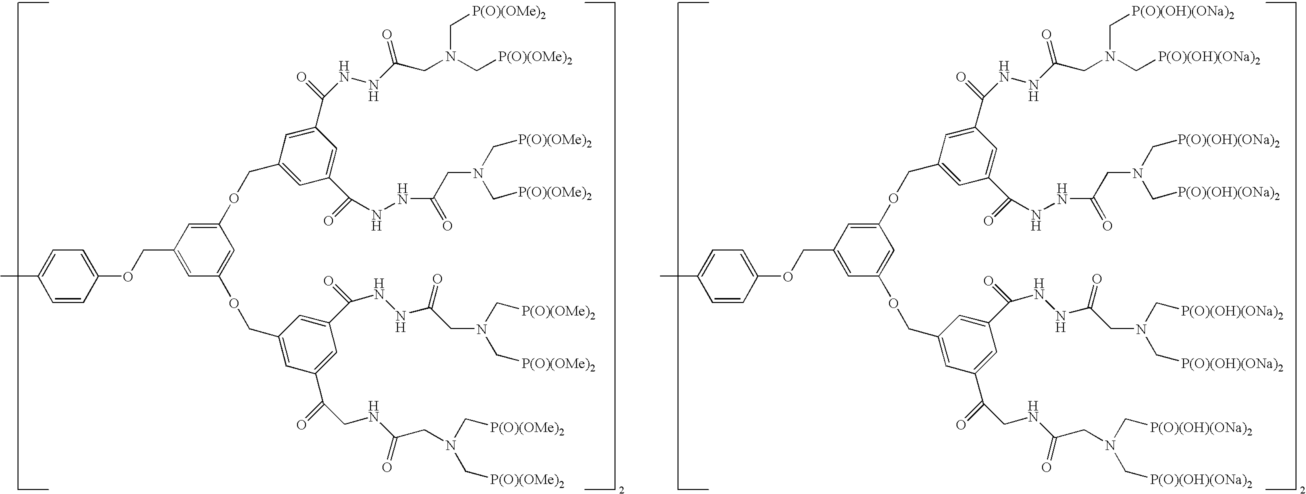

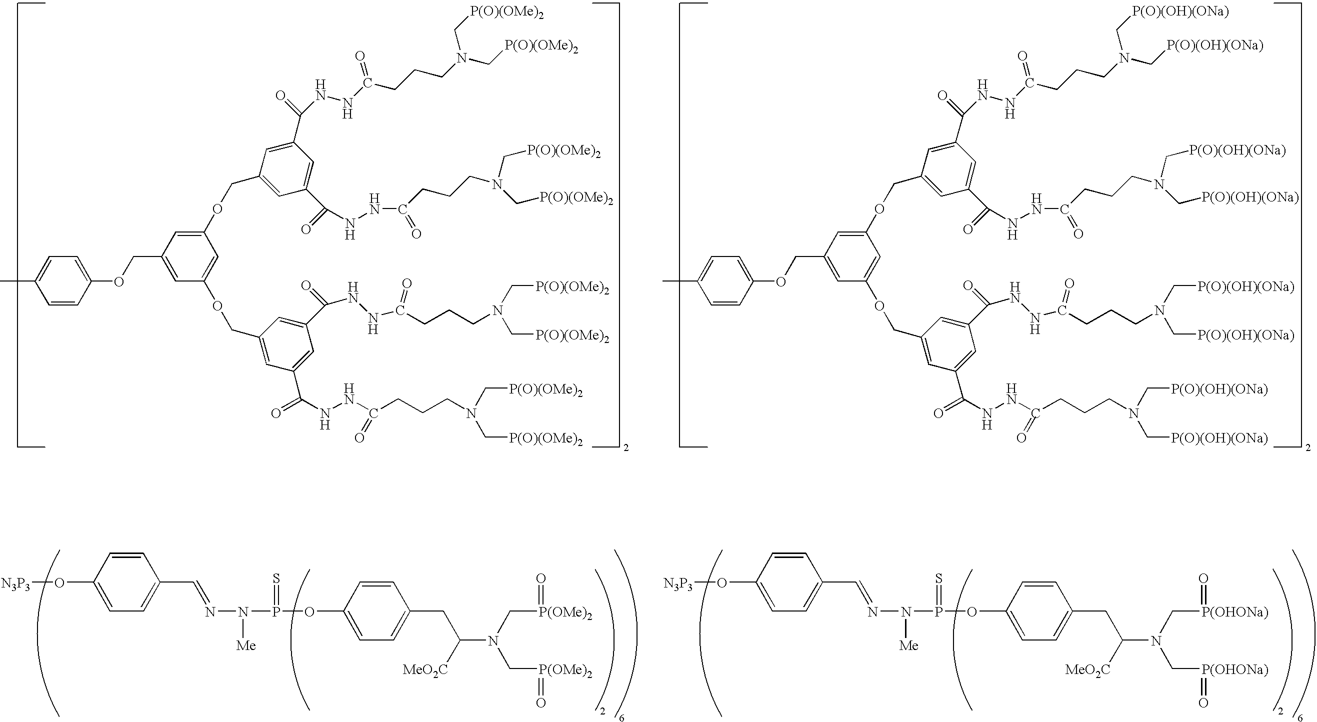

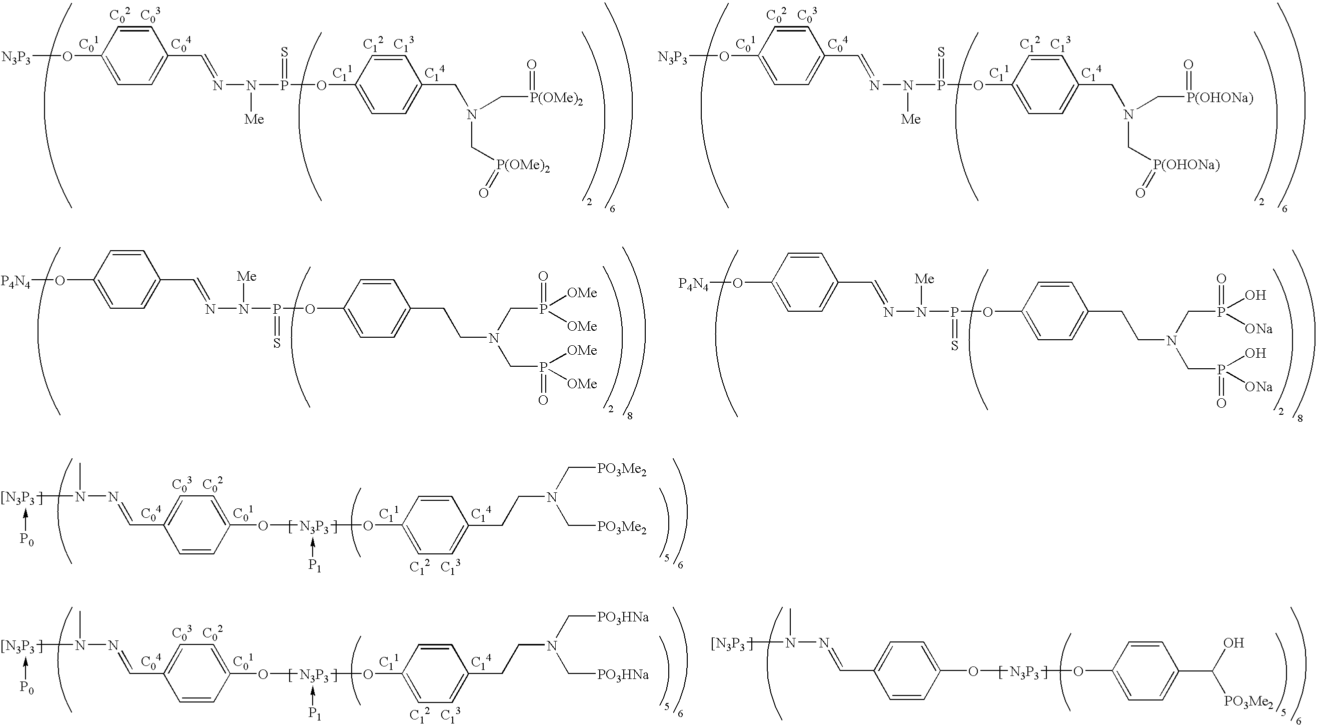

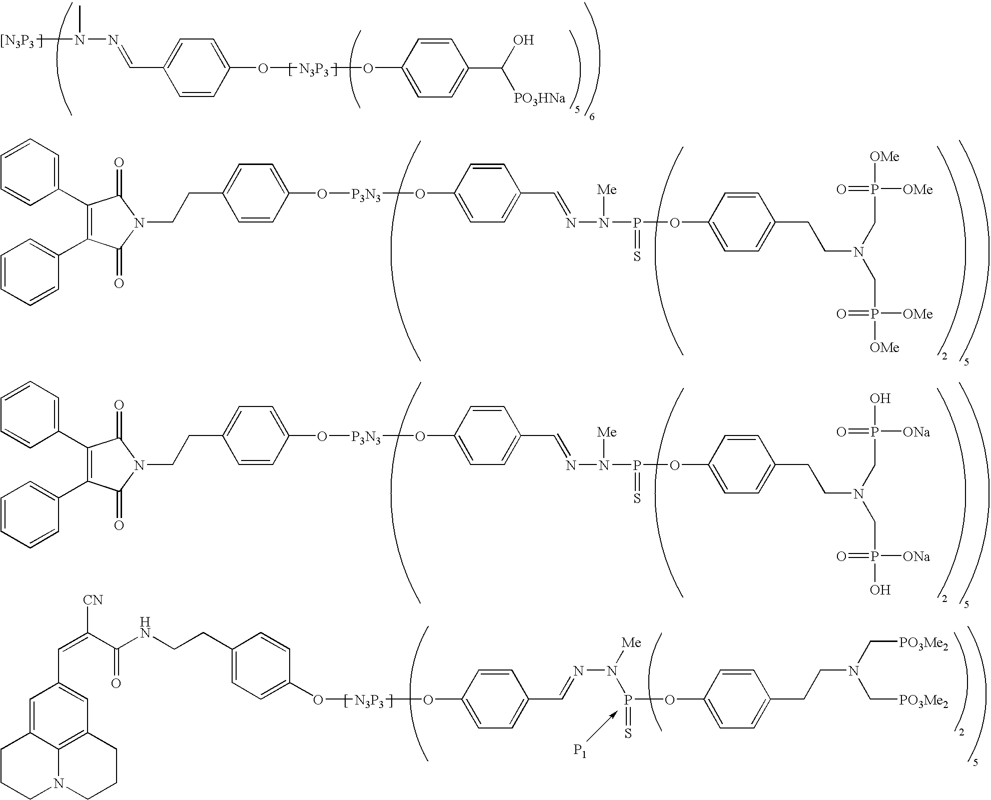

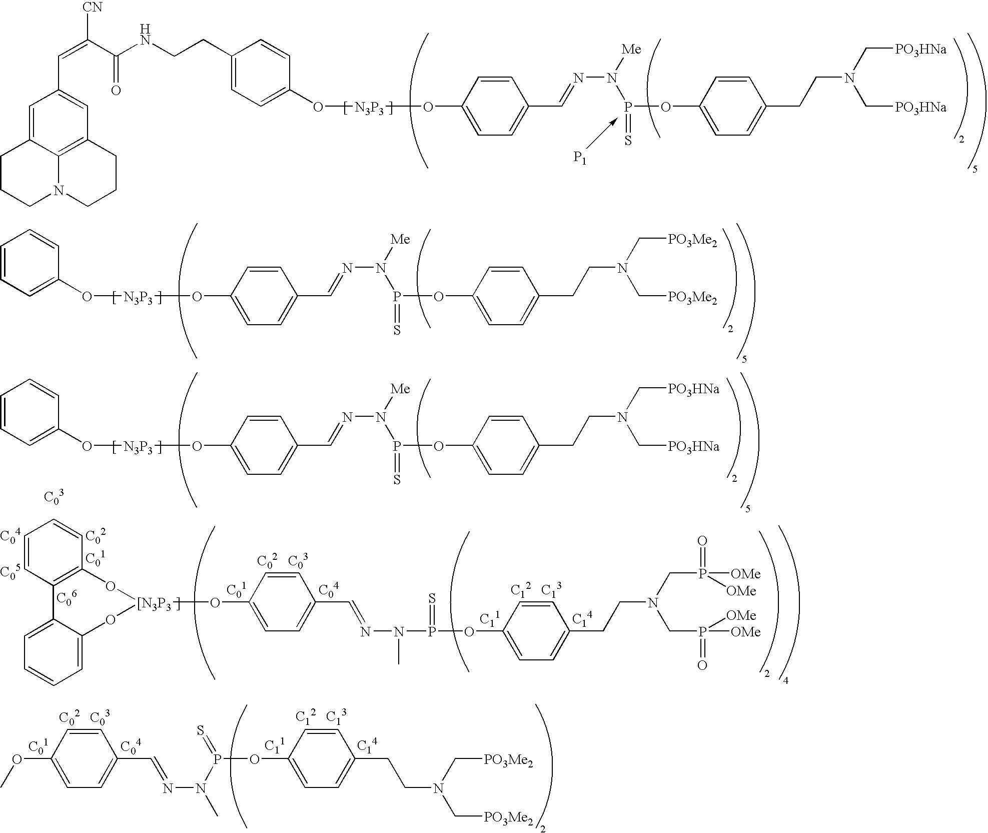

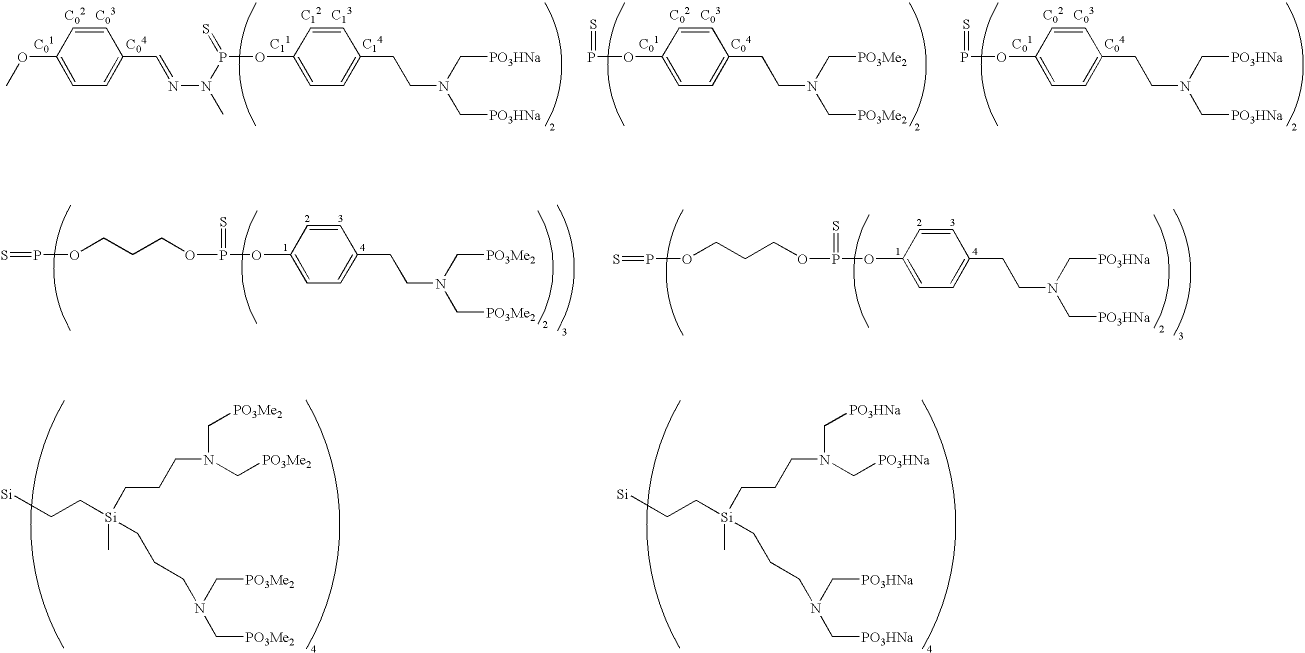

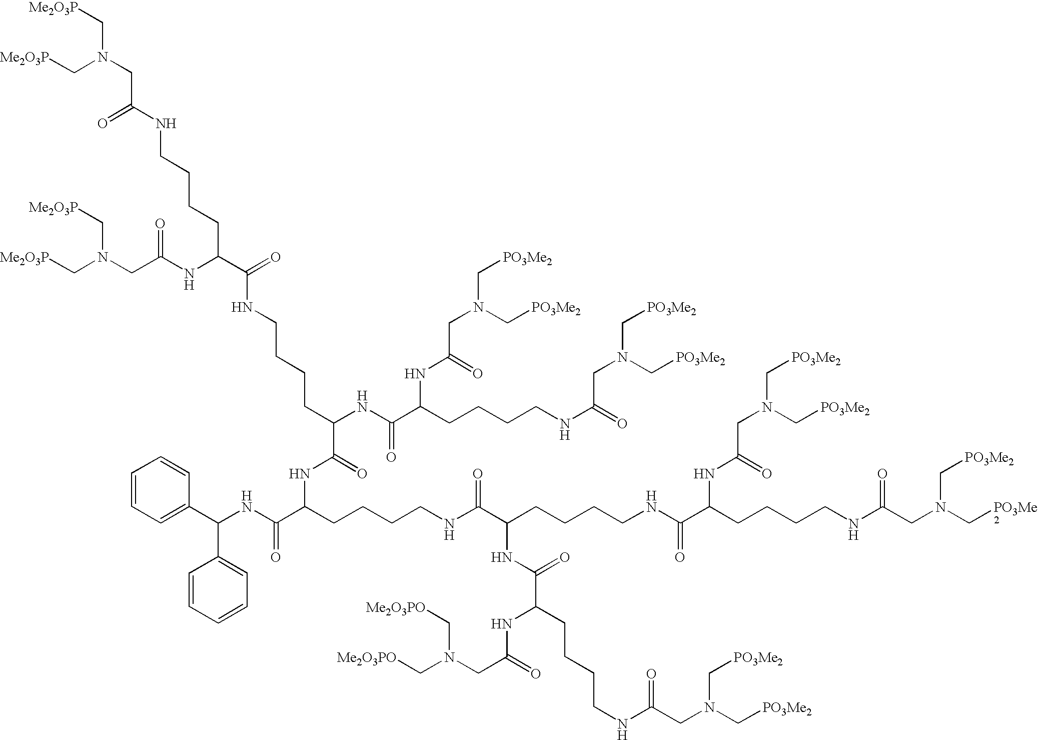

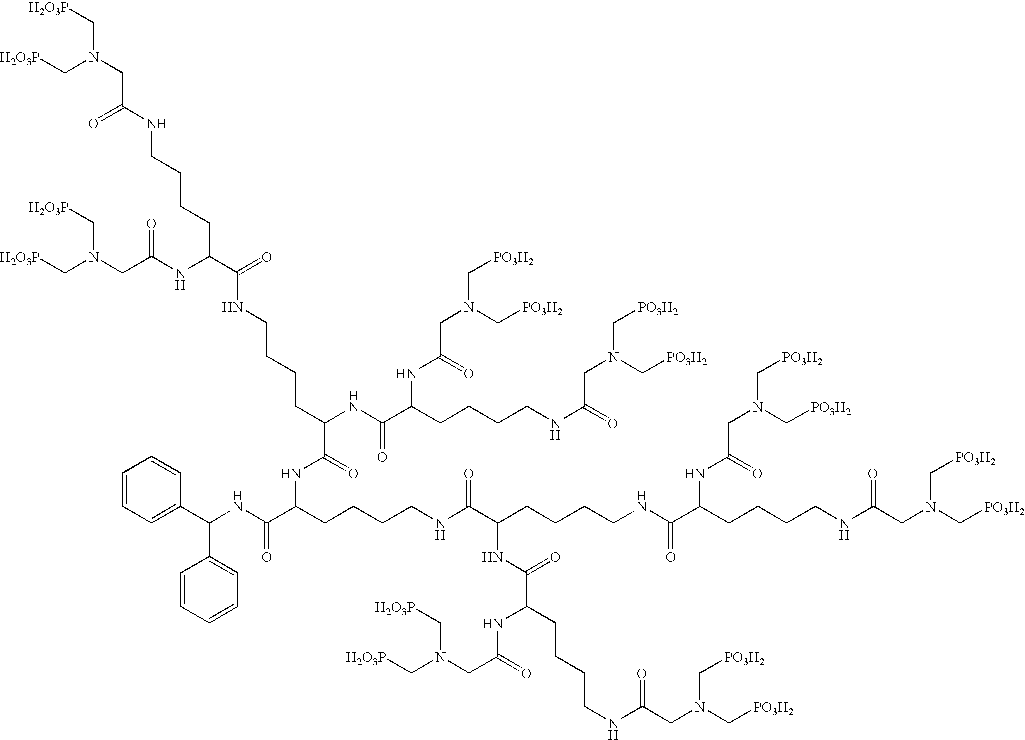

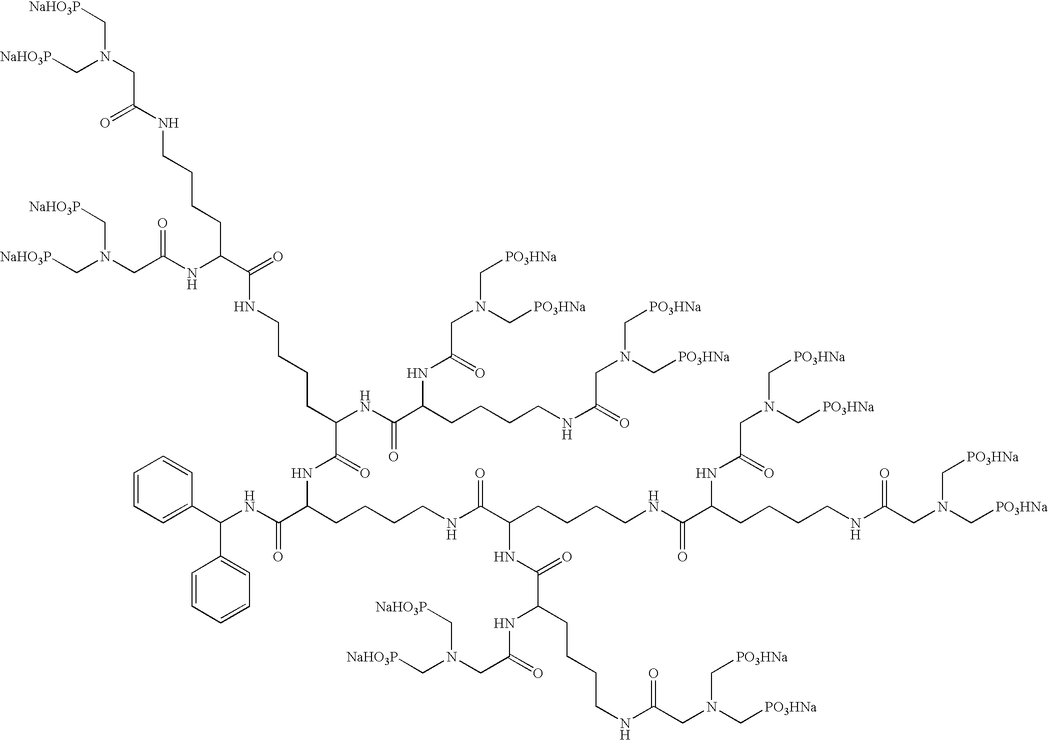

The instant invention also concerns the use of dendrimers with mono- or biphosphonic terminations for the preparation of drugs useful for the treatment of inflammatory diseases, said drugs acting through an anti-inflammatory type activation of monocytes.

The present invention also concerns the use of at least one dendrimer with mono- or biphosphonic terminations for the preparation of drugs useful for the treatment of inflammatory diseases, in particular from auto-immune origin.

The present invention also deals with a method for treating inflammatory diseases through an anti-inflammatory type activation of monocytes, said method comprising the administration to a patient in need thereof of an effective quantity of at least one dendrimer with mono- or biphosphonic terminations.

The present invention also deals with a method for treating inflammatory diseases comprising the administration to a patient in need thereof of an effective quantity of at least one dendrimer with mono- or biphosphonic terminations in association with a pharmaceutical acceptable carrier.

According to the present invention, inflammatory diseases are selected from the group comprising chronic inflammatory diseases, chronic inflammatory diseases of auto-immune origin, pro-inflammatory and inflammatory conditions in case of cancers.

In an advantageous embodiment of the invention, the chronic inflammatory disease is selected from the group comprising rheumatoid arthritis, psoriasis and juvenile idiotypical arthritis.

The present invention also concerns pharmaceutical compositions containing as active substance at least one dendrimer with monophosphonic or bisphosphonic terminations associated with a pharmaceutically acceptable carrier for the treatment of inflammatory diseases, particularly those of auto-immune origin.

In another advantageous embodiment of the instant invention, the dendrimers with monophosphonic or bisphosphonic terminations may be associated with other active substances, in particular with other classical steroid or non-steroïd anti-inflammatory compounds as a combined preparation for simultaneous, separate or sequential use in the treatment of inflammatory diseases.

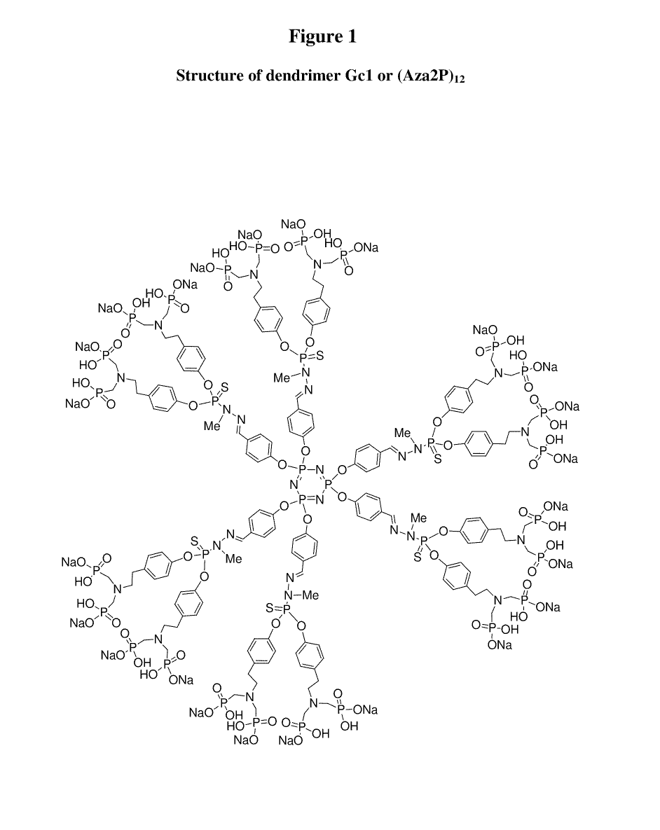

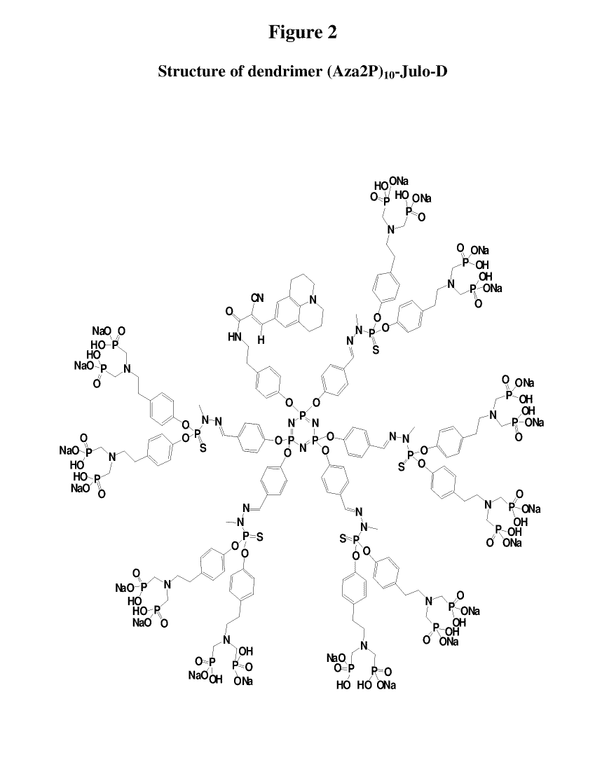

Examples 1 to 9 and FIGS. 1 to 15 which follow illustrate the invention.

FIGS. 1 to 3 show the structure of the bisphosphonic-capped dendrimers tested in examples 5 to 7.

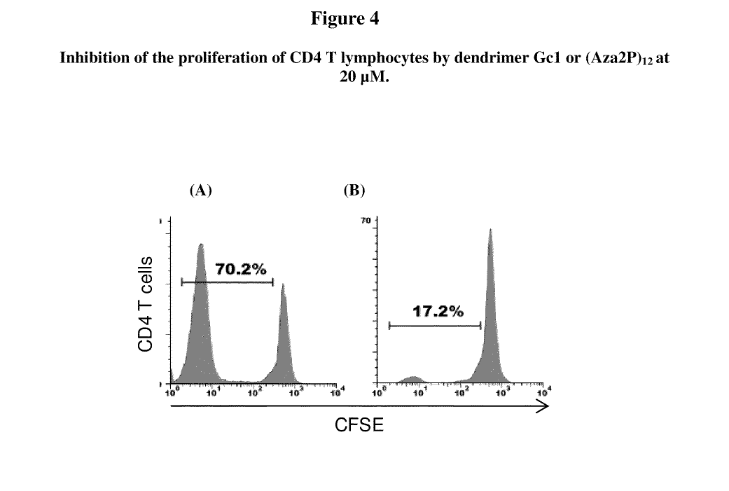

FIG. 4 shows the inhibitory properties of dendrimer Gc1 (or (Aza2P)₁₂) at 20 μM toward the proliferation of CD4 T lymphocytes in PBMC induced by IL2; (A) proliferation with IL2 alone (positive control); (B) proliferation with IL2 and Gc1 at 20 μM.

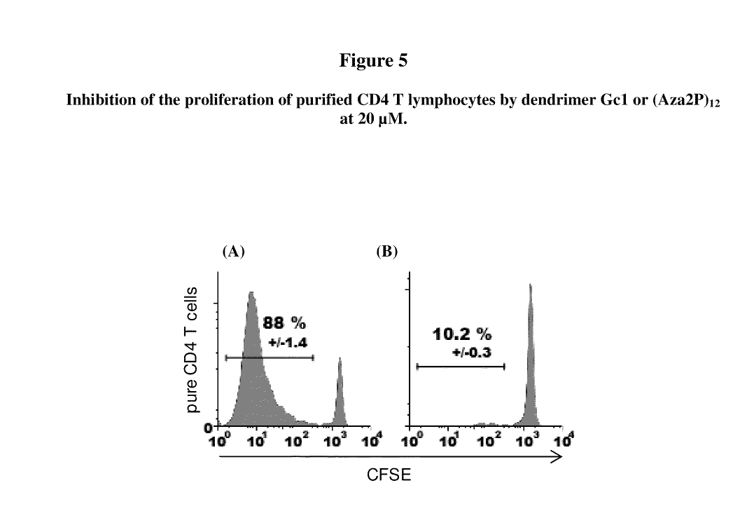

FIG. 5 shows the inhibitory properties of dendrimer Gc1 (or (Aza2P)₁₂) at 20 μM toward the proliferation of purified CD4 T lymphocytes stimulated by anti-CD3/anti-CD28 monoclonal antibodies (mAbs) and IL2; (A) proliferation with mAbs and IL2; (B) proliferation with mAbs and IL2 in presence of Gc1 at 20 μM.

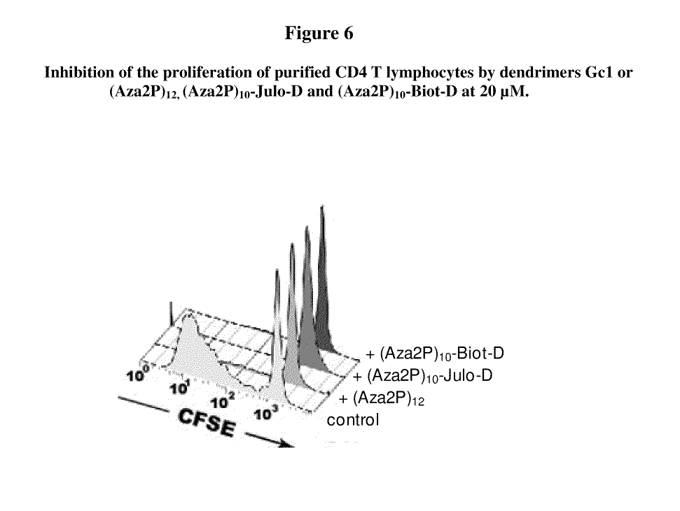

FIG. 6 shows the inhibitory properties of dendrimer Gc1 (or (Aza2P)₁₂) and of its analogs (Aza2P)₁₁-Julo-D and (Aza2P)₁₀-Biot-D at 20 μM toward the proliferation of purified CD4 T lymphocytes stimulated by anti-CD3/anti-CD28 mAbs and IL2.

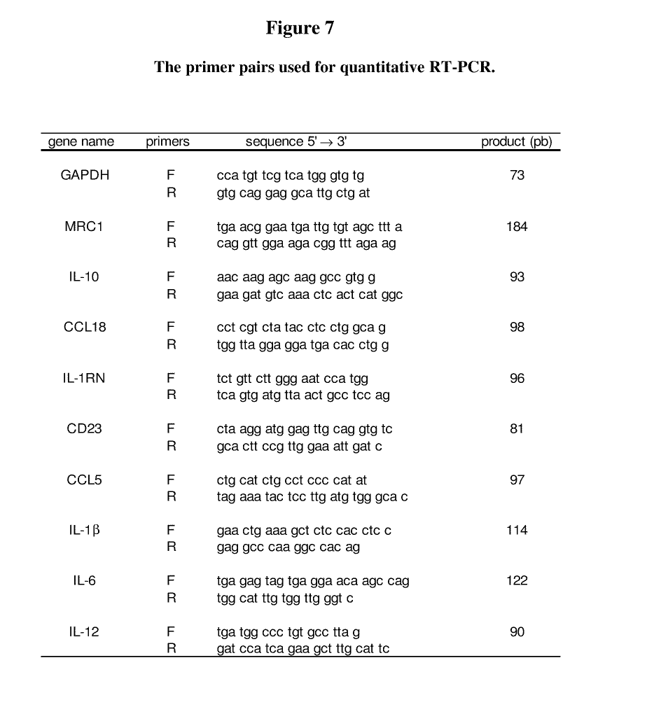

FIG. 7 shows the primer pairs used for quantitative RT-PCR. F: forward primer, R: reverse primer. Products (pb) correspond to the expected RT-PCR product size.

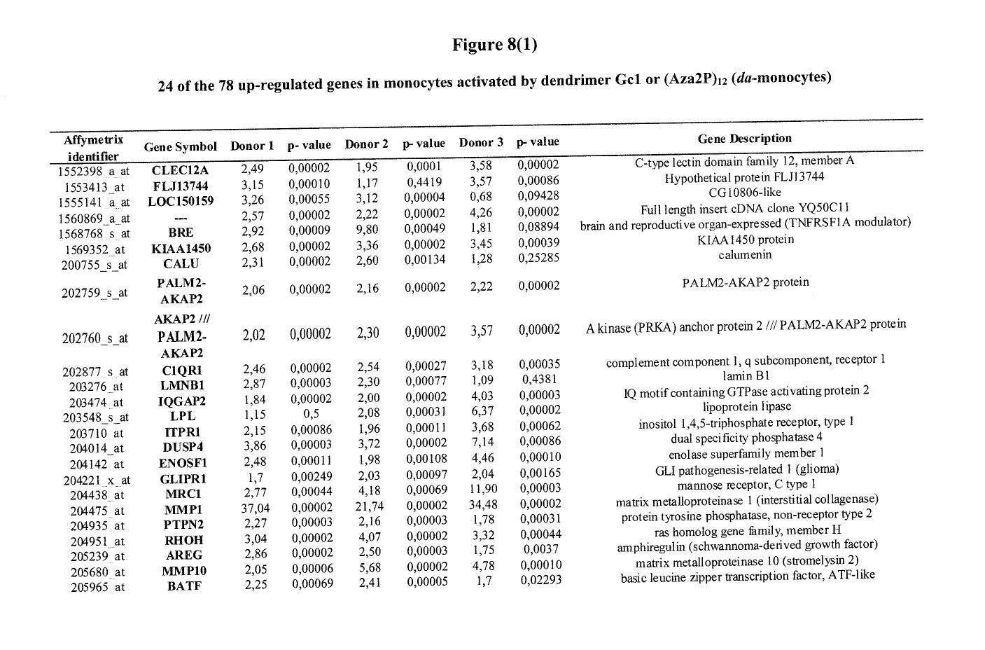

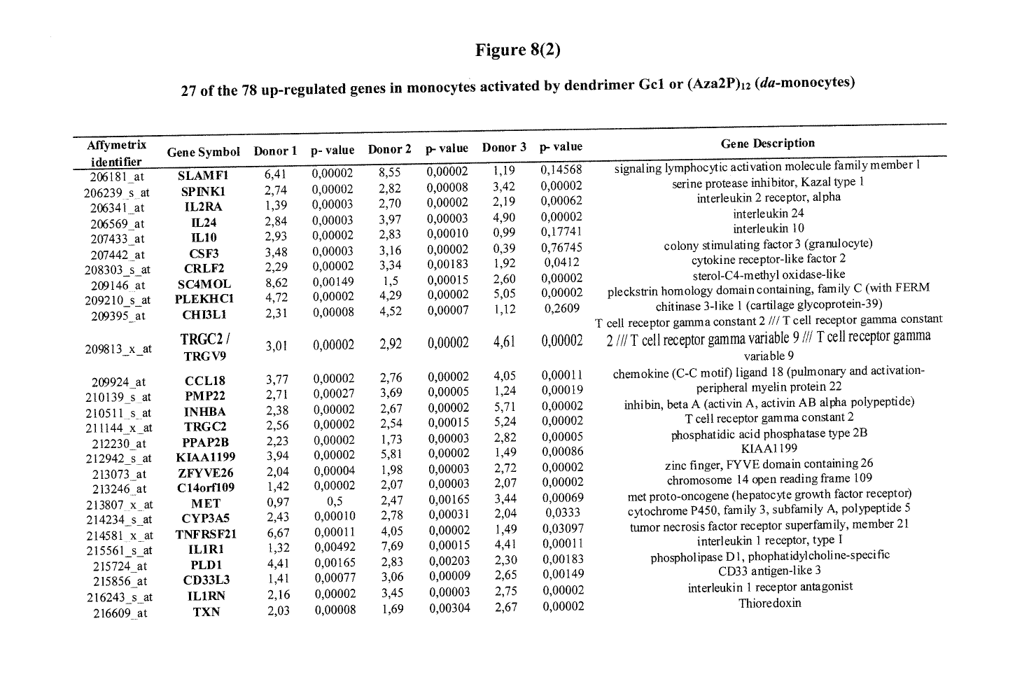

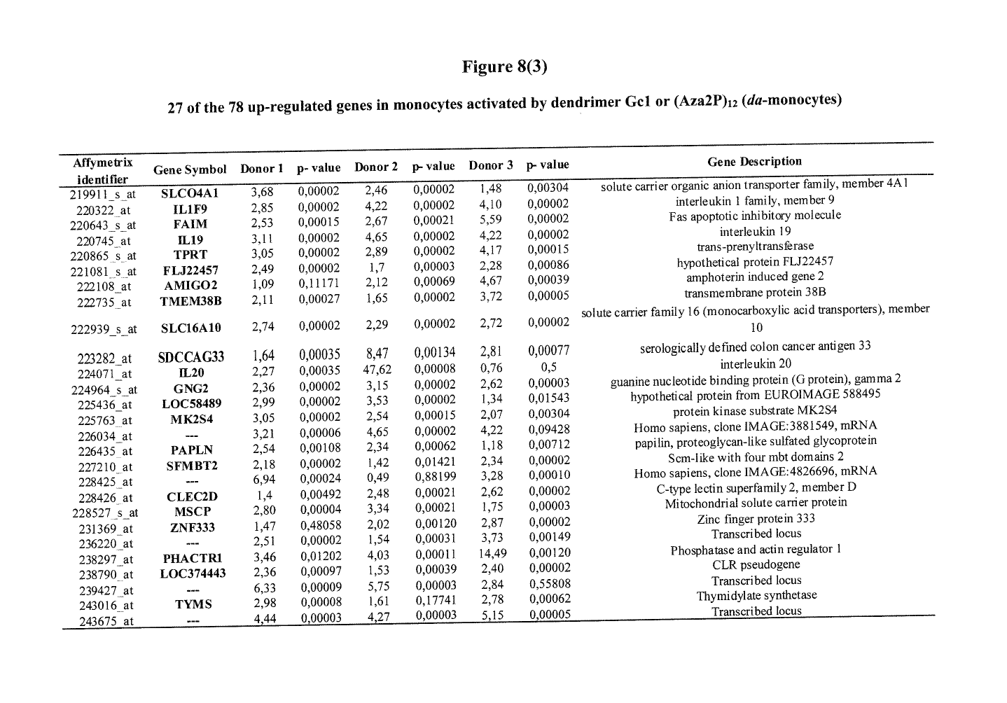

FIGS. 8(1), 8(2) and 8(3) show the 78 up-regulated genes in monocytes activated by dendrimer Gc1 (or (Aza2P)₁₂) (da-monocytes).

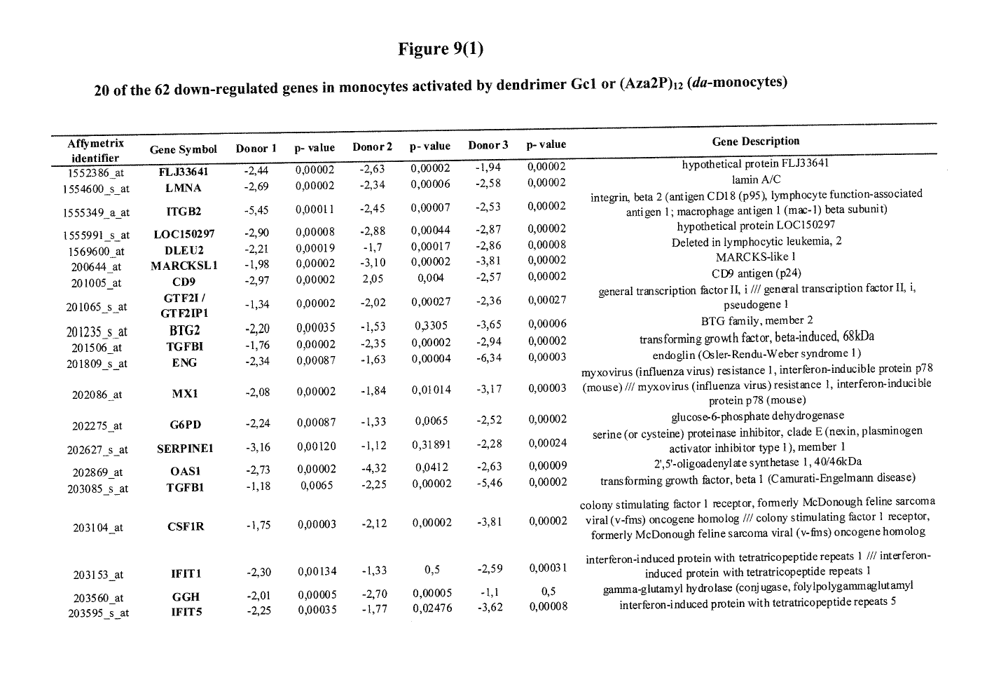

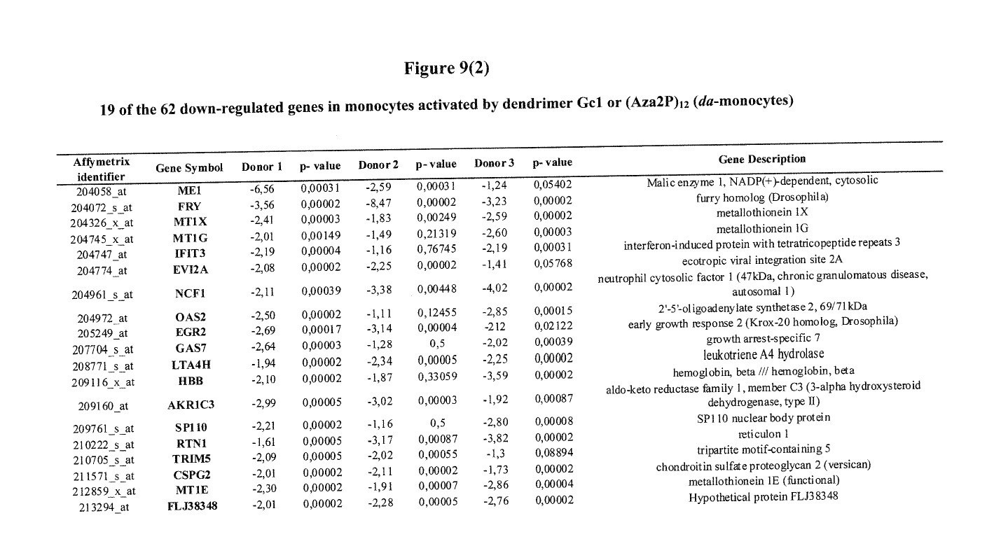

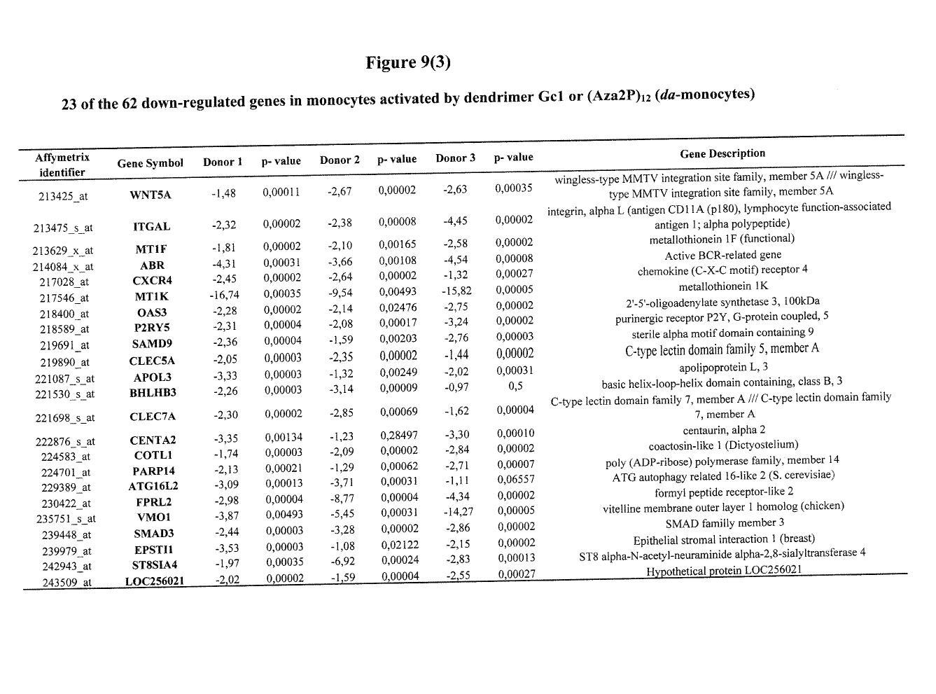

FIGS. 9(1), 9(2) and 9(3) show the 62 down-regulated genes in da-monocytes.

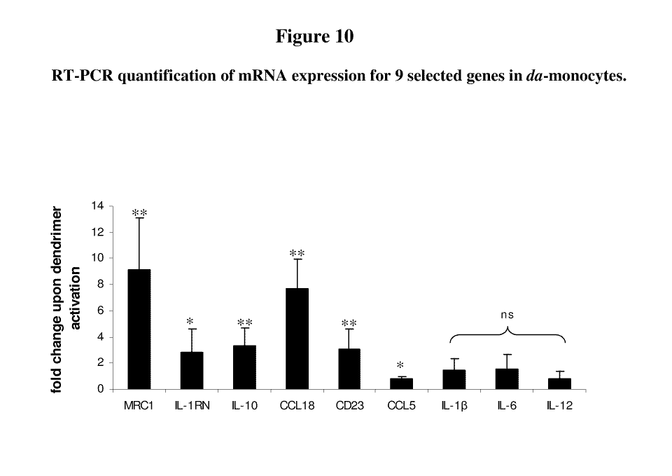

FIG. 10 shows the RT-PCR quantification of mRNA expression for 9 selected genes in da-monocytes. Expression levels are normalized to the GAPDH mRNA. Relative expressions are calculated using the AACt method and results represent the relative fold change induced by the dendrimer in comparison to non-stimulated monocytes. Data are expressed as mean±SD from 6 independent experiments. *p<0.05, **p<0.005, one tailed t-test.

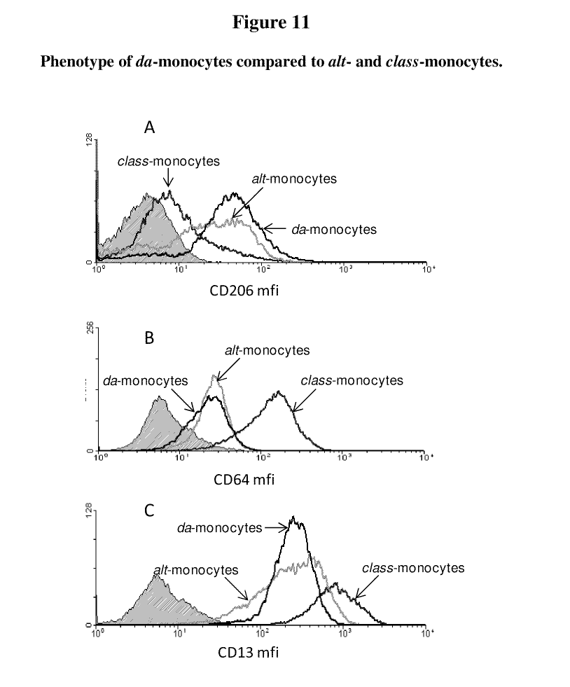

FIG. 11 shows the phenotype of da-monocytes compared to alt- and class-monocytes. Cell surface expression levels for (A) CD206 (the mannose receptor MRC1), (B) CD64 (a F_{c}γRI) and (C) CD13 (the aminopeptidase N) are analyzed by flow cytometry. Mfi detected on class-monocytes labelled by isotype-matched mAbs are represented in grey as negative control. Data shown are representative of 3 independent experiments.

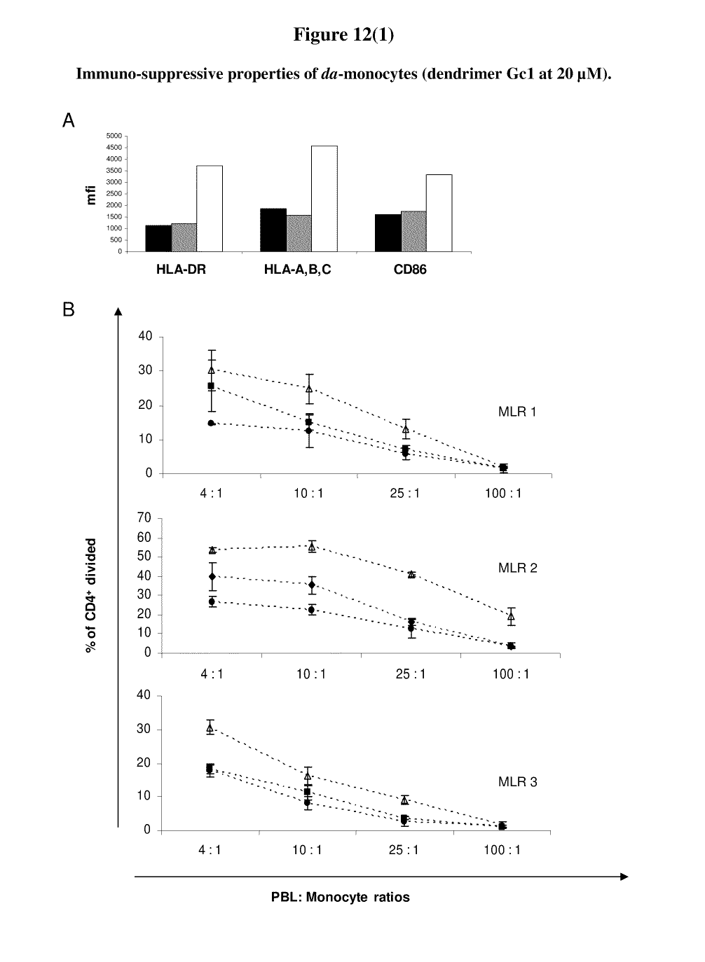

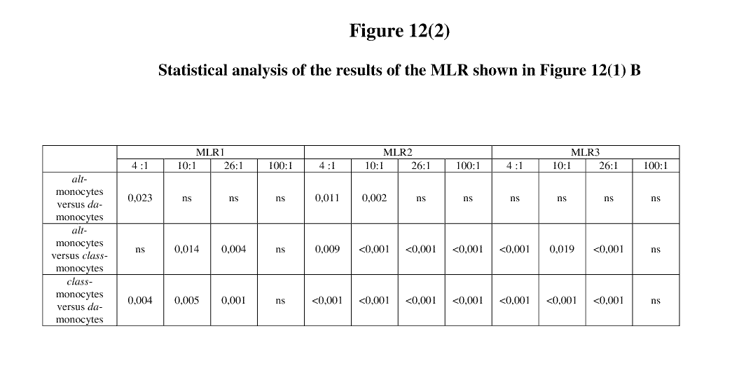

FIGS. 12(1) and 12(2) show the immuno-suppressive properties of da-monocytes (dendrimer Gc1 at 20 μM). FIG. 12(1) A shows flow cytometry analysis (showed as mfi) of the cell surface expression levels of antigen-presenting molecules (HLA-DR and HLA-A,B,C) and the co-stimulatory molecule CD86 on da- (black bars), alt- (grey bars) and class-monocytes (white bars). Data shown are representative of 3 independent experiments. In FIG. 12(1) B, CD4⁺ T lymphocytes are gated to quantify CFSE dilution by flow cytometry after MLR. For the different PBL:monocyte (•da-monocytes, □alt-monocytes and Δclass-monocytes) ratios, the percentages of divided CD4⁺ T cells represent cells having undergone at least one division. Each point is the mean of percentages of divided CD4⁺ T cells±SD from triplicates. The results for 3 different MLR are shown. FIG. 12(2) gives the statistical analysis of the results shown in FIG. 12(1)B.

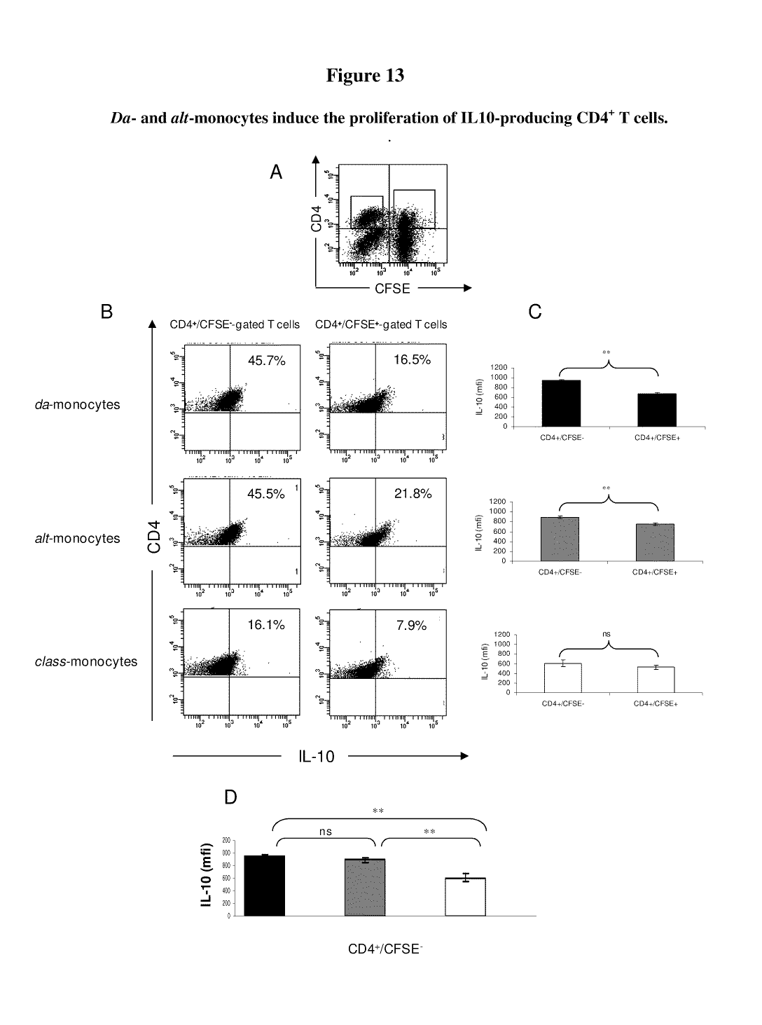

FIG. 13 shows that da- and alt-monocytes, but not class-monocytes, induce the proliferation of IL10-producing CD4⁺ T cells. A) After MLR and a 5 hour re-stimulation, divided CD4⁺/CFSE⁻ T lymphocytes and non-divided CD4⁺/CFSE⁻ T lymphocytes are gated to quantify intracellular IL10 by flow cytometry. B) Flow cytometry analysis of intracellular IL10 (% of IL10⁺ CD4⁺ T cells) in divided (CD4⁺/CFSE⁻-gated, left column) and non-divided (CD4⁺/CFSE⁺-gated, right column) T cells. Presented results are representative of 3 independent MLR. C) Comparison (mean of mfi±SD from triplicates) of intracellular IL10 in divided and non-divided CD4⁺ T cells in MLR with da- (upper black bars), alt- (middle grey bars) and class-monocytes (lower white bars). **p<0.005, Student's t-test. D) Comparison (mean of mfi±SD from triplicates) of intracellular IL10 in divided CD4⁺ T cells in MLR with da- (left black bar), alt- (middle grey bar) and class-monocytes (right white bar). **p<0.001, one-way ANOVA. In flow cytometry dot plots, all quadrants are set using appropriate isotype controls.

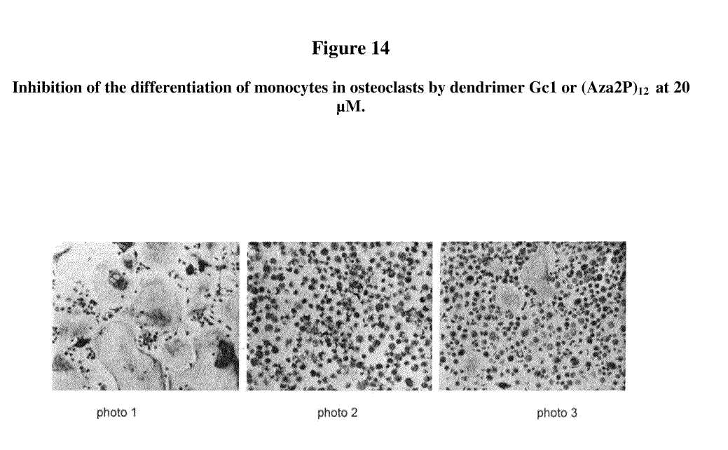

FIG. 14 shows the inhibition of the differentiation of human monocytes in osteoclasts by dendrimer Gc1 or (Aza2P)₁₂ at 20 μM, as in example 3. Picture 1: differentiation in control medium (without dendrimer Gc1); picture 2: differentiation in the presence of dendrimer Gc1 or (Aza2P)₁₂ at 20 μM; picture 3: differentiation after pre-incubation (6 hours) with dendrimer Gc1 or (Aza2P)₁₂ at 20 μM.

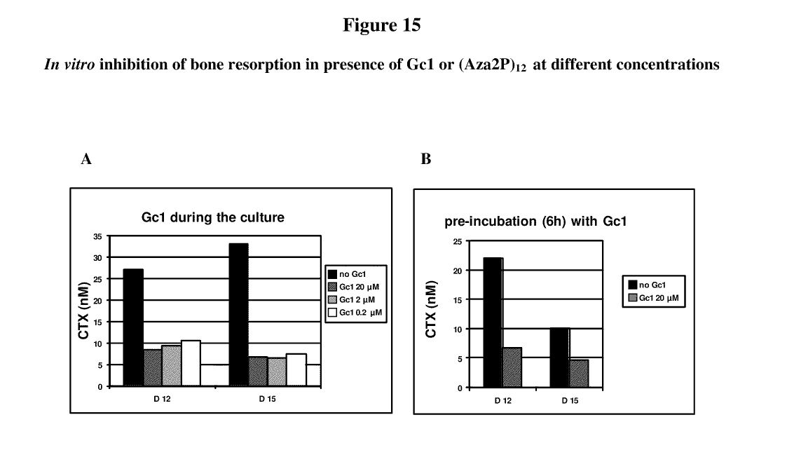

FIG. 15 shows the in vitro inhibition of bone resorption in presence of dendrimer Gc1 or (Aza2P)₁₂ at different concentrations as explained in example 4; A) Gc1 or (Aza2P)₁₂ added in the culture medium at different concentrations; B) after pre-incubation (6 hours) in Gc1 or (Aza2P)₁₂ at 20 μM.

Examples 1 to 4 illustrate the synthesis of some dendrimers.

### General Points

The reactions were carried out under a dry argon atmosphere (argon U, Air Liquide).

The following solvents were dried and distilled under argon immediately before use according to the techniques described by Perrin et al, Purification of Laboratory Chemicals, Third Edition; Press, P., Ed.: Oxford, 1988: tetrahydrofuran, dichloromethane, acetonitrile, pentane, toluene, diethyl ether, chloroform, triethylamine, pyridine.

Thin layer chromatography analyses were carried out on aluminium plates coated with silica of the Merck Kieselgel 60F₂₅₄ type.

The NMR spectra were recorded on Brüker devices (AC200, AM250, DPX 300). The chemical shifts are expressed in parts per million (ppm) relative to phosphoric acid at 85% in water for the ³¹P NMR and relative to tetramethylsilane for the ¹H and ¹³C NMR. The following abbreviations were used in order to express the multiplicity of signals: s (singlet), d (doublet), bd (broad doublet), dd (doublet of doublets), AB syst. (AB system), t (triplet), dt (doublet of triplets), q (quadruplet), hept (heptuplet), m (unresolved multiplet).

Infrared vibrational spectroscopy was carried out on a Perkin Elmer FT 1725× spectrometer. The UV-visible spectroscopy was carried out on an HP 4852A device. The thermogravimetric measurements were carried out on a Netzch DSC 204 or Setaram TGA 92-16.18 device.

### Numbering Used for the NMR Attribution:

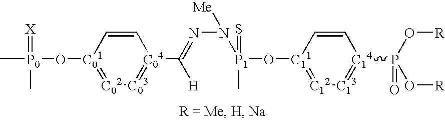

### Example of Numbering for a First-Generation Dendrimer

### EXAMPLE 1

### Synthesis of a Dendrimer of Polyether Type of First Generation with Azabis Phosphonic Acid Ends Derived from Tyramine

### 1.1. Synthesis of Phenol aza-bis-dimethyl-phosphonate Derived from Tyramine

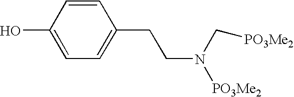

Tyramine (6 g, 43.7 mmol) and dimethyl-phosphite (10.32 ml, 112.5 mmol) are mixed at 0°, then a 37% solution of formaldehyde in water (12.6 ml) is slowly added, still at 0° C. The mixture is taken to ambient temperature for 30 minutes and refluxed for 1 hour with magnetic stirring. Finally the crude reaction product is placed under reduced pressure in order to evaporate the excess of formaldehyde. The product is extracted with a chloroform/water mixture (4/1) (3×100 ml of chloroform). The organic phase is recovered then subjected to chromatography on silica using acetone as eluent. The final product is isolated with a yield of 65%.

³¹P-{¹H} NMR (CDCl₃): δ=30.2 (s, P(O)(OMe)₂) ppm.

¹H NMR (CDCl₃): δ=2.68 (deformed t, ³J_{HH}=7.2 Hz, 2H, —CH₂—CH₂—N); 3.05 (deformed t, ³J_{HH}=7.2 Hz, 2H, —CH₂—CH₂—N—); 3.20 (d, ²J_{HP}=8.9 Hz, 4H, N—CH₂—P); 3.75 (d, ³J_{HP}=10.7 Hz, 12H, —OMe); 6.6-7.1 (m, 4H, CHₐᵣₒₘ); 8.16 (broad s, 1H, —OH) ppm.

¹³C-{¹H} NMR (CDCl₃): δ=32.7 (s, C₅); 49.4 (dd, ³J_{CP}=6.8 Hz, ¹J_{CP}=158.5 Hz, C₇); 52.8 (d, ²J_{CP}=3 Hz, C₈); 58.8 (t, ³J_{CP}=7.5 Hz, C₆); 115.4 (s, C₃); 129.8 (s, C₂); 129.8 (s, C₄); 155.9 (s, C₁) ppm.

### 1.2. Synthesis of a Dendrimer with Azabis Phosphonate Terminations Derived from Tyramine

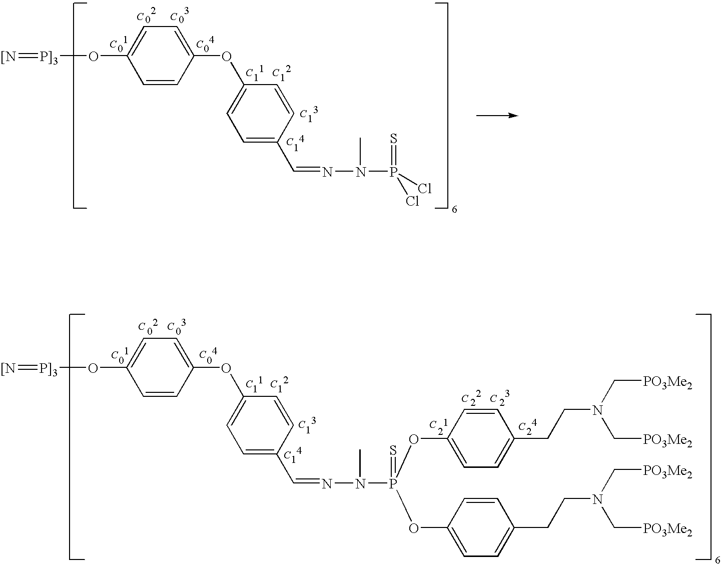

Tyramine aza-bisphosphonate of Example 1.1 (388 mg, 1.020 mmol) and caesium carbonate (565 mg, 1.734 mmol) are added to a solution of a dendrimer of polyether type of first generation with PSCl₂ terminations (177 mg, 0.074 mmol) in a mixture of aceton/THF (10 mL/3 mL). The reaction mixture is stirred at ambient temperature during 12 h, centrifuged and the resulting clear solution is evaporated to dryness under reduced pressure.

The obtained oil is purified by chromatography on silica gel (gradient acetone/triethylamine (100:0 à 90:10), Rf=0.97 in aceton/triethylamine (90:10)) to afford the dendrimer with dimethylphosphonate ends as a pale yellow solid (yield: 91%).

³¹P-{¹H} NMR (CDCl₃, 121.5 MHz): δ=9.27 (s, N₃P₃); 26.78 (s, PO₃Me₂); 63.18 (s, P₁); ¹H NMR (CDCl₃, 300.13 MHz): δ=2.74 (t, ³J_{HH}=7.2 Hz, 24H, CH₂—CH₂—N); 3.05 (t, ³J_{HH}=7.2 Hz, 24H, CH₂—CH₂—N); 3.17 (d, ²J_{HP}=9.3 Hz, 48H, N—CH₂—P); 3.29 (d, ³J_{HP}=10.2 Hz, 18H, CH₃—N—P₁); 3.71 (dd, ³J_{HP}=10.5 Hz, ⁷J_{HP}=1.5 Hz, 144H, OMe); 6.93 (m, 12H, C₀³—H); 6.94 (m, 12H, C₁²—H); 7.00 (m, 12H, C₀²—H); 7.10 (m, 24H, C₂²—H); 7.16 (m, 24H, C₂³—H); 7.60 (br s, 6H, CH═N); 7.67 (m, 12H, C₁³—H); ¹³C-{¹H} NMR (CDCl₃, 75.6 MHz): δ=32.87 (br s, CH₃—N—P₁); 33.04 (s, CH₂—CH₂—N); 49.48 (dd, ¹J_{CP}=157.5 Hz, ³J_{CP}=7.3 Hz, N—CH₂—P); 52.62 (d, ²J_{CP}=3.4 Hz, OMe); 52.66 (d, ²J_{CP}=3.4 Hz, OMe); 58.08 (t, ³J_{CP}=7.5 Hz, CH₂—CH₂—N); 118.50 (s, C₁²); 120.40 (s, C₀³); 121.27 (d, ³J_{CP}=4.5 Hz, C₂²); 122.23 (s, C₀²); 128.61 (s, C₁³); 129.87 (s, C₂³); 130.21 (s, C₁⁴); 136.47 (d, ⁵J_{CP}=1.8 Hz, C₂⁴); 138.89 (d, ³J_{CP}=13.8 Hz, CH═N); 146.37 (td, ²J_{CP}=5.2 Hz, ⁴J_{CP}=2.5 Hz, C₀¹); 148.96 (d, ²J_{CP}=7.0 Hz, C₂¹); 153.58 (s, C₀⁴); 158.32 (s, C₁¹) ppm.

### 1.3. Synthesis of the Dendrimer with Sodium Salt of Azabisphosphonic Acid Ends Derived from Tyramine

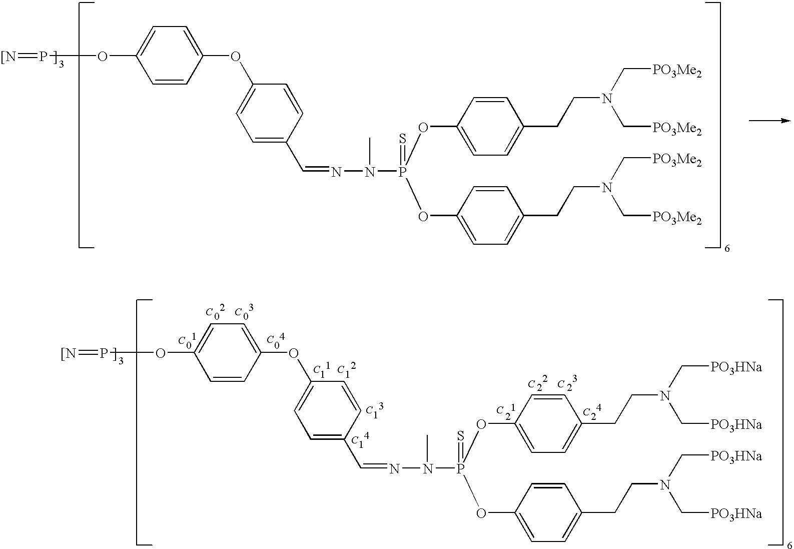

Trimethylsilyl bromide (535 μL, 4.00 mmol) is added to a solution of dendrimer dimethylphosphonate ends (440 mg, 5.75 10⁻² mmol) (obtained in example 1.2.) in acetonitrile (10 mL) at 0° C. The mixture is stirred at 25° C. during 12 h then evaporated to dryness under reduced pressure. The obtained residue is treated with methanol (2×15 mL), washed with ether (20 mL) and suspended in water (1 mL/100 mg) in the presence of one equivalent of NaOH for one phosphonic end. The solution is lyophilised to afford the dendrimer with sodium salt of phosphonic acid ends as a white solid (yield: 85%).

³¹P-{¹H} NMR (D₂O/CD₃CN 7:3, 121.5 MHz): δ=6.82 (s, PO₃HNa); 10.43 (s, N₃P₃); 13.82 (s, PO₃Na₂); 64.56 (s, P₁); ¹H NMR (H₂O/CD₃CN 7:3; 300.13 MHz): δ=3.25 (AA′ part of a AA′BB′ system, m, 24H, CH₂—CH₂—N); 3.51 (d, ²J_{HP}=11.7 Hz, 48H, N—CH₂—P and 18H, CH₃—N—P₁); 3.82 (BB′ part of a AA′BB′ system, m, 24H, CH₂—CH₂—N); 7.06 (m, 36 H, C₀²—H, C₀³—H, C₁²—H); 7.30 (m, 24H, C₂²—H); 7.52 (m, 24H, C₂³—H); 7.79 (br s, 6H, CH═N); 7.98 (m, 12H, C₁³—H); ¹³C-{¹H} NMR (D₂O/CD₃CN 7:3, 75.6 MHz): δ=29.01 (s, CH₂—CH₂—N); 32.74 (br s, CH₃—N—P₁); 52.74 (br s, N—CH₂—P); 57.78 (s, CH₂—CH₂—N); 120.45 (s, C₁²); 121.35 (s, C₀³); 121.27 (d, ³J_{CP}=4.5 Hz, C₂²); 122.53 (s, C₀²); 128.94 (s, C₁³); 130.50 (s, C₁⁴); 130.85 (s, C₂³); 134.55 (s, C₂⁴); 141.00 (br s, CH═N); 146.01 (m, C₀¹); 149.36 (d, ²J_{CP}=6.1 Hz, C₂¹); 153.79 (s, C₀⁴); 158.17 (s, C₁¹) ppm.

### EXAMPLE 2

### Synthesis of a Dendrimer of Carbosilane Type of First Generation with Azabis Phosphonic Acid Ends Derived from Tyramine

### 2.1: Synthesis of the Dendrimer with Azabisphosphonate Ends Derived from Tyramine

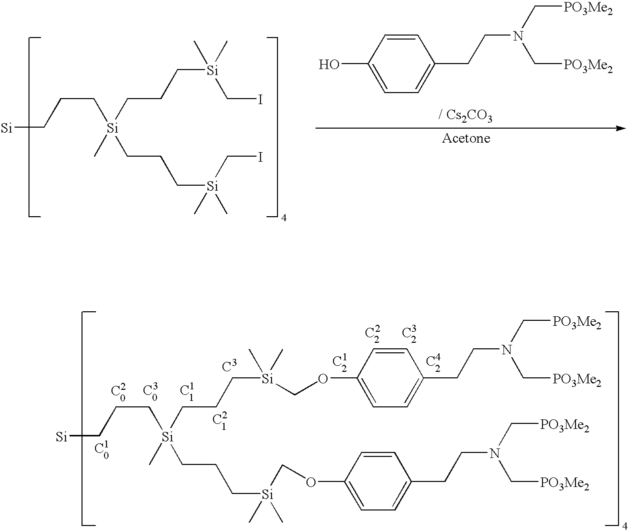

Tyramine aza-bisphosphonate obtained according to example 1.1 (320 mg, 0.830 mmol) and caesium carbonate (540 mg, 1.66 mmol) are added to a solution of carbosilane type dendrimer of first generation with SiCH₂I ends (200 mg, 8.7.10⁻⁵ mmol) in acetone (2 mL). The mixture is stirred at 40° C. during 20 h, centrifuged and the resulting clear solution is evaporated to dryness under reduced pressure. The obtained oil is purified by chromatography on silica gel (gradient acetone/methanol (100:0 to 0:100), Rf=0.38 in acetone/methanol (90:10)) to afford the dendrimer with dimethylphosphonate ends as a pale yellow solid (yield: 65%).

³¹P-{¹H} NMR (acetone-d6, 162.0 MHz): δ=26.44 (s, PO₃Me₂); ¹H NMR (CDCl₃, 400.13 MHz): δ=0.00 (s, 12H, Si—CH₃); 0.14 (s, 48H, Si—(CH₃)₂); 0.67 (br s, 32H, C₀¹—H, C₀³—H, C₁¹—H); 0.77-0.80 (m, 16H, C₁³—H); 1.45-1.52 (m, 24H, C₀²—H, C₁²—H); 2.75 (AA′ part of a AA′BB′ system, m, 16H, CH₂—CH₂—N); 3.03 (BB′ part of a AA′BB′ system, m, 16H, CH₂—CH₂—N); 3.17 (d, 2J_{HP}=9.3 Hz, 32H, N—CH₂—P); 3.61 (s, 16H, Si—CH₂—O); 3.72 (d, ³J_{HP}=10.4 Hz, 96H, OMe); 6.89 (m, 16H, C₂²—H); 7.18 (m, 16H, C₂³—H); ¹³C-{¹H} NMR (CDCl₃, 100.6 MHz): δ=−5.25 (s, Si—CH₃); −5.12 (s, Si—(CH₃)₂); 17.60 (s, C₀²); 18.32 (s, C₁²); 18.37 (C₁³); 18.37 (C₁¹); 18.71 (s, C₀'); 18.97 (s, C₀³); 32.23 (s, CH₂—CH₂—N); 49.98 (dd, ¹J_{CP}=156.9 Hz, ³J_{CP}=7.7 Hz, N—CH₂—P); 51.94 (br s, OMe); 58.74 (t, ³J_{CP}=7.0 Hz, CH₂—CH₂—N); 59.99 (s, Si—CH₂—O); 113.88 (s, C₂²); 129.65 (s, C₂³); 131.48 (s, C₂⁴); 160.02 (s, C₂¹); ²⁹Si NMR (CDCl₃; 79.5 MHz): δ=−0.30 (s, Si—CH₂-0); 1.07 (s, Si—CH₃); 3.98 (s, Si at the core) ppm.

### 2.2: Synthesis of the Dendrimer with Sodium Salt of Azabisphosphonic Acid Ends Derived from Tyramine

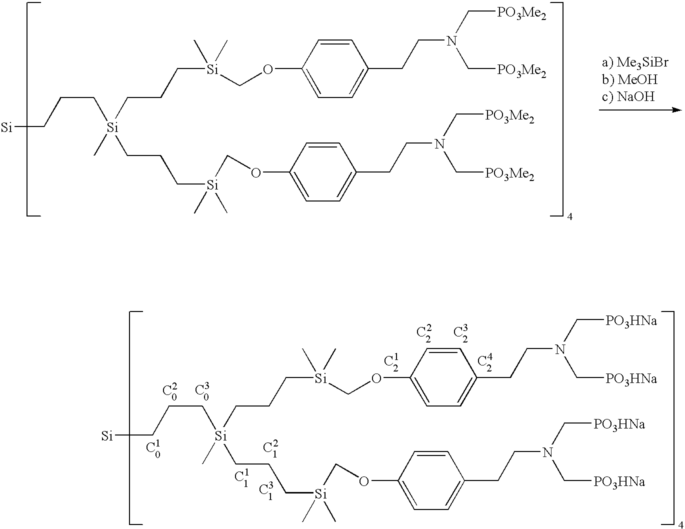

Trimethylsilyle bromide (120 μL, 8.56.10⁻¹ mmol) is added to a solution of dendrimer with dimethylphosphonate end obtained in example 2.1 (92.9 mg, 2.14 10⁻² mmol) in acetonitrile (2.5 mL) at 0° C. The mixture is stirred at 25° C. during 12 h then evaporated to dryness under reduced pressure. The obtained residue is treated with methanol (2×15 mL), washed with ether (20 mL) and suspended in water (1 mL/100 mg) in the presence of one equivalent of NaOH for one phosphonic end. The solution is lyophilised to afford the dendrimer with sodium salt phosphonic acid ends as a white solid (yield: 85%).

³¹P-{¹H} NMR (D₂O/acetone-d6 7:3, 162.0 MHz): δ=6.64 (s, PO₃HNa); ¹H NMR (D₂O/acetone-d6 7:3; 400.13 MHz): δ =−0.14 (br s, 60H, Si—CH₃ and Si—(CH₃)₂); 0.45 (br s, 48H, C₀¹—H, C₀³—H, C₁¹—H, C₁³—H); 1.23 (br s, 24H, C₀²—H, C₁²—H); 2.83 (AA′ part of a AA′BB′ system, br s, 16H, CH₂—CH₂—N); 3.11 (br s, 32H, N—CH₂—P); 3.31 (BB′ part of a AA′BB′ system, br s, 32H, CH₂—CH₂—N and Si—CH₂—O); 6.65 (m, 16H, C₂²—H); 7.05 (m, 16H, C₂³—H); ¹³C-{¹H} NMR (D₂O/acetone-d6 7:3, 100.6 MHz): δ=−5.04 (br s, Si—CH₃ and Si—(CH₃)₂); 17.5-19.2 (C₀¹, C₀², C₀³, C₁¹, C₁², C₁³); 29.30 (s, CH₂—CH₂—N); 53.74 (d, ¹J_{CP}=122.7 Hz, N—CH₂—P); 57.69 (s, CH₂—CH₂—N); 60.08 (s, CH₂—CH₂—N); 114.14 (s, C₂²); 128.57 (s, C₂⁴); 130.14 (s, C₂³); 159.86 (s, C₂¹).

### EXAMPLE 3

### Synthesis of Salamonczyk-Type of Second Generation with Azabis Phosphonic Acid Ends Derived from Tyramine

### 3.1. Synthesis of the Diphenoxyamino Phosphine Derived from Tyramine

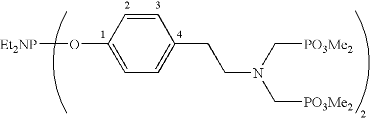

The experimental protocol used for preparing this molecule was inspired by that used by Salamonczyk in order to create his dendrimers (Tetrahedron Lett. 2000, 41, 1643). The aza bis phosphonate tyramine derivative obtained in Example 1.1 is weighed in a Schlenk tube under argon (2.3 g) and dissolved in 10 mL of distilled THF. The diethylaminodichlorophosphine is introduced into another Schlenk tube (0.5 mL) and placed in solution in 5 mL of distilled THF. The two Schlenk tubes are taken to −70° C. 1.4 mL of triethylamine are then added to the dichlorophosphine solution then the tyramine aza bis phosphonate solution is cannulated onto the mixture still at −70° C. The stirring is continued for half an hour at a low temperature then for 4 hours at ambient temperature. The mixture is then filtered on celite under argon then the solvent is eliminated under reduced pressure. The dry residue is kept under argon at a low temperature and used without other treatment in the rest of the synthesis.

³¹P{'H} NMR (CDCl₃): δ=30.4 (s, PO₃Me₂); 144.5 (s, Et₂NP) ppm.

¹H NMR (CDCl₃): δ=1.00 (t, ³J_{HH}=7.2 Hz, 6H, CH₃CH₂); 2.70 (m, 4H, N—CH₂CH₂); 3.00 (m, 4H, CH₂CH₂P); 3.11-3.23 (m, 12H, CH₂P, CH₃CH₂); 3.69 (d, ³J_{HP}=6.9 Hz, 24H, CH₃O); 6.90 (d, ³J_{HH}=8.4 Hz, 4H, C²H); 6.97 (d, ³J_{HH}=8.4 Hz, 4H, C³H) ppm.

### 3.2: Synthesis of the Dendrimer with Azabisphosphonate Ends Derived from Tyramine

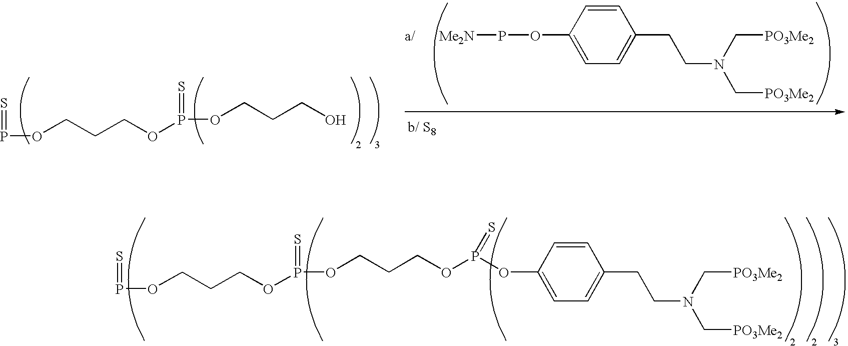

The phosphoramide derived from tyramine aza-bisphosphonate obtained as described in example 3.1 (160 mg, 0.17 mmol solubilized in dichloromethane/acetonitrile (1 mL/1 mL)) and tetrazole (180 mg, 2.60 mmol) are added to the dendrimer Salamonczyk with hydroxyle ends (1.64 g, 1.9 mmol) in solution in dichloromethane (4 mL). The mixture is stirred at 25° C. during 3 h 30 before addition of S₈ (512 mg, 2 mmol). The heterogeneous mixture is strired during 12 h at 25° C. then filtered. The filtrate is evaporated to dryness under reduced pressure and purified by chromatography on silica gel (elution gradient: dichloromethane/acetone (50:50 à 0:100) then acetone/methanol (100:0 à 0:100), Rf=0.95 in methanol) to afford the dendrimer with dimethyphosphonate ends as a white solid (yield: 43%).

³¹P-{¹H} NMR (acetone-d6, 101.25 MHz): δ=26.53 (s, PO₃Me₂), 58.72 (s, P²), 68.23 (s, P⁰ and P¹); ¹H NMR (acetone-d6, 300.13 MHz): δ=2.15 (m, 18H, C₁²—H and C₀²—H); 2.85 (AA′ part of a AA′BB′ system, m, 24H, CH₂—C₂—N); 3.17 (BB′ part of a AA′BB′ system, m, 24H, CH₂—CH₂—N); 3.25 (d, ²J_{H}=9.9 Hz, 48H, N—CH₂—P); 3.72 (d, ³J_{HP}=10.5 Hz, 144H, OMe); 4.24 (m, 24H, C₀¹—H, C₀³—H and C₁¹—H); 4.44 (m, 12H, C₁³—H); 7.19 (m, 24H, C₂²—H); 7.36 (m, 24H, C₂³—H); ¹³C-{¹H} NMR (acetone-d6, 75.5 MHz): δ=30.62 (m, C₀² and C₁²); 32.26 (s, CH₂—CH₂—N); 49.06 (dd, ¹J_{CP}=157.6 Hz, ³J_{CP}=8.2 Hz, N—CH₂—P); 52.13 (d, ²J_{CP}=5.0 Hz, OMe); 58.13 (t, ³J_{CP}=7.8 Hz, CH₂—CH₂—N); 64.49 (m, C₀¹, C₀³ ); and C₁³); 65.90 (m, ²J_{CP}=6.0 Hz, C₁¹); 120.82 (d, ³J_{CP}=4.5 Hz, C₂²); 130.28 (s, C₂³); 137.54 (s, C₂⁴); 148.99 (d, ²J_{CP}=7.6 Hz, C₂¹).

### 3.3. Synthesis of the Dendrimer with Sodium Salt Azabis Phosphonic Acid Ends Derived from Tyramine

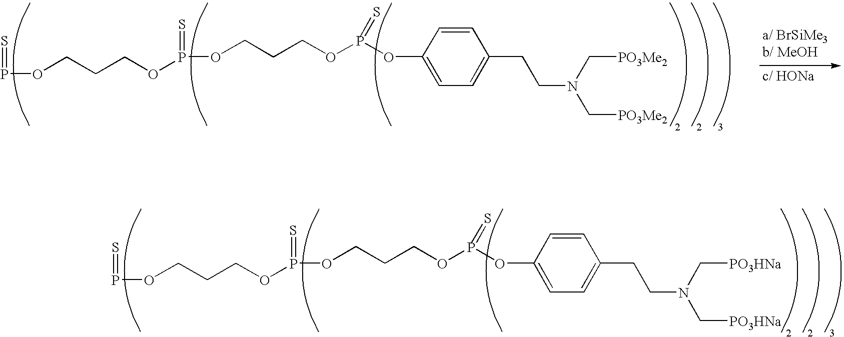

Trimethylsilyle bromide (190 μL , 1.44 mmol) is added to a solution of dendrimer with dimethylphosphonate ends obtained in example 3.2 (140 mg, 2.39 10⁻² mmol) in acetonitrile (3.5 mL) at 0° C. The mixture is stirred at 25° C. during 12 h then evaporated to dryness under reduced pressure. The residue thus obtained is treated with methanol (2×15 mL), washed with ether (20 mL) and suspended in water (1 mL/100 mg) in presence of one equivalent of NaOH for one phosphonic end. The obtained solution is lyophilised to afford the dendrimer with sodium salt of phosphonic acid ends as a white solid (yield: 70%).

³¹P-{H} NMR (D₂O/CD₃CN 9:1, 81.0 MHz): δ=10.19 (s, PO₃HNa), 14.63 (s, PO₃Na₂), 62.78 (s, P²), 70.58 (s, P⁰ and P¹); ¹H NMR (D₂O/CD₃CN 9:1, 300.13 MHz): δ=2.03 (m, 18H, C₁²—H and C₀²—H); 3.07 (AA′ part of a AA′BB′ system, m, 24H, CH₂—CH₂—N); 3.40 (br s, 48H, N—CH₂—P); 3.63 (BB′ part of a AA′BB′ system, m, 24H, CH₂—CH₂—N); 4.13 (br s, 24H, C₀¹—H, C₀³—H and C₁¹—H); 4.38 (br s, 12H, C₁³—H); 7.12 (m, 24H, C₂²—H); 7.31 (m, 24H, C₂³—H); ¹³C-{¹H} RMN (CDCl₃; 75.5 MHz): δ=29.12 (s, CH₂—CH₂—N); 3 (m, C₀² and C₁²); 52.64 (d, ¹J_{CP}=131.7 Hz, N—CH₂—P); 58.03 (br s, CH₂—CH₂—N); 64.90 (m, C₀¹, C₀³, C₁¹ and C₁³); 121.45 (s, C₂²); 130.83 (s, C₂³); 134.51 (s, C₂⁴); 149.31 (d, ²J_{CP}=7.2 Hz, C₂¹).

### EXAMPLE 4

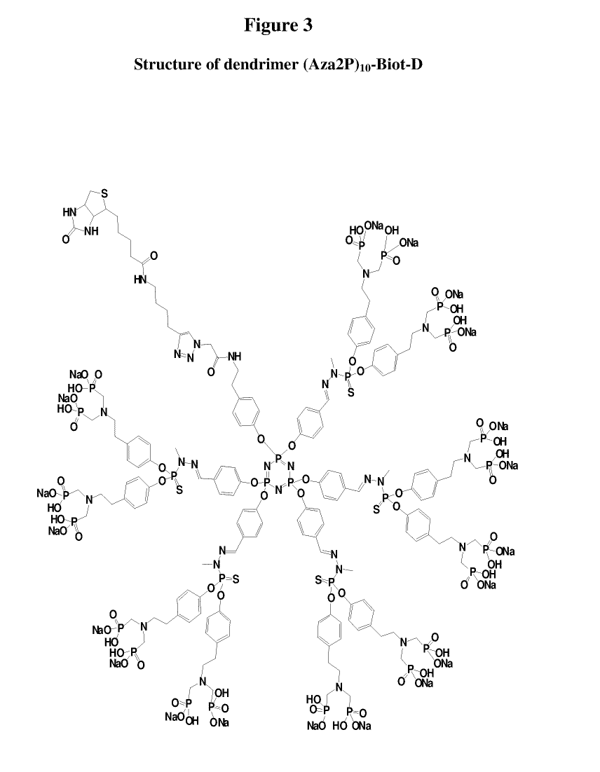

### Synthesis of Dimethylphosphonate Terminated Dendrimer with a Biotinylated Spacer (Aza 2P)₁₀-Biot-D (Voir FIG. 3)

### 4.1. Synthesis of 2-chloro-N-[2-(4-hydroxy-phenyl)-ethyl]-acetamide

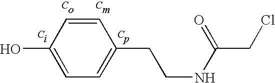

To a solution of tyramine (1.543 g, 11.25 mmol) in 25 mL of dichloromethane/saturated aqueous sodium carbonate mixture (1:1) was added chloroacetyl chloride (0.896 mL, 11.25 mmol) and the mixture was stirred at room temperature for 2 h. It was then diluted in water (50 mL) and extracted with dichloromethane (150 mL). The organic phase was dried over magnesium sulfate, filtered and evaporated to give a white solid which was purified by column chromatography (silica, dichloromethane/methanol, 97:3) to give 2-chloro-N-[2-(4-hydroxy-phenyl)-ethyl]-acetamide as a white solid (yield=75%).

¹H NMR (DMSO-d6, 300.1 MHz): δ=2.61 (t, ³J_{HH}=7.5 Hz, 2H, C₆H₄—CH₂—CH₂—NH), 3.24 (td, ³J_{HH}=7.5 Hz, ³J_{HH}=5.1 Hz, 2H, C₆H₄—CH₂—CH₂—NH), 4.03 (s, 2H, CO—CH₂), 6.68 (m, 2H, Cₒ—H), 6.99 (d, ³J_{HH}=8.4 Hz, 2H, Cₘ—H), 8.23 (t, ³J_{HH}=5.1 Hz, 1H, NH), 9.17 (s, 1H, OH); ¹³C{¹H} NMR (CDCl₃, 50.3 MHz): δ=34.3 (s, C₆H₄—CH₂—NH), 41.2 (s, CO—CH₂—Cl), 42.5 (s, C₆H₄—CH₂—CH₂—NH), 115.5 (s, Cₒ), 128.9 (s, Cₚ), 129.5 (s, Cₘ), 155.7 (s, Cᵢ), 165.9 (s, CO) ppm. DCI-MS (NH₃): m/z=231 [M+NH₄]⁺, 214 [M+H]⁺.

### 4.2. Synthesis of 2-Azido-N-[2-(4-hydroxy-phenyl)-ethyl]-acetamide

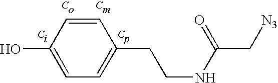

To a solution of 2-chloro-N-[2-(4-hydroxy-phenyl)-ethyl]-acetamide obtained in example 4.1 (250 mg, 1.17 mmol) in DMSO (3 mL) was added sodium azide (152 mg, 2.34 mmol) and the mixture was stirred at room temperature for 12 h. The reaction mixture was then diluted in water (70 mL) and extracted with ethyl acetate (140 mL) The organic phase was dried over magnesium sulfate, filtered and evaporated to give 2-Azido-N-[2-(4-hydroxy-phenyl)-ethyl]-acetamide as a viscous solid (yield=90%) that crystalises upon standing.

¹H NMR (CDCl₃, 300.1 MHz): δ=2.75 (t, ³J_{HH}=6.9 Hz, 2H, C₆H₄—CH₂—CH₂—NH), 3.51 (td, ³J_{HH}=6.9 Hz, ³J_{HH}=6.3 Hz, 2H, C₆H₄—CH₂—CH₂—NH), 3.95 (s, 2H, CO—CH₂), 6.51 (broad s, 1H, NH), 6.82 (m, 2H, Cₒ—H), 7.02 (m, 2H, Cₘ—H), 7.38 (broad s, 1H, OH); ¹³C{¹} NMR (CDCl₃, 75.5 MHz): δ=34.6 (s, C₆H₄—CH₂—CH₂—NH), 40.9 (s, C₆H₄—CH₂—CH₂—NH), 52.6 (s, CO—CH₂—N), 115.7 (s, Cₒ), 129.5 (s, Cₚ), 129.7 (s, Cₘ), 155.3 (s, Cᵢ), 167.1 (s, CO) ppm. DCI-MS (NH₃): m/z=238 [M+NH₄]⁺.

### 4.3. Synthesis of 2-{4-[4-(1,3-Dioxo-1,3-dihydro-isoindol-2-yl)-butyl]-[1,2,3]triazol-1-yl}-N-[2-(4-hydroxy-phenyl)-ethyl]-acetamide

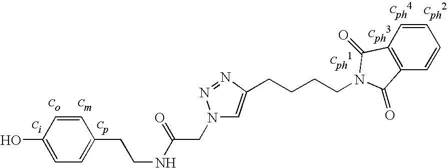

To 2.4 mL of tert-butanol/water mixture (1:1) were suspended 2-Azido-N-[2-(4-hydroxy-phenyl)-ethyl]-acetamide obtained in example 4.2 (385 mg, 1.750 mmol), N-(5-hexynyl)phthalimide (398 mg, 1.750 mmol), sodium ascorbate (35 mg, 0.175 mmol) and coppper sulfate (140 mg, 0.088 mmol). The reaction mixture was stirred at room temperature for 12 h and was then diluted in water, and filtered. The solid was washed with water and ether to give 2-{4-[4-(1,3-Dioxo-1,3-dihydro-isoindol-2-yl)-butyl]-[1,2,3]triazol-1-yl-N-2-(4-hydroxy-phenyl)-ethyl]-acetamide as a white solid (yield=90%). ¹H NMR (CD₃CN, 300.1 MHz): δ=1.71 (m, 4H, CH₂—CH₂—CH₂—CH₂—N, CH₂—CH₂—CH₂—CH₂—N), 2.70 (m, 4H, C₆H₄—CH₂—CH₂—NH, CH₂—CH₂—CH₂—CH₂—N), 3.37 (q, ³J_{HH}=6.9 Hz, ³J_{HH}=6.3 Hz, 2H, C₆H₄—CH₂—CH₂—NH), 3.67 (t, ³J_{HH}=6.3 Hz, 2H, CH₂—CH₂—CH₂—CH₂—N), 4.92 (s, 2H, CO—CH₂—N), 6.59 (broad s, 1H, NH), 6.73 (d, ³J_{HH}=8.1 Hz, 2H, Cₒ—H), 7.02 (d, ³J_{HH}=8.1 Hz, 2H, Cₘ—H), 7.50 (s, 1H, N—CH═C), 7.79 (m, 4H, Cₚₕ—H); ¹³C{¹H} NMR (CD₃CN, 75.5 MHz): δ=24.7 (s, CH₂—CH₂—CH₂—CH₂—N), 26.5 (s, CH₂CH₂—CH₂—CH₂—N), 27.7 (s, CH₂—CH₂—CH₂—N), 34.1 (s, C₆H₄—CH₂—CH₂—NH), 37.4 (s, CH₂—CH₂—CH₂—CH₂—N), 40.8 (s, C₆H₄—CH₂—CH₂—NH), 52.1 (s, CO—CH₂—N), 115.1 (s, Cₒ), 122.7 (s, Cₚₕ⁴), 122.9 (s, N—CH═C), 129.8 (s, Cₘ), 130.2 (s, Cₚ), 132.3 (s, Cₚₕ³), 134.1 (s, Cₚₕ²), 147.5 (N—CH═C), 155.4 (s, Cᵢ), 165.6 (HN—CO—CH₂), 168.5 (s, Cₚₕ¹) ppm. DCI-MS (NH₃): m/z=448 [M+NH₄]⁺.

### 4.4. Synthesis of Tert-Butyl 6-heptynoate ester

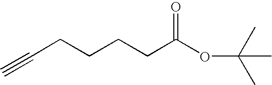

To a solution of 6-heptynoic acid (0.750 mL, 5.970 mmol), tert-butanol (1.7 mL, 17.91 mmol), and DMAP (73 mg, 0.597 mmol) in dichloromethane (12 mL) was added N,N′-dicyclohexylcarbodiimide (1.416 g, 6.864 mmol). The reaction mixture was stirred for 12 h at rt, and then the dicyclohexylurea was filtrated off and washed with dichloromethane (60 mL) and diethyl ether (30 mL). The organic phases were combined, dried over sodium sulfate and evaporated to dryness. The residue was purified by column chromatography (silica, pentane/ether, 98:2) to afford the tert-butyl 6-heptynoate ester as a colourless oil (yield=30%). ¹H NMR (CDCl₃, 300.1 MHz): δ=1.45 (s, 9H, CH₃), 1.57 (m, 2H, CH₂—CH₂—CO), 1.70 (m, 2H, CH₂—CH₂—CH₂—CO), 1.96 (t, ⁴J_{HH}=2.6 Hz, 1H, CH), 2.23 (m, 2H, CH₂—CO, C—CH₂); ¹³C{¹H} NMR (CDCl₃, 62.9 MHz): δ=18.2 (C—CH₂), 24.1 (s, CH₂—CH₂—CO), 27.8 (s, CH₂—CH₂—CH₂—CO), 28.1 (s, CH₃), 35.0 (s, CH₂—CO), 68.5 (s, CH), 84.1 (s, C—CH₂), 172.8 (s, CO) ppm.

### 4.5. Synthesis of 5-(1-{[2-(4-Hydroxy-phenyl)-ethylcarbamoyl]-methyl}-1H-[1,2,3]triazol-4-yl)-pentanoic acid tert-butyl ester

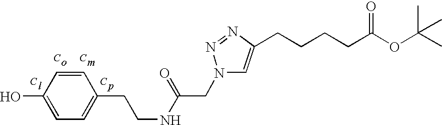

To 1.5 mL of tert-butanol/water mixture (1:1) were suspended 2-Azido-N-[2-(4-hydroxy-phenyl)-ethyl]-acetamide obtained in example 4.2 (254 mg, 1.152 mmol), tert-butyl 6-heptynoate ester obtained in example 4.4 (210 mg, 1.152 mmol), sodium ascorbate (23 mg, 0.015 mmol) and coppper sulfate (9 mg, 0.057 mmol). The reaction mixture was stirred at room temperature for 12 h. It was then diluted in 0.6 mL of THF and 10 mL of water, stirred for 10 min, and extracted with ethyl acetate (60 mL). The organic phase was washed with brine (30 mL), dried over sodium sulfate, filtered and evaporated to give a sticky solid which was purified by column chromatography (silica, ether/acetone, 1:0 to 8:2) to give 5(1-{[2-(4-Hydroxy-phenyl)-ethylcarbamoyl]-methyl}-1H-[1,2,3]triazol-4-yl)-pentanoic acid tert-butyl ester as a sticky solid (yield=85%). ¹H NMR (CDCl₃, 300.1 MHz): δ=1.47 (s, 9H, CH₃), 1.69 (m, 4H, CH₂—CH₂—CH₂—CH₂—CO, CH₂—CH₂—CH₂—CH₂—CO), 2.30 (t, ^{J}_{HH}=6.4 Hz, 2H, CH₂—CH₂—CH₂—CH₂—CO), 2.71 (m, 4H, C₆H₄—CH₂—CH₂—NH, CH₂—CH₂—CH₂—CH₂—CO), 3.47 (q, ³J_{HH}=6.3 Hz, 2H, C₆H₄—CH₂—CH₂—NH), 4.95 (s, 2H, CO—CH₂—N), 6.13 (t, ³J_{HH}=5.5 Hz, 1H, NH), 6.76 (d, ³J_{HH}=8.4 Hz, 2H, Cₒ—H), 6.89 (d, ³J_{HH}=8.4 Hz, 2H, Cₘ—H), 7.31 (s, 1H, OH), 7.61 (s, 1H, N—CH═C); ¹³C{¹H} NMR (CDCl₃, 75.5 MHz): δ=24.6(s, CH₂—CH₂—CH₂—CH₂—CO), 25.6 (s, CH₂—CH₂—CH₂—CH₂—CO), 28.1 (s, CH₃), 28.4 (s, CH₂—CH₂—CH₂—CH₂—CO), 34.0 (s, C₆H₄—CH₂—CH₂—NH), 35.2 (s, CH₂—CH₂—CH₂—CH₂—CO), 40.8 (s, C₆H₄—CH₂—CH₂—NH), 53.0 (s, CO—CH₂—N), 80.8 (s, C(CH₃)), 115.6 (s, Cₒ), 122.3 (s, N—CH═C), 129.3 (s, Cₚ), 129.7 (s, Cₘ), 148.5 (N—CH═C), 155.4 (s, Cᵢ), 165.4 (HN—CO—CH₂), 173.8 (s, CO₂) ppm. FAB-MS (>0): m/z=403 [M+H]⁺, 347 [M−C₄H₉+2H]⁹.

### 4.6. Synthesis of penta(4-formylphenoxy)-chlorocyclotriphosphazene

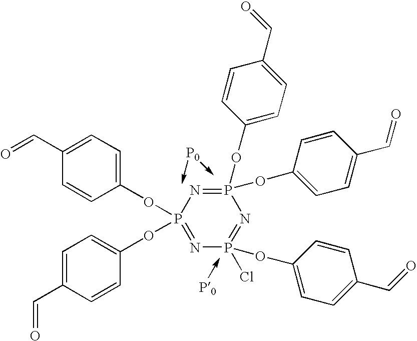

2591 mg of 4-hydroxybenzaldehyde sodium salt (18 mmol) are added at 0° C. and under an inert atmosphere to a solution containing 1.2 g of hexachlorocyclotriphosphazene (3.45 mmol) in THF (300 mL). The reaction medium is stirred for 12 hours while the temperature is allowed to gradually return to ambient temperature. The crude reaction product is evaporated to dryness then purified by “flash” chromatography on a silica column The product is isolated in the form of translucent oil with a yield of 70%.

³¹P{¹H} NMR (CDCl₃, 81 MHz): δ=9.2 (d, ²J_{PP}=86.6 Hz, P₀); 24.3 (t, ²J_{PP}=86.6 Hz, P′₀) ppm.

### 4.7. Synthesis of Persubstituted Cyclotriphosphazene Derivative with a Protected Amine Terminated Spacer

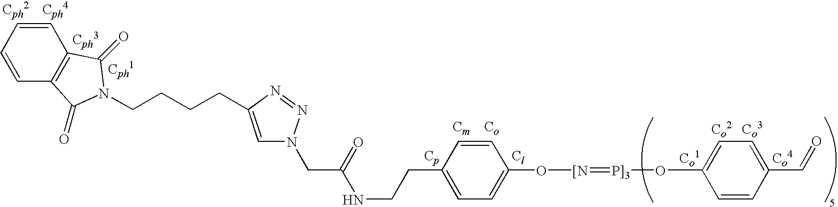

To a mixture of 2-{4-[4-(1,3-Dioxo-1,3-dihydro-isoindol-2-yl)-butyl]-[1,2,3,]triazol-1-yl}-N-[2-(4-hydroxy-phenyl)-ethyl[-acetamide obtained in example 4.3 (173 mg, 0.386 mmol) and of compound of example 4.6 (272 mg, 0.351 mmol) in THF (15 mL) was added caesium carbonate (126 mg, 0.386 mmol), and the mixture was stirred at rt for 12 h. The reaction mixture was centrifugated, filtered and evaporated. The residue was purified by column flash chromatography (silica, pentane/ethyl acetate, 1:1) to give the title compound as a white oil (yield=85%). ³¹P{¹H} NMR (CDCl₃, 121.5 MHz): δ=7.4 (s, N₃P₃); ¹H NMR (CDCl₃, 300.1 MHz): δ=1.74 (m, 4H, CH₂—CH₂—CH₂—CH₂—N, CH₂—CH₂—CH₂—CH₂—N), 2.76 (m, 4H, C₆H₄—CH₂—CH₂—NH, CH₂—CH₂—CH₂—CH₂—N), 3.46 (q, ³J_{HH}=HH 6.7 Hz, 2H, C₆H₄—CH₂—CH₂—NH), 3.71 (t, ³J_{HH}=6.5 Hz, 2H, CH₂—CH₂—CH₂—CH₂—N), 4.99 (s, 2H, CO—CH₂—N), 6.32 (t, ³J_{HH}=5.7 Hz, 1H, NH), 6.91 (d, ³J_{HH}=8.6 Hz, 2H, Cₒ—H), 6.98 (d, ³J_{HH}=8.6 Hz, 2H, Cₘ—H), 7.14 (m, 10H, C₀²—H), 7.44 (s, 1H, N—CH═C), 7.73 (m, 12H, C₀³—H, Cₚₕ—H), 7.82 (m, 2H, Cₚₕ—H), 9.94 (s, 5H, CHO); ¹³C{¹H} NMR (CDCl₃, 75.5 MHz): δ=25.0 (s, CH₂—CH₂—CH₂—CH₂—N), 26.4 (s, CH₂—CH₂—CH₂—CH₂—N), 27.9 (s, CH₂—CH₂—CH₂—CH₂—N), 3.47 (s, C₆H₄—CH₂—CH₂—NH), 37.5 (s, CH₂—CH₂—CH₂—CH₂—N), 40.8 (s, C₆H₄—CH₂—CH₂—NH) 53.0 (s, CO—CH₂—N), 120.7 (s, Cₒ), 121.3 (2 s, C₀²), 122.6 (s, N—CH═C), 123.2 (s, Cₚₕ⁴), 129.9 (s, Cₘ), 131.4 (s, C₀³), 132.1 (s, Cₚₕ³), 133.6 (2 s, C₀⁴), 133.7 (s, C₀⁴), 134.0 (s, Cₚₕ²) 136.0 (s, Cₚ), 148.3 (s, N—CH═C), 148.7 (m, Cᵢ), 154.6 (s, C₀¹), 154.7 (s, C₀¹), 165.4 (s, HN—CO—CH₂—N), 168.4 (s, Cₚₕ¹), 190.4 (s, CHO), 190.5 (s, CHO), 190.6 (s, CHO) ppm. FAB-MS: m/z=1187 [M+H]⁺.

### 4.8. Synthesis of Chlorinated Dendrimer with a Protected Amine Terminated

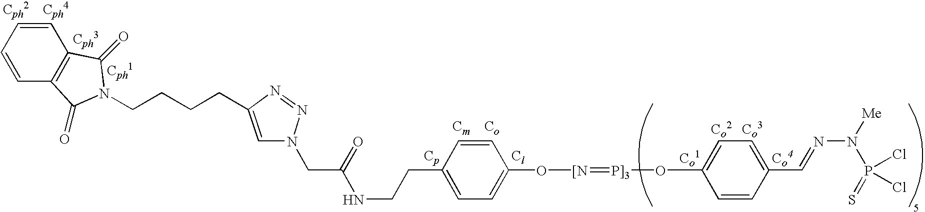

To an ice-cooled solution of N-methyldichlorothiophosphorhydrazide 2 (0.969 mmol) in chloroform (6.4 mL) was added the compound obtained in example 4.7 (200 mg, 0.169 mmol) and the mixture was stirred at rt for 2 h. After the evaporation of the solvent, the residue was diluted in the minimum of chloroform and precipitated by the addition of a large amount of pentane. This purification step was repeated twice to give dendrimer the title compound as a white solid (yield=80%). ³¹P{¹H} NMR (CDCl₃, 121.5 MHz): δ=8.3 (broad s, N₃P₃), 62.4 (2 s, P═S), 62.5 (s, P═S); ¹H NMR (CDCl₃, 300.1 MHz): δ=1.76 (broad s, 4H, CH₂—CH₂—CH₂—CH₂—N, CH₂—CH₂—CH₂—CH₂—N), 2.77 (m, 4H, C₆H₄—CH₂—CH₂—NH, CH₂—CH₂13 CH₂—CH₂—N), 3.43 (m, 2H, C₆H₄—CH₂—CH₂—NH), 3.49 (d, ³J_{HP}=14.0 Hz, 9H, N—CH₃), 3.50 (d, ³J_{HP}=14.0 Hz, 6H, N—CH₃), 3.71 (t, ³J_{HH}=6.3 Hz, 2H, CH₂—CH₂—CH₂—CH₂—N), 5.00 (s, 2H, CO—CH₂—N), 6.41 (broad s, 1H, NH), 6.90 (d, ³J_{HH}=8.5 Hz, 2H, Cₒ—H), 6.98 (m, 7H, Cₘ—H, C_{O}²—H), 7.04 (m, 5H, C₀²—H), 7.49 (s, 1H, N—CH═C), 7.61 (m, 13H, C₀³—H, CH═N), 7.70 (m, 4H, CH═N, Cₚₕ—H), 7.82 (m, 2H, Cₚₕ—H); ¹³C{¹H} NMR (CDCl₃, 75.5 MHz): δ=24.7 (s, CH₂—CH₂—CH₂—CH₂—N), 26.3 (s, CH₂—CH₂—CH₂—CH₂—N), 27.9 (s, CH₂—CH₂—CH₂—CH₂—N), 32.0 (2 d, ²J_{CP}=12.9 Hz, N—CH₃), 34.8 (s, C₆H⁴—CH₂—CH₂—NH), 37.5 (s, CH₂—CH₂—CH₂—CH₂—N), 41.0 (s, C₆H₄—CH₂—CH₂—NH), 53.2 (s, CO—CH₂—N), 121.1 (s, Cₒ), 121.4 (s, C₀²), 123.1 (s, N—CH═C), 123.2 (s, Cₚₕ⁴), 128.6 (s, C₀³), 129.8 (s, Cₘ), 131.3 (s, Cₚₕ³), 132.0 (s, C₀⁴), 134.0 (s, Cₚₕ²), 135.3 (s, Cₚ), 140.7 (m, CH═N), 147.8 (s, N—CH═C), 148.9 (m, Cᵢ), 151.7 (broad s, C₀¹), 165.0 (s, HN—CO—CH₂—N), 168.4 (s, Cₚₕ¹) ppm.

### 4.9. Synthesis of Dimethylphosphonate Terminated Dendrimer with a Protected Amine Terminated Spacer

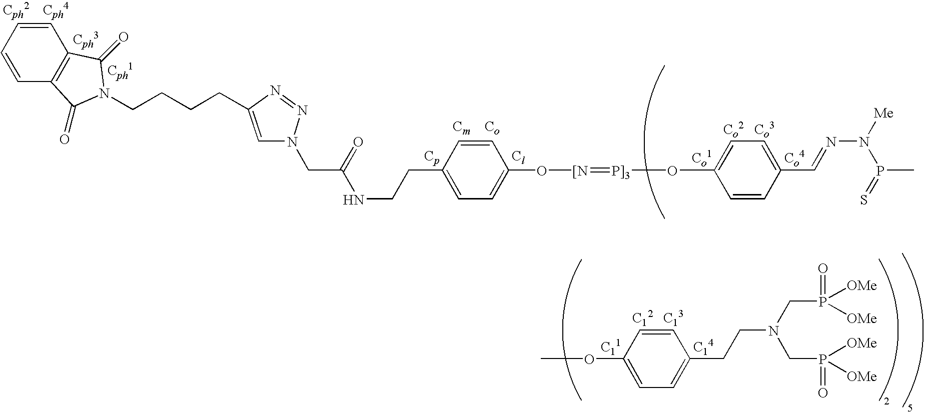

To a solution of dendrimer obtained in example 4.7 (316 mg, 0.159 mmol) in acetone (17 mL) were added phenol obtained in example 1.1 (666 mg, 1.747 mmol) and caesium carbonate (569 mg, 1.747 mmol) and the mixture was stirred at rt for 12 h. The reaction mixture was centrifugated, filtered and evaporated. The resulting crude oil was eluted on a plug of silica with acetone to remove the unreacted phenol then with acetone/methanol/water mixture (7:2:1). The resulting dendrimer solution was concentrated to dryness under reduced pressure, dissolved in 10 mL of dichloromethane, dried over sodium sulfate, filtered (micropore, 0.2 μm) and finally evaporated to dryness under reduced pressure to afford the title compound as a colourless oil (yield=85%) ³¹P{¹H} NMR (CDCl₃, 121.5 MHz): δ=8.4 (s, N₃P₃), 26.7 (2 s, PO₃Me₂), 26.8 (2 s, PO₃Me₂), 63.1 (2 s, P═S), 63.2 (s, P═S); ¹H NMR (CDCl₃, 300.1 MHz): δ=1.70 (broad s, 4H, CH₂—CH₂—CH₂—CH₂—N, CH₂—CH₂—CH₂—CH₂—N), 2.69 (m, 24H, C₆H₄—CH₂—CH₂—N, C₆H₄—CH₂—CH₂—NH, CH₂—CH₂—CH₂—CH₂—N), 3.03 (m, 20H, C₆H₄—CH₂—CH₂—N), 3.16 (d, ²J_{HP}=9.3 Hz, 40H, N—CH₂—P), 3.26 (m, 17H, N—CH₃, C₆H₄—CH₂—CH₂—NH), 3.71 (broad d, ³J_{HP}=10.4 Hz, 122H, P(O)(OCH₃), CH₂—CH₂—CH₂—CH₂—N), 4.87 (s, 2H, CO—CH₂—N), 6.84 (d, ³J_{HH}=8.3 Hz, 2H, Cₒ—H), 6.97 (m, 7H, Cₘ—H, C₀²—H), 7.06 (m, 15H, C₀²—H, C₁²—H), 7.15 (d, ³J_{HH}=8.3 Hz, 20H, C₁³—H), 7.41 (s, 1H, N—CH═C), 7.28 (m, 17H, C₀³—H, CH═N, Cₚₕ—H), 7.79 (m, 2H, Cₚₕ—H); ¹³C{¹H} NMR (CDCl₃, 75.5 MHz): δ=25.1 (s, CH₂—CH₂—CH₂—CH₂—N), 26.6 (s, CH₂—CH₂—CH₂—CH₂—N), 28.1 (s, CH₂—CH₂—CH₂—CH₂—N), 32.9 (m, C₆H₄—CH₂—CH₂—N, N—CH₃), 34.7 (s, C₆H₄—CH₂—CH₂—NH), 37.6 (s, CH₂—CH₂—CH₂—CH₂—N), 40.9 (s, C₆H₄—CH₂—CH₂—CH₂—NH), 49.5 (dd, ¹J_{CP}=157.5 Hz, ³J_{CP}=7.3 Hz, N—CH₂—P), 52.4 (s, CO—CH₂—N), 52.6 (m, P(O)(OCH₃)), 58.1 (t, ³J_{CP}=7.6 Hz, C₆H₄—CH₂—CH₂—N), 121.0 (s, Cₒ), 121.2 (broad d, ³J_{CP}=4.2 Hz, C₁², C₀²), 122.4 (s, N—CH═C), 123.2 (s, Cₚₕ⁴), 128.2 (s, C₀³), 128.3 (s, C₀³), 129.6 (s, Cₘ), 129.9 (s, C₁³), 132.1 (s, C₀⁴ or Cₚₕ³), 132.2 (s, C₀⁴ or Cₚₕ³), 133.9 (s, Cₚₕ²), 135.6 (s, Cₚ), 136.6 (s, C₁⁴), 138.7 (m, CH═N), 147.8 (s, N—CH═C), 148.9 (broad d, ²J_{CP}=7.1 Hz, C₁¹, Cᵢ), 151.2 (m, C₀¹), 165.4 (s, HN—CO—CH₂—N), 168.3 (s, Cₚₕ¹) ppm.

### 4.10. Synthesis of Persubstituted Cyclotriphosphazene Derivative with a Protected Acid Terminated Spacer

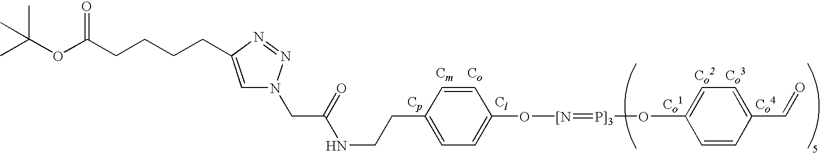

To a mixture of the compound obtained in example 4.5 (140 mg, 0.348 mmol) and compound of example 4.6 (270 mg, 0.348 mmol) in THF (5 mL) was added caesium carbonate (113 mg, 0.348 mmol), and the mixture was stirred at room temperature for 12 h. The reaction mixture was centrifugated, filtered and evaporated. The residue was purified by column flash chromatography (silica, pentane/ethyl acetate, 1:1) to give the title compound as a colourless viscous oil (yield=87%). ³¹P{¹H} NMR (CDCl₃, 101.3 MHz): δ=7.4 (s, N₃P₃); ¹H NMR (CDCl₃, 200.1 MHz): δ=1.41 (s, 9H, CH₃), 1.65 (m, 4H, CH₂—CH₂—CH₂—CH₂—CO, CH₂—CH₂—CH₂—CH₂—CO), 2.22 (t, ³J_{HH}=6.8 Hz, 2H, CH₂—CH₂—CH₂—CH₂—CO), 2.72 (broad t, ³J_{HH}=7.0 Hz, 4H, C₆H₄—CH₂—CH₂—NH, CH₂—CH₂—CH₂—CH₂—CO), 3.47 (q, ³J_{HH}=6.8 Hz, 2H, C₆H₄—CH₂—CH₂—NH), 4.97 (s, 2H, CO—CH₂—N), 6.37 (t, ³J_{HH}=5.4 Hz, 1H, NH), 6.91 (m, 4H, Cₒ—H, Cₘ—H), 7.11 (m, 10H, C₀²—H), 7.42 (s, 1H, N—CH═C), 7.70 (m, 10H, C₀³—H), 9.92 (s, 5H, CHO); ¹³C{¹H} NMR (CDCl₃, 62.9 MHz): δ=24.5(s, CH₂—CH₂—CH₂—CH₂—CO), 25.3 (s, —CH₂—CH₂—CH₂—CH₂—CO), 28.1 (s, CH₃), 28.6 (s, CH₂—CH₂—CH₂—CH₂—CO), 34.7 (s, C₆H₄—CH₂—CH₂—NH), 35.1 (s, CH₂—CH₂—CH₂—CH₂—CO), 40.8 (s, C₆H₄—CH₂—CH₂—NH), 53.0 (s, CO—CH₂—N), 80.2 (s, C(CH₃)), 120.7 (s, Cₒ), 121.3 (broad s, C₀²), 122.5 (s, N—CH═C), 129.9 (s, Cₘ), 130.9 (s, C₀³), 133.7 (2 s, C₀⁴), 135.9 (s, Cₚ), 148.7 (broad s, N—CH═C, Cᵢ), 154.6 (s, C₀¹), 165.4 (s, HN—CO—CH₂), 172.9 (s, CO₂), 190.4 (s, CHO), 190.5 (s, CHO), 190.6 (s, CHO) ppm.

### 4.11. Synthesis of Fluorescent Dendrimer with a PSCl₂ Termination

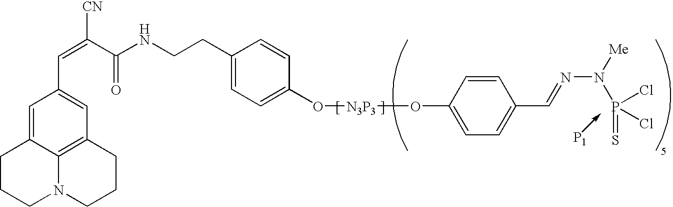

100 mg of the compound obtained in Stage 5 (0.05 mmol) is added at 0° C. to a solution of dichlorothiophospho-(N-methyl)-hydrazide (0.3 mmol) in chloroform (1.5 mL). The reaction mixture is stirred for 12 hours. After evaporation of the reaction solvent, the product is diluted in a minimum amount of dichloromethane and precipitated by addition of a large volume of pentane. This treatment is carried out three times. The product is isolated with a yield of 90%.

NMR ³¹P{¹H} (CDCl₃, 81.02 MHz): δ=11.8 (bs, N₃P₃); 65.9 (s, P₁); 66.0 (s, P₁); 66.1 (s, P₁) ppm.

### 4.12. Synthesis of Chlorinated Dendrimer with a Protected Acid Terminated Spacer

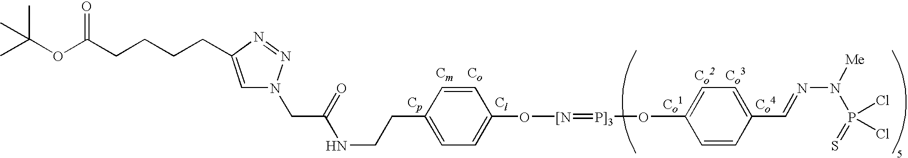

To an ice-cooled solution of N-methyldichlorothiophosphorhydrazide of example 4.11 (0.655 mmol) in chloroform (4.3 mL) was added the compound of example 4.10 (130 mg, 0.114 mmol) and the mixture was stirred at room temperature for 2 h. After the evaporation of the solvent, the residue was diluted in the minimum of chloroform and precipitated by the addition of a large amount of pentane. This purification step was repeated twice to give the title compound as a white solid (yield=85%). ³¹P{¹H} NMR (CDCl₃, 121.5 MHz): δ=8.3 (broad s, N₃P₃), 62.4 (2 s, P═S), 62.5 (s, P═S); ¹H NMR (CDCl₃, 300.1 MHz): δ=1.45 (s, 9H, C(CH₃)), 1.71 (m, 4H, CH₂—CH₂—CH₂—CH₂—CO, CH₂—CH₂—CH₂—CH₂—CO), 2.27 (t, ³J_{HH}=7.1 Hz, 2H, CH₂—CH₂—CH₂—CH₂—CO), 2.79 (m, 4H, C₆H₄—CH₂—NH, CH₂—CH₂—CH₂—CH₂—CO), 3.45 (broad s, 2H, C₆H₄—CH₂—CH₂—NH), 3.50 (d, ³J_{HP}=14.0 Hz, 9H, N—CH₃), 3.51 (d, ³J_{HP}=14.0 Hz, 6H, N—CH₃), 5.07 (broad s, 2H, CO——CH₂—N), 6.68 (broad s, 1H, NH), 6.90 (d, ³J_{HH}=8.0 Hz, 2H, Cₒ—H), 6.99 (broad d, ³J_{HH}=8.6 Hz, 6H, Cₘ—H, C₀²—H), 7.05 (d, ³J_{HH}=8.6 Hz, 6H, C₀²—H), 7.47 (s, 1H, N—CH═C), 7.60 (d, 10H, C₀³—H), 7.64 (broad s, 3H, CH═N), 7.68 (broad s, 2H, CH═N); ¹³C{¹H} NMR (CDCl₃, 75.5 MHz): δ=24.4 (s, CH₂—CH₂—CH₂—CH₂—CO), 24.9 (s, CH₂—CH₂—CH₂—CH₂—CO), 28.1 (s, C(CH₃)), 28.4 (s, CH₂—CH₂—CH₂—CH₂—CO), 32.0 (d, ²J_{CP}=12.9 Hz, N—CH₃), 32.1 (d, ²J_{CP}=12.9 Hz, N—CH₃), 34.8 (s, C₆H₄—CH₂—CH₂—NH), 35.1 (s, CH₂—CH₂—CH₂—CH₂—CO), 41.1 (s, C₆H₄—CH₂—CH₂—NH), 53.4 (s, CO—CH₂—N), 80.3 (s, C(CH₃)), 121.1 (s, Cₒ), 121.4 (broad s, C₀²), 123.5 (s, N—CH═C), 128.6 (s, C₀³), 129.7 (s, Cₘ), 131.3 (broad s, C₀⁴), 135.3 (s, Cₚ), 140.7 (m, CH═N), 147.7 (s, N—CH═C), 148.9 (m, Cᵢ), 151.7 (m, C₀¹), 164.7 (s, HN—CO—CH₂), 172.8 (s, CO₂) ppm.

### 4.13 Synthesis of Dimethylphosphonate Terminated Dendrimer with a Protected Acid Terminated Spacer

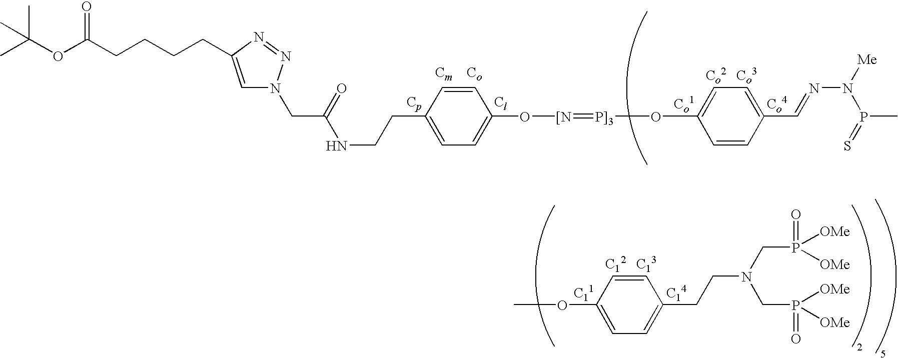

To a solution of compound obtained in example 4.12 (315 mg, 0.162 mmol) in acetone (17 mL) were added phenol of example 1.1 (680 mg, 1.783 mmol) and caesium carbonate (581 mg, 1.783 mmol) and the mixture was stirred at room temperature for 12 h. The reaction mixture was centrifugated, filtered and evaporated. The resulting crude oil was eluted on a plug of silica with acetone to remove the unreacted phenol then with acetone/methanol/water mixture (7:2:1). The resulting dendrimer solution was concentrated to dryness under reduced pressure, dissolved in 10 mL of dichloromethane, dried over sodium sulfate, filtered (micropore, 0.2 μm) and finally evaporated to dryness under reduced pressure to afford the title compound as a sticky solid (yield=90%) ³¹P{¹H} NMR (CDCl₃, 121.5 MHz): δ=8.4 (broad s, N₃P₃), 26.7 (2 s, PO₃Me₂), 26.8 (s, PO₃Me₂), 63.0 (s, P═S), 63.1 (s, P═S); ¹H NMR (CDCl₃, 300.1 MHz): δ=1.38 (s, 9H, C(CH₃)), 1.62 (m, 4H, CH₂—CH₂—CH₂—CH₂—CO, CH₂—CH₂—CH₂—CH₂—CO), 2.19 (t, ³J_{HH}=7.1 Hz, 2H, CH₂—CH₂—CH₂—CH₂—CO), 2.70 (m, 24H, C₆H₄—CH₂—CH₂—NH, —CH₂—CH₂—CH₂—CH₂—CO, C₆H₄—CH₂—CH₂—N), 2.99 (m, 20H, C₆H₄—CH₂—CH₂—N), 3.13 (d, ²J_{HP}=9.3 Hz, 40H, N—CH₂—P), 3.23 (m, 17H, N—CH₃, C₆H₄—CH₂—CH₂—NH), 3.67 (d, ³J_{HP}=10.5 Hz, 120H, P(O)(OCH₃)), 4.85 (broad s, 2H, CO—CH₂—N), 6.81 (d, ³J_{HH}=8.3 Hz, 2H, Cₒ—H), 6.99 (m, 52H, Cₘ—H, C₀²—H, C₁²13 H, C₁³—H), 7.39 (s, 1H, N—CH═C), 7.58 (m, 15H, C₀³—H, CH═N); ¹³C{¹H} NMR (CDCl₃, 75.5 MHz): δ=24.5 (s, CH₂—CH₂—CH₂—CH₂—CO), 25.3 (s, CH₂—CH₂—CH₂—CH₂—CO), 28.1 (s, C(CH₃)), 28.6 (s, CH₂—CH₂—CH₂—CH₂—CO), 32.9 (broad s, C₆H₄—CH₂—CH₂—N, N—CH₃), 34.6 (s, C₆H₄—CH₂—CH₂—NH), 35.1 (s, CH₂—CH₂—CH₂—CH₂—CO), 40.9 (s, C₆H₄—CH₂—C₂—CH₂—NH), 49.4 (dd, ¹J_{CP}=157.6 Hz, ³J_{CP}=7.2 Hz, N—CH₂—P), 52.7 (m, P(O)(OCH₃)), 53.4 (s, CO—CH₂—N), 58.1 (t, ³J_{CP}=7.5 Hz, C₆H₄—CH₂—CH₂—N), 80.1 (s, C(CH₃)), 121.0 (s, Cₒ), 121.2 (broad d, ³J_{CP}=4.1 Hz, C₁², C₀²), 122.4 (s, N—CH═C), 128.3 (s, C₀³), 129.6 (s, Cₘ), 129.9 (s, C₁³), 132.1 (d, ⁴J_{CP}=5.3 Hz, C₀⁴), 135.6 (s, Cₚ), 136.5 (s, C₁⁴), 138.7 (d, ³J_{CP}=13.9 Hz, CH═N), 147.9 (s, N—CH═C), 148.9 (broad d, ²J_{CP}=6.9 Hz, C₁¹, Cᵢ), 151.2 (m, C₀¹), 165.4 (s, HN—CO—CH₂), 172.9 (s, CO₂) ppm.

### 4.14. Synthesis of Dimethylphosphonate Terminated Dendrimer with a Protected Acid Terminated Spacer

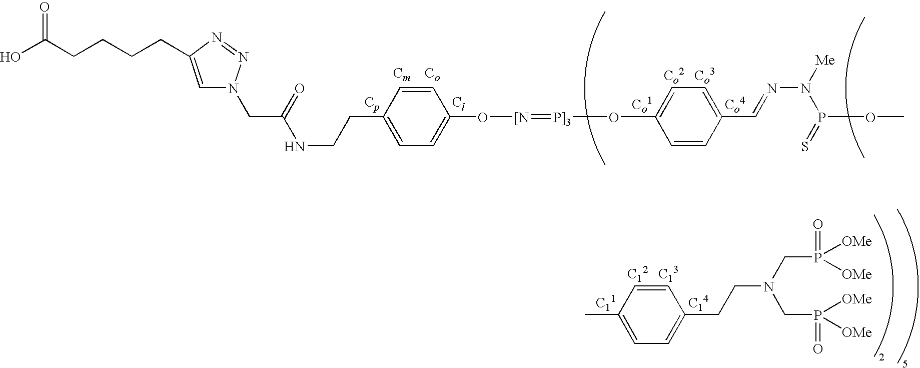

Compound of example 4.13 (107 mg, 0.020 mmol) was dissolved in a solution of 30% of TFA in dichloromethane, and the reaction mixture was allowed to stir at rt for 1.5 h and evaporated to dryness. This sequence was repeated 6 times and the residue was suspended into ethyl acetate so that remaining traces of TFA were removed upon evaporation to dryness. The residue was purified by column chromatography (silica, dichloromethane/methanol, 85:15) to give title compound as a sticky solid (yield=85%). ³¹P{¹H} NMR (CDCl₃, 121.5 MHz): δ=8.4 (broad s, N₃P₃), 26.8 (s, PO₃Me₂), 63.1 (s, P═S), 63.2 (s, P═S); ¹H NMR (CDCl₃, 300.1 MHz): δ=1.63 (m, 4H, CH₂—CH₂—CH₂—CH₂—CO, CH₂—CH₂—CH₂—CH₂—CO), 2.25 (m, 2H, CH₂—CH₂—CH₂—CH₂—CO), 2.73 (m, 24H, C₆H₄—CH₂—CH₂—NH, CH₂—CH₂—CH₂—CH₂—CO, C₆H₄—CH₂—CH₂—N), 3.03 (m, 20H, C₆H₄—CH₂—CH₂—N), 3.17 (d, ²J_{HP}=9.3 Hz, 40H, N—CH₂—P), 3.26 (m, 17H, N—CH₃, C₆H₄—CH₂—CH₂—NH), 3.70 (d, ³J_{HP}=10.5 Hz, 120H, P(O)(OCH₃)), 4.82 (broad s, 2H, CO—CH₂—N), 6.88 (d, ³J_{HH}=8.3 Hz, 2H, Cₒ—H), 7.08 (m, 52H, Cₘ—H, C₀²—H, C₁²—H, C₁³—H), 7.30 (s, 1H, N—CH═C), 7.61 (m, 15H, C₀³—H, CH═N); ¹³C{¹H} NMR (CDCl₃, 75.5 MHz): δ=24.3 (s, CH₂—CH₂—CH₂—CH₂—CO), 25.2 (s, CH₂—CH₂—CH₂—CH₂—CO), 28.5 (s, CH₂—CH₂—CH₂—CH₂—CO), 32.9 (broad s, C₆H₄—CH₂—CH₂—N, N—CH₃), 33.6 (s, CH₂—CH₂—CH₂—CH₂—CO), 34.5 (s, C₆H₄—CH₂—CH₂—NH), 40.7 (s, C₆H₄—C₂—CH₂—NH), 49.4 (dd, ¹J_{CP}=157.9 Hz, ³J_{CP}=7.1 Hz, N—CH₂—P), 52.7 (broad s, P(O)(OCH₃)), 53.4 (s, CO—CH₂—N), 58.1 (t, ³J_{CP}=7.4 Hz, C₆H₄—CH₂—CH₂—N), 120.4 (s, Cₒ), 121.2 (broad d, ³J_{CP}=4.1 Hz, C₁², C₀²), 122.5 (s, N—CH═C), 128.3 (s, C₀³), 129.7 (s, Cₘ), 129.9 (s, C₁³), 132.2 (d, ⁴J_{CP}=5.5 Hz, C₀⁴), 135.6 (s, 136.5 (s, C₁⁴), 138.8 (m, CH═N), 147.9 (s, N—CH═C), 148.9 (broad d, ²J_{CP}=7.4 Hz, C₁¹, Cᵢ), 151.4 (broad s, C₀¹), 165.3 (s, HN—CO—CH₂), 175.1 (s, CO₂) ppm.

### 4.15. Synthesis of Amine Terminated Spacer

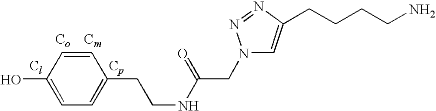

To a solution of 2-{4-[4-(1,3-Dioxo-1,3-dihydro-isoindol-2-yl)-butyl]-[1,2,3]triazol-1-yl}-N-[2-(4-hydroxy-phenyl)-ethyl]-acetamide (200 mg, 0.047 mmol) in ethanol (5 mL) was added hydrazine hydrate (0.070 mL, 2.23 mmol) and the reaction mixture was refluxed for 3 h. The mixture was evaporated to dryness to remove solvent and excess of hydrazine, resulting in a residue made of the title compound and by-product phthalhydrazide. The crude product was used without further purification. ¹H NMR (CD₃OD, 200.3 MHz): δ=1.70 (m, 4H, CH₂—CH₂—CH₂—CH₂—N, CH₂—CH₂—CH₂—CH₂—N), 2.72 (m, 4H, C₆H₄—CH₂—CH₂—NH, CH₂—CH₂—CH₂—CH₂—N), 2.86 (m, 2H, C₆H₄—CH₂—CH₂—NH), 3.41 (m, 2H, CH₂—CH₂—CH₂—CH₂—N), 5.07 (s, 2H, CO—CH₂—N), 6.72 (d, ³J_{HH}=8.5 Hz, 2H, Cₒ—H), 7.02 (d, ³J_{HH}=8.5 Hz, 2H, Cₘ—H), 7.68 (s, 1H, N—CH═C) ppm.

### 4.16 Synthesis of the Biotinylated Spacer

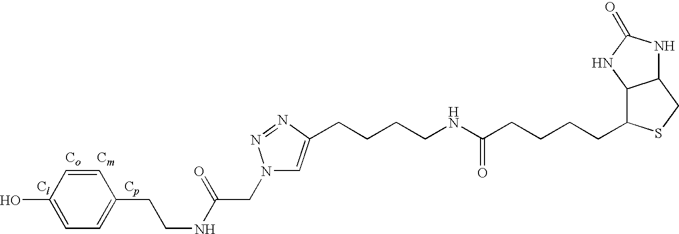

To a solution of biotin (65 mg, 0.265 mmol) and DIPEA (0.046 mL, 0.265 mmol) in DMF (2 mL) were added a solution of TBTU (89 mg, 0.277 mmol) in DMF (3 mL) and a solution of the compound of example 4.15 (80 mg, 0.252 mmol) in DMF (5 mL). The reaction mixture was stirred at room temperature for 24 h. The solvent was evaporated to dryness and the residue was purified by column chromatography (silica, dichloromethane/methanol, 9:1) to give title compound as a colourless oil (yield=70%). ¹H NMR (CD₃OD, 500.3 MHz): δ=1.45 (m, 2H, CO—CH₂—CH₂—CH₂—CH₂), 1.67 (m, 8H, CO—CH₂—CH₂—CH₂—CH₂, CO—CH₂—CH₂—CH₂—CH₂, CH₂—CH₂—CH₂—CH₂—NH, CH₂—CH₂—CH₂—CH₂—NH), 2.21 (t, ³J_{HH}=7.3 Hz, 2H, CO—CH₂—CH₂—CH₂—CH₂), 2.74 (m, 5H, C₆H₄—CH₂—CH₂—NH, CH₂—CH₂—CH₂—CH₂—NH, CH₂—S), 2.93 (dd, ²J_{HH}=5.0 Hz, ³J_{HH}=12.7 Hz, 1H, CH₂—S), 3.22 (m, 3H, CH₂—CH₂—CH₂—CH₂—NH, CH—S), 3.43 (t, ³J_{HH}=7.2 Hz, 2H, C₆H₄—CH₂—CH₂—NH), 4.30 (m, 1H, CH—CH—NH), 4.50 (m, 1H, CH₂—CH—NH), 5.06 (s, 2H, CO—CH₂—N), 6.72 (m, 2H, Cₒ—H), 7.03 (broad d, ³J_{HH}=8.4 Hz, 2H, Cₘ—H), 7.69 (s, 1H, N—CH═C); ¹³C{¹H} NMR (CD₃OD, 125.8 MHz): δ=24.5 (s, CH₂—CH₂—CH₂—CH₂—NH), 25.5 (s, CO—CH₂—CH₂—CH₂—CH₂), 26.3 (s, CH₂—CH₂—CH₂—CH₂—NH), 28.1 (s, CO—CH₂—CH₂—CH₂—CH₂), 28.4 (2 s, CO—CH₂—CH₂—CH₂—CH₂, CH₂—CH₂—CH₂—CH₂—NH), 34.1 (s, C₆H₄—CH₂—CH₂—NH), 35.4 (s, CO—CH₂—CH₂—CH₂—CH₂), 38.6 (s, CH₂—CH₂—CH₂—CH₂—NH), 39.6 (s, CH₂—S), 41.0 (s, C₆H₄—CH₂—CH₂—NH), 51.8 (s, CO—CH₂—N), 55.6 (s, CH—S), 60.2 (s, CH₂—CH—NH), 61.9 (s, CH—CH—NH), 114.9 (s, Cₒ), 123.3 (s, N—CH═C), 129.4 (s, Cₘ), 129.5 (s, Cₚ), 147.5 (N—CH═C), 155.6 (s, Cᵢ), 164.7 (s, HN—CO—NH), 166.4 (s, HN—CO—CH₂—N), 174.6 (s, (CH₂)₄—NH—CO) ppm.

### 4.17. Synthesis of Persubstituted Cyclotriphosphazene Derivative with a Biotinylated Spacer

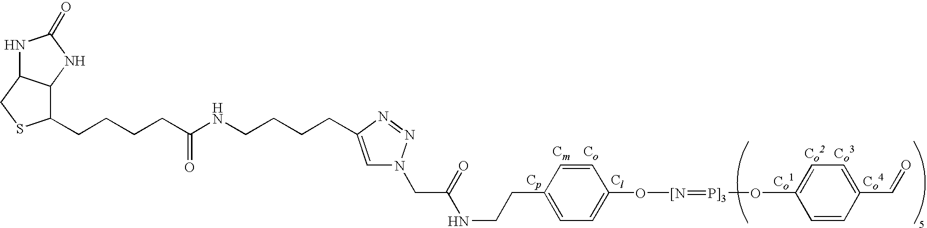

To a mixture of the compound of example 4.16 (65 mg, 0.120 mmol) and compound of example 4.6 (93 mg, 0.120 mmol) in DMF (5 mL) was added caesium carbonate (39 mg, 0.120 mmol), and the mixture was stirred at room temperature for 12 h. The reaction mixture was centrifugated, filtered and evaporated. The residue was purified by column flash chromatography (silica, dichloromethane/methanol, 9:1) to give title compound as a white oil (yield=85%). ³¹P{¹H} NMR (CDCl₃, 81.0 MHz): δ=8.4 (s, N₃P₃); ¹H NMR (CDCl₃, 300.1 MHz): δ=1.42 (broad s, 2H, CO—CH₂—CH₂—CH₂—CH₂), 1.72 (m, 8H, CO—CH₂—CH₂—CH₂—CH₂, CO—CH₂—CH₂—CH₂—CH₂, CH₂—CH₂—CH₂—CH₂—NH, CH₂—CH₂—CH₂—CH₂—NH), 2.24 (broad s, 2H, CO—CH₂—CH₂—CH₂—CH₂), 2.72 (m, 5H, C₆H₄—CH₂—CH₂—NH, CH₂—CH₂—CH₂—CH₂—NH, CH₂—S), 2.88 (m, 1H, CH₂—S), 3.18 (m, 3H, CH₂—CH₂—CH₂—CH₂—NH, CH—S), 3.43 (broad s, 2H, C₆H₄—CH₂—CH₂—NH), 4.32 (broad s, 1H, CH—CH—NH), 4.49 (broad s, 1H, CH₂—CH—NH), 5.03 (broad s, 2H, CO—CH₂—N), 6.53 (broad s, 1H, CH—CH—NH—CO or CH₂—CH—NH—CO), 6.73 (broad s, 1H, CH₂—CH—NH—CO), 6.88 (broad s, 2H, Cₒ—H), 7.00 (broad s, 2H, Cₘ—H), 7.12 (m, 10H, C₀²—H), 7.29 (m, 1H, (CH₂)₄—NH—CO), 7.51 (broad s, 1H, NH—CO—CH₂—N), 7.55 (broad s, 1H, N—CH═C), 7.72 (m, 10H, C₀³—H), 9.93 (2 s, 5H, CHO); ¹³C{¹H} NMR (CDCl₃, 75.5 MHz): δ=24.9 (s, CH₂—CH₂—CH₂—CH₂—NH), 25.7 (s, CO—CH₂—CH₂—CH₂—CH₂), 26.4 (s, CH₂—CH₂—CH₂—CH₂—NH), 28.1 (s, CO—CH₂—CH₂—CH₂—CH₂), 28.5 (2 s, CO—CH₂—CH₂—CH₂—CH₂, CH₂—CH₂—CH₂—CH₂—NH), 34.6 (s, C₆H₄—CH₂—CH₂—NH), 35.9 (s, CO—CH₂—CH₂—CH₂—CH₂), 39.0 (s, CH₂—CH₂—CH₂—CH₂—NH), 40.6 (s, CH₂—S), 41.0 (s, C₆H₄—CH₂—CH₂—NH), 52.8 (s, CO—CH₂—N), 55.7 (s, CH—S), 60.2 (s, CH₂—CH—NH), 61.9 (s, CH—CH—NH), 120.7 (s, Cₒ), 121.3 (s, C₀²), 123.0 (s, N—CH═C), 129.9 (s, Cₘ), 133.4 (s, C₀³), 133.6 (s, C₀⁴), 133.7 (s, C₀⁴), 136.3 (s, Cₚ), 148.0 (s, N—CH═C)), 148.5 (s, Cᵢ), 154.6 (broad s, C₀¹), 164.0 (s, HN—CO—NH), 165.7 (s, HN—CO—CH₂—N), 173.4 (s, (CH₂)₄—NH—CO), 190.5 (s, CHO), 190.6 (s, CHO), 190.8 (s, CHO) ppm.

### 4.18. Synthesis of Chlorinated Dendrimer with a Biotinylated Spacer

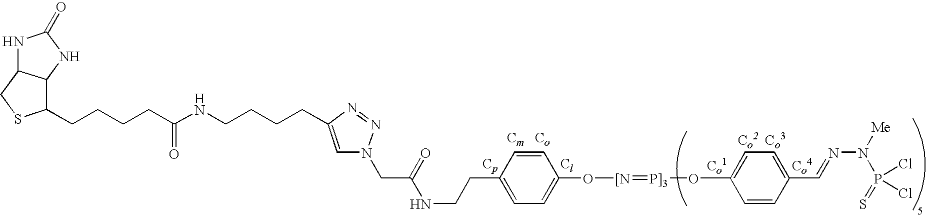

To an ice-cooled solution of N-methyldichlorothiophosphorhydrazide of example 4.11 (0.355 mmol) in chloroform (2.3 mL) was added the compound of example 4.17 (70 mg, 0.055 mmol) and the mixture was stirred at room temperature for 2 h. After the evaporation of the solvent, the residue was diluted in the minimum of chloroform and precipitated by the addition of a large amount of pentane. This purification step was repeated twice to give the title compound as a white solid (yield=90%). ³¹P{¹H} NMR (CDCl₃, 101.3 MHz): δ=8.3 (broad s, N₃P₃), 62.3 (s, P═S), 62.6 (s, P═S); ¹H NMR (CDCl₃, 300.1 MHz): δ=1.42 (m, 2H, CO—CH₂—CH₂—CH₂—CH₂), 1.70 (m, 8H, CO—CH₂—CH₂—CH₂—CH₂, CO—CH₂—CH₂—CH₂—CH₂, CH₂—CH₂—CH₂—CH₂—NH, CH₂—CH₂—CH₂—CH₂—NH), 2.28 (m, 2H, CO—CH₂—CH₂—CH₂—CH₂), 2.83 (broad s, 6H, C₆H₄—CH₂—CH₂—NH, CH₂—CH₂—CH₂—CH₂—NH, CH₂—S), 3.12 (m, 3H, CH₂—CH₂—CH₂—CH₂—NH, CH—S), 3.48 (broad d, ³J_{HP}=13.8 Hz, 17H, N—CH₃, C₆H₄—CH₂—CH₂—NH), 4.34 (broad s, 1H, CH—CH—NH), 4.52 (broad s, 1H, CH₂—CH—NH), 5.31 (broad s, 2H, CO—CH₂—N), 6.95 (m, 16H, Cₒ—H, Cₘ—H, C₀²—H, CH—CH—NH—CO, CH₂—CH—NH—CO), 7.63 (m, 17H, N—CH═C, C₀³—H, CH═N), 7.96 (s, 1H, (CH₂)₄—NH—CO), 8.28 (s, 1H, NH—CO—CH₂—N); ¹³C{¹H} NMR (CDCl₃, 75.5 MHz): δ=24.1 (s, CH₂—CH₂—CH₂—CH₂—NH), 25.9 (broad s, CO—CH₂—CH₂—CH₂—CH₂, CH₂—CH₂—CH₂—CH₂—NH), 28.0 (s, CO—CH₂—CH₂—CH₂—CH₂), 28.3 (broad s, CO—CH₂—CH₂—CH₂—CH₂, CH₂—CH₂—CH₂—CH₂—NH), 32.0 (d, ²J_{CP}=12.8 Hz, N—CH₃), 32.1 (d, ²J_{CP}=12.8 Hz, N—CH₃), 34.7 (s, C₆H₄—CH₂—CH₂—NH), 35.4 (s, CO—CH₂—CH₂—CH₂—CH₂), 39.1 (s, CH₂—CH₂—CH₂—CH₂—NH), 40.7 (s, CH₂—S), 41.2 (s, C₆H₄—CH₂—CH₂—NH), 53.3 (s, CO—CH₂—N), 55.7 (s, CH—S), 60.5 (s, CH₂—CH—NH), 61.9 (s, CH—CH—NH), 121.0 (s, Cₒ), 121.3 (s, C₀²), 123.0 (s, N—CH═C), 128.9 (s, C₀³), 129.8 (s, Cₘ), 131.3 (s, C₀⁴), 135.8 (s, Cₚ), 140.9 (m, CH═N), 145.7 (N—CH═C), 148.7 (broad s, Cᵢ), 151.7 (s, C₀¹), 164.2 (s, HN—CO—NH), 164.6 (HN—CO—CH₂—N), 174.6 (s, (CH₂)₄—NH—CO) ppm.

### 4.19. Synthesis of Dimethylphosphonate Terminated Dendrimer with a Biotinylated Spacer

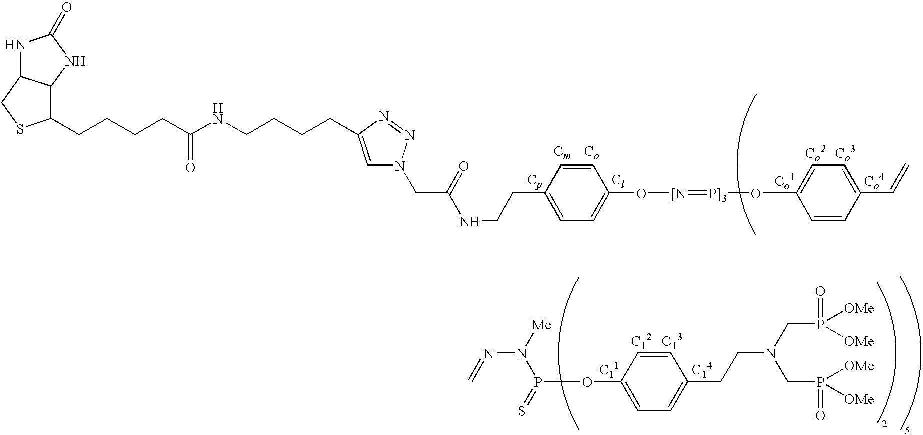

To a solution of the compound of example 4.18 (48 mg, 0.023 mmol) in acetone (5 mL) were added phenol of example 1.1 (92 mg, 0.241 mmol) and caesium carbonate (79 mg, 0.241 mmol) and the mixture was stirred at room temperature for 12 h. The reaction mixture was centrifugated, filtered and evaporated. The resulting crude oil was eluted on a plug of silica with acetone to remove the unreacted phenol then with acetone/methanol/water mixture (7:2:1). The resulting dendrimer solution was concentrated to dryness under reduced pressure, dissolved in 10 mL of dichloromethane, dried over sodium sulfate, filtered (micropore, 0.2 μm) and finally evaporated to dryness under reduced pressure to afford title compound as a colourless oil (yield=90%). ³¹P{¹H} NMR (acetone-d6, 202.5 MHz): δ=8.9 (s, N₃P₃), 26.3 (2 s, PO₃Me₂), 26.4 (2 s, PO₃Me₂), 62.8 (2 s, P═S); ¹H NMR (acetone-d6, 500.3 MHz): δ=1.42 (m, 2H, CO—CH₂—CH₂—CH₂—CH₂), 1.53 (m, 2H, CH₂—CH₂—CH₂—CH₂—NH), 1.62 (m, 2H, CO—CH₂—CH₂—CH₂—CH₂), 1.67 (m, 2H, CH₂—CH₂—CH₂—CH₂—NH), 1.74 (m, 2H, CO—CH₂—CH₂—CH₂—CH₂), 2.16 (broad t, ³J_{HH}=7.2 Hz, 2H, CO—CH₂—CH₂—CH₂—CH₂), 2.67 (m, 3H, CH₂—CH₂—CH₂—CH₂—NH, CH₂—S), 2.75 (m, 2H, C₆H₄—CH₂—CH₂—NH), 2.80 (m, 20H, C₆H₄—CH₂—CH₂—N), 2.89 (m, 1H, CH₂—S), 3.06 (m, 20H, C₆H₄—CH₂—CH₂—N), 3.20 (2 broad d, ²J_{HP}=12.7 Hz, 43H, N—CH₂—P, CH₂—CH₂—CH₂—CH₂—NH, CH—S), 3.48 (m, 17H, N—CH₃, C₆H₄—CH₂—CH₂—NH), 3.69 (2 d, 120H, ³J_{HP}=10.5 Hz, P(O)(OCH₃)), 4.27 (m, 1H, CH—CH—NH), 4.45 (m, 1H, CH₂—CH—NH), 5.07 (s, 2H, CO—CH₂—N), 5.90 (broad s, 1H, CH—CH—NH—CO), 6.10 (s, 1H, CH₂—CH—NH—CO), 6.90 (d, ³J_{HH}=8.3 Hz, 2H, Cₒ—H), 7.04-7.17 (m, 32H, Cₘ—H, C₀²—H, C₁²—H), 7.27 (m, 20H, C₁³—H), 7.32 (t, ³J_{HH}=5.6 Hz, 1H, (CH₂)₄—NH—CO), 7.65 (s, 1H, N—CH═C), 7.73 (m, 10H, C₀³—H), 7.88 (broad s, 3H, CH═N), 7.94 (broad s, 2H, CH═N), 8.00 (t, ³J_{HH}=5.5 Hz, 1H, NH—CO—CH₂—N); ¹³C{¹H} NMR (acetone-d6, 125.8 MHz): δ=25.0 (s, CH₂—CH₂—CH₂—CH₂—NH), 25.6 (s, CO—CH₂—CH₂—CH₂—CH₂), 26.6 (s, CH₂—CH₂—CH₂—CH₂—NH), 28.2 (broad s, CO—CH₂—CH₂—CH₂—CH₂, CO—CH₂—CH₂—CH₂—CH₂), 28.2 (s, CH₂—CH₂—CH₂—CH₂—NH), 32.2 (s, C₆H₄—CH₂—CH₂—N), 32.8 (2 d, ²J_{CP}=12.2 Hz, N—CH₃), 34.5 (s, C₆H₄—CH₂—CH₂—NH), 35.5 (s, CO—CH₂—CH₂—CH₂—CH₂), 38.5 (s, CH₂—CH₂—CH₂—CH₂—NH), 40.2 (s, CH₂—S), 40.7 (s, C₆H₄—CH₂—CH₂—NH), 49.1 (dd, ¹J_{CP}=157.3 Hz, ³J_{CP}=8.2 Hz, N—CH₂—P), 52.0 (m, P(O)(OCH₃), CO—CH₂—N), 55.7 (s, CH—S), 58.1 (t, ³J_{CP}=7.7 Hz, C₆H₄—CH₂—CH₂—N), 59.9 (s, CH₂—CH—NH), 61.5 (s, CH—CH—NH), 120.8 (s, Cₒ), 121.0 (d, ³J_{CP}=4.0 Hz, C₁²), 121.3 (m, C₀²), 122.8 (s, N—CH═C), 128.4 (broad s, C₀³), 129.9 (s, Cₘ), 130.1 (s, C₁³), 132.6 (m, C₀⁴), 136.6 (s, Cₚ), 137.3 (s, C₁⁴), 139.5 (broad t, ³J_{CP}=4.9 Hz, CH═N), 147.2 (s, N—CH═C), 148.9 (m, C₁¹), 151.2 (m, Cᵢ, C₀¹), 162.8 (s, HN—CO—NH), 165.7 (HN—CO—CH₂—N), 172.1 (s, (CH₂)₄—NH—CO) ppm.

### 4.20. Synthesis of Dimethylphosphonate Terminated Dendrimer with a Biotinylated Spacer [(Aza2P)₁₀-Biot-D]

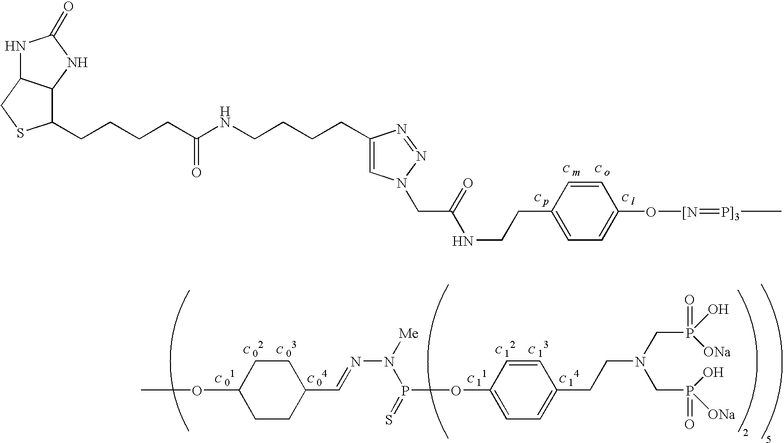

To a vigorously stirred solution of the compound of example 4.19 (36 mg, 0.007 mmol) in dry dichloromethane (2 mL) was added at 0° C., under a dry argon atmosphere, bromotrimethylsilane (40 μL, 0.299 mmol). The reaction mixture was stirred at room temperature overnight and then evaporated to dryness under reduced pressure. The crude residue was washed twice with methanol (2 mL) for 1 h at rt and evaporated to dryness under reduced pressure. The resulting white solid was washed once with diethylether (4 mL) and then transformed into its sodium salt as follows: the dendrimer was suspended in water (1 mL/100 mg) and one equivalent of sodium hydroxide per terminal phosphonic acid was added. The resulting solution was lyophilised to afford title compound as a white powder (yield=85%). ³¹P{¹H} NMR (D₂O/CD₃CN 7:1, 202.5 MHz): δ=7.1 (s, PO₃HNa), 9.5 (s, N₃P₃), 64.5 (s, P═S), 64.6 (s, P═S); ¹H NMR (D₂O/CD₃CN 7:1, 500.3 MHz): δ=1.64 (m, 2H, CO—CH₂—CH₂—CH₂—CH₂)), 1.82 (m, 4H, CO—CH₂—CH₂—CH₂—CH₂, CH₂—CH₂—CH₂—CH₂—NH), 1.93 (m, 4H, CH₂—CH₂—CH₂—CH₂—NH, CO—CH₂—CH₂—CH₂—CH₂), 2.47 (s, 2H, CO—CH₂—CH₂—CH₂—CH₂), 3.10 (m, 7H, CH₂—CH₂—CH₂—CH₂—NH, CH₂—S, C₆H₄—CH₂—CH₂—NH, CH—S), 3.54 (broad s, 22H, C₆H₄—CH₂—CH₂—N, CH₂—CH₂—CH₂—CH₂—NH), 3.75 (broad d, ²J_{HP}=10.1 Hz, 42H, N—CH₂—P, C₆H₄—CH₂—CH₂—NH), 3.81 (m, 15H, N—CH₃), 4.13 (m, 20H, C₆H₄—CH₂—CH₂—N), 4.54 (broad s, 1H, CH—CH—NH), 4.70 (broad s, 1H, CH₂—CH—NH), 5.33 (broad s, 2H, CO—CH₂—N), 7.26 (broad s, 2H, Cₒ—H), 7.42 (m, 12H, Cₘ—H, C₀²—H), 7.60 (m, 20H, C₁²—H), 7.69 (s, 1H, N—CH═C), 7.82 (m, 20H, C₁³—H), 8.06 (m, 10H, C₀³—H), 8.29 (broad s, 3H, CH═N), 8.33 (broad s, 2H, CH═N) ppm. ¹³C{¹H} NMR (D₂O/CD₃CN 7:1, 125.8 MHz): δ=24.8 (s, CH₂—CH₂—CH₂—CH₂—NH), 25.8 (s, CO—CH₂—CH₂—CH₂—CH₂), 26.4 (s, CH₂—CH₂—CH₂—CH₂—NH), 28.3 (broad s, CO—CH₂—CH₂—CH₂—CH₂), 28.5 (s, CO—CH₂—CH₂—CH₂—CH₂, CH₂—CH₂—CH₂—CH₂—NH), 29.3 (s, C₆H₄—CH₂—CH₂—N), 33.1 (d, ²J_{CP}=8.9 Hz, N—CH₃), 34.3 (s, C₆H₄—CH₂—CH₂—NH), 36.1 (s, CO—CH₂—CH₂—CH₂—CH₂), 39.3 (s, CH₂—CH₂—CH₂—CH₂—NH), 40.3 (s, CH₂—S), 40.6 (s, C₆H₄—CH₂—CH₂—NH), 52.2 (s, CO—CH₂—N), 55.4 (broad d, ¹J_{CP}=125.6 Hz, N—CH₂—P), 55.8 (s, CH—S), 57.9 (broad s, C₆H₄—CH₂—CH₂—N), 60.6 (s, CH₂—CH—NH), 62.3 (s, CH—CH—NH), 121.0 (s, Cₒ), 121.9 (broad s, ³J_{CP}=4.0 Hz, C₀², C₁²), 122.5 (s, N—CH═C), 128.9 (s, C₀³), 130.7 (s, Cₘ), 131.1 (s, C₁³), 133.0 (m, C₀⁴), 135.1 (broad s, Cₚ, C₁⁴), 141.1 (broad m, CH═N), 148.7 (s, N—CH═C), 149.7 (broad s, C₁¹), 151.1 (m, Cᵢ, C₀¹), 164.2 (s, HN—CO—NH), 167.2 (HN—CO—CH₂—N), 175.9 (s, (CH₂)₄—NH—CO) ppm.

### Example 5

### Inhibition of the Proliferation of CD4 T Lymphocytes by Dendrimer Gc1

### 5.1. Methods

Peripheral blood samples are obtained from adult healthy volunteers through the Etablissement Français du Sang (Toulouse, France). Peripheral Blood Mononuclear Cells (PBMC) are isolated by Ficoll density gradient (Amersham Biosciences AB).

PBMC are cultured at a final concentration of 1.5×10⁶ cells/mL in complete RPMI 1640 medium, i.e. supplemented with 10% of heat inactivated fetal calf serum (FCS) (Invitrogen), 1 mM sodium pyruvate (Invitrogen), 2 mM L-glutamine, 100 U/mL penicillin and 100 μg/mL streptomycin (Cambrex Bioscience). Proliferation is activated by 400 U/mL of interleukin 2 (IL2) (Sanofi-Aventis) (a “growth factor” for lymphocytes). Dendrimer Gc1 or (Aza2P)₁₂ is made soluble in sterile water at 2 mM and filtered through a 0.2 μm membrane before use at 20 μM in culture.

Proliferation of CD4 T lymphocytes is evaluated by flow cytometry by analyzing the intracellular dilution of a fluorescent probe: the 5,6-carboxyfluorescein diacetate N-succinimidylester (CFSE). Briefly, PBMC are labelled with 1 μM CFSE in serum free PBS for 8 minutes at 37° C. and then labelling is stopped by adding an equal volume of FCS. The unconjugated CFSE is eliminated by 2 washes in PBS, and the CFSE-labelled PBMC are cultured for 6 days. Then, CFSE dilution is analyzed by flow cytometry to measure the proliferation CD4 T cells. After gating for CD4 positive cells (mouse anti-human CD4-PE-Cy5 clone 13B8.2, Beckman-Coulter), CFSE fluorescence intensities are analyzed, and the percentage of CD4 T cells which have proliferated during the culture is taken into account. Each condition is done in triplicate. The overall differences are evaluated by analysis of variance test (ANOVA) with Sigma Stat software (Systat Software).

### 5.2. Results

They are shown in FIG. 4.

T lymphocytes, especially CD4 T lymphocytes, proliferate in presence of IL2. We observe that, after a few days with IL2 alone, 70.2% of CD4 T lymphocytes have divided (left peak in FIG. 4A, the right peak corresponds to non-divided CD4 T lymphocytes).

In the same conditions but with 20 μM of Gc1, only 17.2% of CD4 T lymphocytes have divided (left signal FIG. 4B, the right peak corresponds to non-divided CD4 T lymphocytes).

### Example 6

### Inhibition of the Proliferation of Purified CD4 T Lymphocytes by Bisphosphonic Dendrimers

### 6.1. Methods

PBMC from adult healthy volunteers are prepared as described in example 1.

CD4 T lymphocytes are then purified from PBMC by positive selection using microbeads conjugated with anti-CD4 mAb, according to the manufacter's instructions (Miltenyi Biotec). The cells obtained after the purification are >98% pure (as verified by flow cytometry).

Purified CD4 T lymphocytes are cultured at a final concentration of 1.5×10⁶ cells/mL in complete RPMI 1640 medium (as described in example 1.1). Then, their proliferation is triggered by microbeads coated with anti-CD3 and anti-CD28 mAbs (Dynabeads®, Dynal Biotech ASA). Dendrimer Gc1 or (Aza2P)₁₂ and its analogs are made soluble in sterile water at 2 mM and filtered through a 0.2 μm membrane before use at 20 μM in culture.

Proliferation of purified CD4 T lymphocytes is evaluated by flow cytometry by analyzing the intracellular dilution of a fluorescent probe: the CFSE.

### 6.2. Results

They are shown in FIGS. 5 and 6.

CD4 T lymphocytes strongly proliferate after triggering by microbeads coated with anti-CD3 and anti-CD28 mAbs in presence of IL2. We observe that, after a few days with microbeads and IL2, 88% of purified CD4 T lymphocytes have divided (FIG. 5A).

In the same conditions but with 20 μM of Gc1, only 10.2% of purified CD4 T lymphocytes have divided (FIG. 5B).

We observe the same inhibitory effect with the julolidin ((Aza2P)₁₀-Julo-D) and the biotinylated ((Aza2P)₁₀-Biot-D) analogs of Gc1 (FIG. 6).

These results show a direct inhibitory effect of azabisphosphonic dendrimers on the proliferation of CD4 T lymphocytes.

### Example 7

### Transcriptomic Study of Monocytes Activated by Dendrimer Gc1

### 7.1. Methods

### 7.1.1. Cell Preparation and Purification

Peripheral Blood Mononuclear Cells (PBMC) from adult healthy volunteers are prepared as described in example 1.

Monocytes are then purified by positive selection using microbeads conjugated with anti-CD14 mAb, according to the manufacter's instructions (Miltenyi Biotec). The cells obtained after the purification are >95% pure. The CD14 negative fraction is collected as Peripheral Blood Lymphocytes (PBL).

### 7.1.2. Monocyte Culture and Activation

Monocytes are cultured at a final concentration of 1×10⁶ cells/mL in complete RPMI 1640 medium (as described in example 1.1).

For genechip analysis, 10×10⁶ monocytes from 3 donors (and from 3 more donors for quantitative PCR) are cultured in 25 cm² dishes. They are stimulated with 20 μM of dendrimer for 6 hours or remain untreated (control cells).

For the functional experiments, freshly purified monocytes are cultured in 6-well plates. The cells are stimulated for 24 hours (immunophenotyping by flow cytometry analyses, MLR) or 96 hours (CD206 detection) with different stimuli or remain untreated (control). Dendrimer-activated monocytes (da-monocytes) are obtained with 20 μM of the dendrimer Gc1 or (Aza2P)₁₂, alternatively-activated monocytes (alt-monocytes) with 10 U/mL recombinant human IL4 (Peprotech). The classical activation of monocytes is obtained by priming the cells over night (18 hours) with 100 U/mL of human recombinant IFN-γ (R&D Systems) and, after a washing step in PBS, by stimulation with 10 ng/mL LPS from E. coli 011/B4 strain (InvivoGen) for the remaining 6 hours or 78 hours.

7.1.3. RNA Extraction and Complementary RNA (cRNA) Synthesis

Total RNA are extracted from 10×10⁶ monocytes using TRIzol™ Reagent (Invitrogen), according to the manufacter's instructions. The quality and integrity of the RNA obtained are assessed by using an Agilent 2100 Bioanalyser (Agilent Technologies) after a denaturating step at 70° C. for 2 minutes. cRNA are prepared according to one-Cycle Target Labelling protocol (Affymetrix) starting from 1 μg of total RNA.

### 7.1.4. Affymetrix Genechip Analysis

cRNA are fragmented and hybridized to Affymetrix HG-U133 plus 2.0 arrays. The chips are then washed and scanned, according to the manufacter's instructions. HG-U133 plus 2.0 arrays contain 54,675 sets of oligonucleotide probes that correspond to ≈39,000 unique human genes or predicted genes. GeneChip operating Software, Version 1.1 (Affymetrix), is used for the primary image analysis of the array, for the normalization (global scaling method, target value of 100), and for the comparisons between control and dendrimer-treated samples. Monocytes from 3 donors are analyzed after 6 hour incubation in the presence or absence of the dendrimer Gc1 or (Aza2P)₁₂. Genes are considered to be differentially regulated in da-monocytes compared to control cells if they have a fold change of ≧2.0 or ≦−2.0 for at least two donors of the three. The annotation tool, which is an automated method for the functional annotation of gene lists, is performed with the Affymetrix NetAffx data base (http://www.affymetrix.com/analysis/netaffx/index.affx). Gene Ontology data mining for biological process at level 4 and 5 is conducted on line via the DAVID website (http://david.abcc.ncifcrf.gov/).

### 7.1.5. Quantitative RT-PCR

Total RNA are purified as described above, and 5 μg are used to synthesize single-strand cDNA using M-MLV reverse transcriptase RNase H Minus (Promega), according to the manufacter's instructions. Quantitative RT-PCR is then performed using the Platinum SYBR Green qPCR SuperMix UDG (Invitrogen) with forward and reverse primers at a final concentration of 300 nM. The primers are designed using Primer Express software from mRNA sequences submitted to Ensembl data base, and are listed in FIG. 7. Quantification of the different mRNA is performed on a 7000 Sequence Detection System (Applied Biosystems). PCR amplification begins with 1 cycle of 50° C. for 2 minutes and 95° C. for 10 minutes, followed with 40 cycles of denaturation at 95° C. for 15 seconds and annealing/extension at 60° C. for 60 seconds. All experiments are performed in duplicate. For each donor, the relative expression is calculated as 2^{-ΔΔCt} where ΔΔCt is the difference between ΔCt of the sample and the ΔCt of the reference consisting of the control monocytes. ΔCt is the difference between the Ct of the target gene and the GAPDH gene. For statistical analysis, a one-tailed paired t test is performed comparing the ΔCt values for the da-monocytes and the corresponding control monocytes with Sigma Stat software (Systat Software).

### 7.1.6. Flow Cytometry

Flow cytometry is performed on a LSR-II cytometer (BD Biosciences). The data are analyzed with FACSDiva (BD Biosciences) or WinMDI softwares. The expression of surface markers is performed using mouse anti-human fluorochrome-conjugated mAbs specific for MHC class II-FITC (clone Tü36), CD86-PE (clone 2331), MHC class I-PE-Cy5 (clone G46-2.6) and CD13-PE (clone WM15) from BD Biosciences and specific for CD64-FITC (clone 22) and CD206-PE (clone 3.29B1.10) from Beckman-Coulter. Appropriate isotype-matched antibodies are used as negative control.

### 7.1.7. Allogenic Mixed Leucocyte Reactions (MLR)

MLR are performed in 96-well round bottom plates in a total volume of 200 μL in complete RPMI 1640 medium. Stimulation is assayed by incubating responder PBL (10⁵ cells) with different numbers of allogenic stimulating monocytes (activated with the different stimuli for 24 hours) (PBL:monocyte ratios ranging from 4:1 to 100:1). The proliferation of the CD4 T cells is analyzed by measuring the cytoplasmic dilution of CFSE (as described in 5.1. Methods). The CFSE-labelled PBL and the differently activated monocytes are then co-cultured for 6 days. Then, CFSE dilution is analyzed by flow cytometry to measure the proliferation of alloantigen-induced CD4 T cells. After gating for CD4 positive cells CFSE fluorescence intensities are analyzed, and the percentage of CD4 T cells which have proliferated during the MLR is taken into account. Each condition is done in triplicate. The overall differences are evaluated by analysis of variance test (ANOVA) with Sigma Stat software (Systat Software).

### 7.1.8. Intracellular Detection of IL10

Six days following the primary stimulation with one of the differently activated monocytes at a ratio of PBL:monocytes of 4:1, cells were restimulated with 10000 anti-CD3/anti-CD28 beads for 5 hours (according to the technical description by J. P. Edwards et al., (2006) J Leukoc Biol 80, 1298-1307) and with 10 μg/mL Brefeldin A (Sigma-Aldrich) to inhibit cytokine secretion. Cells are then harvested, washed with PBS and incubated 15 minutes at 4° C. with mouse mAb anti-human CD4-PE (clone 13B8.2, Beckman-Coulter) in PBS with 5% FCS. Cells washed twice, are then fixed by 2% paraformaldehyde in PBS and permeabilized with 1% saponin in PBS. Intracellular staining is performed with AlloPhycoCyanin-conjugated rat anti-human IL10 (clone JES3-19F1, BD Bioscience) for 30 minutes at 4° C. in PBS with 1% saponin and analyzed by flow cytometry. Each condition is done in triplicate and results are expressed as the mean±SD. Intracellular IL10 mean fluorescence intensity (mfi) means across the differently activated monocytes are compared by one-way analysis of variance, the comparisons between CD4⁺/CFSE⁻ and CD4⁺/CFSE⁺ cells are done with Student's t-test using Sigma Stat software (Systat Software).

### 7.2. Results

### 7.2.1. Human Monocyte Transcriptional Profile After Activation by Dendrimer Gc1 or (Aza2P)₁₂

Results are shown in FIGS. 8 and 9.

78 genes were found over-expressed and 62 genes were found under-expressed by da-monocytes. On the one hand, up-regulation of genes coding for proteins characterizing the alternative activation of monocytes/macrophages such as the mannose receptor (MRC), the IL1 receptor antagonist (IL1 RN), the immuno-suppressive cytokine IL10, and the chemokine CCL18 (also called AMAC-1 for Alternative Macrophage Activation-associated CC-chemokine-1) is detected. C1QR1 (important for the anti-inflammatory phagocytosis of apoptotic cells), CHI3L1, matrix metalloproteinases (of which MMP1) and SLAMF1 are also found over-expressed. On the other hand, down-regulation of genes coding for CXCR4 (receptor for the pro-inflammatory CXL12 chemokine), metallothioneins and IFITs (gene clusters induced by IFN-γ) is detected. These genes are likely to be expressed after IFN-γ and/or LPS treatment. Moreover, we detect a down-regulated expression of adhesion molecules such as CD9, CD11a and CD18. CD11a/CD18 also know as lymphocyte function antigen 1 (LFA-1) are implicated in mechanisms of leukocyte recruitment on inflamed tissue (42).

### 7.2.2. Validation of the Alternative-Like Activation of Human Monocytes by Quantitative RT-PCR and Analysis of Da-Monocyte Phenotype

Results are shown in FIG. 10.

9 gene transcripts of high relevance for the classical activation pathway (one pro-inflammatory chemokine: CCL5 and 3 pro-inflammatory cytokines: IL1β, IL6, IL12) or for the alternative activation pathway (MRC1, IL1 RN, IL10, CCL18 and CD23) are quantified. Results of quantitative RT-PCR are compared in da-monocytes and untreated monocytes of the 3 donors enlisted for the transcriptional study plus 3 supplementary donors. 5 genes whose products indicate an alternative activation (MRC1, IL1 RN, IL10, CCL18 and CD23) are significantly up-regulated in da-monocytes, whereas the expression levels of 3 genes out of 4 selected to indicate a classical activation (IL1β, IL6 and IL12) are not significantly modified and the fourth gene (coding for CCL5) is significantly under-expressed. These results confirm those of example 3.2.1.

### 7.2.3. Phenotype of Da-Monocytes

Results are shown in FIG. 11.

Expression of the mannose receptor MRC1 (CD206) is strongly enhanced by alt-monocytes and by da-monocytes, whereas this marker is weakly expressed by class-monocytes. On the contrary, CD64 (a FcγRI) is overexpressed in class-monocytes, but not in alt- or da-monocytes. Finally, FIG. 11 shows that CD13, a cell membrane-associated aminopeptidase N, also discriminates between the different types of monocyte activation. Class-monocytes express a higher level of CD13 than alt- and da-monocytes. Taken together, the quantitative study of gene transcripts and the analysis of the phenotype of differently activated monocytes strengthen the hypothesis of an alternative activation of human monocytes by bisphosphonic dendrimers.

### 7.2.4. The Activation of Monocytes by the Dendrimer Gc1 or (Aza2P)12 is Anti-Inflammatory and Immuno-Suppressive

Results are shown in FIGS. 12(1) and (2).

After 24 hours stimulations, the phenotypes of alt-, class- and da-monocytes are compared for the expression of HLA-DR, HLA-A,B,C (antigen presenting molecules) and CD86 (co-stimulatory molecule) markers by flow cytometry (FIG. 12(1)A). As expected, class-monocytes express high levels of antigen-presenting molecules and CD86, whereas alt-monocytes express lower levels of these molecules and appear as poor antigen-presenting cells (APC). In this respect, da-monocytes are closely related to alt-monocytes and also appear as poor APC. To compare the stimulatory capacity of differently activated monocytes, MLR with PBL and allogeneic activated-monocytes are performed. MLR activation is conducted for 4 different PBL/monocyte ratios from 4:1 to 100:1. MLR are measured as the percentage of divided CD4⁺ T lymphocytes among the PBL by using the dilution of the fluorescent dye CFSE. The results for 3 representative donors are compiled in FIG. 12(1)B with alt-, class- and da-monocytes as activators of MLR. As awaited, MLR activation by class-monocytes is always significantly higher than activation by alt-monocytes, except for the 4:1 ratio of donor 1. Statistical analysis is given in FIG. 12(2). It is noteworthy that da-monocytes systematically give the lowest activating effect on MLR. In detail, MLR activations by da-monocytes are always lower than activations by class-monocytes. When compared to activations by alt-monocytes, activations by da-monocytes are also significantly lower, except for donor 2, 25:1 ratio of donor 1 and 10:1 and 25:1 ratios of donor 3.

### 7.2.5. Intracellular Detection of IL10

Results are shown in FIG. 13.

Intracellular labelling of IL10 is compared in divided (CFSE⁻) and in non-divided (CFSE⁺) CD4⁺ T cells (FIG. 13(A)) resulting from the different MLR contexts after re-stimulation for 5 hours with anti-CD3/anti-CD28 mAbs coated on microbeads. The results from a representative donor out of 3 show an increase of the percentage of IL10⁺ CD4⁺ dividing cells in all MLR (FIG. 13(B)). FIG. 13(C) shows a comparison of the production of IL10 in divided and non-divided CD4⁺ T cells for the three MLR contexts independently. Statistical analysis of the mfi detected after labelling of IL10 gained in experiments with 3 donors indicates a significantly increased production of IL10 in divided (CFSE⁻) CD4⁺ T lymphocytes in MLR with da- and alt-monocytes. Conversely, the divided and non-divided CD4⁺ T cells in MLR with class-monocytes do not show significantly different levels of IL10 production. FIG. 13(D) shows the comparison of the production of IL10 in divided cells aroused from the three different MLR. The production of IL10 by CD4⁺ T cells is significantly enhanced in MLR with da- and alt-monocytes when compared to MLR with class-monocytes.

Thus, alt- and da-monocytes are poor inducers of CD4 T cell proliferation. What is more, da-monocytes induced IL10-producing CD4 T cells.

### Example 8

### Inhibition of the Differentiation of Monocytes in Osteoclasts by 20 μM of Dendrimer Gc1 or (Aza2P)₁₂

### 8.1. Introduction

The combination of cytokines such as RANK-Ligand and M-CSF induces the differentiation of human monocytes in osteoclasts. In bone biology, osteoclasts are responsible for bone resorption whereas osteoblasts rebuilt it. In some pathological contexts, the balance between the activities of osteoclasts and osteoblasts is compromised in the direction of the osteoclastic activity resulting in bone resorption.

### 8.2. Methods

PBMC from adult healthy volunteers are prepared as described in example 1.

Monocytes are then purified by negative selection using a kit containing a mixture of mouse anti-human mAbs and superparamagnetic polystyrene beads coated with a human anti-mouse IgG mAbs, according to the manufacter's instructions (Dynal). The cells obtained after the purification are >90% pure.

Pure monocytes are activated or not during 6 hours with 20 μM of dendrimer Gc1 or (Aza2P)₁₂. After three washings, activated and not activated monocytes are cultured for 21 days in α-MEM complete medium, i.e. supplemented with 10% of heat inactivated fetal calf serum (FCS) (Invitrogen), 2 mM L-glutamine, 100 U/mL penicillin and 100 μg/mL streptomycin (Cambrex Bioscience) in presence or not of 20 μM of dendrimer Gc1 or (Aza2P)₁₂. To generate osteoclasts, 50 ng/mL of M-CSF (PeproTech) and 30 ng/mL of sRANK-Ligand (PeproTech) are added to the culture medium. Each three days, half medium is changed and 25 ng/mL of M-CSF and 100 ng/mL of sRANK-Ligand are added. At day 21, the cells are fixed and stained with the leucocyte phosphatase acid kit according to the manufacter's instructions (Sigma-Aldrich).

### 8.3. Results

They are shown in FIG. 14.

When added at 20 μM in the culture medium, dendrimer Gc1 or (Aza2P)₁₂ inhibits the differentiation of human monocytes in osteoclasts (picture 1 of FIG. 14 shows osteoclasts as giant multinuclear cells whereas picture 2 shows nucleus of undifferentiated monocytes).

When monocytes are pre-incubated with 20 μM of dendrimer Gc1 or (Aza2P)₁₂ during 6 hours, then rinsed before stimulation with RANK-Ligand and M-CSF, we also observe an inhibition of their differentiation in osteoclasts (picture 3 of FIG. 14 shows only a few osteoclasts among undifferentiated monocytes).

### Example 9

### Inhibition of Bone Resorption in Vitro

### 9.1. Methods

PBMC from adult healthy volunteers are prepared as described in example 1.

Monocytes are then purified as described in section 4.2.

Monocytes were cultured in bone matrix 96-well plates (OsteoAssay Human bone plate, Lonza, USA) and differentiated into osteoclasts in the presence of M-CSF and sRANK-Ligand as described in section 4.2. Supernatants were collected and bone resorption was evaluated using a Crosslaps assay (Nordicbioscience, Danemark) which measures collagen degradation fragments.

### 9.2. Results

They are shown in FIG. 15.

Results show the release of fragments of bone matrix collagen (CTX, in nM) measured at days (D) 12 and 16. Dendrimer Gc1 or (Aza2P)₁₂ inhibits the in vitro resorption of bone when added directly in the differentiating medium (FIG. 15(A)) or when monocytes have been pre-incubated for 6 hours and rinsed before stimulation by RANK-Ligand et M-CSF (FIG. 15B). Dendrimer Gc1 or (Aza2P)₁₂ at 200 nM inhibits approximatively 60% of bone resorption at day 12, and 80% at day 15 (FIG. 15(A)).

When monocytes have been pre-incubated with the dendrimer Gc1 or (Aza2P)₁₂ before induction of the differentiation, inhibition of bone resorption is around 80% at day 12 and 50% at day 15 (FIG. 15(B)).

Taken together, these results show that bisphosphonic dendrimers activate human monocytes toward an <<alternative-like>> response and thus can be used as drugs for the treatment of uncontrolled inflammatory disorders in chronic or acute diseases such as psoriasis, rheumatoid arthritis or auto-immune disorders.

1-24. (canceled)

25. A method for treating inflammatory diseases through an anti-inflammatory type activation of monocytes, said method comprising the administration to a patient in need thereof of an effective quantity of at least one dendrimer with mono- or biphosphonic terminations.

26. A method according to claim 25 wherein the dendrimers with monophosphonic or bisphosphonic terminations are of generation n and comprise a central core § with a valency of m which can establish m−2 bonds, providing that m is greater than 2, or m−1 bonds, providing that m is greater than 1, or m bonds with linkage chains, preferably identical to each other, m representing an integer from 1 to 20, in particular of 1 to 10 and more particularly of 1 to 8 and n representing an integer from 0 to 12, which linkage chains are constituted by:
generation chains attached in a tree-like structure around the core on each of the bonds, when n is greater than or equal to 1, a generation chain of a given generation being linked to
a generation chain of the generation immediately below the given generation, or to the core when the given generation is 1, and to
at least 2 generation chains of the generation immediately above the given generation, or optionally to at least one intermediate chain when the given generation is n,
a terminal group being attached to the end of each generation chain of generation n, or if appropriate to the end of each intermediate chain, or
intermediate chains attached around the core on each of the bonds, when n is 0, a terminal group being attached to the end of each intermediate chain;
said terminal group being represented by the formula: 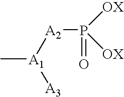
where
A₁ represents N; a P═Y group, where Y represents O, S, or no atom; an N—R group; or a C—R group; R representing H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a —NR′R″ group, an alkoxy group of 1 to 16 carbon atoms, an alkyl group of 1 to 16 carbon atoms, a halogen atom, a —NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R′ and R″ representing independently of each other H or an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
A₂ represents a single bond or a linear, branched or cyclic hydrocarbon chain with 1 to 6 members, each of said members optionally containing one or more heteroatoms, said heteroatoms being preferably chosen from a sulphur, oxygen, phosphorus or nitrogen atom, more preferably nitrogen, and being optionally substituted by at least one substituent chosen from H, an alkyl group of 1 to 16 carbon atoms, a halogen, a NO₂ group, a NRR′ group, a —CN group, a —CF₃ group, a hydroxyl group, an alkoxy group of 1 to 16 carbon atoms, an aryl or heteroaryl group of 1 to 24 carbon atoms, the heteroelement being preferably chosen from oxygen, nitrogen or sulphur, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R and R′ representing independently of each other H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
A₃ represents H, or a linear, branched or cyclic hydrocarbon chain with 1 to 6 members, each of said members being optionally chosen from a heteroatom, said heteroatom being preferably chosen from sulphur, nitrogen, phosphorus or silicon, more preferably nitrogen, each member being able to be optionally substituted by at least one group chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹ or 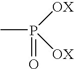
in particular A₃ can represent 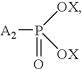
each A₂ being identical or different;
each OX, identical or different for each phosphonic group, represents OH, Oalkyl, where the alkyl group comprises from 1 to 16 carbon atoms, Oaryl, where the aryl group comprises from 6 to 24 carbon atoms, Oaralkyl, where the aralkyl group comprises from 7 to 24 carbon atoms, Oalkylaryl, where the alkylaryl group comprises from 7 to 24 carbon atoms, OSiR′₁R′₂R′₃, where R′₁, R′₂ and R′₃, identical or different, represent an alkyl group of 1 to 16 carbon atoms, or O⁻M⁺, where M⁺ is a cation of elements of group IA, IB, IIA, IIB or IIIA, IIIB of the periodic table of the elements, preferably M is chosen from the cations of the sodium, potassium, copper, calcium, barium, zinc, magnesium, lithium and aluminum atoms, or hydrocarbon groups of 1 to 100 carbon atoms, or nitrogenous groups of 0 to 100 carbon atoms, such as NR₁R₂R₃R₄⁺, where, independently of each other R₁, R₂, R₃ and R₄ represent H or a linear, branched or cyclic hydrocarbon chain with 1 to 16 members, optionally containing one or more heteroatoms, said heteroatoms being preferably chosen from oxygen, sulphur, nitrogen, phosphorus, silicon and/or one or more double or triple bonds, each of said members being optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹,
m represents an integer from 1 to 20, in particular from 1 to 10 and more particularly from 1 to 8;
n represents an integer from 0 to 12,
said central core § representing a group comprising from 1 to 500 atoms, and optionally containing one or more heteroatoms, said heteroatoms being preferably chosen from oxygen, sulphur, nitrogen, phosphorus or silicon.

27. A method according to claim 26 wherein the core § establishes m bonds with m identical linkage chains constituted:
either by generation chains attached in a tree-like structure around the core on each of the bonds, the end of each chain generation furthest from the central core being attached either to a terminal group or to an intermediate chain, the end of each intermediate chain being attached to a terminal group,
or by intermediate chains attached around the core on each of the bonds, the end of each intermediate chain being attached to a terminal group.

28. A method according to claim 27 wherein the core establishes m−2 or m−1 bonds, m representing an integer from 3 to 20, in particular from 3 to 10 and more particularly from 3 to 8, with respectively m−2 or m−1 identical linkage chains constituted:
either by generation chains attached in a tree-like structure around the core on each of the bonds, the end of each chain generation furthest from the central core being attached either to a terminal group or to an intermediate chain, and the end of each intermediate chain being attached to a terminal group,
or by intermediate chains attached around the core on each of the bonds, the end of each intermediate chain being attached to a terminal group;
the 1 or 2 remaining bonds being attached to linkage groups, identical or different, optionally linked together, in particular by means of a covalent bond, constituted:
either by part of the linkage chains defined above,
or by a hydrogen atom,
or by hydrocarbon groups comprising from 1 to 500 carbon atoms, said hydrocarbon groups being in particular constituted by H or by a linear, branched or cyclic hydrocarbon chain with 1 to 200 members, optionally containing one or more double or triple bonds, each of said members being optionally chosen from a heteroatom, said heteroatom being preferably chosen from a nitrogen, oxygen, phosphorus, silicon or sulphur atom, an aryl group of 6 to 24 carbon atoms, a heteroaryl group of 1 to 24 carbon atoms, a carboxyl group, a >C═NR group, each member being able to be optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, the first member of said hydrocarbon chain preferably being oxygen or nitrogen.

29. A method according to claim 25 wherein the generation chains are chosen from any linear, branched or cyclic hydrocarbon chain with 1 to 12 members, optionally containing one or more double or triple bonds, each of said members being optionally chosen from a heteroatom, said heteroatom being preferably chosen from nitrogen, oxygen, sulphur, phosphorus or silicon, an aryl group of 6 to 24 carbon atoms, a heteroaryl group of 1 to 24 carbon atoms, the heteroelement being preferably chosen from oxygen, nitrogen or sulphur, a carboxyl group, a >C═NR group, each member being able to be optionally substituted by at least one substituent chosen from an alkyl group of 1 to 16 carbon atoms, a halogen atom, an —NO₂ group, an —NRR′ group, a —CN group, a —CF₃ group, a hydroxyl group, an alkoxy group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹.

30. The method according to claim 25 wherein the intermediate chains are chosen from the groups corresponding to the formula:
-J-K-L-
where
J represents an oxygen or sulphur atom, or an —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹,
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;

31. The method according to claim 25 wherein the core is chosen from:
a nitrogen or silicon atom;
a group of formula 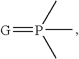
in which G represents an oxygen,
nitrogen, sulphur, selenium, tellurium atom or an ═NR group, R representing H or an alkyl group of 1 to 16 carbon atoms, an aryl group of 6 to 24 carbon atoms, or an aralkyl group of 7 to 16 carbon atoms, such as the
thiophosphoryl group of formula 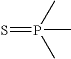
or an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹
a bis-phenyloxy group of formula 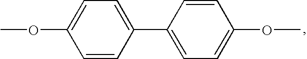
a 1,2-diamino-ethane group of formula 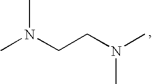
a 1,4-diamino-butane group of formula 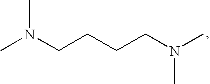
a cyclotriphosphazene group of formula 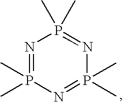
also denoted N₃P₃ or P₃N₃,
a cyclotetraphosphazene group of formula 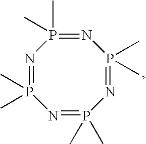
also denoted N₄P₄ or P₄N₄.

32. The method according to claim 25 wherein the dendrimers with monophosphonic or bisphosphonic terminations are of structure PAMAM, DAB or PMMH.

33. The method according to claim 32 wherein the dendrimer corresponds to the structure PAMAM for which n=4 and m=4 which is the following:

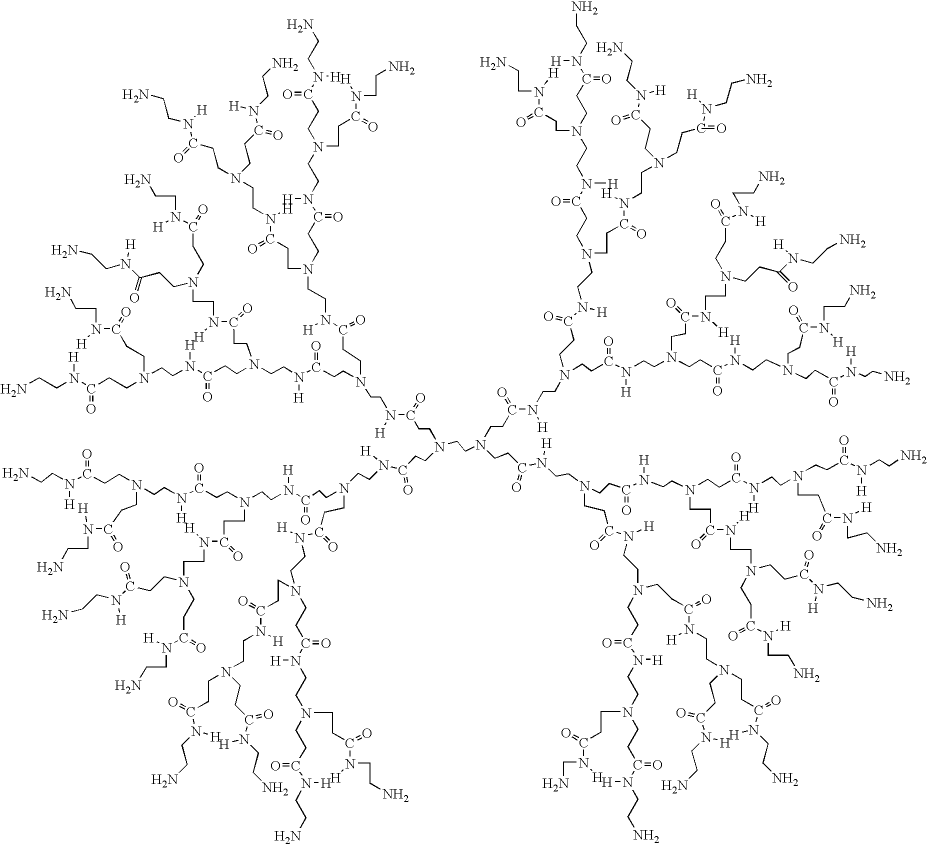

34. The method according to claim 32 wherein the dendrimer corresponds to the structure of type DAB, for which n=5 and m=4 which has the following formula:

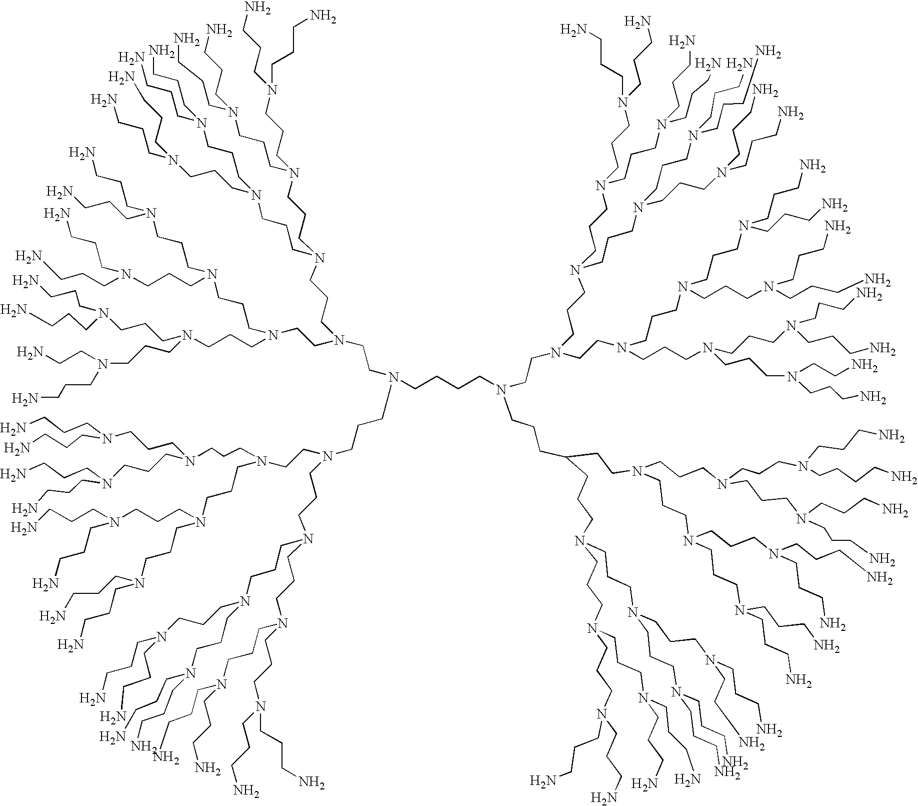

35. The method according to claim 32 wherein the dendrimers is of type PMMH which are selected from the group comprising:
a compound with a thiophosphoryl core, for which n=4 and m=3 represented below: 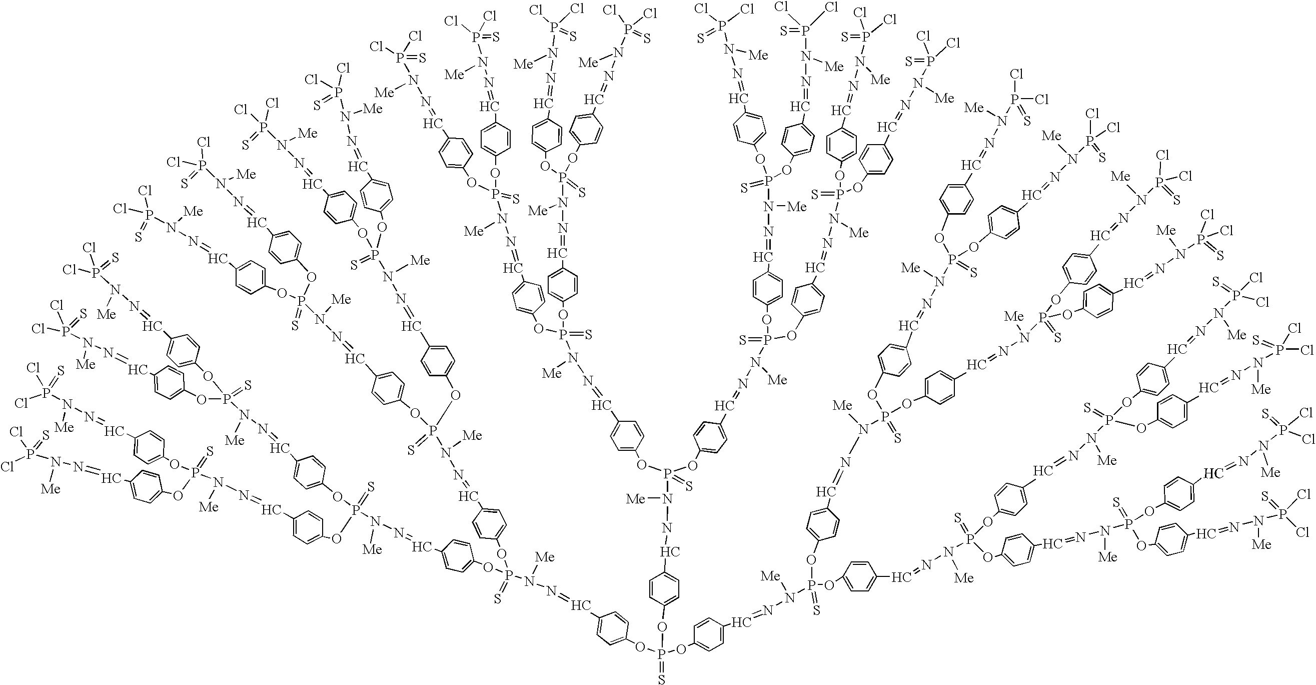
a compound with a cyclotriphosphazene core, for which n=2 and m=6, without an intermediate chain, represented below: 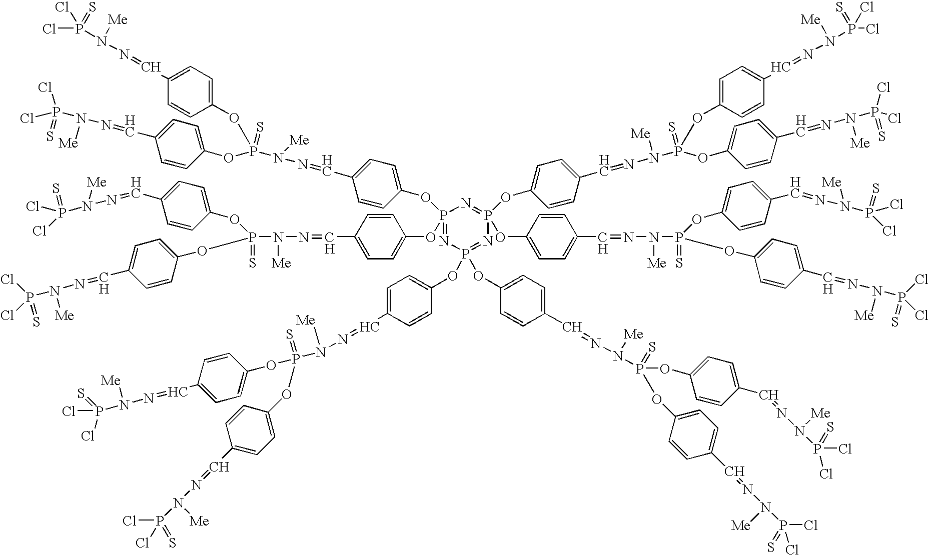
a compound with a cyclotriphosphazene core, for which n=2 and m=6, with an intermediate chain, represented below:

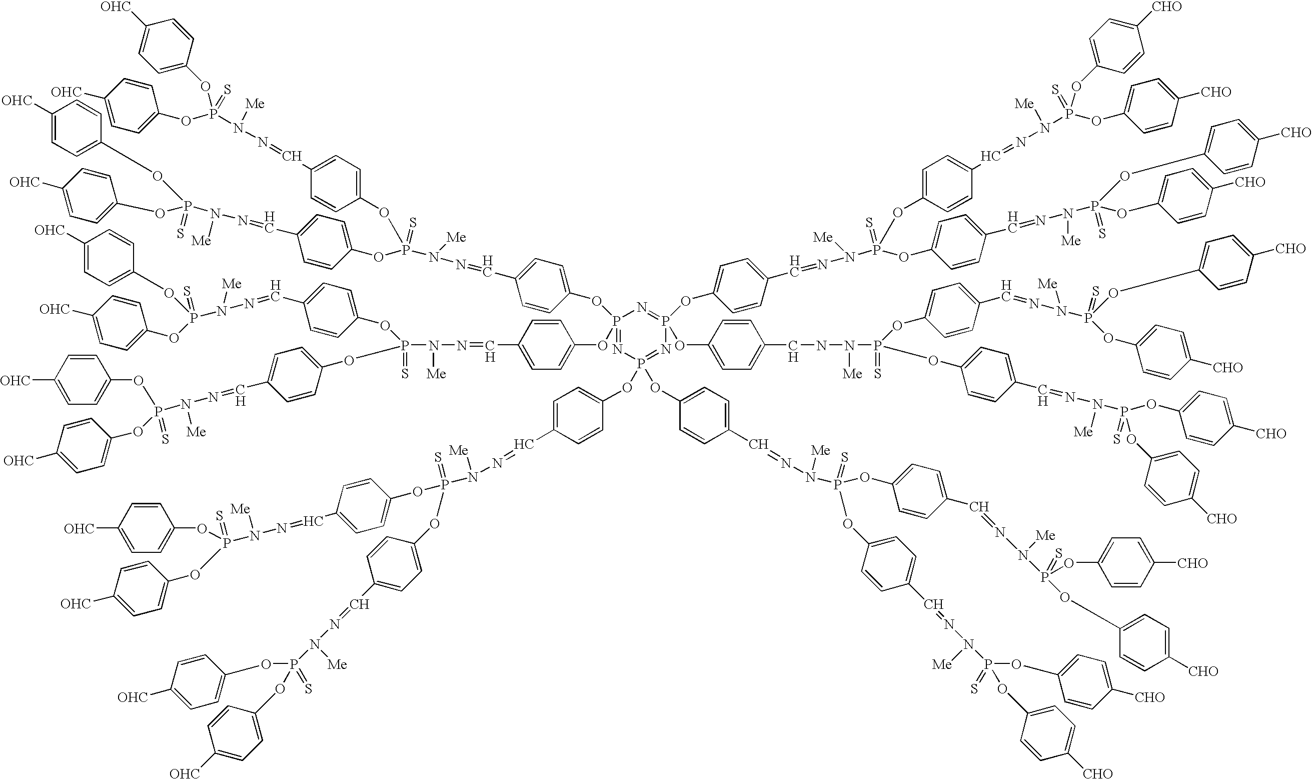

36. The method according to claim 25 wherein the dendrimers with monophosphonic or bisphosphonic terminations correspond to the following general formula (1a): 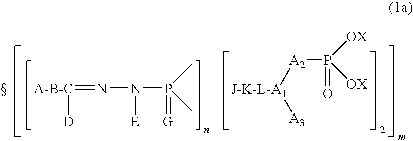
where n represents an integer from 0 to 3, selected from the group comprising the compounds where
when n=0, formula (1a) corresponds to the following formula (2a), 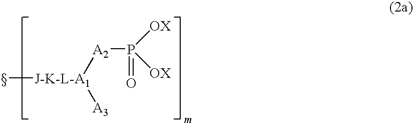
when n=1, formula (1a) corresponds to the following formula (3a), 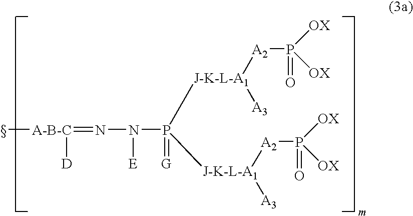
when n=2, formula (1a) corresponds to the following formula (4a), 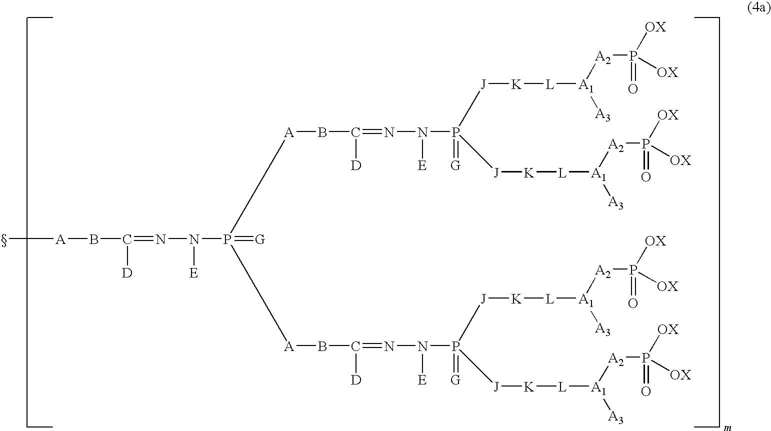
and when n=3, formula (1a) corresponds to the following formula (5a), 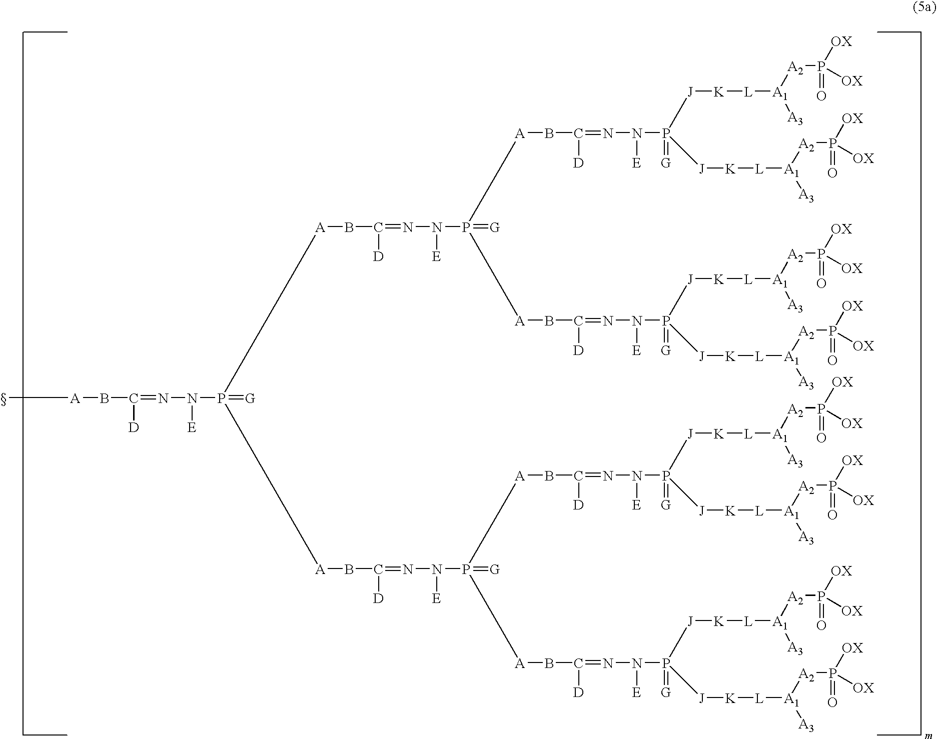
wherein the
the central core § is chosen from the following groups: 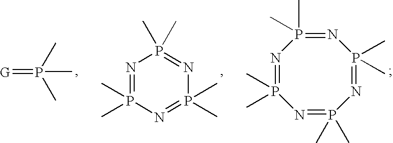
the generation chain corresponds to the formula: 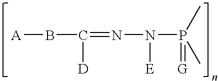
where
A represents an oxygen, sulphur, phosphorus atom or an —NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a —NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OE group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:
-J-K-L-
where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, optionally containing one or more double or triple bonds, each of said members being able to be optionally a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula: 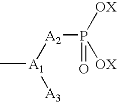
wherein A₁, A₂, A₃ and X are as previously defined, each X being identical or different.

37. The method according to claim 36 wherein the dendrimers with monophosphonic or bisphosphonic terminations correspond to the following general formula (1a) in which A₃ represents: 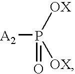
said general formula (1a) then corresponding to the following general formula (1): 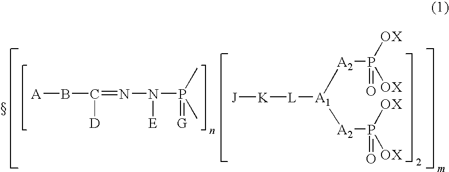
§, A, B, C, D, E, G, J, K, L, A₁, A₂, X, m and n being as previously defined.

38. The method according to claim 36 wherein the dendrimers correspond to general formula (1) wherein n represents an integer from 0 to 3, selected from the group comprising the compounds wherein
when n=0, formula (1) corresponds to the following formula (2), 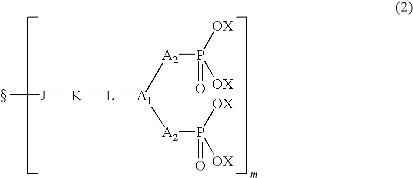
when n=1, formula (1) corresponds to the following formula (3), 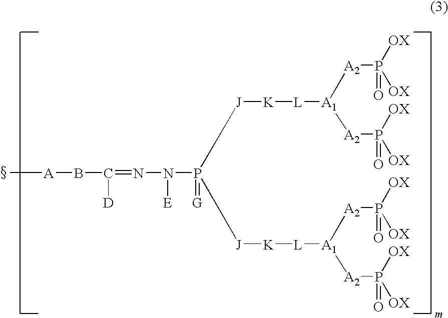
when n=2, formula (1) corresponds to the following formula (4), 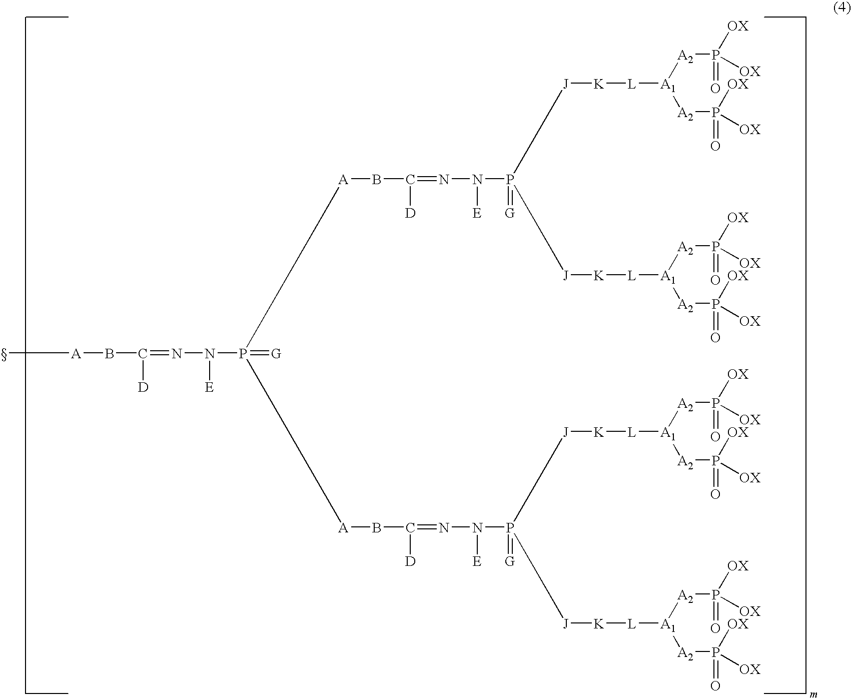
and when n=3, formula (1) corresponds to the following formula (5), 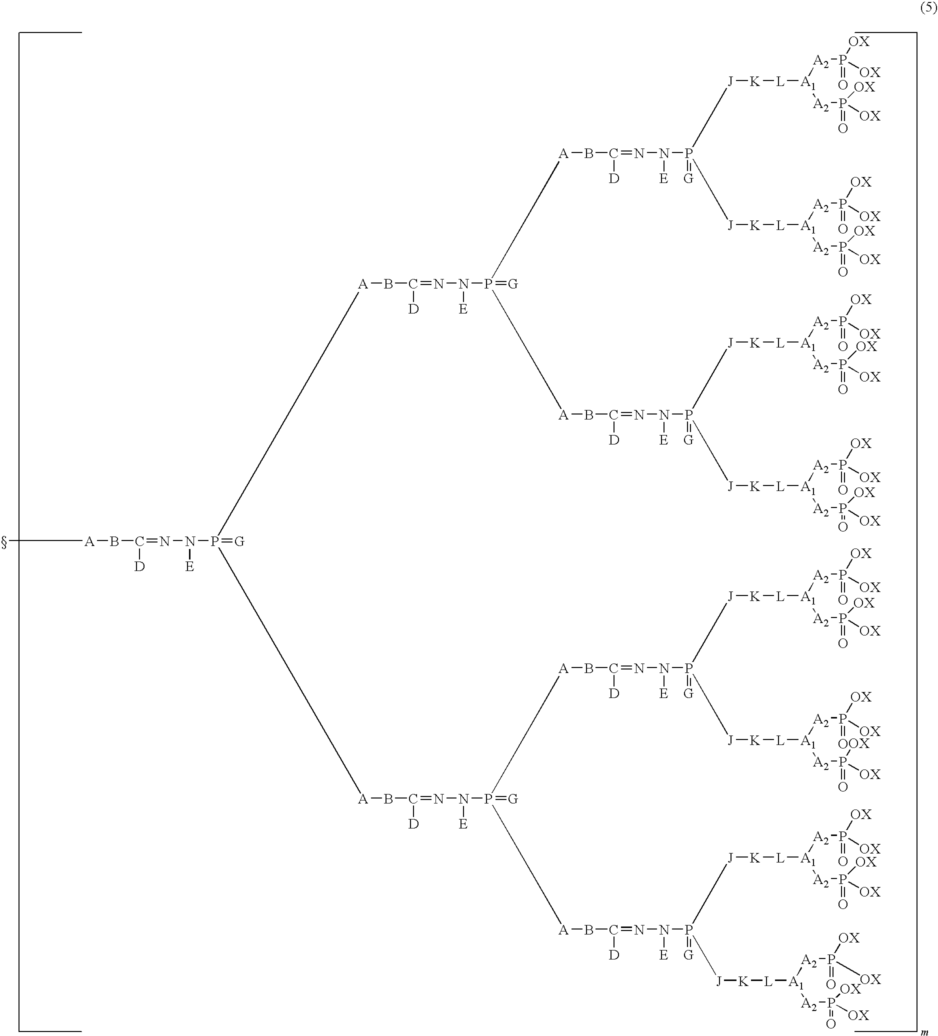
and wherein
the central core § is chosen from the following groups: 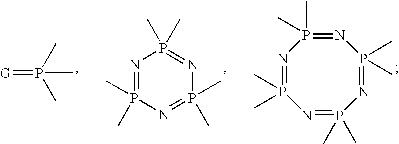
m represents 3, 6 or 8;
the generation chain corresponds to the formula:
where 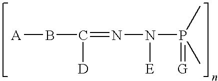
A represents an oxygen, sulphur, phosphorus atom or an —NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹; each being able to be optionally substituted by a halogen atom or a NO₂, —NRR′, —CN, —CF₃, —OE group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OE group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:
-J-K-L-
where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula: 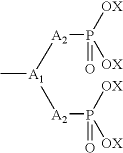
where A₁, A₂ and X being as previously defined, each X being identical or different.

39. The method according to claim 36 wherein the dendrimers correspond to general formula (1) of PMMH structure wherein:
§ represents 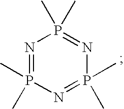
m represents 6;
n represents 0, 1, or 2;
A represents an oxygen atom;
B represents a benzene group;
D represents hydrogen;
E represents a methyl group;
G represents a sulphur atom;
J represents an oxygen atom;
K represents a benzene group;
L represents a non-substituted linear saturated hydrocarbon chain with two carbon atoms;
A₁ represents a nitrogen atom;
A₂ represents a CH₂ group;
X represents a methyl group, or a hydrogen or sodium atom;
said dendrimer being designated GCn, n being defined above.

40. The method according to claim 25 wherein the dendrimers are selected from the group comprising: 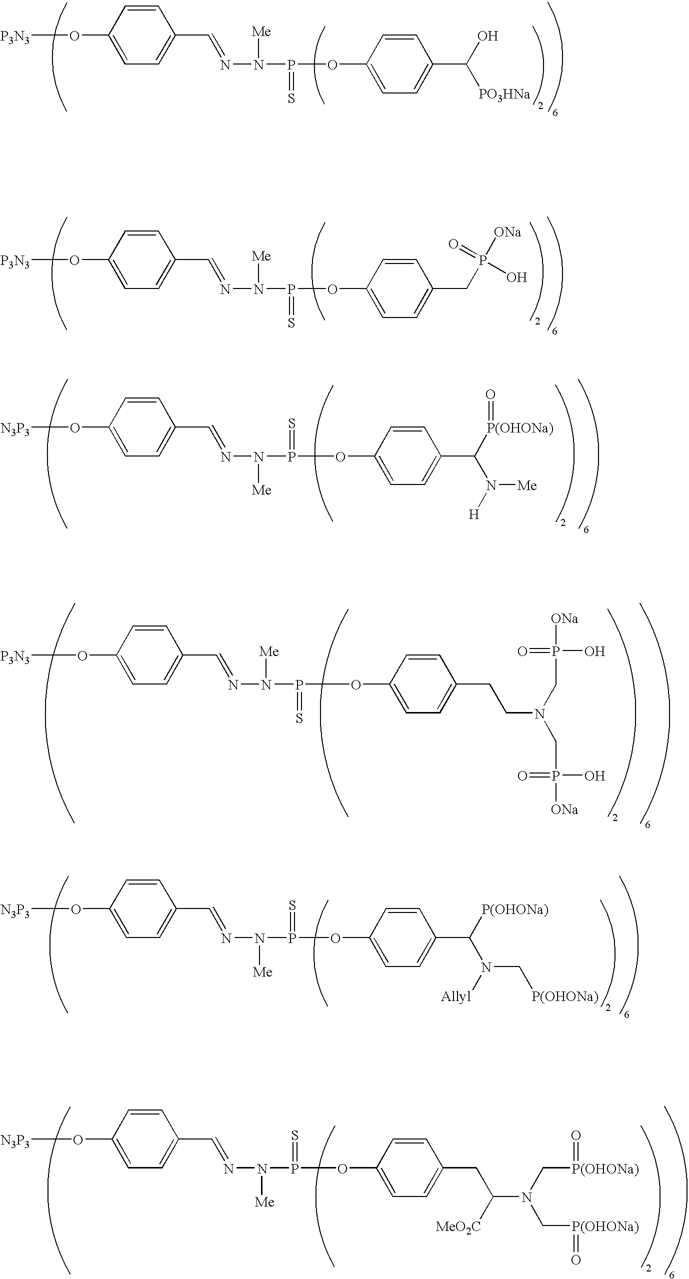 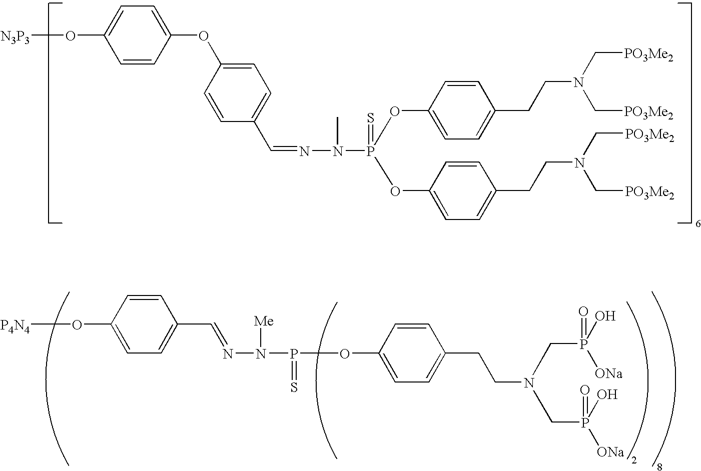
or of GC1 compounds of the following formulae:

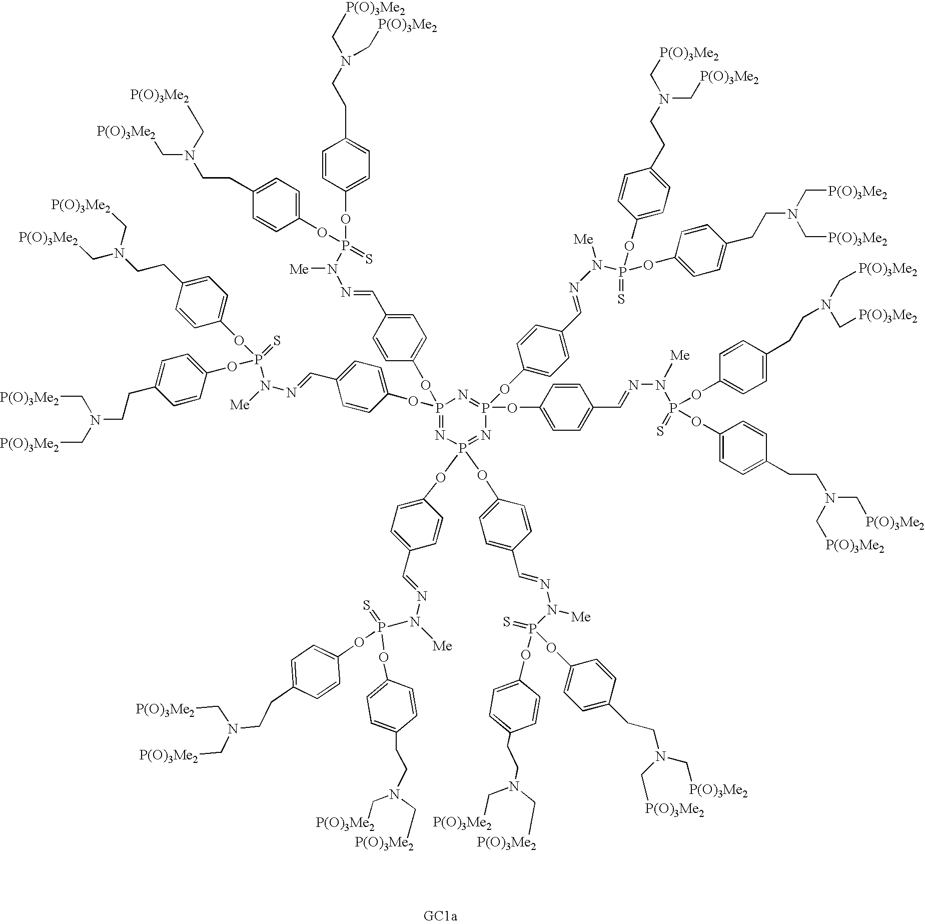

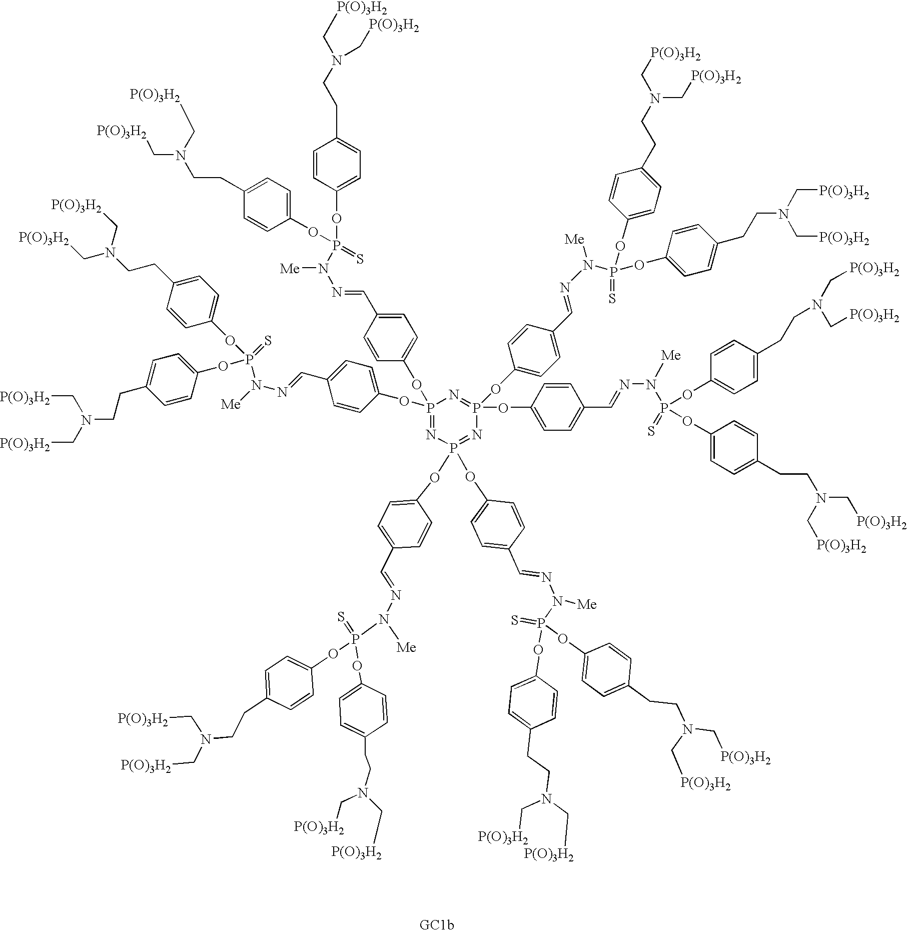

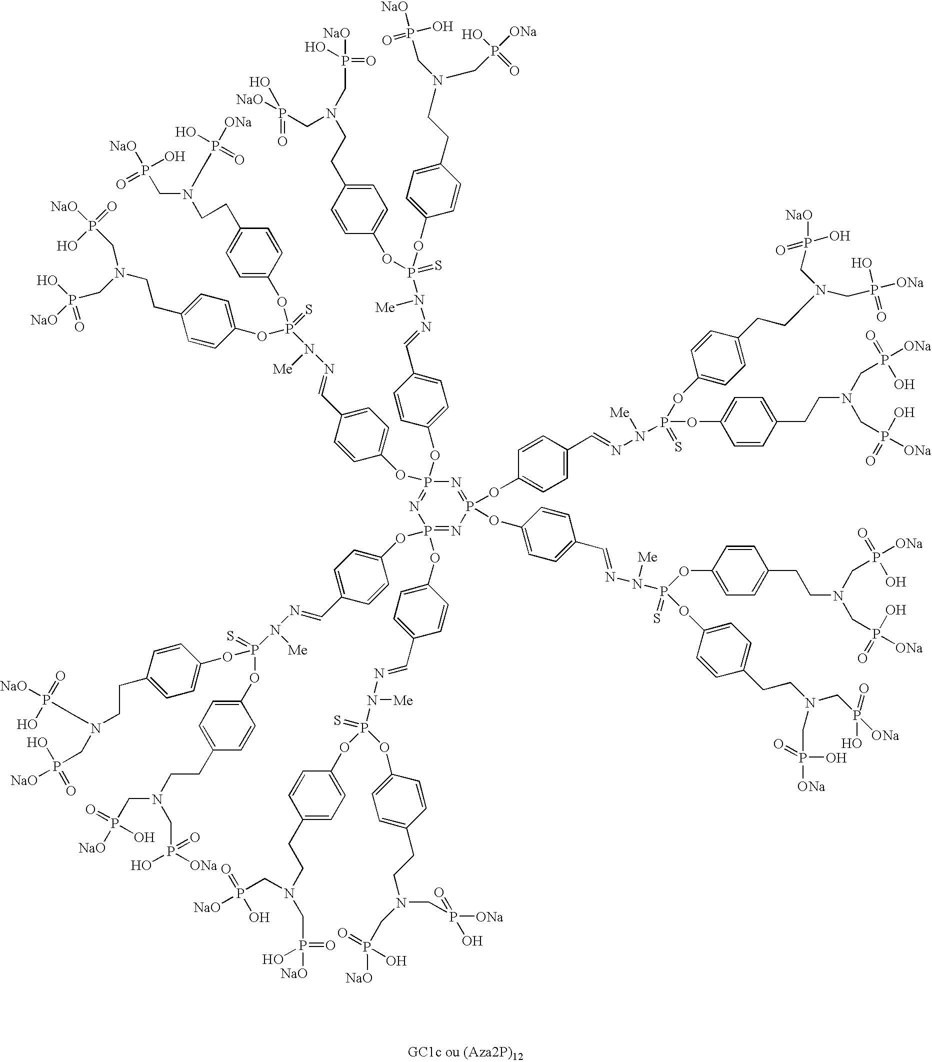

41. The method according to claim 26 wherein the dendrimers with bisphosphonic terminations correspond to the following general formula (7): 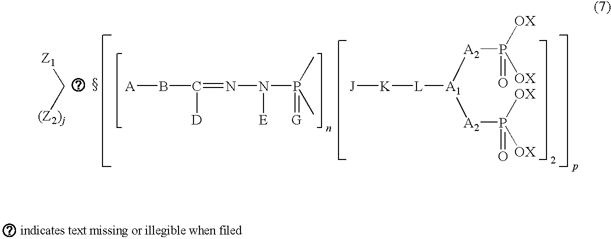
wherein
n represents an integer from 0 to 3, m represents 3, 6 or 8, p represents m−1 or m−2, and j represents 0 when p represents m−1 and 1 when p represents m−2, selected from the group comprising the compounds wherein
when p=m−1, formula (7) corresponds to the following formula (8): 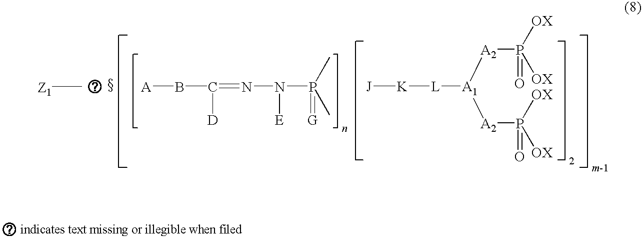
when p=m−2, formula (7) corresponds to the following formula (9): 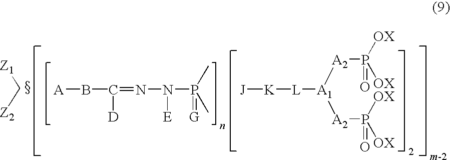
and in which formulae:
the central core § is chosen from the following groups: 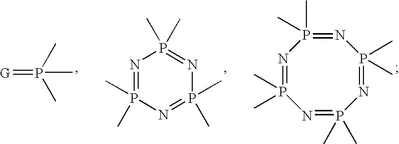
the generation chain corresponds to the formula: 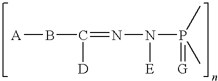
where
A represents an oxygen, sulphur, phosphorus atom or an —NR— group;
B represents an aryl group of 6 to 24 carbon atoms, a heteroaryl group of 1 to 24 carbon atoms, an alkyl group of 1 to 16 carbon atoms, an oligoethyleneglycol group, a polyethyleneglycol group, two aryl groups of 6 to 24 carbon atoms linked by an oxygen, a nitrogen, a sulphur, an alkyl group of 1 to 16 carbon atoms, an heteroalkyl group of 1 to 24 carbon atoms, an oligoethyleneglycol group, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
D represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OE group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
E represents a hydrogen atom, an alkyl group of 1 to 16 carbon atoms, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OE group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
G represents an oxygen, nitrogen, sulphur, selenium, tellurium atom or an ═NR group;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the intermediate chain corresponds to the formula:
-J-K-L-
where
J represents an oxygen atom, a sulphur atom, or a —NR— group;
K represents an aryl group of 6 to 24 carbon atoms, a heteroaryl group of 1 to 24 carbon atoms, the heteroelement being preferably chosen from oxygen, nitrogen or sulphur, an alkyl group of 1 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, each being able to be optionally substituted by a halogen atom or a NO₂, —NRR′, —CN, —CF₃, —OH group, an alkyl group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
L represents a linear, branched or cyclic hydrocarbon chain with 0 to 10 members, in particular with 0 to 6 members, optionally containing one or more double or triple bonds, each of said members optionally being able to be a heteroatom, said heteroatom being preferably chosen from oxygen, sulphur, nitrogen, phosphorus, silicon, each member being able to be optionally substituted by at least one substituent chosen from an alkyl group of 1 to 16 carbon atoms, a halogen, an oxygen atom, —NO₂, —NRR′, —CN, —CF₃, —OH, an alkoxy group of 1 to 16 carbon atoms, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
R and R′ representing independently of each other H or an alkyl group of 1 to 16 carbon atoms, an aryl group of 6 to 24 carbon atoms, or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹;
the terminal group corresponds to the formula: 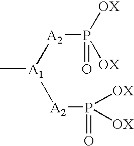
where A1, A2 and X are as previously defined, each X being identical or different;
Z₁ and Z₂ being identical or different, optionally linked together, in particular by means of a covalent bond, and representing H or a linear, branched or cyclic hydrocarbon chain with 1 to 16 members, optionally containing one or more double or triple bonds, each of said members being optionally chosen from a heteroatom, said heteroatom being preferably chosen from a nitrogen, oxygen, phosphorus, silicon or sulphur atom, an aryl group of 6 to 24 carbon atoms, a heteroaryl group of 1 to 24 carbon atoms, a carboxyl group, a >C═NR group, each member being able to be optionally substituted by at least one substituent chosen from a hydroxyl group, a NR″R′″ group, an alkoxy group of 1 to 16 carbon atoms, an alkyl group of 1 to 16 carbon atoms, a halogen atom, a NO₂ group, a —CN group, a —CF₃ group, an aryl group of 6 to 24 carbon atoms, an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, R″ and R′″ representing independently of each other H or an alkyl group of 1 to 16 carbon atoms, an aryl group of 6 to 24 carbon atoms or an aralkyl group of 7 to 16 carbon atoms, an oligoethyleneglycol group comprising preferentially 1 to 12 ethyleneglycol moieties, a polyethyleneglycol group having a molecular weight ranging preferentially from 300 g.mol⁻¹ to 3000 g.mol⁻¹, the first member of said hydrocarbon chain preferably being oxygen or nitrogen.

42. The method according to claim 41 wherein the dendrimers are of general formula (8) of structure PMME, 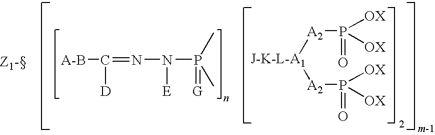
in which
§ represents 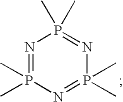
m represents 6;
p represents 5;
n represents 0, 1, or 2;
A represents an oxygen atom;
B represents a benzene group;
D represents hydrogen;
E represents a methyl group;
G represents a sulphur atom;
J represents an oxygen atom;
K represents a benzene group;
L represents a non-substituted linear saturated hydrocarbon chain with two carbon atoms;
A₁ represents a nitrogen atom;
A₂ represents a CH₂ group;
X represents a methyl group, or a hydrogen or sodium atom;
Z₁ represents a phenyloxy group;
said dendrimer being designated GCn′, n being defined above.

43. The method according to claim 25 wherein the dendrimers are selected from the group comprising the compounds of the following formulae: 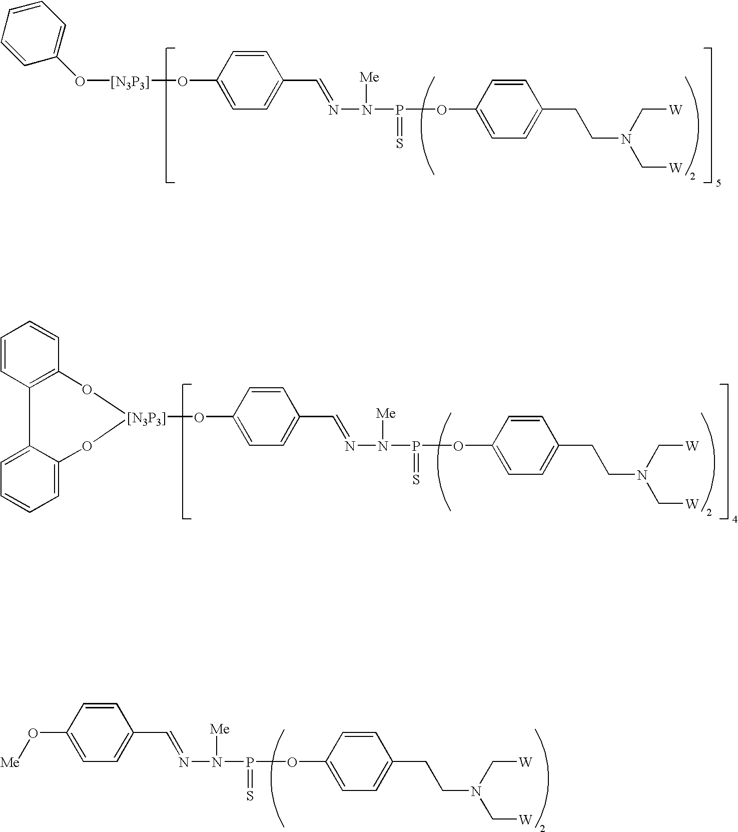
in which W represents PO₃Me₂, PO₃HNa, PO₃H₂, said compounds corresponding, in particular, to compound GC1′ of the following formula (10): 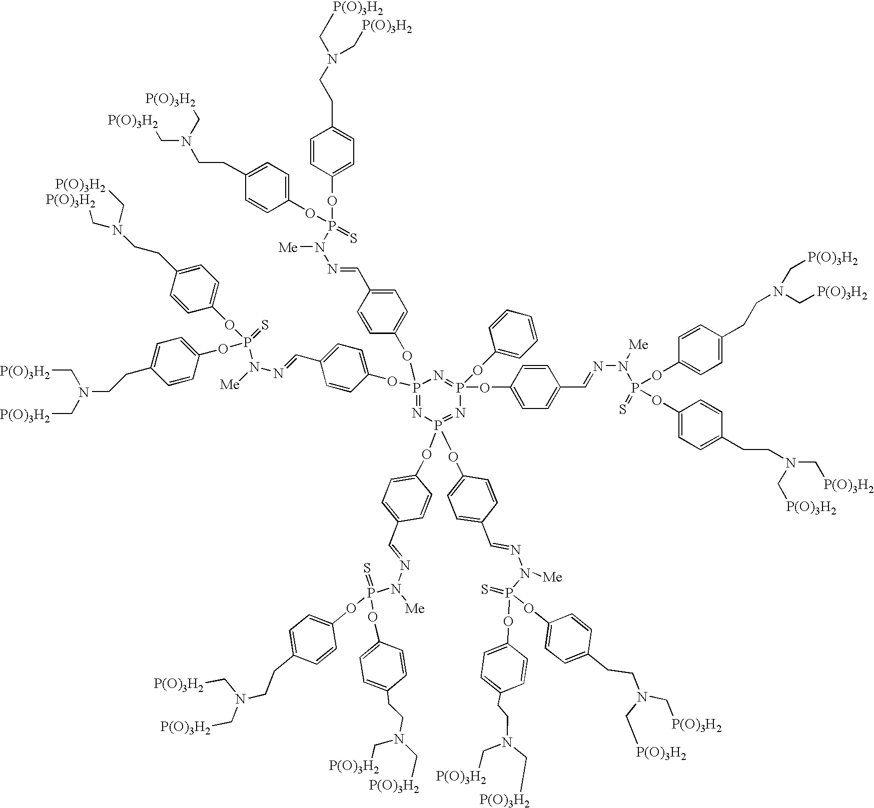
or of the formula (10a) 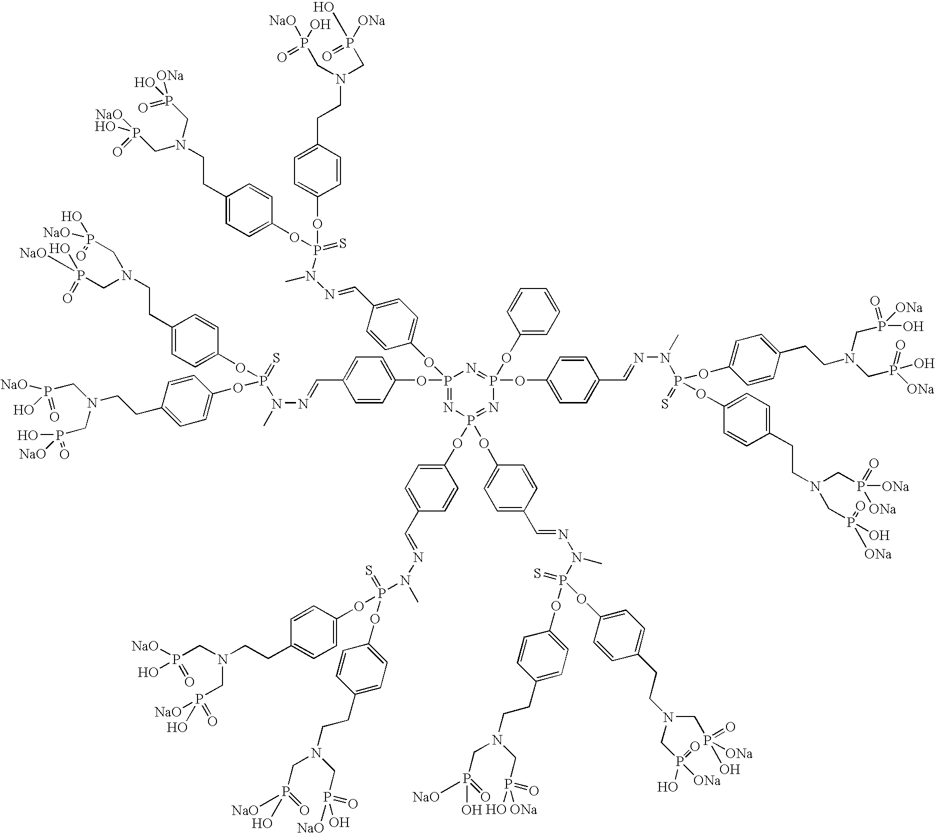
or compounds of the following formula: 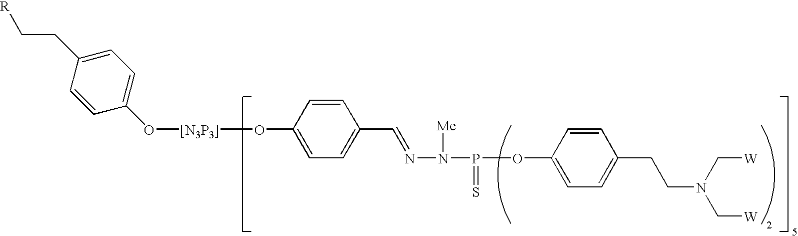
in which W represents PO₃Me₂, PO₃HNa, or PO₃H₂ and R is selected from the group comprising (a) fluorescent groups chosen from: 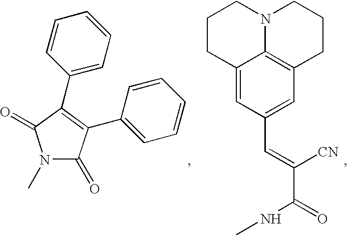
and (b) a biotinyl group of formula 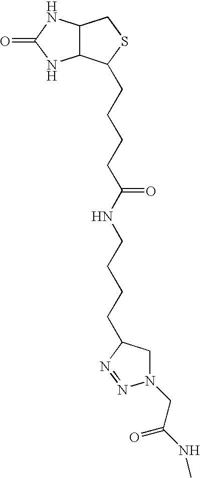
said compounds corresponding in particular to the compounds of the following formulae:

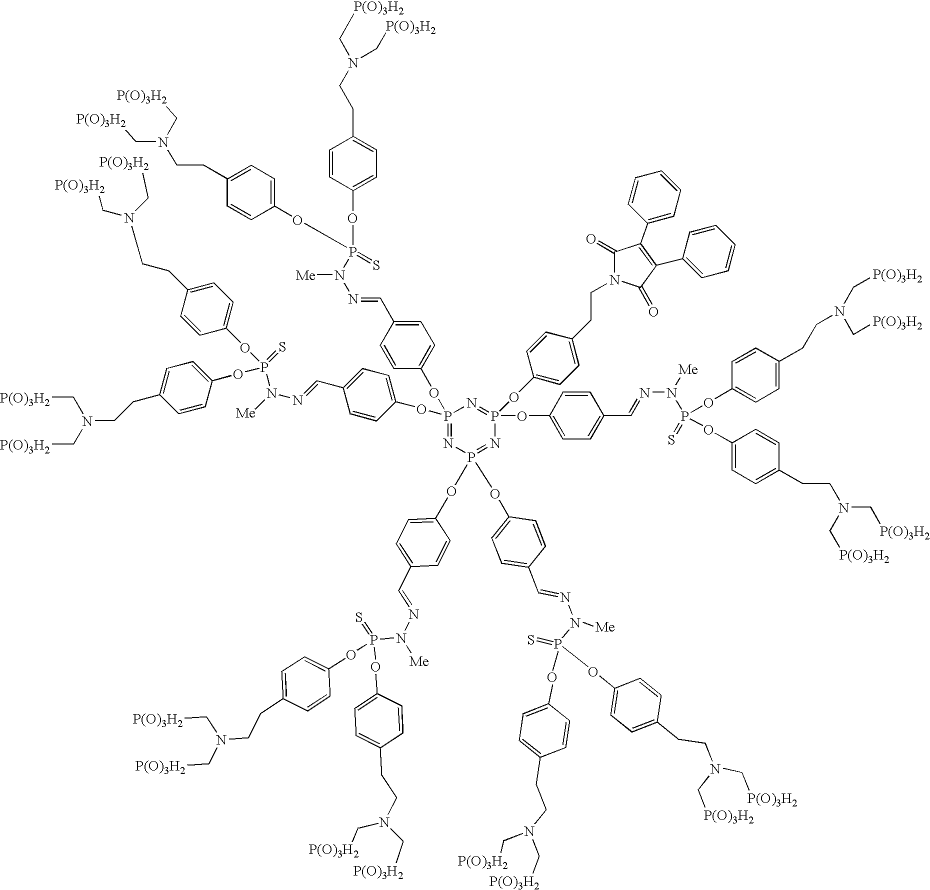

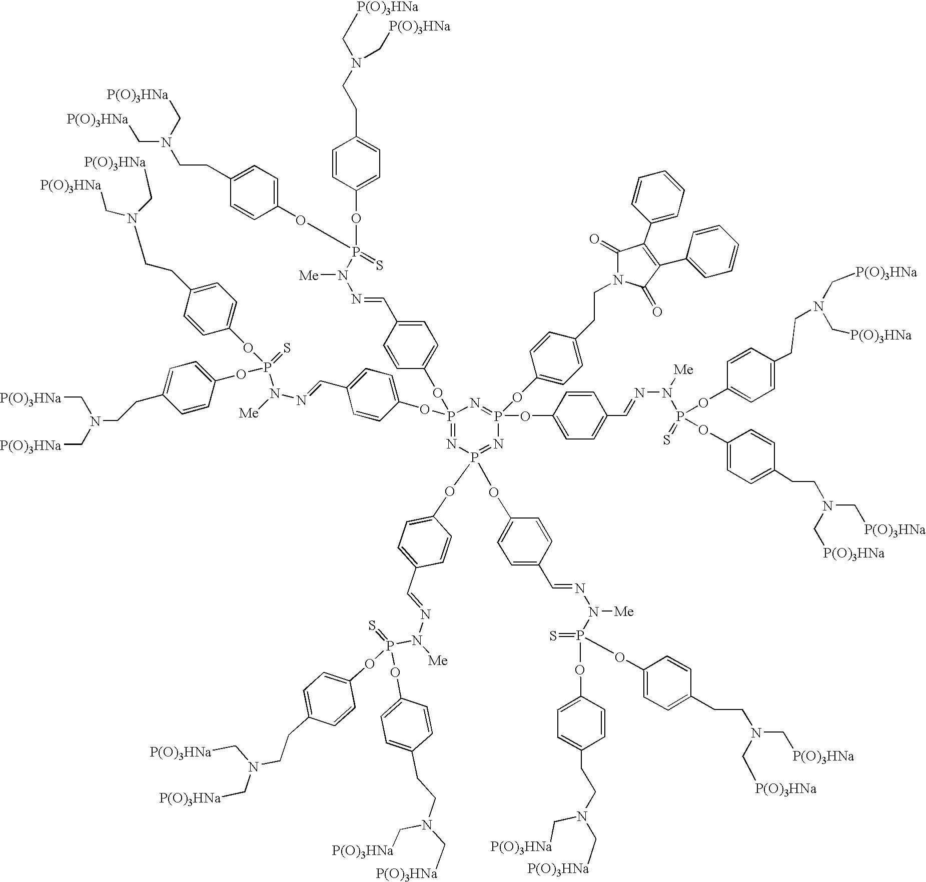

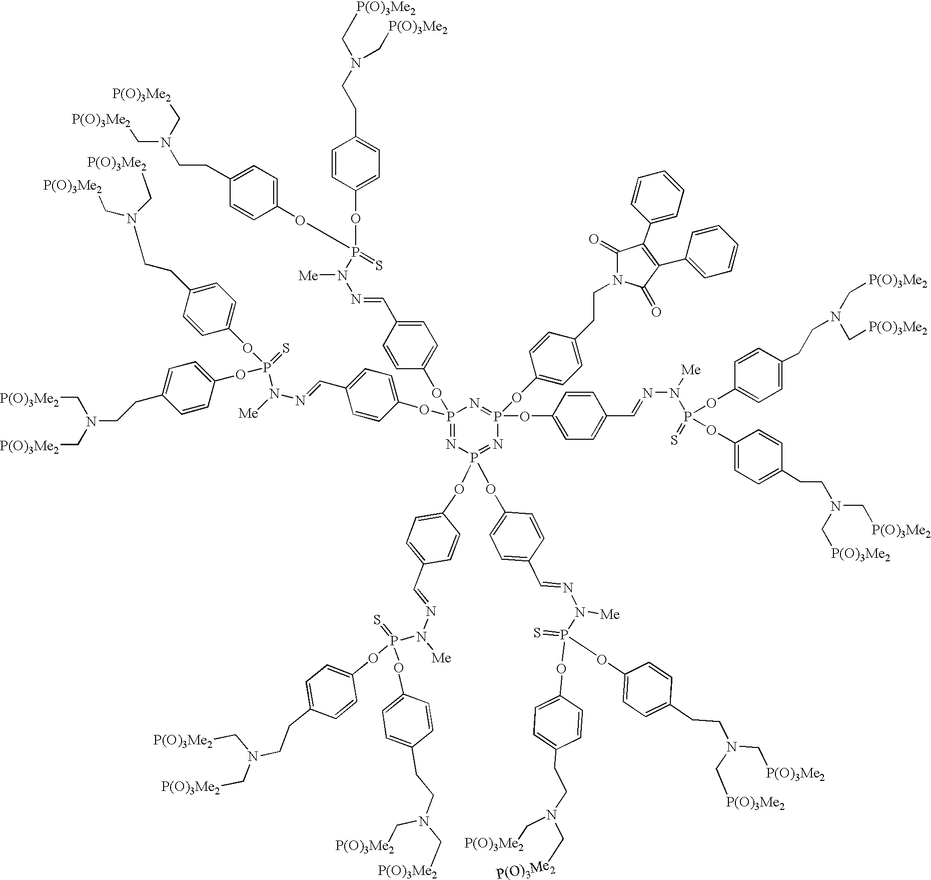

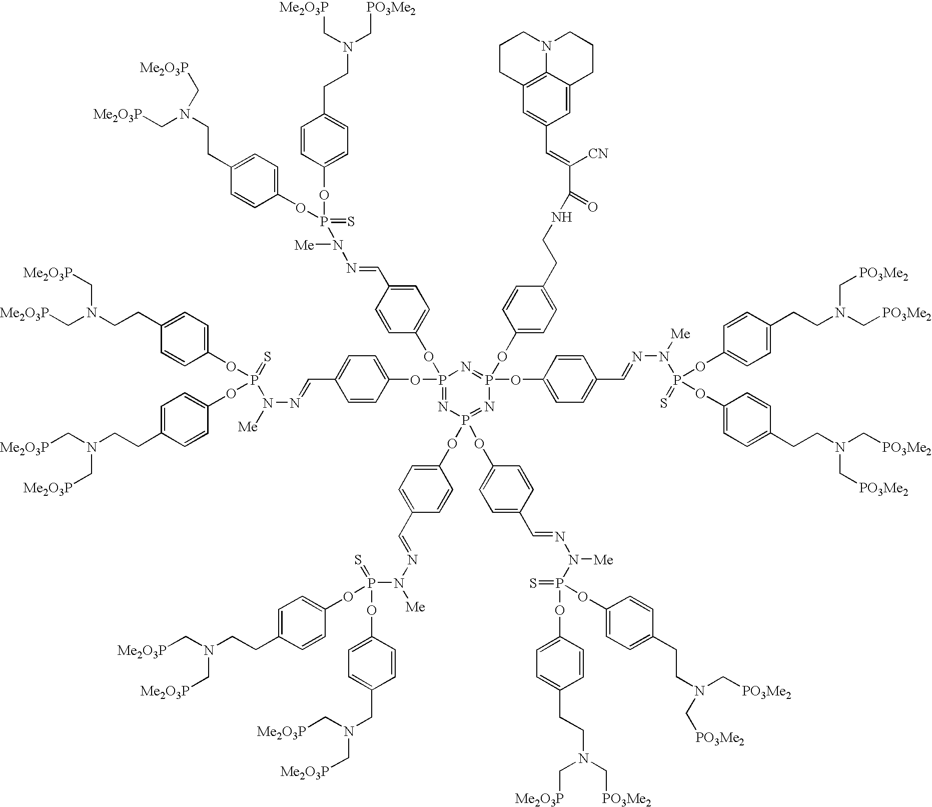

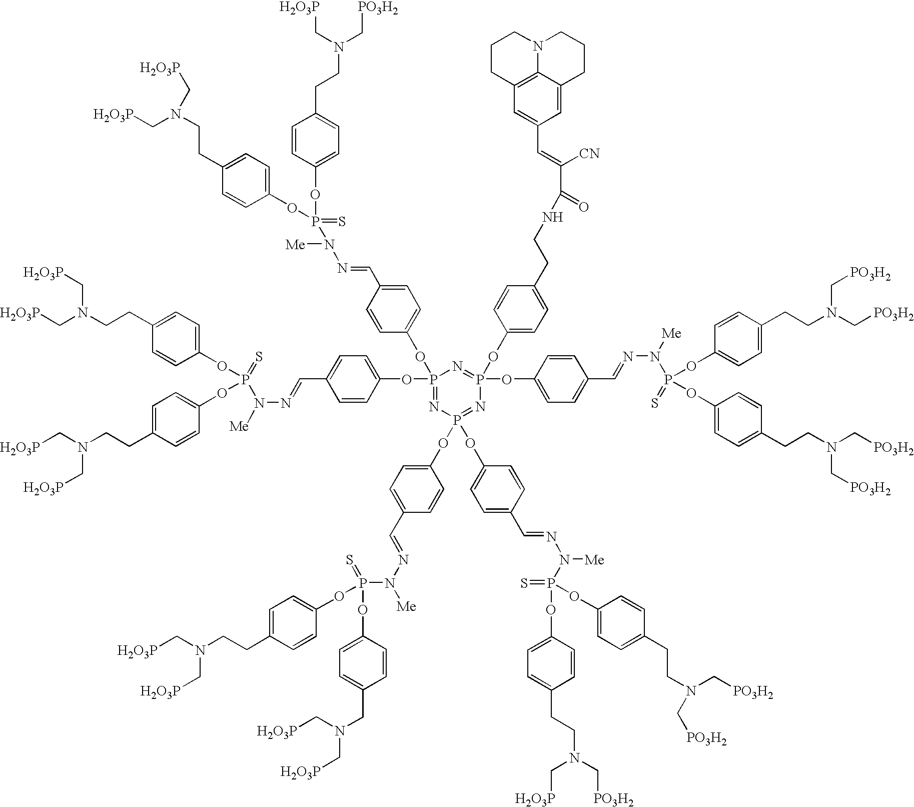

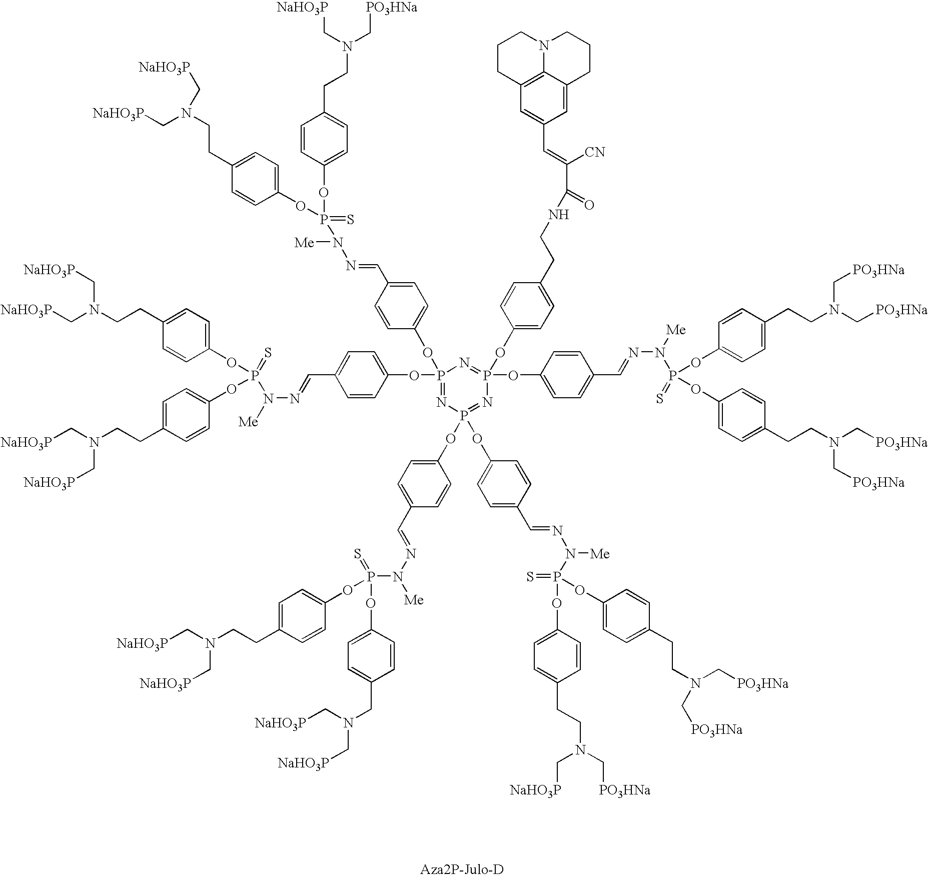

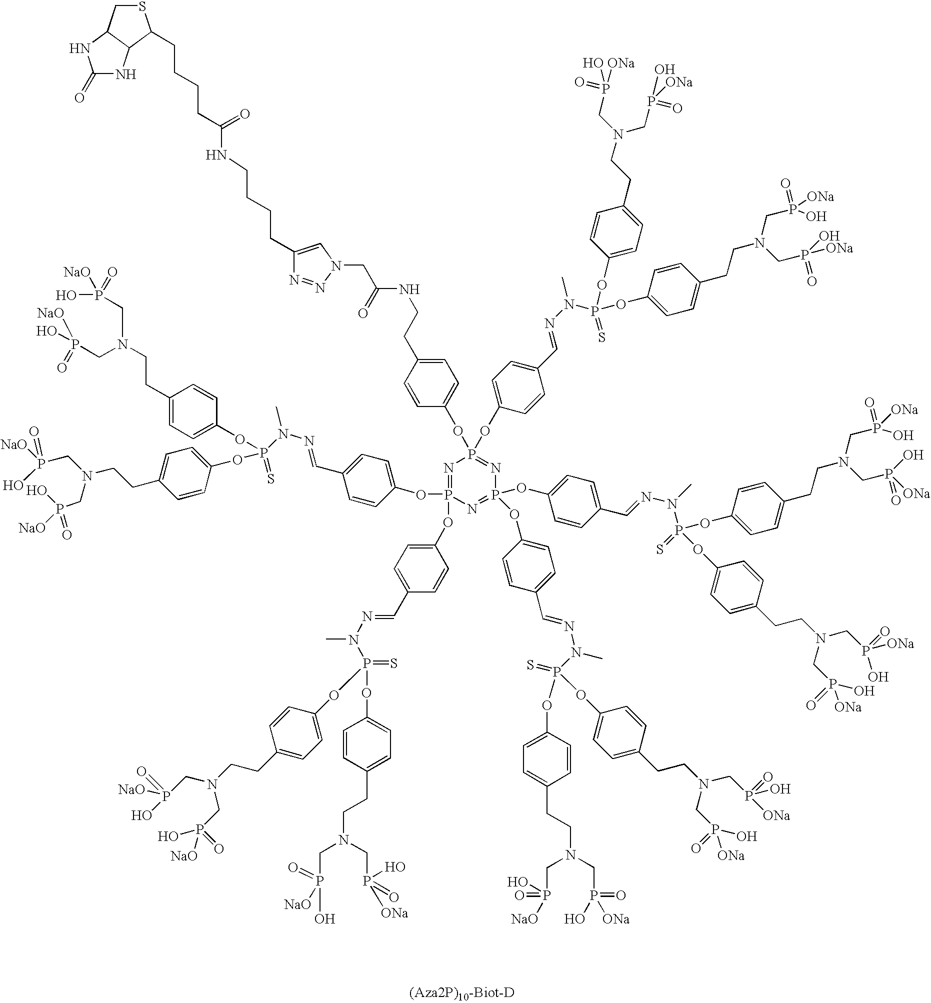

44. The method according to claim 26 wherein the dendrimers are selected from the group comprising: 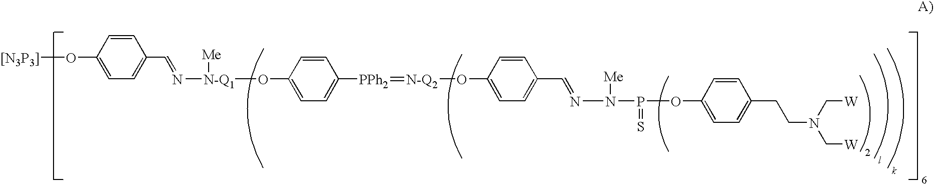
in which W represents PO₃Me₂, PO₃HNa, or PO₃E₂, Q₁ and Q₂, identical or different, represent P═S or cyclotriphosphazene (N₃P₃), 1 represents 2 when Q₂ represents P═S or 5 when Q₂ represents N₃P₃ and k represents 2 when Q₁ represents P═S or 5 when Q₁ represents N₃P₃, said dendrimers being in particular represented by the following formulae: 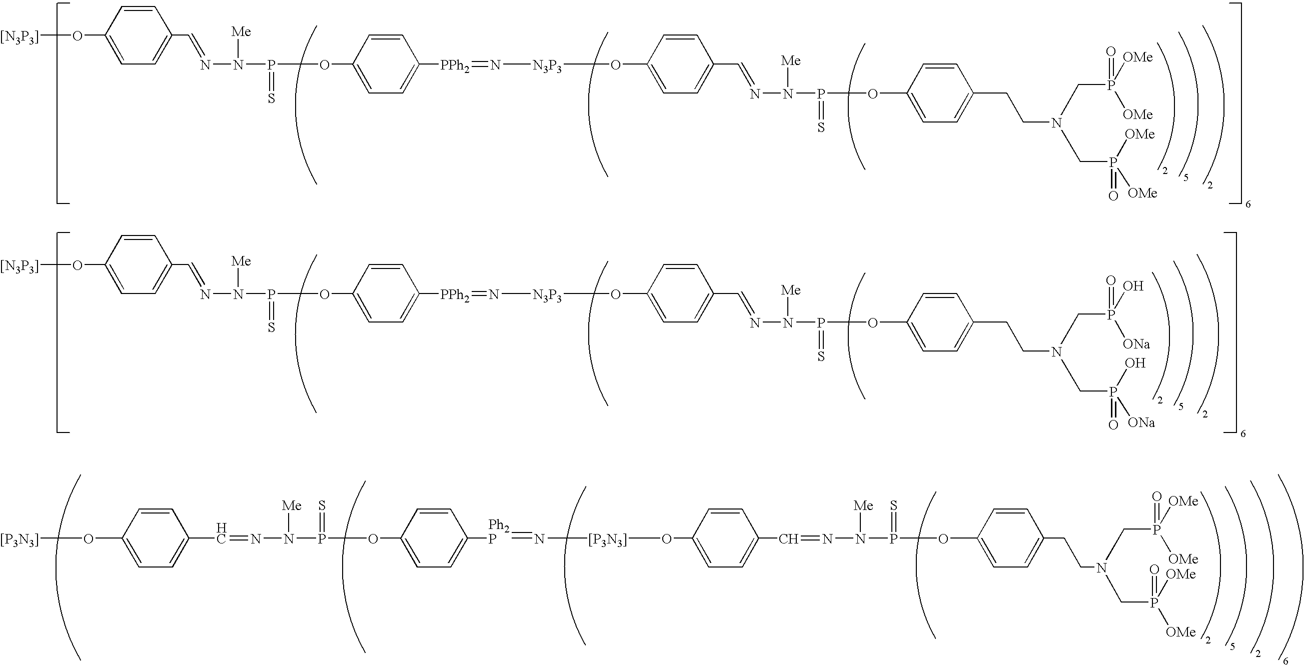 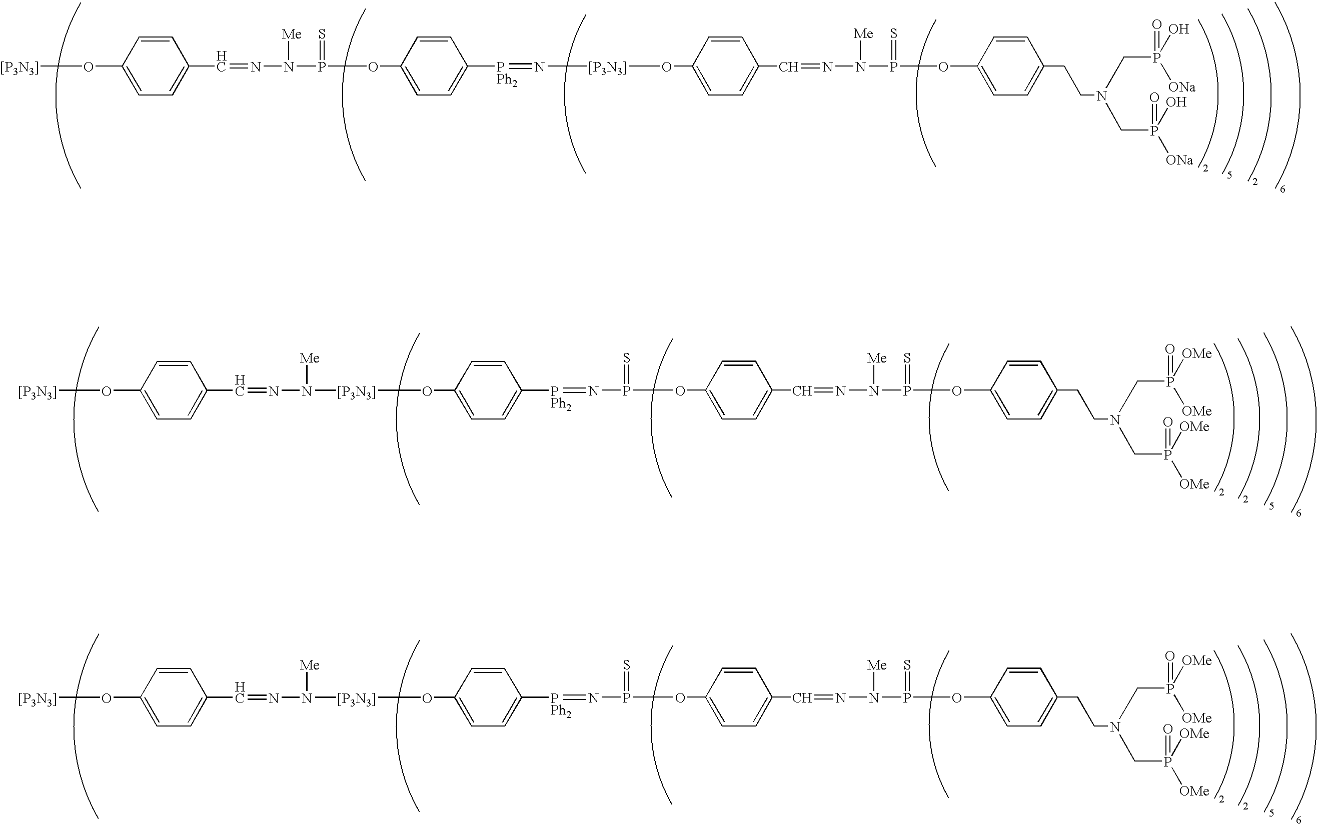 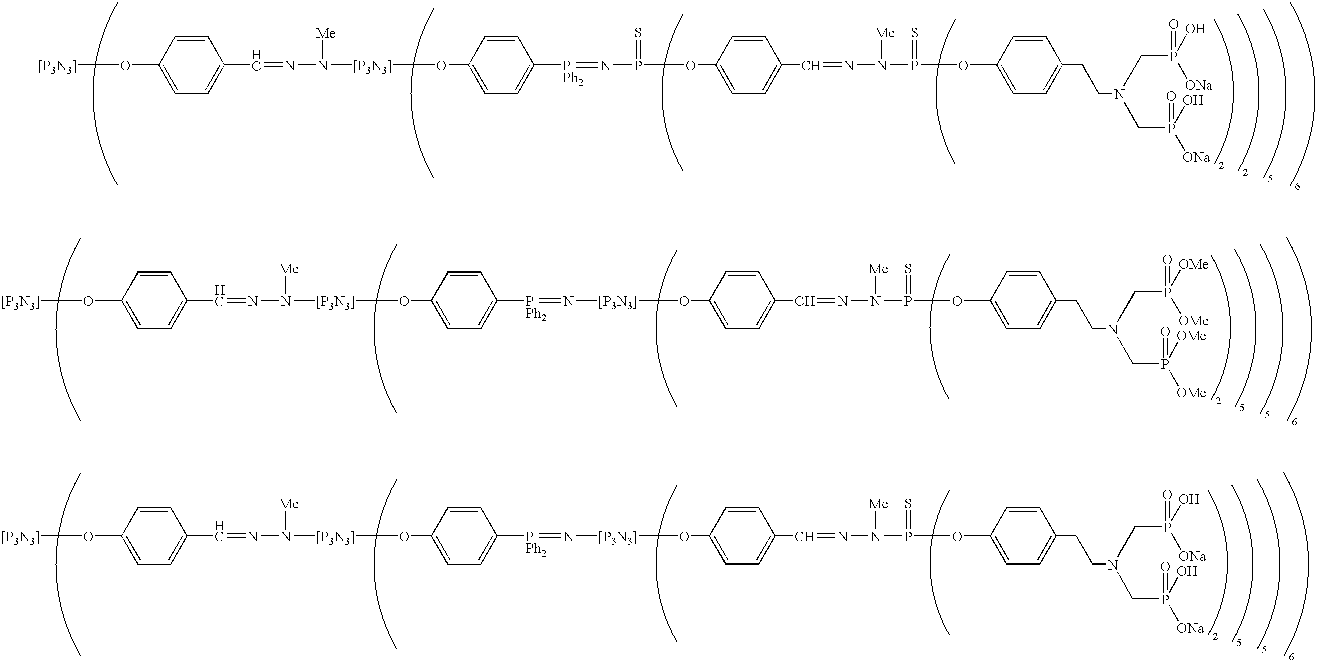 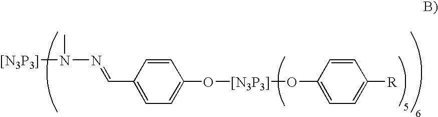
in which R represents a group chosen from 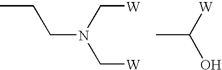
where W represents PO₃Me₂, PO₃HNa, or PO₃H₂. 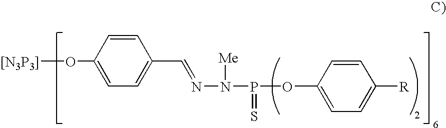
in which R represents a group chosen from: 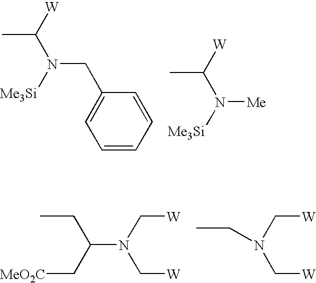
where W represents PO₃Si₂Me₆, PO₃Me₂, PO₃ENa, or PO₃H₂. 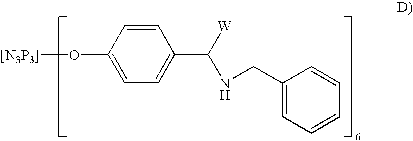
in which W represents PO₃Me₂, PO₃HNa, or PO₃H₂. 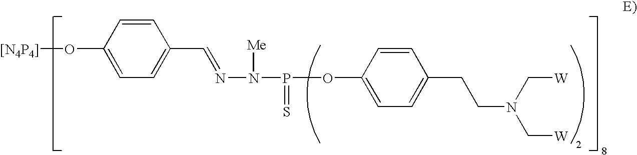
in which W represents PO₃Me₂, PO₃HNa, or PO₃H₂. 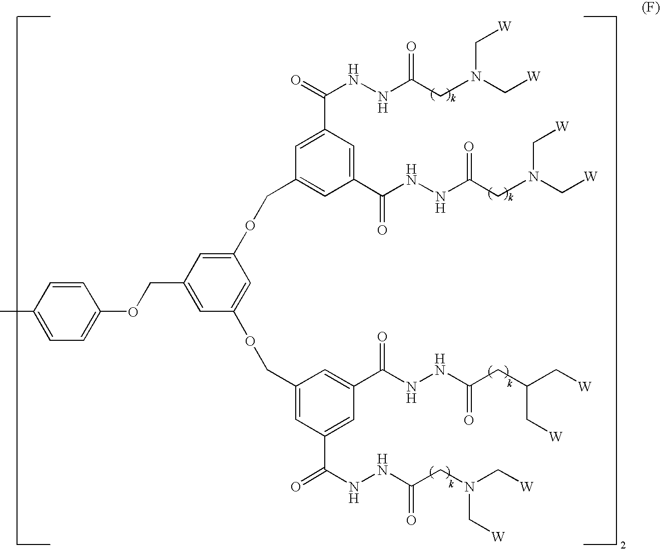
in which W represents PO₃Me₂, PO₃HNa, or PO₃H₂ and k represents 1, 2 or 3. 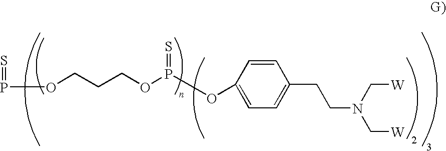
in which W represents PO₃Me₂, PO₃HNa, or PO₃H₂ and n represents 0, 1 or 2. 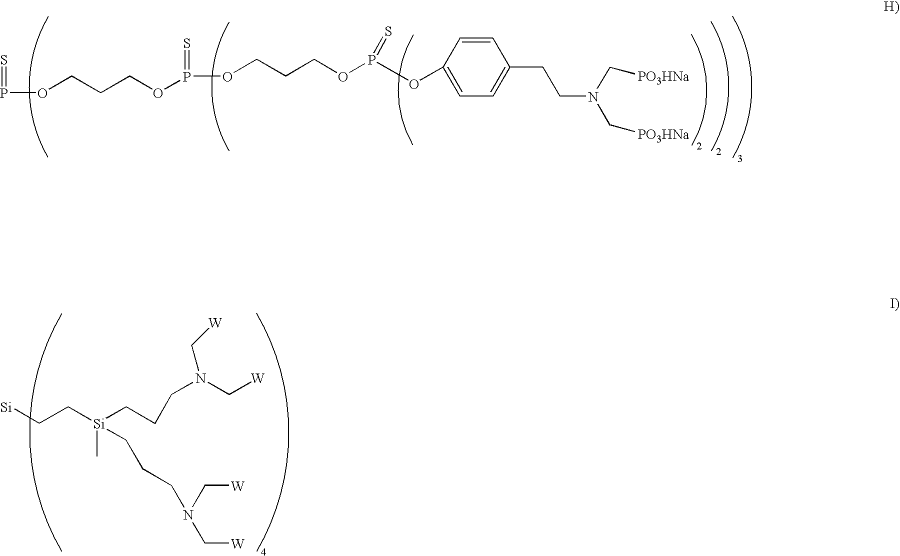
in which W represents PO₃Me₂, PO₃HNa, or PO₃H₂, in particular compound of the following formula: 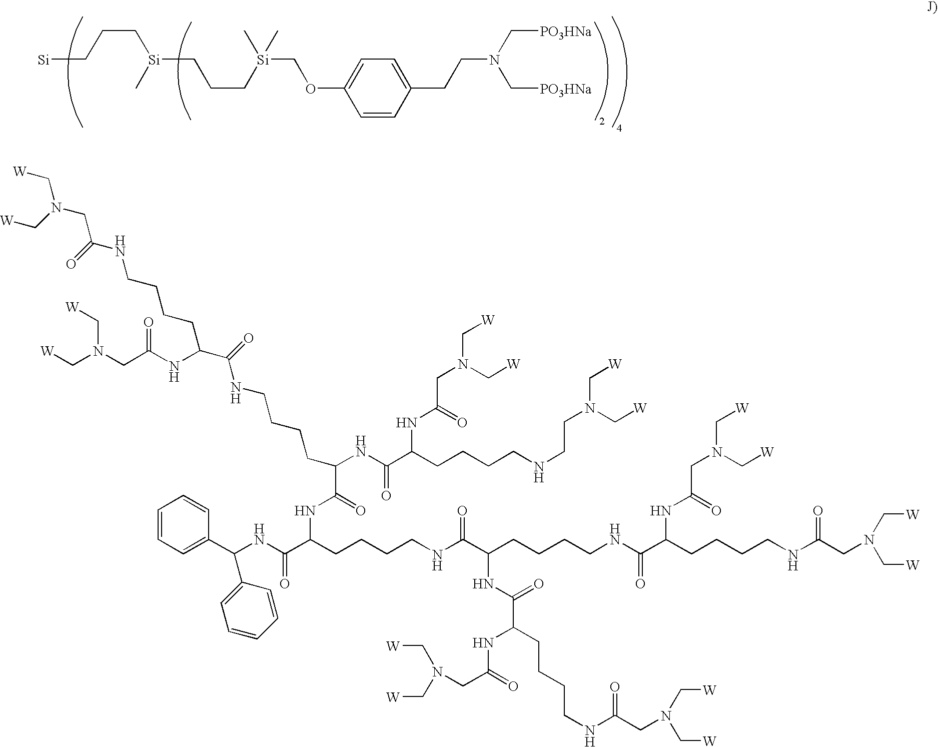
in which W represents PO₃Me₂, PO₃HNa, or PO₃H₂.

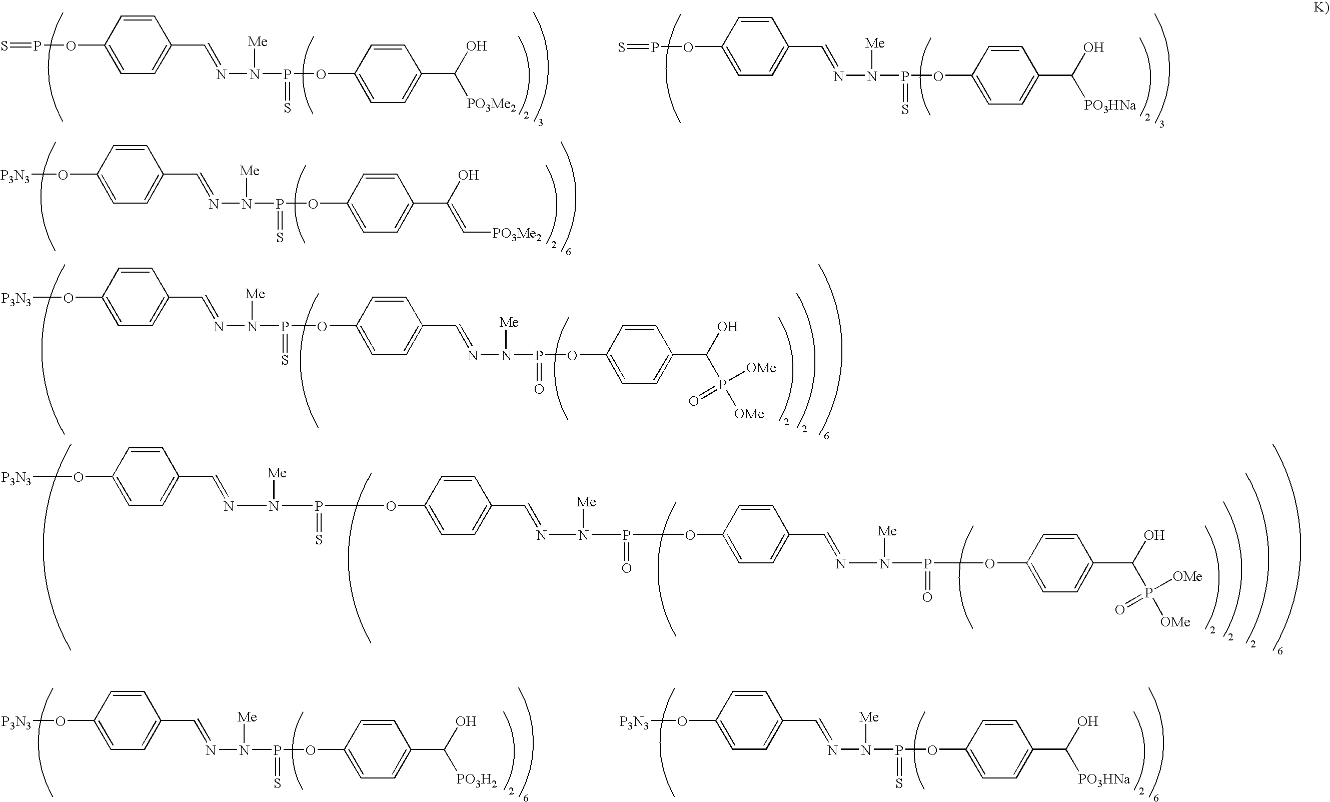

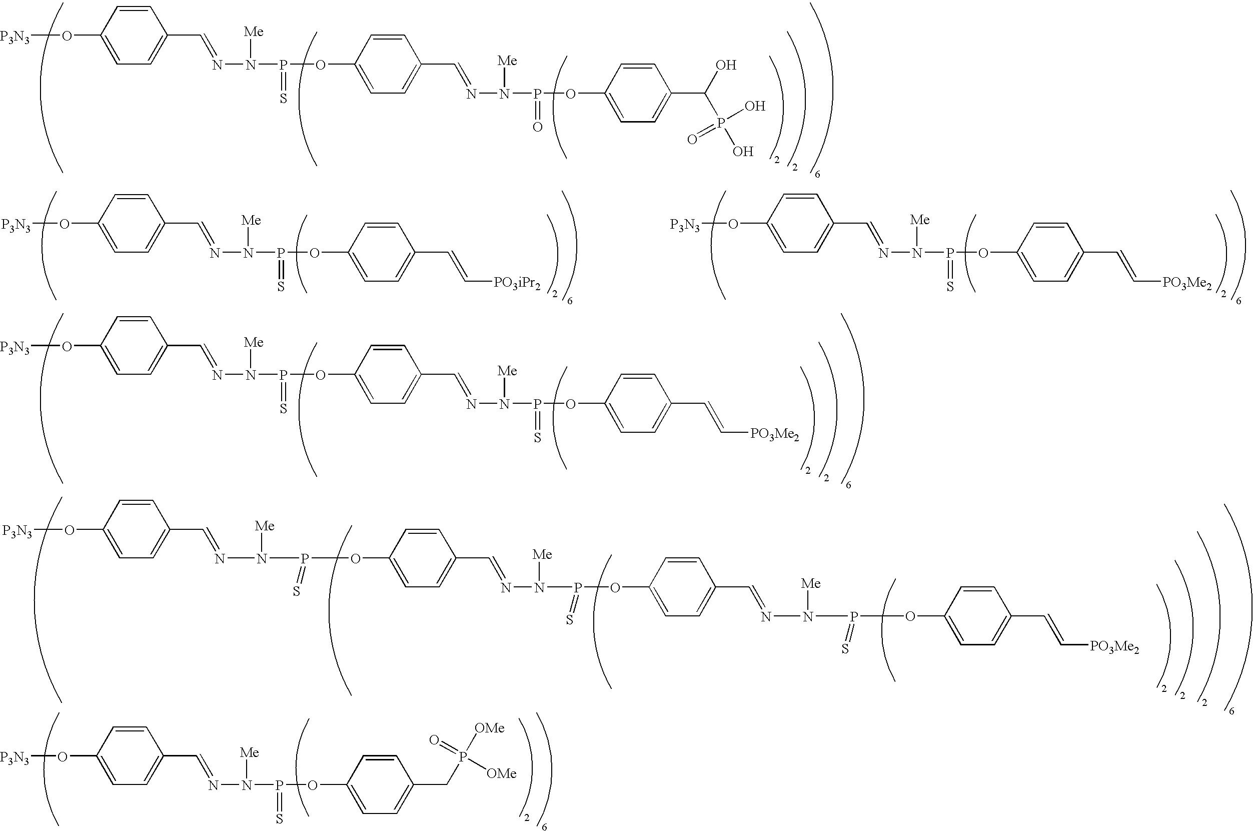

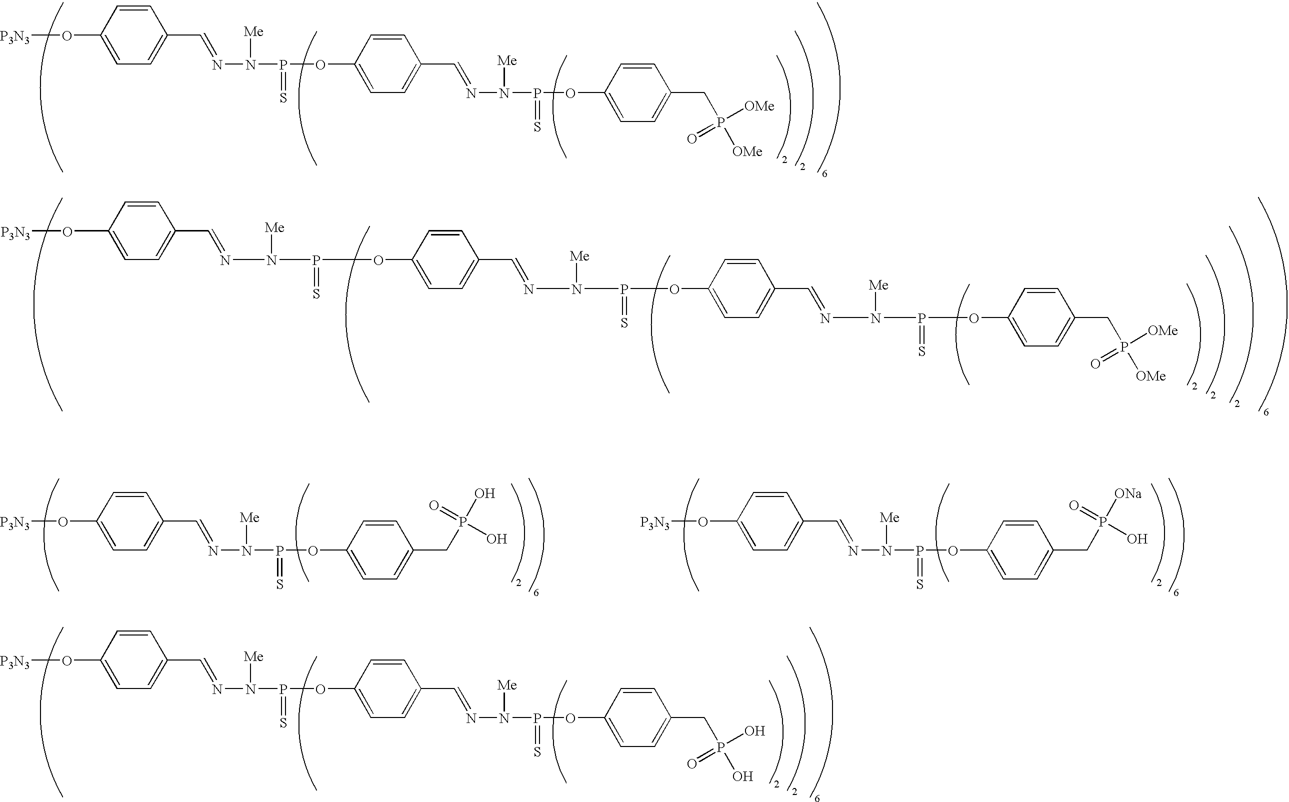

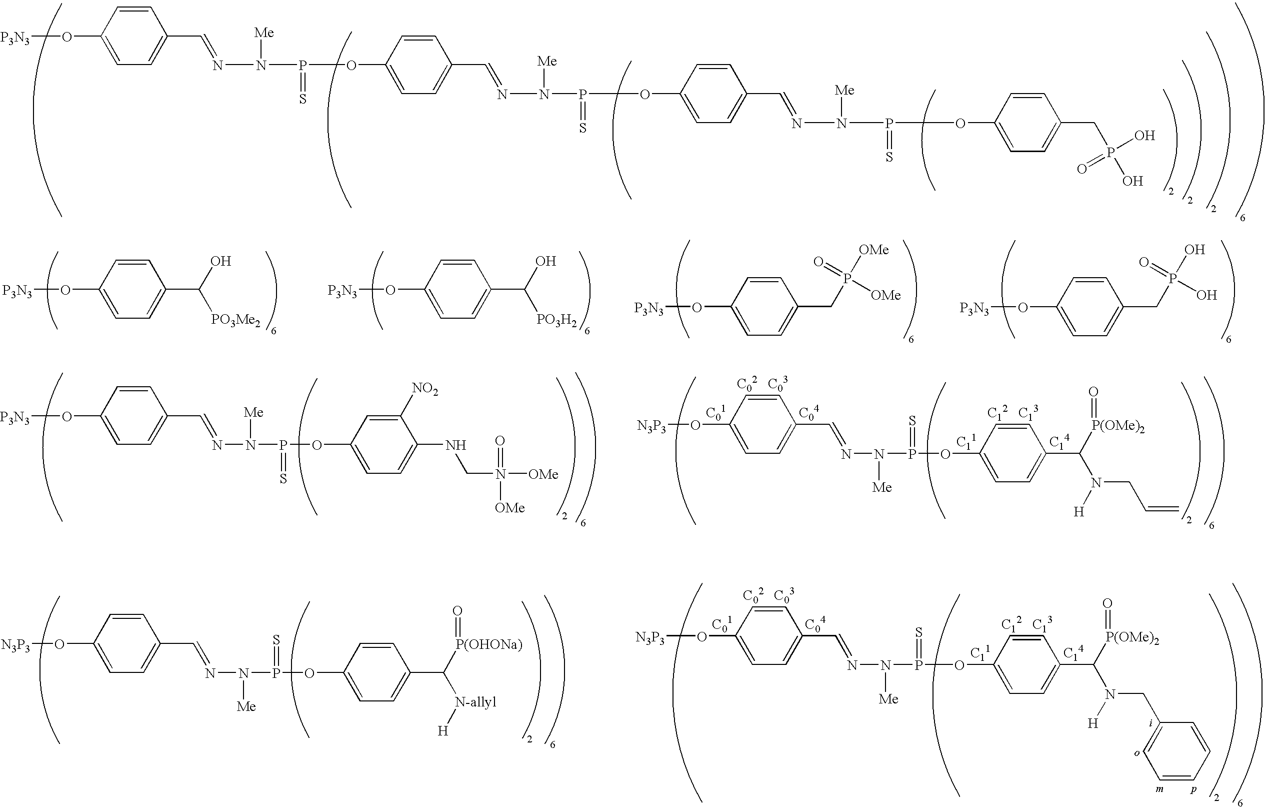

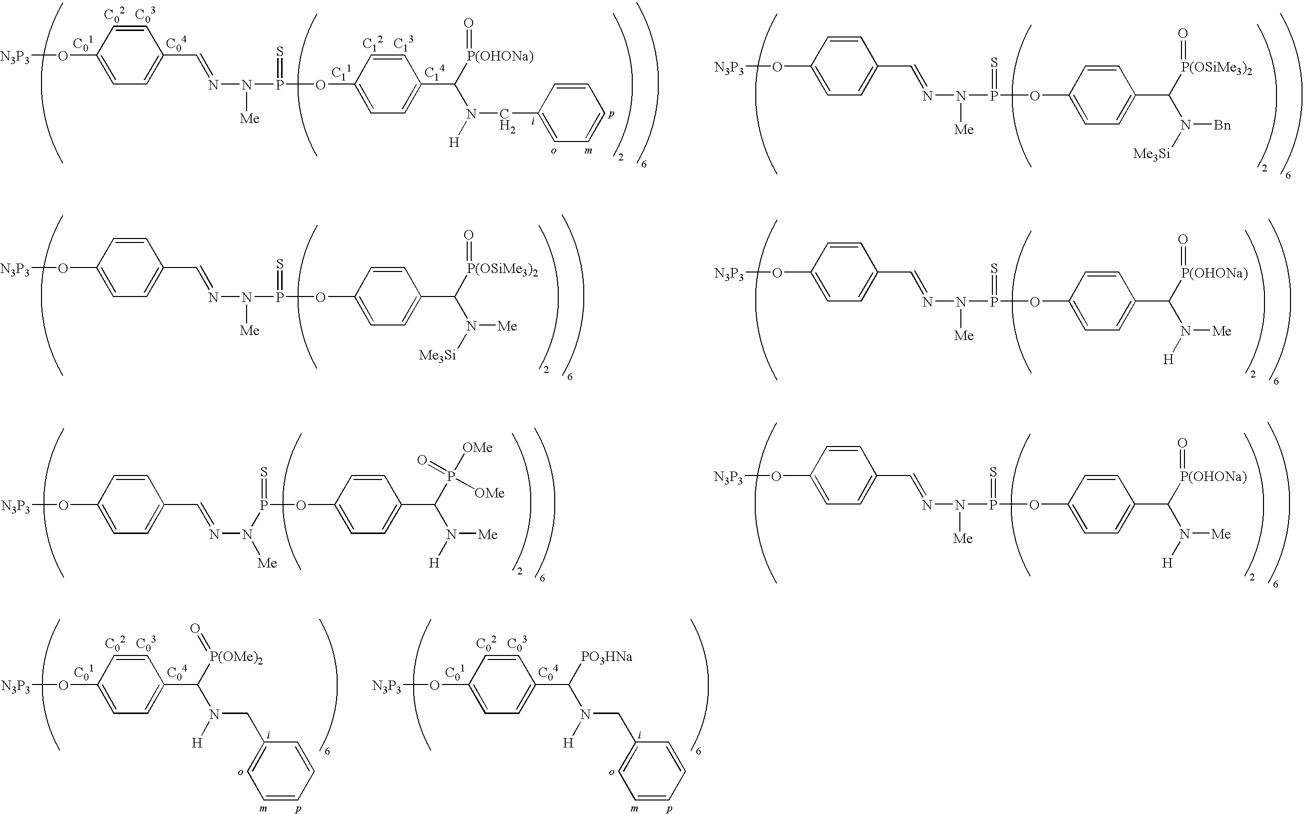

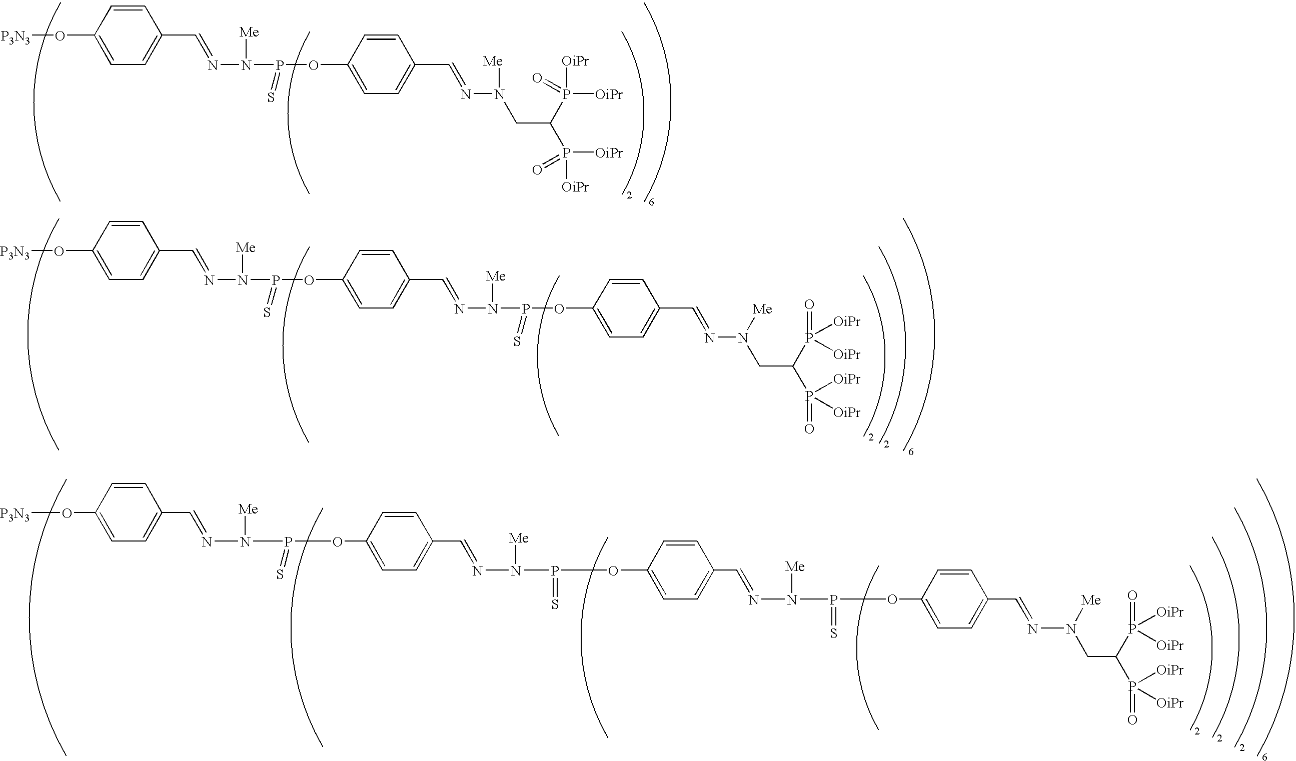

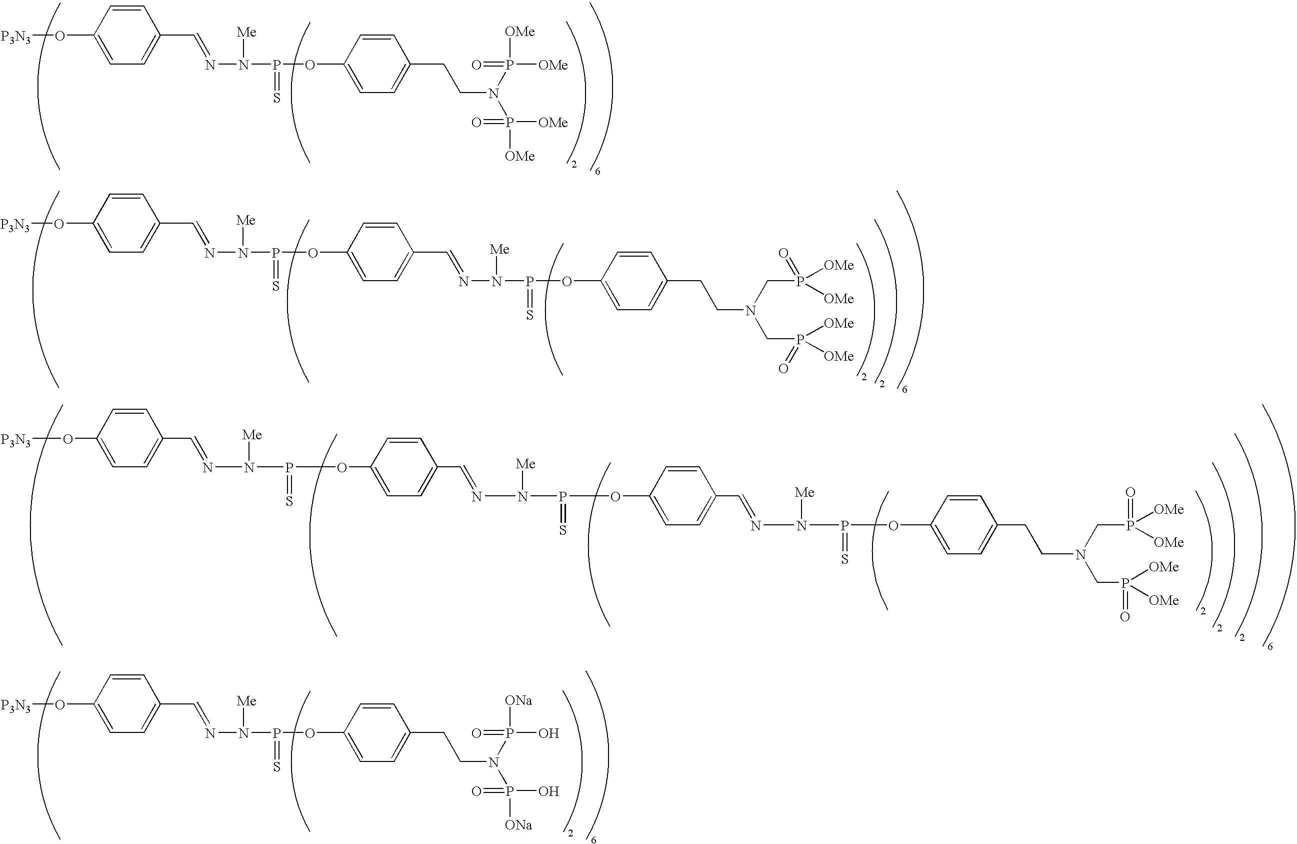

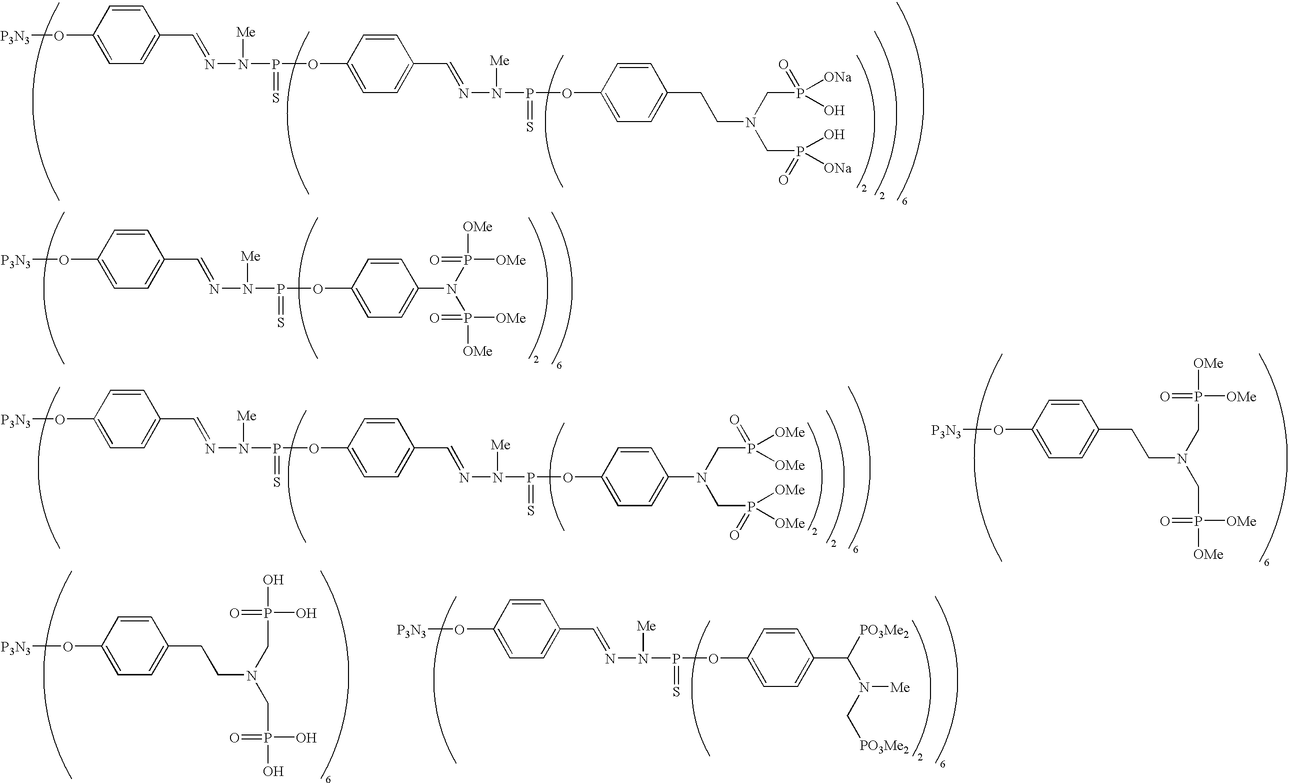

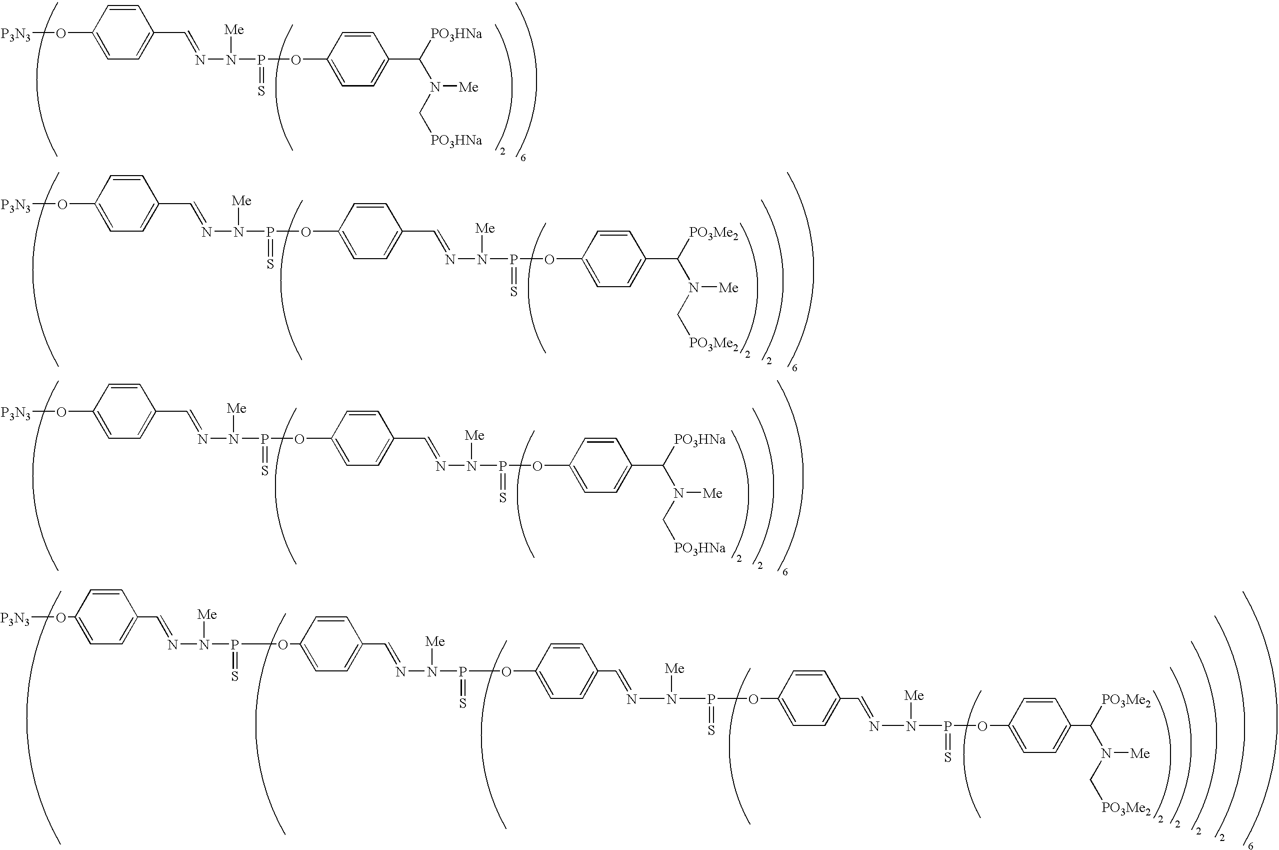

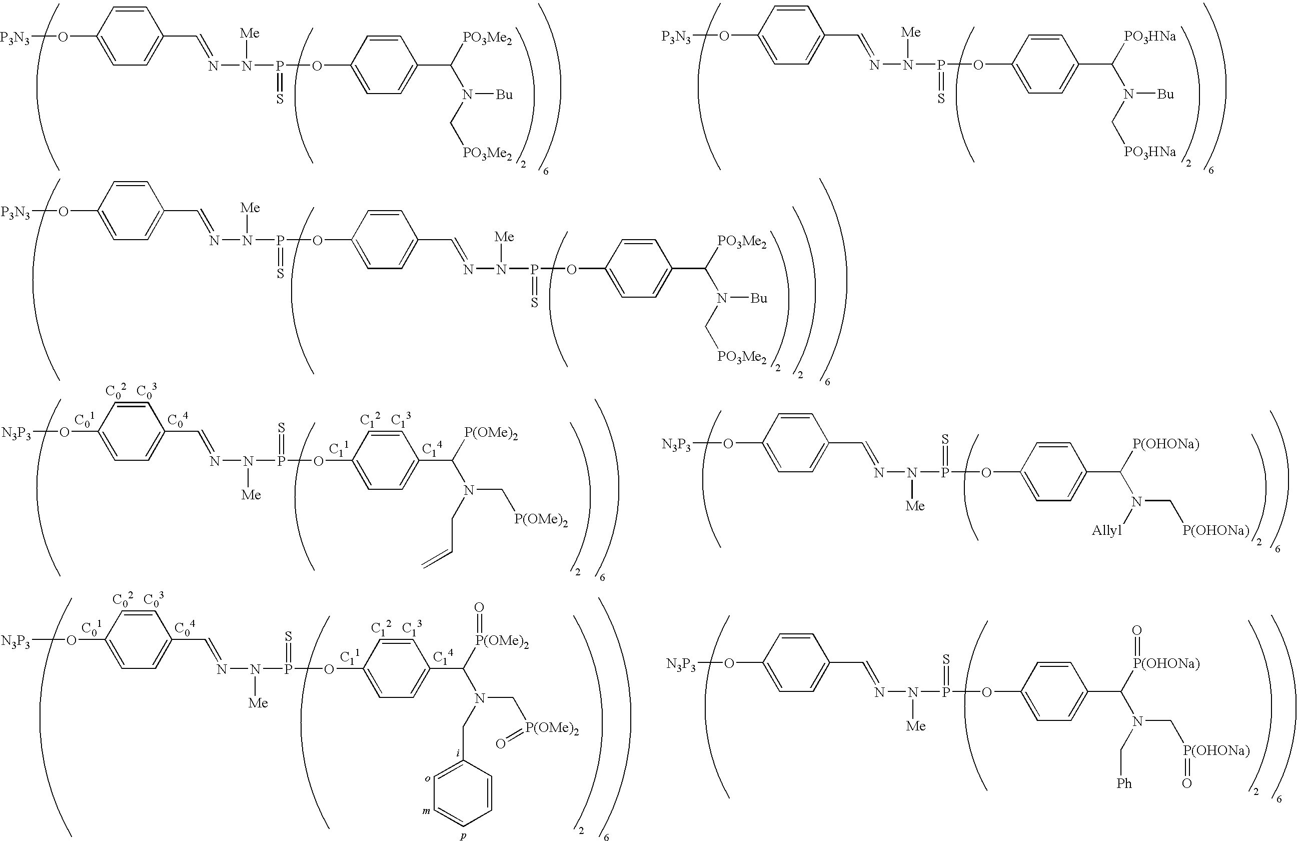

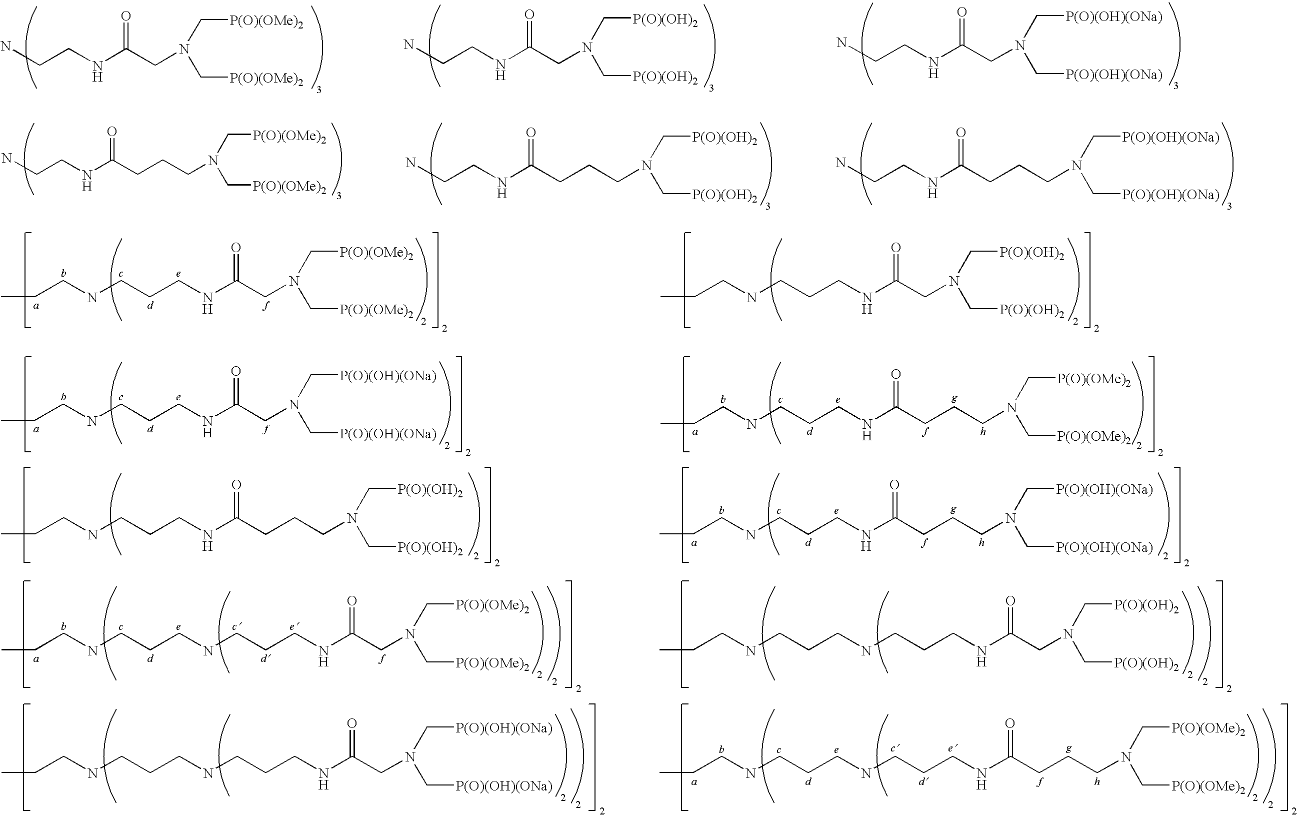

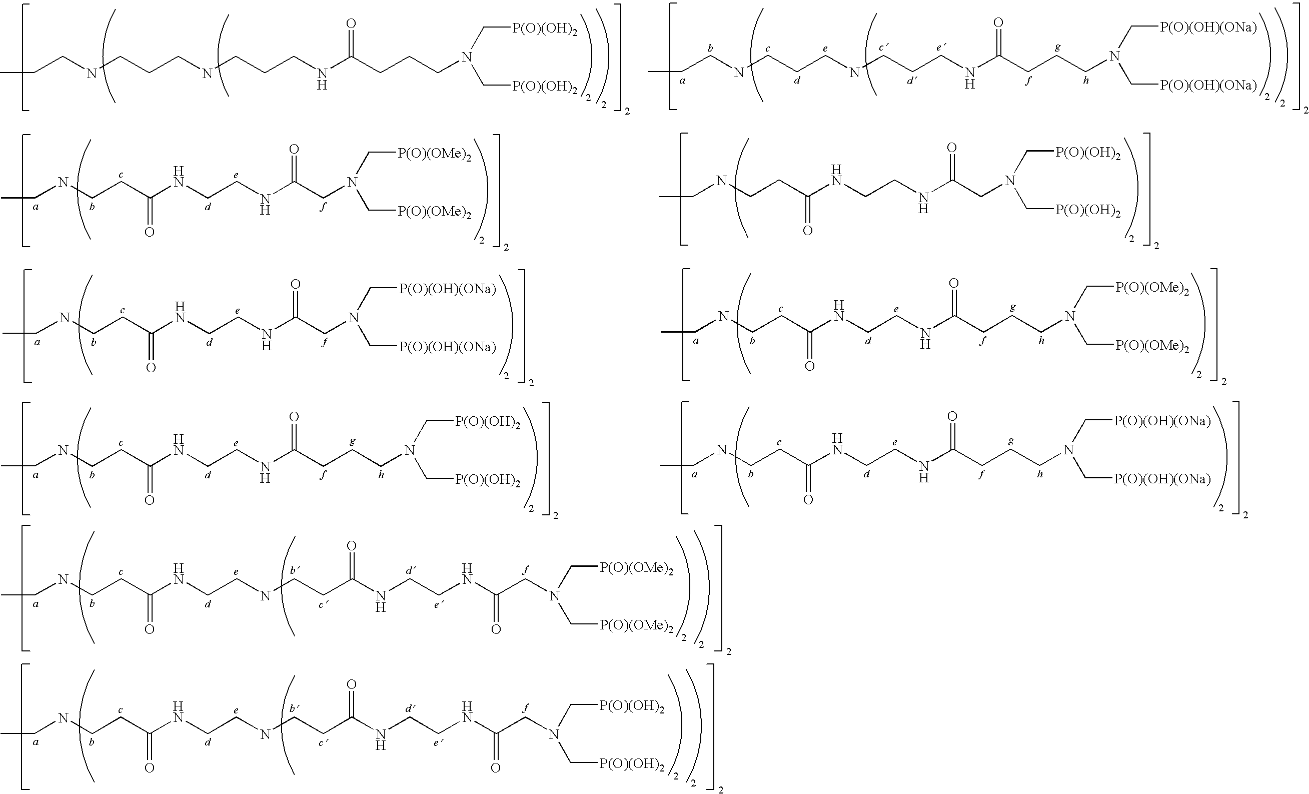

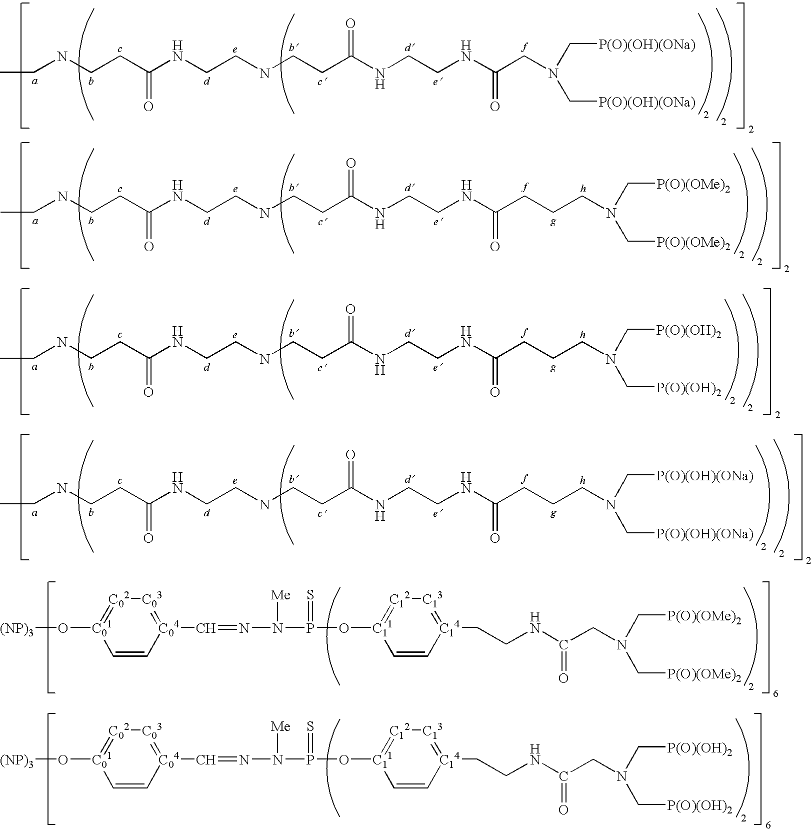

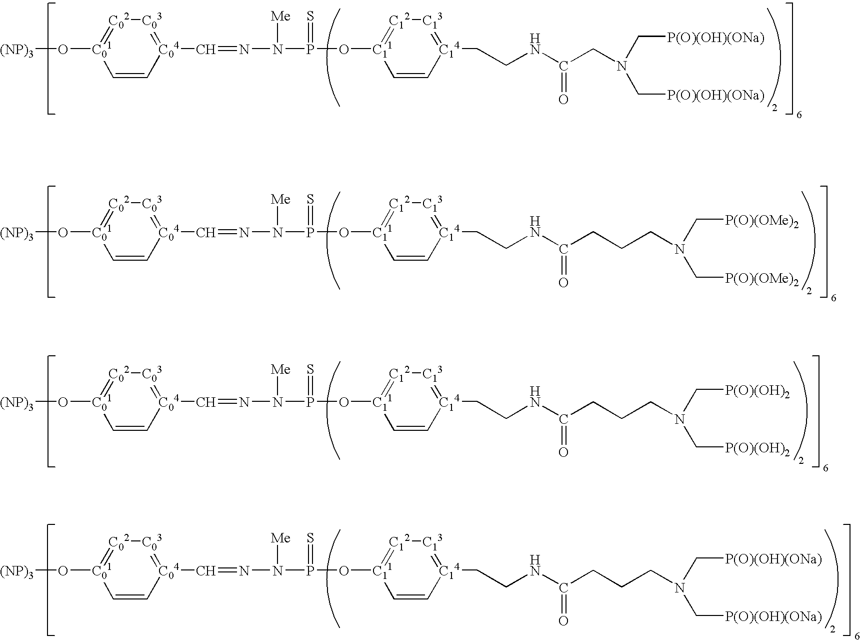

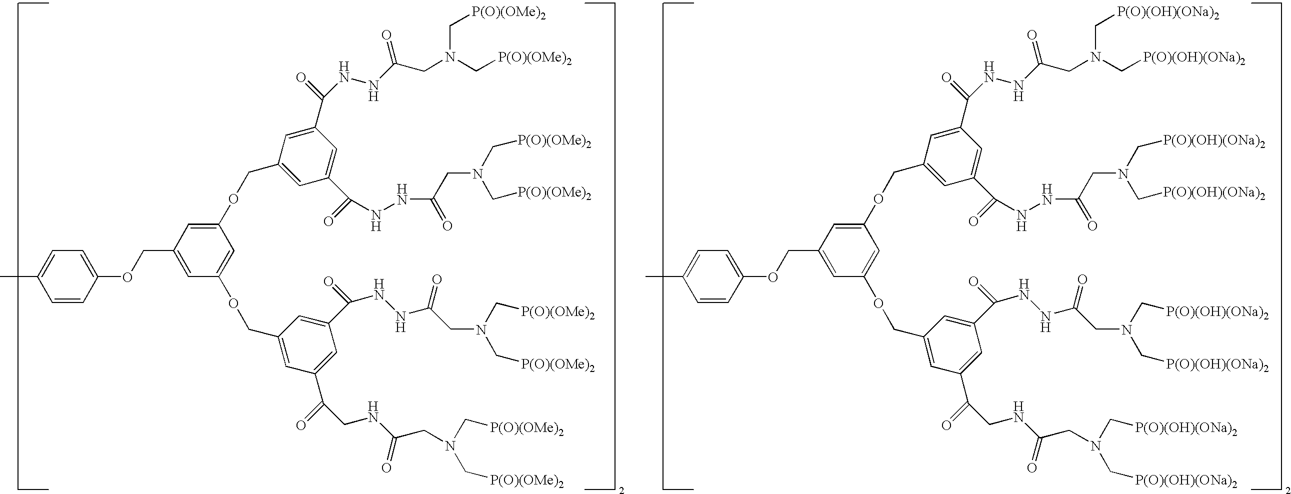

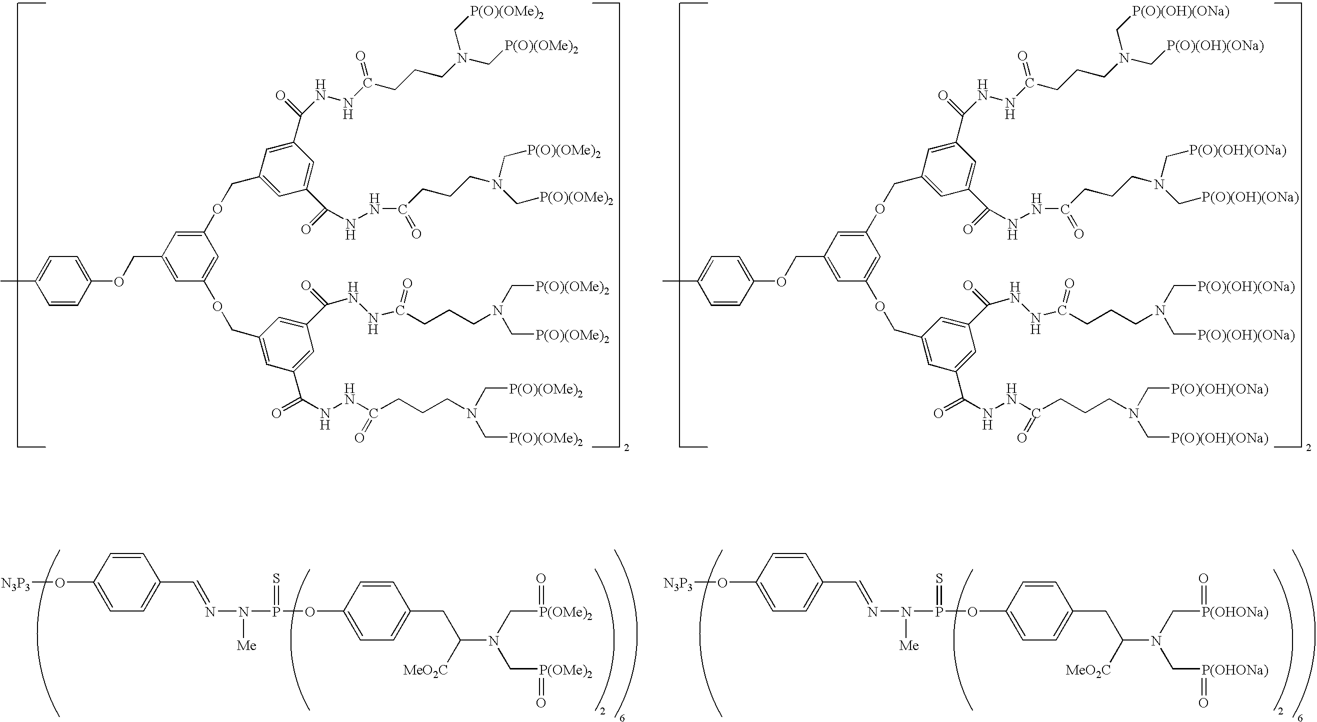

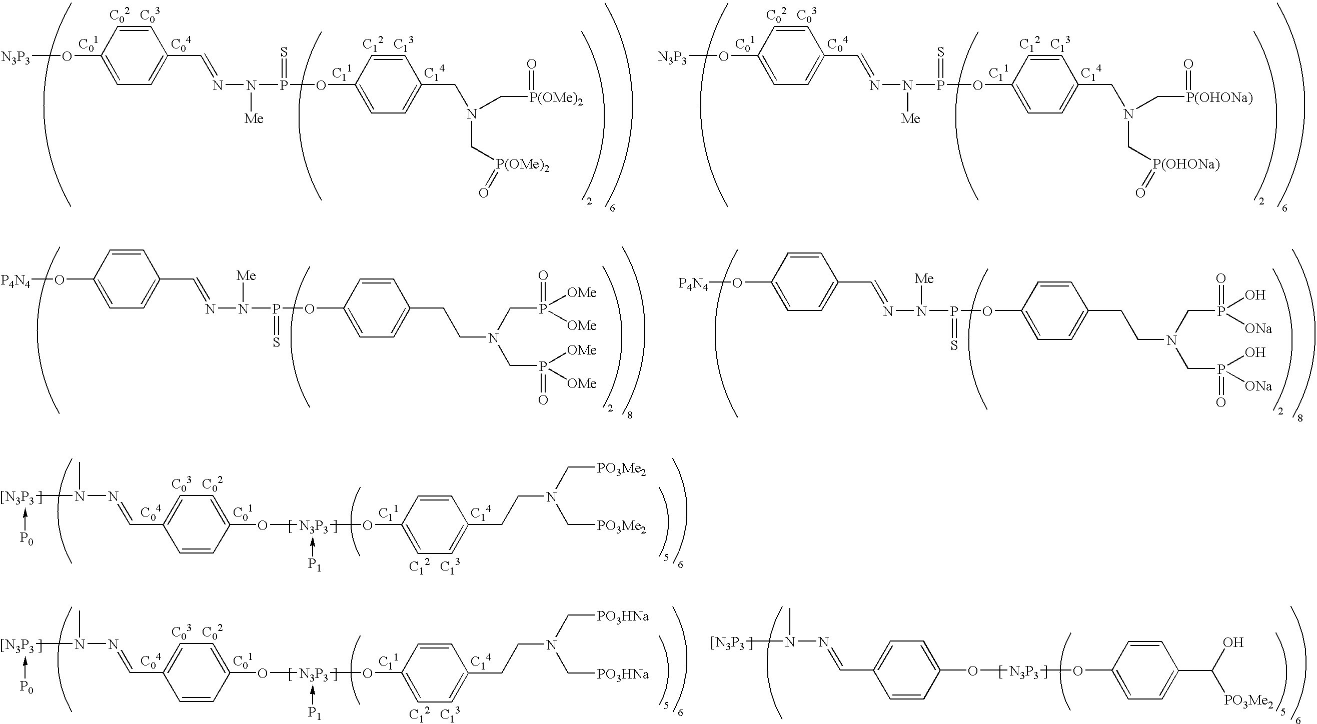

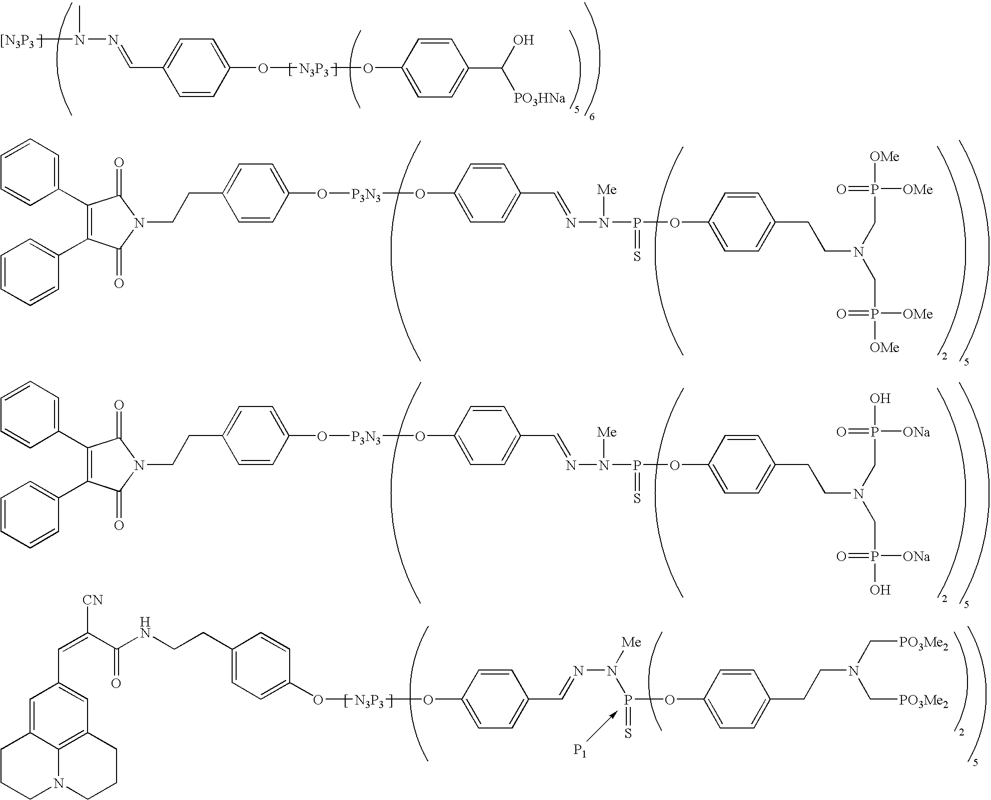

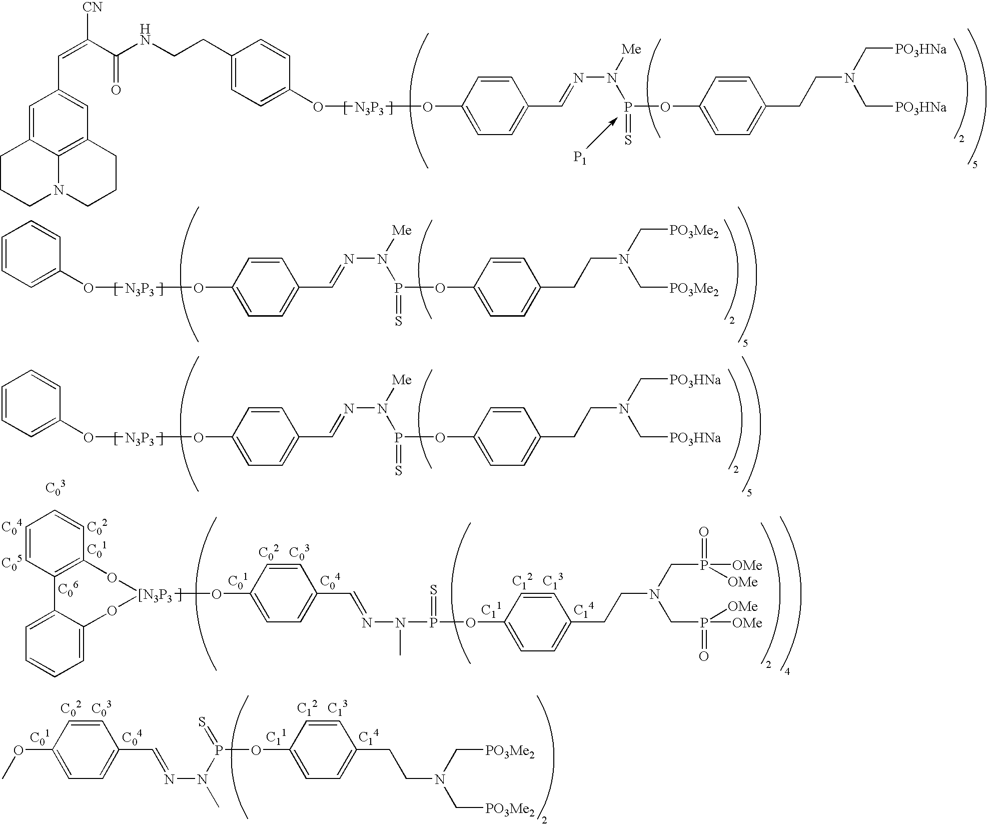

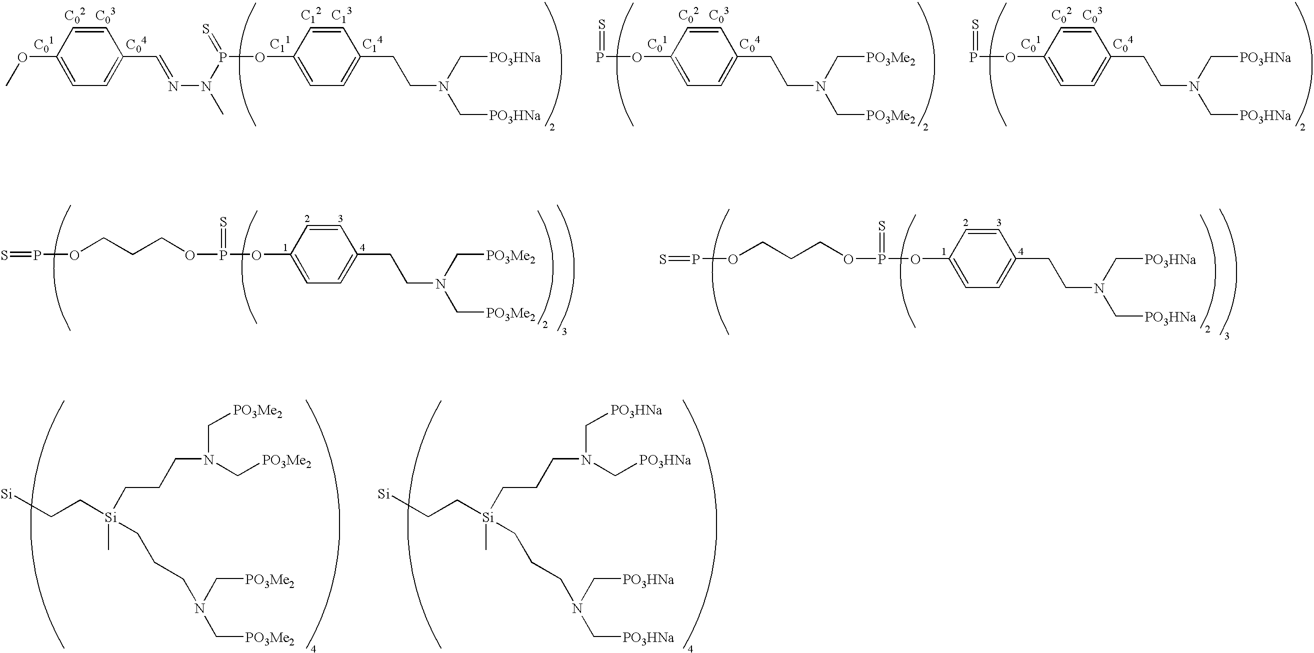

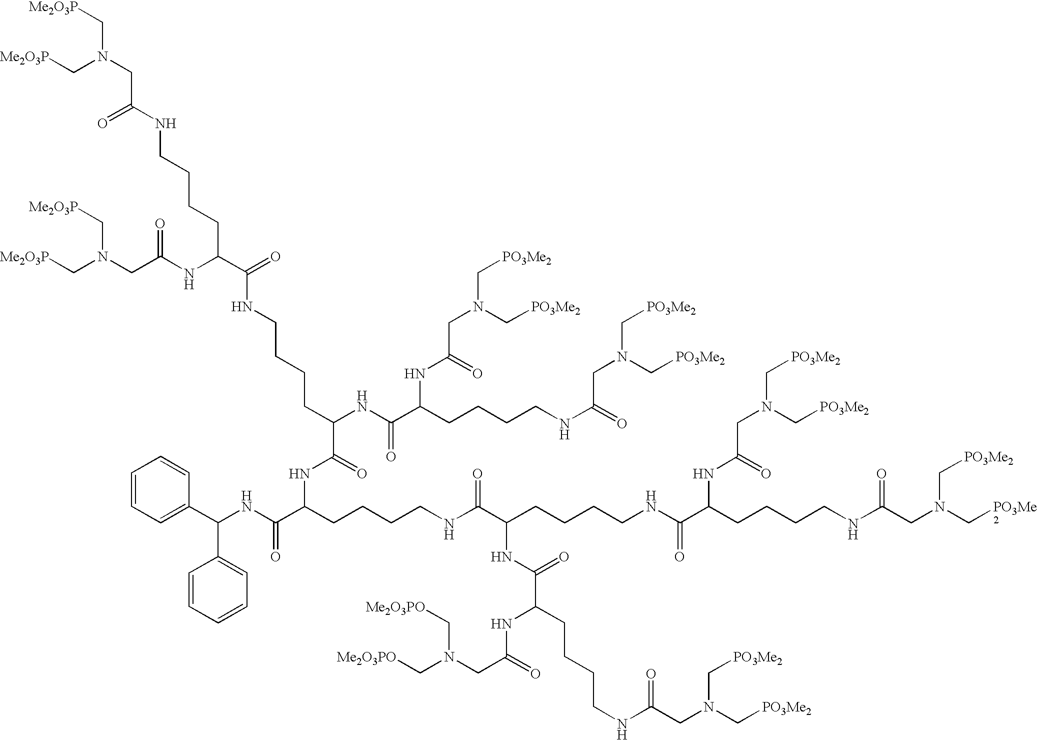

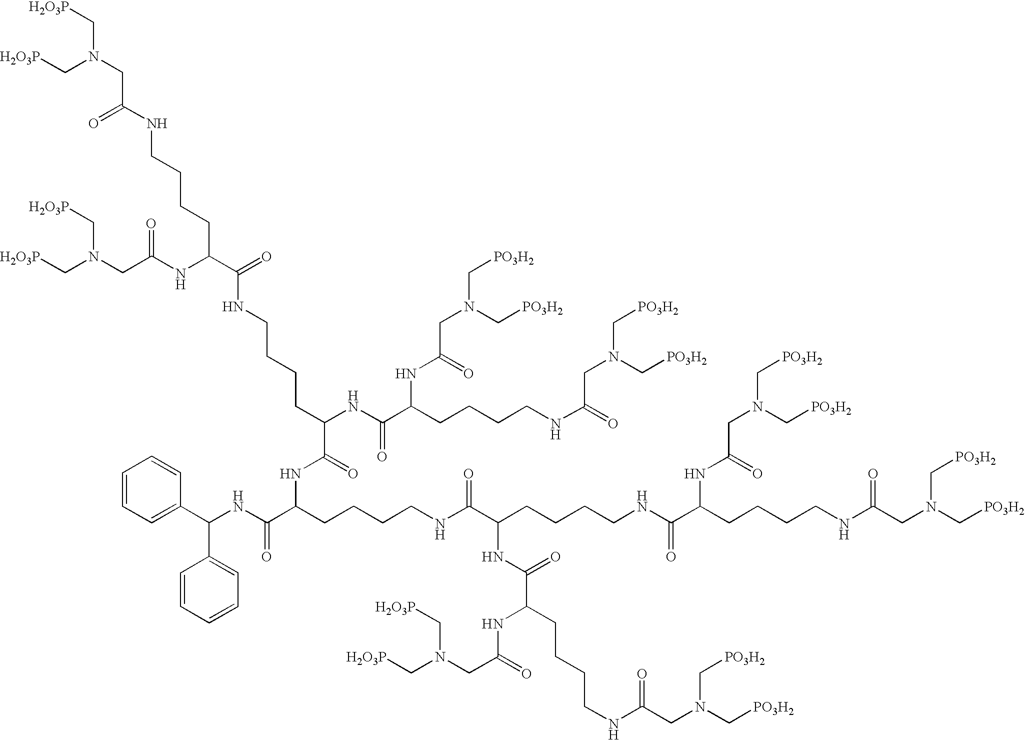

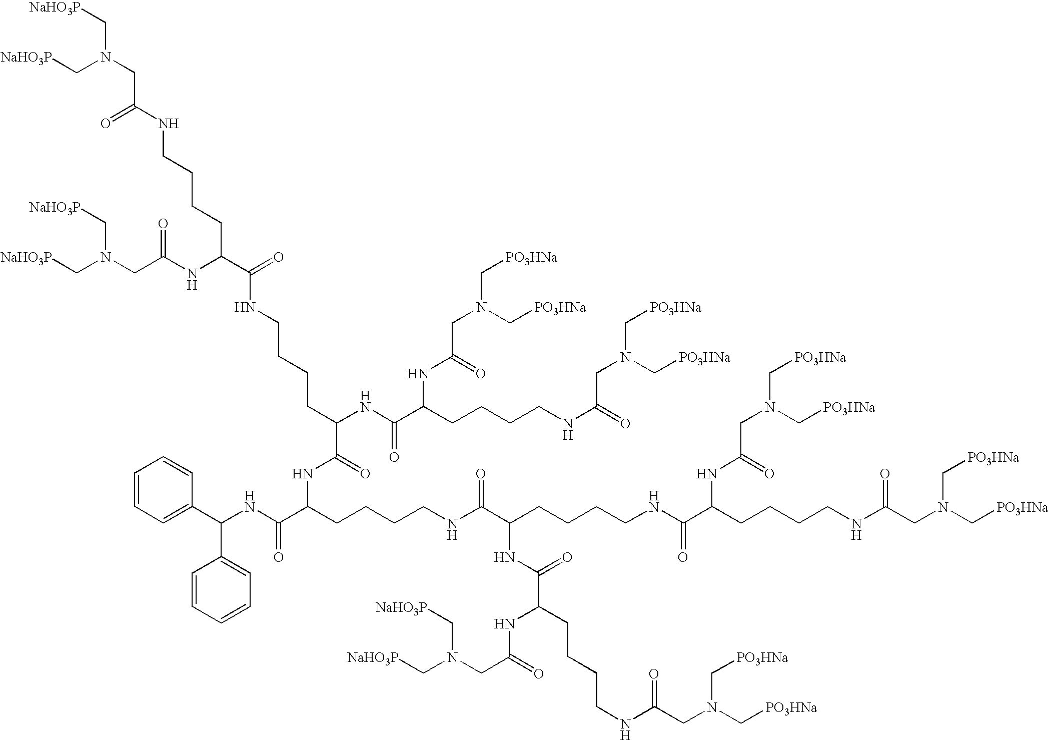

45. The method according to claim 25 wherein the inflammatory diseases are selected from the group comprising chronic inflammatory diseases, chronic inflammatory diseases of auto-immune origin, pro-inflammatory and inflammatory conditions in case of cancers.

46. The method according to claim 25 wherein the chronic inflammatory disease is selected from the group comprising rheumatoid arthritis, psoriatic arthritis and juvenile idiotypical arthritis.

47. The method according to claim 25 comprising the administration to a patient in need thereof of an effective quantity of a pharmaceutical composition containing as active substance at least one dendrimer with monophosphonic or bisphosphonic terminations associated with a pharmaceutically acceptable carrier.
